SEQUENCE LISTING (30 sequences)

SEQ ID NO: 1 - RNA, 4443 nt, synthetic construct
  misc_feature 1..4443: note = Synthetic polynucleotide
  source 1..4443: mol_type = other RNA
atgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaactcgatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctgtccctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtgtccctggcacctcaagcgaatcttaccgagctcgacatctactcccggagactgtcgcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaa

SEQ ID NO: 2 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 3 - RNA, 140 nt, synthetic construct
  misc_feature 1..140: note = Synthetic polynucleotide
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 4 - RNA, 105 nt, synthetic construct
  misc_feature 1..105: note = Synthetic polynucleotide
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 5 - RNA, 105 nt, synthetic construct
  misc_feature 1..105: note = Synthetic polynucleotide
  source 1..105: mol_type = other RNA
gggtggcatccctgtgacccctccccagtgcctctcctggccctggaagttgccactccagtgcccaccagccttgtcctaataaaattaagttgcatcaaagct

SEQ ID NO: 6 - RNA, 4688 nt, synthetic construct
  misc_feature 1..4688: note = Synthetic polynucleotide
  source 1..4688: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaactcgatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctgtccctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtgtccctggcacctcaagcgaatcttaccgagctcgacatctactcccggagactgtcgcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaacgggtggcatccctgtgacccctccccagtgcctctcctggccctggaagttgccactccagtgcccaccagccttgtcctaataaaattaagttgcatcaagct

SEQ ID NO: 7 - RNA, 4688 nt, synthetic construct
  misc_feature 1..4688: note = Synthetic polynucleotide
  source 1..4688: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaactcgatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctgtccctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtgtccctggcacctcaagcgaatcttaccgagctcgacatctactcccggagactgtcgcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaagggtggcatccctgtgacccctccccagtgcctctcctggccctggaagttgccactccagtgcccaccagccttgtcctaataaaattaagttgcatcaaagct

SEQ ID NO: 8 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 9 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 10 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 11 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgccaactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 12 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 13 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgccaactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 14 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtccgtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaagccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 15 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 16 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtccgtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagagtctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 17 - DNA, 4443 bp, synthetic construct
  misc_feature 1..4443: note = Synthetic polynucleotide
  source 1..4443: mol_type = other DNA
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 18 - DNA, 4443 bp, synthetic construct
  misc_feature 1..4443: note = Synthetic polynucleotide
  source 1..4443: mol_type = other DNA
atgcagagaagccccctggagaaggcctctgtggtgagcaagctgttcttcagctggaccagacccatcctgagaaagggctacagacagagactggagctgtctgacatctaccagatcccctctgtggactctgccgacaacctgtctgagaagctggagagagagtgggacagagagctggccagcaagaagaaccccaagctgatcaatgccctgagaagatgcttcttctggagattcatgttctatggcatcttcctgtacctgggagaggtgaccaaggccgtgcagcccctgctgctgggcaggatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggccggcctgggcagaatgatgatgaagtacagagaccagagagccggcaagatctctgagagactggtgatcacctctgagatgattgagaacatccagtctgtgaaggcctactgctgggaggaggccatggagaagatgattgagaacctgagacagacagagctgaagctgaccaggaaggccgcctatgtgagatacttcaacagctctgccttcttcttctctggcttctttgtggtgttcctgtctgtgctgccctatgccctgatcaagggcatcatcctgaggaagatcttcaccaccatcagcttctgcattgtgctgaggatggccgtgaccaggcagttcccctgggccgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggagagctgtttgagaaggccaagcagaacaacaacaacagaaagaccagcaatggagatgacagcctgttcttcagcaacttcagcctgctgggcacccctgtgctgaaggacatcaacttcaagattgagaggggccagctgctggccgtggccggcagcacaggagccggcaagaccagcctgctgatggtgatcatgggagagctggagccctctgagggcaagatcaagcactctggcagaatcagcttctgcagccagttcagctggatcatgcctggcaccatcaaggagaacatcatctttggggtgagctatgatgagtacaggtacagatctgtgatcaaggcctgccagctggaggaggacatctccaagtttgccgagaaggacaacattgtgctgggggagggaggcatcaccctgtctgggggccagagagccagaatcagcctggccagagccgtgtacaaggatgccgacctgtacctgctggacagcccctttggctacctggatgtgctgacagagaaggagatctttgagagctgtgtgtgcaagctgatggccaacaagaccaggatcctggtgaccagcaagatggagcacctgaagaaggccgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacaggttcagcctggagggggatgcccctgtgagctggacagagaccaagaagcagagcttcaagcagacaggagagtttggggagaagaggaagaacagcatcctgaaccccatcaacagcatcaggaagttcagcattgtgcagaagacccccctgcagatgaatggcattgaggaggactctgatgagcccctggagagaagactgagcctggtgccagactctgagcagggagaggccatcctgcccaggatctctgtgatcagcacaggccccaccctgcaggccagaagaagacagtctgtgctgaacctgatgacccactctgtgaaccagggccagaatatccacagaaagaccacagccagcaccagaaaggtgagcctggccccccaggccaacctgacagagctggacatctacagcagaaggctgagccaggagacaggcctggagatctctgaggagatcaatgaggaggacctgaaggagtgcttctttgatgacatggagagcatccctgccgtgaccacctggaacacctacctgagatacatcacagtgcacaagagcctgatctttgtgctgatctggtgcctggtgatcttcctggccgaggtggccgccagcctggtggtgctgtggctgctgggcaacacccccctgcaggacaagggcaacagcacccacagcagaaacaacagctatgctgtgatcatcaccagcaccagcagctactatgtgttctacatctatgtgggagtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctggaggcatcctgaacagattcagcaaggacattgccatcctggatgacctgctgcccctgaccatctttgacttcatccagctgctgctgattgtgattggagccattgccgtggtggccgtgctgcagccctacatctttgtggccacagtgcctgtgattgtggccttcatcatgctgagggcctacttcctgcagaccagccagcagctgaagcagctggagtctgagggcagaagccccatcttcacccacctggtgaccagcctgaagggcctgtggaccctgagggcctttggcagacagccctactttgagaccctgttccacaaggccctgaacctgcacacagccaactggttcctgtacctgagcaccctgagatggttccagatgaggattgagatgatctttgtgatcttcttcattgccgtgaccttcatcagcatcctgaccacaggggagggcgagggcagagtgggcatcatcctgaccctggccatgaacatcatgagcaccctgcagtgggccgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctggaggccagatgacagtgaaggacctgacagccaagtacacagaggggggcaatgccatcctggagaacatcagcttcagcatcagccctggccagagggtgggcctgctgggcagaacaggctctggcaagagcaccctgctgtctgccttcctgaggctgctgaacacagagggagagatccagattgatggggtgagctgggacagcatcaccctgcagcagtggaggaaggcctttggggtgatcccccagaaggtgttcatcttctctggcaccttcaggaagaacctggacccctatgagcagtggtctgaccaggagatctggaaggtggccgatgaggtgggcctgagatctgtgattgagcagttccctggcaagctggactttgtgctggtggatggaggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgccgactgcacagtgatcctgtgtgagcacagaattgaggccatgctggagtgccagcagttcctggtgattgaggagaacaaggtgaggcagtatgacagcatccagaagctgctgaatgagagaagcctgttcagacaggccatcagcccctctgacagagtgaagctgttcccccacaggaacagcagcaagtgcaagagcaagccccagattgccgccctgaaggaggagacagaggaggaggtgcaggacaccagactgtga

SEQ ID NO: 19 - DNA, 4443 bp, synthetic construct
  misc_feature 1..4443: note = Synthetic polynucleotide
  source 1..4443: mol_type = other DNA
atgcagaggagccccctggagaaggccagcgtggtgagcaagctgttcttcagctggaccaggcccatcctgaggaagggctacaggcagaggctggagctgagcgacatctaccagatccccagcgtggacagcgccgacaacctgagcgagaagctggagagggagtgggacagggagctggccagcaagaagaaccccaagctgatcaacgccctgaggaggtgcttcttctggaggttcatgttctacggcatcttcctgtacctgggcgaggtgaccaaggccgtgcagcccctgctgctgggcaggatcatcgccagctacgaccccgacaacaaggaggagaggagcatcgccatctacctgggcatcggcctgtgcctgctgttcatcgtgaggaccctgctgctgcaccccgccatcttcggcctgcaccacatcggcatgcagatgaggatcgccatgttcagcctgatctacaagaagaccctgaagctgagcagcagggtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcaggatgatgatgaagtacagggaccagagggccggcaagatcagcgagaggctggtgatcaccagcgagatgatcgagaacatccagagcgtgaaggcctactgctgggaggaggccatggagaagatgatcgagaacctgaggcagaccgagctgaagctgaccaggaaggccgcctacgtgaggtacttcaacagcagcgccttcttcttcagcggcttcttcgtggtgttcctgagcgtgctgccctacgccctgatcaagggcatcatcctgaggaagatcttcaccaccatcagcttctgcatcgtgctgaggatggccgtgaccaggcagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacaggaagaccagcaacggcgacgacagcctgttcttcagcaacttcagcctgctgggcacccccgtgctgaaggacatcaacttcaagatcgagaggggccagctgctggccgtggccggcagcaccggcgccggcaagaccagcctgctgatggtgatcatgggcgagctggagcccagcgagggcaagatcaagcacagcggcaggatcagcttctgcagccagttcagctggatcatgcccggcaccatcaaggagaacatcatcttcggcgtgagctacgacgagtacaggtacaggagcgtgatcaaggcctgccagctggaggaggacatcagcaagttcgccgagaaggacaacatcgtgctgggcgagggcggcatcaccctgagcggcggccagagggccaggatcagcctggccagggccgtgtacaaggacgccgacctgtacctgctggacagccccttcggctacctggacgtgctgaccgagaaggagatcttcgagagctgcgtgtgcaagctgatggccaacaagaccaggatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagaggaggaacagcatcctgaccgagaccctgcacaggttcagcctggagggcgacgcccccgtgagctggaccgagaccaagaagcagagcttcaagcagaccggcgagttcggcgagaagaggaagaacagcatcctgaaccccatcaacagcatcaggaagttcagcatcgtgcagaagacccccctgcagatgaacggcatcgaggaggacagcgacgagcccctggagaggaggctgagcctggtgcccgacagcgagcagggcgaggccatcctgcccaggatcagcgtgatcagcaccggccccaccctgcaggccaggaggaggcagagcgtgctgaacctgatgacccacagcgtgaaccagggccagaacatccacaggaagaccaccgccagcaccaggaaggtgagcctggccccccaggccaacctgaccgagctggacatctacagcaggaggctgagccaggagaccggcctggagatcagcgaggagatcaacgaggaggacctgaaggagtgcttcttcgacgacatggagagcatccccgccgtgaccacctggaacacctacctgaggtacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcaggaacaacagctacgccgtgatcatcaccagcaccagcagctactacgtgttctacatctacgtgggcgtggccgacaccctgctggccatgggcttcttcaggggcctgcccctggtgcacaccctgatcaccgtgagcaagatcctgcaccacaagatgctgcacagcgtgctgcaggcccccatgagcaccctgaacaccctgaaggccggcggcatcctgaacaggttcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagggcctacttcctgcagaccagccagcagctgaagcagctggagagcgagggcaggagccccatcttcacccacctggtgaccagcctgaagggcctgtggaccctgagggccttcggcaggcagccctacttcgagaccctgttccacaaggccctgaacctgcacaccgccaactggttcctgtacctgagcaccctgaggtggttccagatgaggatcgagatgatcttcgtgatcttcttcatcgccgtgaccttcatcagcatcctgaccaccggcgagggcgagggcagggtgggcatcatcctgaccctggccatgaacatcatgagcaccctgcagtgggccgtgaacagcagcatcgacgtggacagcctgatgaggagcgtgagcagggtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagggtgggcctgctgggcaggaccggcagcggcaagagcaccctgctgagcgccttcctgaggctgctgaacaccgagggcgagatccagatcgacggcgtgagctgggacagcatcaccctgcagcagtggaggaaggccttcggcgtgatcccccagaaggtgttcatcttcagcggcaccttcaggaagaacctggacccctacgagcagtggagcgaccaggagatctggaaggtggccgacgaggtgggcctgaggagcgtgatcgagcagttccccggcaagctggacttcgtgctggtggacggcggctgcgtgctgagccacggccacaagcagctgatgtgcctggccaggagcgtgctgagcaaggccaagatcctgctgctggacgagcccagcgcccacctggaccccgtgacctaccagatcatcaggaggaccctgaagcaggccttcgccgactgcaccgtgatcctgtgcgagcacaggatcgaggccatgctggagtgccagcagttcctggtgatcgaggagaacaaggtgaggcagtacgacagcatccagaagctgctgaacgagaggagcctgttcaggcaggccatcagccccagcgacagggtgaagctgttcccccacaggaacagcagcaagtgcaagagcaagccccagatcgccgccctgaaggaggagaccgaggaggaggtgcaggacaccaggctgtga

SEQ ID NO: 20 - DNA, 4443 bp, synthetic construct
  misc_feature 1..4443: note = Synthetic polynucleotide
  source 1..4443: mol_type = other DNA
atgcagagatcccctctggagaaggcctcagtggtgtccaagcttttcttctcctggaccaggcccattttaagaaagggctacaggcagagacttgagctgtctgacatctatcagatcccttctgtggattctgctgacaatcttagtgaaaaattggaaagggagtgggacagagagctggcaagtaaaaagaaccccaagctgattaatgccctgaggcgctgctttttttggagattcatgttctatggcatattcctctaccttggagaagtaaccaaagctgtacagcctctcctccttggcagaatcattgcctcctatgatcctgataacaaggaggagagaagcatagccatctacctgggcattgggctgtgcctcttgtttattgtgaggacccttctcttgcaccctgccatctttggccttcatcacattggcatgcaaatgagaatagcaatgtttagtcttatttacaaaaaaacattaaaactctcttccagggtgttggacaagatcagtattggacaactggtcagcctgctgagcaacaacctgaacaagtttgatgaaggactggccctggcccactttgtctggattgccccccttcaggtggctcttttgatgggcctgatctgggaactcctgcaggcctctgccttctgtgggttaggcttcctgatagtgctagctctctttcaggcagggttgggtagaatgatgatgaagtacagagaccagagggctgggaagatatctgagaggctggtcattacttctgaaatgatagaaaacatccagtctgttaaagcttactgctgggaggaggctatggaaaagatgattgagaacttgaggcaaacagagctcaagctgactaggaaggcagcctatgtcaggtatttcaacagcagtgctttcttcttctcaggctttttcgtggtcttcttgagtgttctgccctatgccctcatcaaggggataattttgagaaagattttcaccactatttccttttgcattgtcctgaggatggctgtcaccaggcaattcccctgggctgtgcagacatggtatgactctctgggggccatcaacaaaatccaagatttcctgcagaagcaggagtacaagaccctggaatacaacctcaccaccacagaagttgtgatggagaatgtgactgcattctgggaggaaggatttggggagctgtttgagaaagcaaaacaaaacaataataacaggaaaaccagcaatggagatgactccctgttcttttccaacttctctttgttgggcacccctgtcctgaaagatataaactttaaaattgaaagagggcagctgttggcagttgctggctccacaggagctggaaaaacttcactactgatggtgatcatgggggagttagaaccctctgaagggaaaataaaacattctgggaggattagtttctgcagccagttcagctggatcatgcctgggaccattaaagaaaatattatatttggagtgagctatgatgaatatagatataggagtgtcatcaaagcctgtcagttggaggaagacatcagcaaatttgcagagaaagacaacattgttctgggtgaaggtggcatcaccctgtcaggagggcaaagggccaggatcagcttggccagagcagtctataaagatgctgatctgtacctcctggatagcccttttggctatctggatgttttgacagagaaggaaatttttgagtcctgtgtctgcaagttaatggcaaataaaacaaggatacttgtgacctcaaaaatggaacacctgaagaaggctgacaaaattctgatcctgcatgagggcagcagctacttttatggaacattttctgaactgcagaatttgcaaccagacttttcatcaaagctcatgggatgtgacagttttgatcagttttctgcagaaaggagaaactccattttgactgagaccctgcacaggttcagtctggagggggatgccccagtgagttggactgagacaaagaaacagagcttcaagcagactggagagtttggagaaaagaggaaaaactcaattctcaatcccatcaatagcatcaggaagttcagcatagttcagaagactcctttgcagatgaatgggattgaagaggactcagatgagcccctggaaaggagactctccttggtgccagattcagagcagggggaagccatactgccaaggatctctgtgatttctacagggcccaccctccaagcaagaaggagacagtcagttttaaacctgatgacccactctgtcaaccagggacagaacattcatagaaagacaacagcatctacaagaaaagtttcactggcccctcaagccaatttaactgaactagatatctacagcaggaggctcagccaagaaacaggcctggagatctcagaagaaataaatgaggaggatttgaaggaatgcttctttgatgatatggagagcatcccagctgtcacaacctggaacacctacctgagatacatcacagtgcacaaatccctcatctttgtacttatatggtgccttgtcatcttcttagctgaggtggctgcttccctggtggtgctgtggctgctgggaaacacacccctccaggataaagggaactctactcacagcaggaacaacagttatgctgtgatcatcaccagtacctcctcctactatgtgttctacatttatgttggagttgcagacacattgcttgccatgggtttttttagaggactccccctggtgcatactctcatcactgtttccaaaatccttcaccacaagatgctgcacagtgtactacaggctcccatgagcaccctcaacactcttaaagcaggaggaatcttgaacagatttagcaaggacattgcaattcttgatgacctgcttccactgaccatctttgacttcatccagcttctgctcattgtaattggtgccattgctgtggtagcagtgctccagccatatatttttgtggccactgtgcctgttattgtggccttcattatgttgagagcctacttcctgcagacctctcagcagctcaagcaacttgaaagtgagggcaggagccccatatttacacacttggtcacttccctcaaaggcctctggacactcagagcttttggaagacaaccttattttgaaactctcttccacaaggctctgaatctccacacagccaactggtttctgtatctttcaacactgcgctggttccagatgaggattgagatgatctttgttatcttcttcatagctgttaccttcatctctattctgacaactggtgagggggaagggagagtaggcatcatcctcacactagccatgaacataatgtctaccttacaatgggccgtgaacagctccatagatgtggacagcctcatgagaagtgtgtcaagagttttcaaattcattgacatgcccacagaaggcaaaccaaccaagagcacaaaaccctacaagaatggccagctgagtaaggtcatgatcattgaaaattctcatgtgaagaaggatgatatttggcccagtgggggccagatgacagtcaaggacctcactgccaaatacacagagggtggaaatgctatcctagagaacatctccttctccatctccccaggccaaagagttggcttgctgggcaggactggcagtggcaagtccaccttgctctcagcatttctcaggcttttaaatacagagggagagattcaaattgatggggtgtcttgggatagtataacacttcaacagtggaggaaagcctttggtgtgattcctcagaaagtgtttatcttctctggcactttcagaaaaaatctggacccctatgaacagtggagtgaccaggaaatctggaaggtggcagatgaagtgggcctaagatcagtcatagagcagtttcctggaaagttggattttgtgcttgtagatggaggctgtgtgctgtcccatggccataaacagctaatgtgcctggctaggtcagtgctgagcaaggccaagatcctgctgttagatgagccttcagcccatctggaccctgtgacataccagattatcagaagaactctgaagcaggcctttgctgactgcactgtcatcctgtgtgagcacagaattgaggccatgctggagtgccagcagttccttgttatagaagagaataaggttaggcagtatgacagcattcagaaactgctaaatgaaagatctctcttcaggcaagctatttcaccatctgatagagtgaaactttttccccacagaaattcctctaaatgtaaatctaagccccagatagctgccttgaaagaggagactgaagaagaagtccaggacaccagactgtga

SEQ ID NO: 21 - DNA, 4443 bp, synthetic construct
  misc_feature 1..4443: note = Synthetic polynucleotide
  source 1..4443: mol_type = other DNA
atgcagagatccccgctggagaaggcatctgtggtgtcaaaactgttctttagctggacaaggcccatccttaggaaagggtacagacagaggttggagctgtcagacatatatcagatcccttcagtggactctgcagacaacctctctgaaaagctggagagggaatgggacagggaactggccagcaaaaaaaaccctaaactgattaatgccctgaggaggtgcttcttttggagattcatgttctatgggatcttcctttacctgggggaggtgactaaagctgttcagcctcttcttctggggaggattattgcctcctatgacccagacaacaaagaagaaagaagcatagccatttacttaggcataggcctctgcttgctcttcatagttagaaccctcctactccacccagccatctttggtctccaccacataggtatgcagatgagaatagcaatgttctccttgatctacaagaagaccctcaagctgtccagcagggtgctggacaagatctccataggccagttagtcagtctactgtccaataacttaaataagtttgatgagggactggcactggcacattttgtgtggattgcccccctccaagtggcccttcttatgggccttatctgggagctgttgcaggcctctgctttctgtggcctgggtttcctcatagtcctagccttattccaggctggactgggcagaatgatgatgaagtatagggaccaaagagcagggaagatttctgaaaggctggttataacttctgagatgattgagaacattcagtcagtgaaagcttactgctgggaagaagctatggaaaaaatgattgaaaatctcagacagactgaattaaagttgaccaggaaagctgcttatgtcagatacttcaactcctcagccttctttttttctggcttctttgttgtattcctttcagtcctcccctatgccctgattaagggcattatcttgaggaaaattttcacaaccatctccttttgtattgtcctcaggatggctgttacaaggcaatttccttgggctgtgcaaacttggtatgatagccttggagcaatcaacaagatccaggatttcctgcaaaagcaggagtacaagacattggaatacaaccttaccaccactgaggtggtgatggaaaatgtgactgccttctgggaggaggggtttggagagctgtttgagaaagccaaacagaacaacaacaatagaaagacctctaatggtgatgattccctgttcttttctaactttagtcttctggggaccccagttctgaaagatattaactttaaaattgaaaggggacagttgctggctgtggctgggtccactggggctgggaagacaagcctgctcatggtgatcatgggagagctggaacccagtgaaggaaagatcaaacactcaggcaggatctccttctgcagccagttctcatggattatgccaggcactattaaagaaaatatcatctttggtgtaagctatgatgagtacaggtatagatctgtaattaaagcctgccagctggaggaagacatctctaagtttgctgagaaggataacattgtgttgggggaagggggcatcaccctttctggtgggcagagggctaggatctcccttgctagggcagtatacaaggatgctgacttgtacctcttggatagtccttttggctacctagatgtgctgacagagaaagaaatatttgaaagctgtgtgtgtaagctcatggctaacaagaccaggatcctggtcaccagtaaaatggaacacctcaaaaaagcagacaagatccttattctccatgagggctcctcctacttctatgggaccttcagtgagctgcagaatctgcagccagacttctcctcaaaacttatgggctgtgactcctttgaccaattctctgcagaaagaaggaatagcatactgacagaaacactgcatagattctccctggaaggagatgccccagtgagttggacagaaaccaaaaagcagagcttcaagcagactggtgagtttggtgaaaagaggaagaattctatcctgaaccccatcaatagcatcaggaaatttagcatagtccaaaagacccccctccagatgaatggaatagaggaggatagtgatgagcctcttgagagaaggctgtccctggttccagacagtgaacagggtgaagccattcttccgaggatcagtgtcatctccactgggcccacattgcaggccagaagaagacagtctgttctgaatttgatgacacattctgtgaatcaaggccagaatatccatagaaaaaccactgccagcaccagaaaagtttctctagccccccaggctaacctgactgagttagacatctacagcagaaggctgagccaagagactggcttggaaatatctgaggagatcaatgaggaggacctcaaggagtgcttctttgatgacatggagtcaatccctgcagtcactacatggaacacttacctaaggtacatcacagttcataagagcctcatctttgtcctcatatggtgtctggtcatctttttagcagaagtggctgccagcctagttgtgctgtggttactgggcaatacacctcttcaggacaaaggcaatagcacacacagcagaaacaactcctatgcagtgatcatcacctctacaagctcttactatgtattctatatatatgtgggagtggcagatactctcctggccatgggattcttcaggggattacctctagttcacacattgatcacagtgtcaaaaattctccaccacaagatgttacacagtgtcctgcaagccccaatgtctactctgaacacacttaaggcaggtggaattttgaataggtttagcaaggacatagctatcctggatgatctcctccctctgaccatctttgacttcatccagttactgctcattgtaattggagccattgcagtggtagcagtcctacagccttacatttttgtggctactgttcctgttattgtggccttcattatgctaagagcttacttcctgcaaacaagccaacagttgaaacagctagaaagtgagggaaggtcccccatcttcacccacctggtgacatcactcaaggggctatggactcttagggcttttgggagacagccgtactttgagaccttattccataaggcccttaacctccatacagcaaactggttcttatacctgagtactctgaggtggtttcaaatgaggattgaaatgatttttgtgatcttcttcattgctgtgaccttcatctcaatcttgaccacaggagagggggagggcagggtgggcatcatactgaccttggccatgaacattatgtcaaccctgcagtgggctgtcaatagctccattgatgtggacagtctgatgaggagtgtctccagggtcttcaagtttattgacatgccaactgagggcaaacccaccaaaagcactaagccatataaaaatggccaactgtccaaagtgatgatcattgaaaattcacatgtaaagaaggatgatatctggccctctggaggacagatgacagtgaaagacctgactgccaagtacacagagggtggtaatgccattcttgagaacattagtttcagtatttccccggggcaaagggtgggcctccttggcagaacaggctctggcaagagtaccctgctgtcagcctttttaagactgttgaacactgagggagaaattcagattgatggtgtctcctgggatagcatcaccctccagcagtggagaaaagcttttggagtgatcccgcaaaaggttttcatcttttcaggcaccttccggaagaacctggacccctatgagcagtggtctgaccaggaaatatggaaggtagctgatgaagttgggcttaggtcagtcatagagcagttcccaggcaaactggactttgtcctggtggatggtggatgtgtactgagtcatgggcacaaacagctgatgtgcctagccaggtctgtgctcagcaaggcaaagatattgctgcttgatgaacccagtgcccatctggacccagtcacatatcagatcatcagaagaacattgaagcaggcctttgctgattgcacagttatcctctgtgagcacaggattgaggccatgctggagtgccagcagtttctggtgattgaggagaataaagtaaggcagtatgactccatccagaagctgctcaatgaaagaagcctctttagacaagctatctccccctcagacagggtcaaattgttccctcacagaaacagcagcaagtgcaagagcaagccccaaattgcagccttgaaagaggagacagaggaagaggtgcaggacaccagactctga

SEQ ID NO: 22 - DNA, 4443 bp, synthetic construct
  misc_feature 1..4443: note = Synthetic polynucleotide
  source 1..4443: mol_type = other DNA
atgcagagaagccccctggagaaggccagcgtggtgagcaagctgttcttcagctggaccagacccatcctgagaaagggctacagacagagactggagctgagcgacatctaccagatccccagcgtggacagcgccgacaacctgagcgagaagctggagagagagtgggacagagagctggccagcaagaagaaccccaagctgatcaacgccctgagaagatgcttcttctggagattcatgttctacggcatcttcctgtacctgggcgaggtgaccaaggccgtgcagcccctgctgctgggcagaatcatcgccagctacgaccccgacaacaaggaggagagaagcatcgccatctacctgggcatcggcctgtgcctgctgttcatcgtgagaaccctgctgctgcaccccgccatcttcggcctgcaccacatcggcatgcagatgagaatcgccatgttcagcctgatctacaagaagaccctgaagctgagcagcagagtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcagaatgatgatgaagtacagagaccagagagccggcaagatcagcgagagactggtgatcaccagcgagatgatcgagaacatccagagcgtgaaggcctactgctgggaggaggccatggagaagatgatcgagaacctgagacagaccgagctgaagctgaccagaaaggccgcctacgtgagatacttcaacagcagcgccttcttcttcagcggcttcttcgtggtgttcctgagcgtgctgccctacgccctgatcaagggcatcatcctgagaaagatcttcaccaccatcagcttctgcatcgtgctgagaatggccgtgaccagacagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacagaaagaccagcaacggcgacgacagcctgttcttcagcaacttcagcctgctgggcacccccgtgctgaaggacatcaacttcaagatcgagagaggccagctgctggccgtggccggcagcaccggcgccggcaagaccagcctgctgatggtgatcatgggcgagctggagcccagcgagggcaagatcaagcacagcggcagaatcagcttctgcagccagttcagctggatcatgcccggcaccatcaaggagaacatcatcttcggcgtgagctacgacgagtacagatacagaagcgtgatcaaggcctgccagctggaggaggacatcagcaagttcgccgagaaggacaacatcgtgctgggcgagggcggcatcaccctgagcggcggccagagagccagaatcagcctggccagagccgtgtacaaggacgccgacctgtacctgctggacagccccttcggctacctggacgtgctgaccgagaaggagatcttcgagagctgcgtgtgcaagctgatggccaacaagaccagaatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagagaagaaacagcatcctgaccgagaccctgcacagattcagcctggagggcgacgcccccgtgagctggaccgagaccaagaagcagagcttcaagcagaccggcgagttcggcgagaagagaaagaacagcatcctgaaccccatcaacagcatcagaaagttcagcatcgtgcagaagacccccctgcagatgaacggcatcgaggaggacagcgacgagcccctggagagaagactgagcctggtgcccgacagcgagcagggcgaggccatcctgcccagaatcagcgtgatcagcaccggccccaccctgcaggccagaagaagacagagcgtgctgaacctgatgacccacagcgtgaaccagggccagaacatccacagaaagaccaccgccagcaccagaaaggtgagcctggccccccaggccaacctgaccgagctggacatctacagcagaagactgagccaggagaccggcctggagatcagcgaggagatcaacgaggaggacctgaaggagtgcttcttcgacgacatggagagcatccccgccgtgaccacctggaacacctacctgagatacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcagaaacaacagctacgccgtgatcatcaccagcaccagcagctactacgtgttctacatctacgtgggcgtggccgacaccctgctggccatgggcttcttcagaggcctgcccctggtgcacaccctgatcaccgtgagcaagatcctgcaccacaagatgctgcacagcgtgctgcaggcccccatgagcaccctgaacaccctgaaggccggcggcatcctgaacagattcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagagcctacttcctgcagaccagccagcagctgaagcagctggagagcgagggcagaagccccatcttcacccacctggtgaccagcctgaagggcctgtggaccctgagagccttcggcagacagccctacttcgagaccctgttccacaaggccctgaacctgcacaccgccaactggttcctgtacctgagcaccctgagatggttccagatgagaatcgagatgatcttcgtgatcttcttcatcgccgtgaccttcatcagcatcctgaccaccggcgagggcgagggcagagtgggcatcatcctgaccctggccatgaacatcatgagcaccctgcagtgggccgtgaacagcagcatcgacgtggacagcctgatgagaagcgtgagcagagtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagagtgggcctgctgggcagaaccggcagcggcaagagcaccctgctgagcgccttcctgagactgctgaacaccgagggcgagatccagatcgacggcgtgagctgggacagcatcaccctgcagcagtggagaaaggccttcggcgtgatcccccagaaggtgttcatcttcagcggcaccttcagaaagaacctggacccctacgagcagtggagcgaccaggagatctggaaggtggccgacgaggtgggcctgagaagcgtgatcgagcagttccccggcaagctggacttcgtgctggtggacggcggctgcgtgctgagccacggccacaagcagctgatgtgcctggccagaagcgtgctgagcaaggccaagatcctgctgctggacgagcccagcgcccacctggaccccgtgacctaccagatcatcagaagaaccctgaagcaggccttcgccgactgcaccgtgatcctgtgcgagcacagaatcgaggccatgctggagtgccagcagttcctggtgatcgaggagaacaaggtgagacagtacgacagcatccagaagctgctgaacgagagaagcctgttcagacaggccatcagccccagcgacagagtgaagctgttcccccacagaaacagcagcaagtgcaagagcaagccccagatcgccgccctgaaggaggagaccgaggaggaggtgcaggacaccagactgtga

SEQ ID NO: 23 - DNA, 4443 bp, synthetic construct
  misc_feature 1..4443: note = Synthetic polynucleotide
  source 1..4443: mol_type = other DNA
atgcagcgcagccccctggagaaggccagcgtggtgagcaagctgttcttcagctggacccgccccatcctgcgcaagggctaccgccagcgcctggagctgagcgacatctaccagatccccagcgtggacagcgccgacaacctgagcgagaagctggagcgcgagtgggaccgcgagctggccagcaagaagaaccccaagctgatcaacgccctgcgccgctgcttcttctggcgcttcatgttctacggcatcttcctgtacctgggcgaggtgaccaaggccgtgcagcccctgctgctgggccgcatcatcgccagctacgaccccgacaacaaggaggagcgcagcatcgccatctacctgggcatcggcctgtgcctgctgttcatcgtgcgcaccctgctgctgcaccccgccatcttcggcctgcaccacatcggcatgcagatgcgcatcgccatgttcagcctgatctacaagaagaccctgaagctgagcagccgcgtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggccgcatgatgatgaagtaccgcgaccagcgcgccggcaagatcagcgagcgcctggtgatcaccagcgagatgatcgagaacatccagagcgtgaaggcctactgctgggaggaggccatggagaagatgatcgagaacctgcgccagaccgagctgaagctgacccgcaaggccgcctacgtgcgctacttcaacagcagcgccttcttcttcagcggcttcttcgtggtgttcctgagcgtgctgccctacgccctgatcaagggcatcatcctgcgcaagatcttcaccaccatcagcttctgcatcgtgctgcgcatggccgtgacccgccagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaaccgcaagaccagcaacggcgacgacagcctgttcttcagcaacttcagcctgctgggcacccccgtgctgaaggacatcaacttcaagatcgagcgcggccagctgctggccgtggccggcagcaccggcgccggcaagaccagcctgctgatggtgatcatgggcgagctggagcccagcgagggcaagatcaagcacagcggccgcatcagcttctgcagccagttcagctggatcatgcccggcaccatcaaggagaacatcatcttcggcgtgagctacgacgagtaccgctaccgcagcgtgatcaaggcctgccagctggaggaggacatcagcaagttcgccgagaaggacaacatcgtgctgggcgagggcggcatcaccctgagcggcggccagcgcgcccgcatcagcctggcccgcgccgtgtacaaggacgccgacctgtacctgctggacagccccttcggctacctggacgtgctgaccgagaaggagatcttcgagagctgcgtgtgcaagctgatggccaacaagacccgcatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagcgccgcaacagcatcctgaccgagaccctgcaccgcttcagcctggagggcgacgcccccgtgagctggaccgagaccaagaagcagagcttcaagcagaccggcgagttcggcgagaagcgcaagaacagcatcctgaaccccatcaacagcatccgcaagttcagcatcgtgcagaagacccccctgcagatgaacggcatcgaggaggacagcgacgagcccctggagcgccgcctgagcctggtgcccgacagcgagcagggcgaggccatcctgccccgcatcagcgtgatcagcaccggccccaccctgcaggcccgccgccgccagagcgtgctgaacctgatgacccacagcgtgaaccagggccagaacatccaccgcaagaccaccgccagcacccgcaaggtgagcctggccccccaggccaacctgaccgagctggacatctacagccgccgcctgagccaggagaccggcctggagatcagcgaggagatcaacgaggaggacctgaaggagtgcttcttcgacgacatggagagcatccccgccgtgaccacctggaacacctacctgcgctacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagccgcaacaacagctacgccgtgatcatcaccagcaccagcagctactacgtgttctacatctacgtgggcgtggccgacaccctgctggccatgggcttcttccgcggcctgcccctggtgcacaccctgatcaccgtgagcaagatcctgcaccacaagatgctgcacagcgtgctgcaggcccccatgagcaccctgaacaccctgaaggccggcggcatcctgaaccgcttcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgcgcgcctacttcctgcagaccagccagcagctgaagcagctggagagcgagggccgcagccccatcttcacccacctggtgaccagcctgaagggcctgtggaccctgcgcgccttcggccgccagccctacttcgagaccctgttccacaaggccctgaacctgcacaccgccaactggttcctgtacctgagcaccctgcgctggttccagatgcgcatcgagatgatcttcgtgatcttcttcatcgccgtgaccttcatcagcatcctgaccaccggcgagggcgagggccgcgtgggcatcatcctgaccctggccatgaacatcatgagcaccctgcagtgggccgtgaacagcagcatcgacgtggacagcctgatgcgcagcgtgagccgcgtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagcgcgtgggcctgctgggccgcaccggcagcggcaagagcaccctgctgagcgccttcctgcgcctgctgaacaccgagggcgagatccagatcgacggcgtgagctgggacagcatcaccctgcagcagtggcgcaaggccttcggcgtgatcccccagaaggtgttcatcttcagcggcaccttccgcaagaacctggacccctacgagcagtggagcgaccaggagatctggaaggtggccgacgaggtgggcctgcgcagcgtgatcgagcagttccccggcaagctggacttcgtgctggtggacggcggctgcgtgctgagccacggccacaagcagctgatgtgcctggcccgcagcgtgctgagcaaggccaagatcctgctgctggacgagcccagcgcccacctggaccccgtgacctaccagatcatccgccgcaccctgaagcaggccttcgccgactgcaccgtgatcctgtgcgagcaccgcatcgaggccatgctggagtgccagcagttcctggtgatcgaggagaacaaggtgcgccagtacgacagcatccagaagctgctgaacgagcgcagcctgttccgccaggccatcagccccagcgaccgcgtgaagctgttcccccaccgcaacagcagcaagtgcaagagcaagccccagatcgccgccctgaaggaggagaccgaggaggaggtgcaggacacccgcctgtaa

SEQ ID NO: 24 - DNA, 4443 bp, synthetic construct
  misc_feature 1..4443: note = Synthetic polynucleotide
  source 1..4443: mol_type = other DNA
atgcagagaagccccctggagaaggccagcgtggtgagcaagctgttcttcagctggaccagacccatcctgagaaagggctacagacagagactggagctgagcgacatctaccagatccccagcgtggacagcgccgacaacctgagcgagaagctggagagagagtgggacagagagctggccagcaagaagaaccccaagctgatcaacgccctgagaagatgcttcttctggagattcatgttctacggcatcttcctgtacctgggcgaggtgaccaaggccgtgcagcccctgctgctgggcagaatcatcgccagctacgaccccgacaacaaggaggagagaagcatcgccatctacctgggcatcggcctgtgcctgctgttcatcgtgagaaccctgctgctgcaccccgccatcttcggcctgcaccacatcggcatgcagatgagaatcgccatgttcagcctgatctacaagaagaccctgaagctgagcagcagagtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcagaatgatgatgaagtacagggaccagagagccggcaagatcagcgagagactggtgatcaccagcgagatgatcgagaacatccagagcgtgaaggcctactgctgggaggaggccatggagaagatgatcgagaacctgagacagaccgagctgaagctgaccagaaaggccgcctacgtgagatacttcaacagcagcgccttcttcttcagcggcttcttcgtggtgttcctgagcgtgctgccctacgccctgatcaagggcatcatcctgagaaagatcttcaccaccatcagcttctgcatcgtgctgagaatggccgtgaccagacagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacagaaagaccagcaacggcgacgacagcctgttcttcagcaacttcagcctgctgggcacccccgtgctgaaggacatcaacttcaagatcgagagaggccagctgctggccgtggccggcagcaccggcgccggcaagaccagcctgctgatggtgatcatgggcgagctggagcccagcgagggcaagatcaagcacagcggcagaatcagcttctgcagccagttcagctggatcatgcccggcaccatcaaggagaacatcatcttcggcgtgagctacgacgagtacagatacagaagcgtgatcaaggcctgccagctggaggaggacatcagcaagttcgccgagaaggacaacatcgtgctgggcgagggcggcatcaccctgagcggcggccagagagccagaatcagcctggccagagccgtgtacaaggacgccgacctgtacctgctggacagccccttcggctacctggacgtgctgaccgagaaggagatcttcgagagctgcgtgtgcaagctgatggccaacaagaccagaatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagagaagaaacagcatcctgaccgagaccctgcacagattcagcctggagggcgacgcccccgtgagctggaccgagaccaagaagcagagcttcaagcagaccggcgagttcggcgagaagagaaagaacagcatcctgaaccccatcaacagcatcagaaagttcagcatcgtgcagaagacccccctgcagatgaacggcatcgaggaggacagcgacgagcccctggagagaagactgagcctggtgcccgacagcgagcagggcgaggccatcctgcccagaatcagcgtgatcagcaccggccccaccctgcaggccagaagaagacagagcgtgctgaacctgatgacccacagcgtgaaccagggccagaacatccacagaaagaccaccgccagcaccagaaaggtgagcctggccccccaggccaacctgaccgagctggacatctacagcagaagactgagccaggagaccggcctggagatcagcgaggagatcaacgaggaggacctgaaggagtgcttcttcgacgacatggagagcatccccgccgtgaccacctggaacacctacctgagatacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcagaaacaacagctacgccgtgatcatcaccagcaccagcagctactacgtgttctacatctacgtgggcgtggccgacaccctgctggccatgggcttcttcagaggcctgcccctggtgcacaccctgatcaccgtgagcaagatcctgcaccacaagatgctgcacagcgtgctgcaggcccccatgagcaccctgaacaccctgaaggccggcggcatcctgaacagattcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagagcctacttcctgcagaccagccagcagctgaagcagctggagagcgagggcaggagccccatcttcacccacctggtgaccagcctgaagggcctgtggaccctgagagccttcggcagacagccctacttcgagaccctgttccacaaggccctgaacctgcacaccgccaactggttcctgtacctgagcaccctgagatggttccagatgagaatcgagatgatcttcgtgatcttcttcatcgccgtgaccttcatcagcatcctgaccaccggcgagggcgagggcagagtgggcatcatcctgaccctggccatgaacatcatgagcaccctgcagtgggccgtgaacagcagcatcgacgtggacagcctgatgagaagcgtgagcagagtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagagtgggcctgctgggcagaaccggcagcggcaagagcaccctgctgagcgccttcctgagactgctgaacaccgagggcgagatccagatcgacggcgtgagctgggacagcatcaccctgcagcagtggagaaaggccttcggcgtgatcccccagaaggtgttcatcttcagcggcaccttcagaaagaacctggacccctacgagcagtggagcgaccaggagatctggaaggtggccgacgaggtgggcctgagaagcgtgatcgagcagttccccggcaagctggacttcgtgctggtggacggcggctgcgtgctgagccacggccacaagcagctgatgtgcctggccagaagcgtgctgagcaaggccaagatcctgctgctggacgagcccagcgcccacctggaccccgtgacctaccagatcatcagaagaaccctgaagcaggccttcgccgactgcaccgtgatcctgtgcgagcacagaatcgaggccatgctggagtgccagcagttcctggtgatcgaggagaacaaggtgagacagtacgacagcatccagaagctgctgaacgagagaagcctgttcagacaggccatcagccccagcgacagagtgaagctgttcccccacagaaacagcagcaagtgcaagagcaagccccagatcgccgccctgaaggaggagaccgaggaggaggtgcaggacaccagactgtga

SEQ ID NO: 25 - DNA, 4443 bp, synthetic construct
  misc_feature 1..4443: note = Synthetic polynucleotide
  source 1..4443: mol_type = other DNA
atgcagaggtcacctctggaaaaggctagcgtggtcagcaagctatttttttcctggacccgcccgatactcaggaagggctaccgacagcggctggagctgagtgacatttatcagattccctccgtcgattccgctgacaacctgtctgagaaactggagcgggaatgggatagggaactggcgtccaaaaaaaaccccaaactcatcaatgcactccgcagatgcttcttctggcggtttatgttttatggcatattcctgtatctgggggaggtgacgaaagccgtgcagccgctgctgcttggtcgcattatcgcgtcatacgatccagataacaaggaggaaagaagtatcgctatctatctcgggatagggctgtgcctgctcttcattgtgcggactcttctcttgcaccccgccattttcggtctgcatcatataggtatgcagatgagaattgcgatgttctcattgatttacaaaaaaacgcttaagctaagttcaagggtgctagataagatatcgatcggccagctggtgtctctgcttagcaacaacctcaataaattcgacgaaggccttgcactggcccacttcgtgtggatcgcccctctgcaggtggctctgctgatggggttaatatgggagctgttgcaggcctccgctttttgtggcctggggtttctcatcgtgttggccttgtttcaggcagggctgggacgtatgatgatgaaatatagggatcagagggctggcaaaatctctgagcgcctggttattacgagtgaaatgattgagaacatccagtcagtgaaggcctattgctgggaggaggccatggaaaaaatgattgagaacctacgccagactgagctgaagttaaccagaaaagccgcctatgtgcgctactttaacagtagcgcatttttcttctccggttttttcgtggtgtttcttagtgtgttgccgtatgccttaatcaagggaataatactccggaagattttcactaccatcagcttctgtatcgtgttgcggatggccgtcacccggcagtttccctgggcagtacagacttggtacgattctctcggagcaattaacaaaatccaagactttctacaaaagcaggagtacaagaccctggagtacaatctgaccaccacagaagtcgtaatggagaatgtaactgccttctgggaagagggctttggcgaactctttgaaaaggccaagcagaacaataacaaccggaagacctccaacggggacgacagcttatttttcagcaatttttctttgctcgggacccctgtactgaaagatattaactttaagatcgagcgcggacaactcctggctgtcgccggcagcactggagctggaaaaacatcactgcttatggtgataatgggagaactcgaaccaagcgagggaaaaataaagcactctggacggattagtttttgctcccagttctcgtggataatgcctggcaccattaaggagaatatcatctttggagtgagttacgacgaataccggtaccggtccgttatcaaggcttgtcaactcgaggaggacatttctaaattcgccgaaaaagataatatagtgctgggcgaaggaggcattacactgagcgggggtcagagagctcgaattagcctcgcccgagcagtctataaagacgccgatctttacctgctggattccccttttgggtatttggatgttctgacagagaaggaaatctttgaatcatgtgtctgtaaactgatggccaataagactaggattctagtgacttcgaaaatggagcacctgaaaaaagcggacaaaattctgatactccatgaagggtcttcctacttctacggcaccttctcagagttgcagaacttacaacctgatttttcatctaagcttatggggtgcgactcgtttgaccagttctccgctgaaagacgaaacagcatcttaacggaaactcttcacaggttctcattagagggagatgcgccggtgtcctggacagagacaaaaaaacagtctttcaaacagacaggagagtttggcgagaagagaaaaaactcaatcctcaatcccatcaattctattagaaagtttagcatcgtccaaaaaacaccattgcagatgaatgggattgaggaggacagtgatgagcctttggaacgaagactgtccctggtacccgatagcgaacagggtgaggccatccttcctaggatctcggtcataagtacagggcccacactgcaggccaggcgacgtcaaagtgtcctcaatcttatgacgcacagtgtgaatcaggggcagaacatccatcgtaagacgacagcttcaactcgaaaggtcagtctagctccacaagccaatcttacagagctggacatttattcccgccgcctcagtcaggagaccggattggaaatatcagaggaaattaatgaagaggatctgaaggaatgcttctttgatgacatggaatcgatccccgctgttactacctggaacacatatctgagatatattaccgtccataagagcttaatctttgtactgatatggtgcttggtgattttcctggcagaggttgcggcgagtttggtcgtgctatggctccttggaaacactcccctgcaggataaggggaactccactcatagcaggaataacagctatgccgtgatcatcacctctacctcctcttattacgtgttttacatatacgtcggtgttgcggataccctgttggcaatggggttctttagaggactacccctagttcacaccctgatcaccgtttcgaagatcttgcaccacaagatgcttcatagcgttctccaagctcctatgagcacccttaatacactgaaagcaggaggtatccttaaccgcttttccaaagacatcgctatactcgacgatttgctcccattgaccatcttcgacttcattcagctgctcctcattgtgatcggcgccattgccgtggtcgcagtgttacagccatatattttcgtagccaccgtgcccgtcatcgtggcatttatcatgctgcgcgcatatttcttacagacatctcagcaactgaagcagctggaatctgagggcagatctcctatttttacacacctggttaccagcctgaagggcctgtggaccctgcgtgctttcggtcgccaaccctactttgagactctcttccataaggctctgaatttacatactgccaattggttcctataccttagtacccttcggtggttccagatgcggatagaaatgatcttcgtgattttcttcatcgcagtcactttcatctctattttgacgaccggtgagggcgagggcagggtgggcatcattctgactttggccatgaacattatgtcaacactccagtgggccgttaattcaagcattgatgtggattccttgatgcgttccgtcagcagggtatttaaattcatagacatgcccaccgagggcaagccaacaaaatctaccaagccatacaaaaatggccaactaagcaaggtcatgattatcgagaattctcatgtgaaaaaggacgacatttggccttccgggggtcaaatgactgtaaaggacctgacggctaaatacactgagggcggtaatgctatcttggagaacatctctttcagcatctcccctggccagagagtgggactgctcgggcggacaggctccggaaagtctacgctcctttcagcattccttagacttctgaacaccgaaggtgagattcagattgacggggtctcttgggactccatcacacttcagcaatggaggaaggcattcggtgtaatcccccaaaaggtttttatcttctccggaacatttcgtaagaatctggacccgtacgagcagtggtcagatcaggagatctggaaagtagcagacgaggtcgggctacggagcgttattgaacagtttcctggcaaactggacttcgttttggtggacggaggctgtgtgctgagtcacggccataaacaactgatgtgcttagctaggtctgttctcagcaaggcaaagattttactgctggatgaaccaagcgcccaccttgatccagtgacatatcaaatcatcagaagaactcttaaacaggcgttcgccgactgcacagtgatcctgtgtgagcacagaatagaagccatgctggaatgtcaacagtttctcgtgattgaggagaacaaggtgcgccagtacgatagcatccagaagttactcaatgaaaggtcactcttcaggcaggccatctcacccagcgaccgcgttaagctgtttccacaccgaaacagttccaagtgcaaaagtaagccacagattgctgcactgaaggaagagacagaagaagaagttcaggacactcggctctga

SEQ ID NO: 26 - DNA, 4443 bp, synthetic construct; identical to SEQ ID NO: 8
  misc_feature 1..4443: note = Synthetic polynucleotide
  source 1..4443: mol_type = other DNA

SEQ ID NO: 27 - DNA, 4443 bp, synthetic construct
  misc_feature 1..4443: note = Synthetic polynucleotide
  source 1..4443: mol_type = other DNA
atgcaacggagtcctctggaaaaagcctctgtcgtatctaagcttttcttcagttggacacgcccgattttgagaaagggttatcggcaacgcttggaacttagtgacatctaccaaattccaagtgtagactcagccgataacttgagcgaaaagctcgaacgagagtgggatcgagaactggctagcaaaaaaaatcccaaactcataaatgccctgcgacgctgtttcttttggcgatttatgttttacggtattttcctttatttgggtgaggtcacgaaggctgtacagccactgctgctgggtcgcatcattgcctcttacgaccctgacaacaaagaggagcggtcaatagctatctaccttggtataggactttgcttgctcttcatagtccgcacgttgcttctccaccctgctatatttggtctccatcacattgggatgcaaatgcggatcgcgatgttcagtcttatatataaaaagactcttaaactttccagccgggttctggataagatctctattggtcaactggtatctcttttgtctaacaacctgaataagttcgacgagggccttgcattggcccattttgtatggattgcccctttgcaagtcgccctcctgatgggattgatctgggaactcctgcaagctagtgctttttgcggattgggattcctcatagtccttgcgctctttcaggcgggacttggacgcatgatgatgaagtatcgcgaccaacgagctggcaagatcagtgaacggcttgtaataaccagtgaaatgatagagaacatccagagcgtaaaagcttactgttgggaagaagcgatggaaaagatgattgagaaccttcgccagacagaacttaaacttacacgaaaggccgcttatgtccggtacttcaactcttcagcatttttttttagtggcttctttgtagtgttcctgtccgtccttccgtatgcacttatcaagggtataatacttaggaaaatcttcacaacaatcagtttttgcatagtccttcgcatggcagtaactcgccaatttccctgggcagttcagacgtggtacgactcacttggcgcaattaacaaaattcaagatttcctccaaaagcaagagtataaaaccttggaatacaaccttaccaccacagaagttgtaatggaaaatgtcacagccttctgggaggaaggtttcggcgaactttttgagaaggcgaagcaaaataacaataatcggaaaacatcaaacggtgacgattcactgttcttttctaactttagccttcttgggacgcccgtcctgaaggacataaactttaagattgaacggggtcaacttctcgcggtcgcagggagtactggagcggggaaaacgagcctgctgatggtgataatgggggagttggagccctcagaaggcaagatcaagcatagtggtagaattagcttctgcagtcaatttagttggattatgccgggcacgatcaaagaaaatataatctttggggtatcctacgatgaatacaggtaccgatcagtgataaaagcgtgccagcttgaagaagacatttcaaagtttgctgagaaggataatatcgtacttggagaaggaggtatcaccctgtctgggggtcaacgagcgaggatctccctggcacgcgccgtctacaaggacgcggacctctatctgttggattcaccgttcggatatttggacgtgcttacggagaaagaaatatttgagagctgtgtttgcaagctcatggcaaataaaaccagaatattggttacaagcaagatggagcatcttaagaaagcagataaaatcctgatattgcacgagggctcttcatacttctacgggacgttttctgagttgcagaacctccagccggatttcagctctaagctgatgggctgtgattcctttgatcagtttagtgcggaaagacgaaacagtatactcaccgaaacactgcacaggttctctctggagggcgacgccccggtttcctggacagagacgaagaagcagtccttcaaacagacaggcgagtttggggagaaaaggaaaaatagcatactcaacccgattaacagcattcgcaagttcagtatagtacaaaagaccccgttgcagatgaacggtatagaggaagattctgatgagccactggaaagacggctttctctcgttccggacagtgaacagggagaggcaatactgcctcggatcagcgttatctctacaggacctactttgcaagctcggcgccgacagtcagtcttgaatcttatgactcatagtgttaatcaaggccagaatatccatcgcaagaccaccgcaagtacaaggaaagtgagcttggcacctcaagcaaaccttactgaacttgatatctactcacggcgactttcacaggagaccggacttgaaattagtgaagaaattaacgaggaggacctcaaggagtgcttcttcgatgacatggaatcaatccccgcagtcacaacctggaacacttatctgaggtatataacagttcacaagagcctcatttttgtacttatttggtgtttggtaattttcctggcggaggttgctgcttctttggtcgtcctttggctcctcgggaatacaccgctccaagacaaaggcaactctacccatagtaggaacaattcatatgcagtgattataaccagtacatcatcttattacgttttctatatttatgtcggggtagctgacacgctgttggcgatgggcttctttaggggcctccccttggtacacacccttatcacggtgagtaaaatcctgcatcacaaaatgcttcattctgtactccaagcgccgatgagtacgcttaatacgctgaaagcaggagggatactgaatcggttcagcaaggacatcgccattctggatgacctgcttccattgacaatatttgatttcattcagctccttctcatagttattggagccatagcggtggtggctgtgcttcagccttatatattcgttgccacagttcccgttatagtggcatttataatgctcagggcctactttctccagacttcccagcagttgaagcaactcgaatcagaaggaaggtcacctattttcacacatcttgtgacttccttgaagggcttgtggacgctgcgggccttcggaagacaaccatattttgaaactctcttccacaaagctttgaatcttcatactgcgaactggttcctgtatttgagtactttgcgctggttccagatgaggatagaaatgatattcgttatcttctttatcgcggttacgttcataagtatcctcactacgggggagggtgagggtagagtgggcataatactgaccctcgccatgaacattatgtccaccctgcagtgggcggtaaacagcagcatagatgtggattctttgatgcgcagtgtgagcagggtttttaagtttatcgatatgccgacggaaggaaagcccactaaaagcacgaaaccctataaaaatggacagcttagcaaagtaatgataatcgagaatagccatgtgaaaaaggatgacatatggccttccggaggccaaatgactgttaaagatctgaccgctaaatataccgagggcggcaacgcaatactcgaaaacataagcttttccataagccccggccaacgcgtgggtcttctggggaggactggctccggaaaatcaacgttgcttagcgcgtttttgcggctccttaacactgaaggtgagatccaaatagatggcgttagttgggactctataacactgcaacaatggcggaaagctttcggcgtcatacctcagaaggtgttcatctttagcggaacgttcaggaagaacttggatccctacgaacaatggagtgatcaagaaatatggaaagtggcagatgaggtaggcttgcgcagtgtcattgaacaattcccagggaaactcgactttgtactggtggacggcggttgcgtcttgtcacacgggcacaaacagttgatgtgtttggcccgcagtgttttgtctaaggcgaagattctgttgctcgacgaaccgagtgctcatcttgatcccgtcacctaccaaatcatcagaaggacgttgaagcaagctttcgccgactgcactgtaatcctttgtgagcataggatcgaagcaatgctcgagtgccaacagttcttggttatagaggagaataaggttcggcaatacgactcaatacagaaactgcttaatgagcggtcactctttcgacaagctatctctcctagtgacagggtaaagctttttcctcatcggaattccagcaagtgtaagagtaaaccacagatcgccgcccttaaagaggagaccgaagaagaggtgcaggatacgagactttag

SEQ ID NO: 28 - RNA, 4688 nt, synthetic construct; identical to SEQ ID NO: 6
  misc_feature 1..4688: note = Synthetic polynucleotide
  source 1..4688: mol_type = other RNA

SEQ ID NO: 29 - RNA, 4688 nt, synthetic construct
  misc_feature 1..4688: note = Synthetic polynucleotide
  source 1..4688: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcagcggtccccgctcgaaaaggccagtgtcgtgtccaaactcttcttctcatggactcggcctatccttagaaaggggtatcggcagaggcttgagttgtctgacatctaccagatcccctcggtagattcggcggataacctctcggagaagctcgaacgggaatgggaccgcgaactcgcgtctaagaaaaacccgaagctcatcaacgcactgagaaggtgcttcttctggcggttcatgttctacggtatcttcttgtatctcggggaggtcacaaaagcagtccaacccctgttgttgggtcgcattatcgcctcgtacgaccccgataacaaagaagaacggagcatcgcgatctacctcgggatcggactgtgtttgcttttcatcgtcagaacacttttgttgcatccagcaatcttcggcctccatcacatcggtatgcagatgcgaatcgctatgtttagcttgatctacaaaaagacactgaaactctcgtcgcgggtgttggataagatttccatcggtcagttggtgtccctgcttagtaataacctcaacaaattcgatgagggactggcgctggcacatttcgtgtggattgccccgttgcaagtcgcccttttgatgggccttatttgggagctgttgcaggcatctgccttttgtggcctgggatttctgattgtgttggcattgtttcaggctgggcttgggcggatgatgatgaagtatcgcgaccagagagcgggtaaaatctcggaaagactcgtcatcacttcggaaatgatcgaaaacatccagtcggtcaaagcctattgctgggaagaagctatggagaagatgattgaaaacctccgccaaactgagctgaaactgacccgcaaggcggcgtatgtccggtatttcaattcgtcagcgttcttcttttccgggttcttcgttgtctttctctcggttttgccttatgccttgattaaggggattatcctccgcaagattttcaccacgatttcgttctgcattgtattgcgcatggcagtgacacggcaatttccgtgggccgtgcagacatggtatgactcgcttggagcgatcaacaaaatccaagacttcttgcaaaagcaagagtacaagaccctggagtacaatcttactactacggaggtagtaatggagaatgtgacggctttttgggaagagggttttggagaactgtttgagaaagcaaagcagaataacaacaaccgcaagacctcaaatggggacgattccctgtttttctcgaacttctccctgctcggaacacccgtgttgaaggacatcaatttcaagattgagaggggacagcttctcgcggtagcgggaagcactggtgcgggaaaaactagcctcttgatggtgattatgggggagcttgagcccagcgaggggaagattaaacactccgggcgtatctcattctgtagccagttttcatggatcatgcccggaaccattaaagagaacatcattttcggagtatcctatgatgagtaccgatacagatcggtcattaaggcgtgccagttggaagaggacatttctaagttcgccgagaaggataacatcgtcttgggagaagggggtattacattgtcgggagggcagcgagcgcggatcagcctcgcgagagcggtatacaaagatgcagatttgtatctgcttgattcaccgtttggatacctcgacgtattgacagaaaaagaaatcttcgagtcgtgcgtgtgtaaacttatggctaataagacgagaatcctggtgacatcaaaaatggaacaccttaagaaggcggacaagatcctgatcctccacgaaggatcgtcctacttttacggcactttctcagagttgcaaaacttgcagccggacttctcaagcaaactcatggggtgtgactcattcgaccagttcagcgcggaacggcggaactcgatcttgacggaaacgctgcaccgattctcgcttgagggtgatgccccggtatcgtggaccgagacaaagaagcagtcgtttaagcagacaggagaatttggtgagaaaagaaagaacagtatcttgaatcctattaactcaattcgcaagttctcaatcgtccagaaaactccactgcagatgaatggaattgaagaggattcggacgaacccctggagcgcaggcttagcctcgtgccggattcagagcaaggggaggccattcttccccggatttcggtgatttcaaccggacctacacttcaggcgaggcgaaggcaatccgtgctcaacctcatgacgcattcggtaaaccaggggcaaaacattcaccgcaaaacgacggcctcaacgagaaaagtgtcacttgcaccccaggcgaatttgactgaactcgacatctacagccgtaggctttcgcaagaaaccggacttgagatcagcgaagaaatcaatgaagaagatttgaaagagtgtttctttgatgacatggaatcaatcccagcggtgacaacgtggaacacatacttgcgttacatcacggtgcacaagtccttgattttcgtcctcatctggtgtctcgtgatctttctcgctgaggtcgcagcgtcacttgtggtcctctggctgcttggtaatacgcccttgcaagacaaaggcaattctacacactcaagaaacaattcctatgccgtgattatcacttctacaagctcgtattacgtgttttacatctacgtaggagtggccgacactctgctcgcgatgggtttcttccgaggactcccactcgttcacacgcttatcactgtctccaagattctccaccataagatgcttcatagcgtactgcaggctcccatgtccaccttgaatacgctcaaggcgggaggtattttgaatcgcttctcaaaagatattgcaattttggatgaccttctgcccctgacgatcttcgacttcatccagttgttgctgatcgtgattggggctattgcagtagtcgctgtcctccagccttacatttttgtcgcgaccgttccggtgatcgtggcgtttatcatgctgcgggcctatttcttgcagacgtcacagcagcttaagcaactggagtctgaagggaggtcgcctatctttacgcatcttgtgaccagtttgaagggattgtggacgttgcgcgcctttggcaggcagccctactttgaaacactgttccacaaagcgctgaatctccatacggcaaattggtttttgtatttgagtaccctccgatggtttcagatgcgcattgagatgatttttgtgatcttctttatcgcggtgacttttatctccatcttgaccacgggagagggcgagggacgggtcggtattatcctgacactcgccatgaacattatgagcactttgcagtgggcagtgaacagctcgattgatgtggatagcctgatgaggtccgtttcgagggtctttaagttcatcgacatgccgacggagggaaagcccacaaaaagtacgaaaccctataagaatgggcaattgagtaaggtaatgatcatcgagaacagtcacgtgaagaaggatgacatctggcctagcgggggtcagatgaccgtgaaggacctgacggcaaaatacaccgagggagggaacgcaatccttgaaaacatctcgttcagcattagccccggtcagcgtgtggggttgctcgggaggaccgggtcaggaaaatcgacgttgctgtcggccttcttgagacttctgaatacagagggtgagatccagatcgacggcgtttcgtgggatagcatcaccttgcagcagtggcggaaagcgtttggagtaatcccccaaaaggtctttatctttagcggaaccttccgaaagaatctcgatccttatgaacagtggtcagatcaagagatttggaaagtcgcggacgaggttggccttcggagtgtaatcgagcagtttccgggaaaactcgactttgtccttgtagatgggggatgcgtcctgtcgcatgggcacaagcagctcatgtgcctggcgcgatccgtcctctctaaagcgaaaattcttctcttggatgaaccttcggcccatctggacccggtaacgtatcagatcatcagaaggacacttaagcaggcgtttgccgactgcacggtgattctctgtgagcatcgtatcgaggccatgctcgaatgccagcaatttcttgtcatcgaagagaataaggtccgccagtacgactccatccagaagctgcttaatgagagatcattgttccggcaggcgatttcaccatccgatagggtgaaactttttccacacagaaattcgtcgaagtgcaagtccaaaccgcagatcgcggccttgaaagaagagactgaagaagaagttcaagacacgcgtctttaacgggtggcatccctgtgacccctccccagtgcctctcctggccctggaagttgccactccagtgcccaccagccttgtcctaataaaattaagttgcatcaagct

SEQ ID NO: 30 - RNA, 4688 nt, synthetic construct
  misc_feature 1..4688: note = Synthetic polynucleotide
  source 1..4688: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtgacgggtggcatccctgtgacccctccccagtgcctctcctggccctggaagttgccactccagtgcccaccagccttgtcctaataaaattaagttgcatcaagct

(19) United States
(12) Patent Application Publication
(10) Pub. No.: US 2025/0197463 A1
(43) Pub. Date: Jun. 19, 2025

(54) TREATMENT OF CYSTIC FIBROSIS BY DELIVERY OF NEBULIZED mRNA ENCODING CFTR

(71) Applicant: TRANSLATE BIO, INC., Waltham, MA (US)

(72) Inventors: Shrirang Karve, Waltham, MA (US); Frank DeRosa, Waltham, MA (US); Michael Heartlein, Waltham, MA (US); Zarna Patel, Waltham, MA (US)

(21) Appl. No.: 18/960,025

(22) Filed: Nov. 26, 2024

Related U.S. Application Data
(63) Continuation of application No. 17/693,120, filed on Mar. 11, 2022, now Pat. No. 12,195,505.
Application No. 17/693,120 is a continuation of application No. 16/691,268, filed on Nov. 21, 2019, now abandoned.
(60) Provisional application No. 62/848,401, filed on May 15, 2019.
(60) Provisional application No. 62/846,458, filed on May 10, 2019.
(60) Provisional application No. 62/829,461, filed on Apr. 4, 2019.
(60) Provisional application No. 62/770,596, filed on Nov. 21, 2018.

(51) Int. Cl. C07K 14/47; A61K 9/00; A61K 9/1272; A61K 9/19; A61K 9/51; A61K 38/00; A61K 45/06; A61K 47/26; A61P 11/00; B82Y 5/00

(52) U.S. Cl. CPC C07K 14/4702; A61K 9/0078; A61K 9/1272; A61K 9/19; A61K 9/51; A61K 45/06; A61K 47/26; A61P 11/00; B82Y 5/00; C07K 14/4712; A61K 38/00; Y10S 977/80

(57) ABSTRACT

The present invention provides, among other things, an improved method of treating cystic fibrosis (CF) in a human subject. The method comprises administering a composition comprising an mRNA encoding a Cystic Fibrosis Transmembrane Conductance Regulator (CFTR) protein at a concentration of 0.5 mg/mL or greater to a human subject via nebulization. The composition is aerosolized using a nebulizer and a nominal dose of the mRNA is administered to the human subject via the nebulizer over a period of time, typically at least 30 minutes, at a suitable nebulization rate, e.g., at least 0.2 mL/minute.

### CROSS-REFERENCE TO RELATED APPLICATIONS

This application is a continuation of U.S. patent application Ser. No. 17/693,120, filed Mar. 11, 2022, which is a continuation of U.S. patent application Ser. No. 16/691,268, filed Nov. 21, 2019, which claims priority to U.S. Provisional Patent Application Ser. Nos. 62/848,401, filed May 15, 2019, 62/846,458, filed May 10, 2019, 62/829,461, filed Apr. 4, 2019, and 62/770,596, filed Nov. 21, 2018, the contents of each of which are incorporated herein in their entirety.

### SEQUENCE LISTING

The instant application contains a Sequence Listing which has been submitted electronically in XML format and is hereby incorporated by reference in its entirety. Said XML file, created on Nov. 19, 2024, is named 759469_SA9-858CON2_ST26.xml and is 156,826 bytes in size.

### BACKGROUND

Cystic fibrosis is an autosomal inherited disorder resulting from mutation of the Cystic Fibrosis Transmembrane Conductance Regulator (CFTR gene), which encodes a chloride ion channel believed to be involved in regulation of multiple other ion channels and transport systems in epithelial cells. Loss of function of CFTR results in chronic lung disease, aberrant mucus production, and dramatically reduced life expectancy. See generally Rowe et al., New Engl. J. Med. 352, 1992-2001 (2005).

Currently there is no cure for cystic fibrosis. The literature has documented numerous difficulties encountered in attempting to induce expression of CFTR in the lung. For example, viral vectors comprising CFTR DNA triggered immune responses and CF symptoms persisted after administration. Conese et al., J. Cyst. Fibros. 10 Suppl 2, S114-28 (2011); Rosenecker et al., Curr. Opin. Mol. Ther. 8, 439-45 (2006). Non-viral delivery of DNA, including CFTR DNA, has also been reported to trigger immune responses. Alton et al., Lancet 353, 947-54 (1999); Rosenecker et al., J Gene Med. 5, 49-60 (2003). Furthermore, non-viral DNA vectors encounter the additional problem that the machinery of the nuclear pore complex does not ordinarily import DNA into the nucleus, where transcription would occur. Pearson, Nature 460, 164-69 (2009).

Experiments performed in rodents in which liposome-encapsulated CFTR mRNA was delivered to the lungs of the test animals have yielded promising results (see, e.g., WO2018/089790). The inventors have combined data from rodent and non-human primate experiments with in vitro data obtained with commercially available nebulizers to optimize pulmonary delivery of liposome-encapsulated CFTR mRNA to human adult and pediatric subjects.

### SUMMARY OF THE INVENTION

The present invention provides a particularly effective method of administering liposome-encapsulated CFTR mRNA by nebulization to the lungs of a human subject for the treatment of Cystic Fibrosis. Accordingly, the invention relates to an improved method of treating cystic fibrosis (CF) in a human subject. In particular, the method comprises administering a composition comprising an mRNA encoding a Cystic Fibrosis Transmembrane Conductance Regulator (CFTR) protein at a concentration of 0.5 mg/mL or greater to a human subject via nebulization. The composition is aerosolized using a nebulizer and a nominal dose of the mRNA is administered to the human subject via the nebulizer over a period of time, typically at least 30 minutes, at a suitable nebulization rate, e.g., at least 0.2 mL/minute.

In some embodiments, the concentration of the mRNA encoding the CFTR protein ranges from 0.5 mg/mL to 0.8 mg/mL. In a specific embodiment, the concentration is 0.6 mg/mL.

During the treatment at least 25% of the nominal dose of the mRNA encoding the CFTR protein is delivered to the lungs of the human subject. More typically at least 35% or at least 40% of the mRNA encoding the CFTR protein is delivered to the lungs of the human subject

The period over which the aerosolized composition is administered to the human subject may exceed 35 min. In some embodiments, the treatment time is 45 min, 1 hour, 2 hours or 3 hours. A typical treatment time may be 35 min to 4.5 hours, e.g., 1.5 hours to 2 hours or 2.5 hours to 4 hours.

Generally, the nominal dose administered to the human subject during a single treatment exceeds 5 mg. In some embodiments, the nominal dose is between 4 mg and 40 mg, e.g., between 6 mg and 30 mg. In specific embodiments, the nominal dose administered to the human subject is 8 mg, 16 mg or 24 mg.

The nebulization rate may range from 0.2 mL/minute to 0.5 mL/minute. In some embodiments, the volume of the composition that is aerosolized in a single treatment session ranges from 13.0 mL to 42.0 mL. In certain embodiments, the volume of the composition that is aerosolized does not exceed 20 mL in a single treatment.

A suitable nebulizer for use with the method of the invention produces droplets with an average size between 4 μm and 6 μm.

At least some portion of the mRNA encoding the CFTR protein may be complexed to or encapsulated within nanoparticles. Suitable nanoparticles include liposomes. Typically, the mRNA encoding the CFTR protein is encapsulated within the liposome. In certain embodiments, at least 75% of the mRNA encoding the CFTR protein is encapsulated within a nanoparticle. In certain embodiments, at least 80% of the mRNA encoding the CFTR protein is encapsulated within a nanoparticle. In certain embodiments, at least 85% of the mRNA encoding the CFTR protein is encapsulated within a nanoparticle. In a specific embodiment, at least 90% of the mRNA encoding the CFTR protein is encapsulated within a nanoparticle. In any of the above embodiments, the nanoparticle may be a liposome.

A liposome suitable for use with the present invention may have a size less than about 100 nm. For example, the liposome may have a size ranging from 40 nm to 60 nm.

In some embodiments, a liposome in accordance with the invention comprises one or more cationic lipids, one or more non-cationic lipids, and one or more PEG-modified lipids. In some embodiments, the liposome comprises no more than three distinct lipid components. In certain embodiments, one distinct lipid component is a sterol-based cationic lipid. In other embodiments, the no more than three distinct lipid components are a cationic lipid, a non-cationic lipid and a PEG-modified lipid. In a specific embodiment, the cationic lipid is imidazole cholesterol ester (ICE), the non-cationic lipid is 1,2-dioleoyl-sn-glycero-3-phosphoethanolamine (DOPE), and the PEG-modified lipid is 1,2-dimyristoyl-sn-glycerol, methoxypolyethylene glycol (DMG-PEG-2K). In some embodiments, ICE and DOPE are present at a molar ratio of >1:1. In some embodiments, ICE and DMG-PEG-2K are present at a molar ratio of >10:1. In some embodiments, DOPE and DMG-PEG-2K are present at a molar ratio of >5:1.

In certain embodiments, the mRNA has poly-A tail with an average length of at least 100 bases. In certain embodiments, the mRNA has poly-A tail with an average length of at least 200 bases. In certain embodiments, the mRNA has poly-A tail with an average length of at least 500 bases. For example, a suitable length of the poly-A tail is between 400 and 700 bases. In some embodiments, the mRNA encoding the CFTR protein comprises a polynucleotide sequence at least 85%, at least 90%, at least 95%, or at least 99% identical to SEQ ID NO: 1. In a specific embodiment, the mRNA encoding the CFTR protein comprises a polynucleotide sequence identical to SEQ ID NO: 1. The mRNA encoding the CFTR protein may further comprise a 5′ untranslated region (UTR) sequence of SEQ ID NO: 3 and/or a 3′ untranslated region (UTR) sequence of SEQ ID NO: 4 or SEQ ID NO: 5. In some embodiments, the mRNA encoding the CFTR protein comprises a polynucleotide sequence at least 85%, at least 90% or at least 91% or at least 92% or at least 93% or at least 94% or at least 95% or at least 96% or at least 97% or at least 98% or at least 99% or 100% identical to any one of SEQ ID NO: 6 to SEQ ID NO: 27. In a specific embodiment, the mRNA encoding the CFTR protein comprises a polynucleotide sequence identical to SEQ ID NO: 6.

In some embodiments, a composition for use with the invention is provided in lyophilized form and reconstituted in an aqueous solution prior to nebulization.

In certain embodiments, the composition comprising the mRNA encoding the CFTR protein comprises trehalose. A suitable trehalose concentration is 10% (w/v).

In some embodiments, the human subject is administered the composition at least once per week for a period of at least six months.

In some embodiments, the human subject receives concomitant CFTR modulator therapy.

In some embodiments, the concomitant CFTR modulator therapy is selected from ivacaftor, ivacaftor/lumacaftor, or tezacaftor/lumacaftor. Accordingly, in some embodiments, the concomitant CFTR modulator therapy comprises ivacaftor. In some embodiments, the CFTR modulator therapy comprises ivacaftor/lumacaftor. In some embodiments, the CFTR modulator therapy comprises tezacaftor/lumacaftor.

In some embodiments, the human subject has an F508del mutation.

In some embodiments, the human subject is selected for treatment based on the presence of an F508del mutation.

In some embodiments, the human subject has a heterozygous or homozygous F508del mutation. Accordingly, in some embodiments, the human subject has a heterozygous F508del mutation. In some embodiments, the human subject has a homozygous F508del mutation.

In some embodiments, the subject does not have an F508del mutation.

In some embodiments, the subject is selected for treatment based on the absence of an F508del mutation.

In some embodiments, the human subject has a forced expiratory volume in one second (FEV1) of between about 50% and 90% of predicted normal. For example, the human subject has an FEV1 of about 50%, about 55%, about 60%, about 65%, about 70%, about 80%, about 85%, or about 90% of predicted normal.

In some embodiments, the human subject has an FEV1 of between about 60% and 70% predicted of predicted normal. For example, the human subject has an FEV1 of about 60%, about 61%, about 62%, about 63%, about 64%, about 65%, about 66%, about 67%, about 68%, about 69%, or about 70% of predicted normal.

It is to be understood that all embodiments as described above are applicable to all aspects of the present invention. Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIG. 1 depicts the general structure of the composition comprising an mRNA encoding a CFTR protein and a simplified formulation process.

FIG. 2 depicts an exemplary graph of the number of copies of mRNA/gm in rat lung tissue after the rats were treated with hCFTR mRNA-loaded liposomes.

FIG. 3 depicts an exemplary graph of the number of copies of mRNA in non-human primate (NHP) lung tissue after the NHPs were treated with hCFTR mRNA-loaded liposomes.

FIG. 4 depicts an exemplary graph showing increased chloride channel activity demonstrated after transfection with hCFTR mRNA-loaded liposomes.

FIG. 5 depicts expression of hCFTR protein in murine airway epithelial cells in lungs of CFTR knock out (KO) mice two hours after administration of hCFTR mRNA-loaded liposomes.

FIG. 6 depicts expression of hCFTR protein in murine airway epithelial cells in lungs of CFTR knock out (KO) mice twenty-four hours after administration of hCFTR mRNA-loaded liposomes.

FIG. 7 depicts hCFTR protein expression in lung tissue from a non-human primate (NHP) after a single dose of hCFTR mRNA-loaded liposomes, detected using immunohistochemistry.

FIG. 8 depicts an exemplary graph of fold-increase of copies of CO-hCFTR mRNA over endogenous levels of CFTR mRNA in non-human primate lung tissue after a single dose of a composition comprising an mRNA encoding a CFTR protein.

FIG. 9 depicts an exemplary graph of percent of CO-hCFTR mRNA delivered to non-human primate lung tissue as compared to airway tissue after a single dose of a composition comprising an mRNA encoding a CFTR protein.

FIGS. 10A-10C depict expression of CFTR mRNA-derived protein in non-human primate upper bronchial epithelial cells and colocalization with endogenous ZO1 protein. FIG. 10A depicts microscopic immunostaining images of upper bronchial epithelial cells from control animal treated with 10% trehalose; CFTR staining (left), ZO1 staining (middle) and optical merge of CFTR and ZO1 staining (right). FIG. 10B depicts microscopic immunostaining images of upper bronchial epithelial cells from an animal treated with low dose CFTR mRNA (500 μg/kg); CFTR staining (left), ZO1staining (middle) and optical merge of CFTR and ZO1 staining (right). FIG. 10C shows representative microscopic immunostaining images of upper bronchial epithelial cells from an animal treated with high dose CFTR mRNA (1000 μg/kg); CFTR staining (left), ZO1staining (middle) and optical merge of CFTR and ZO1 staining (right). Arrows indicate positive staining for both CFTR and ZO1.

FIGS. 11A-11C depict expression of CFTR mRNA-derived protein in non-human primate lower airway epithelial cells and colocalization with endogenous ZO1 protein. FIG. 11A depicts microscopic immunostaining images of lower airway epithelial cells from control animal treated with 10% trehalose; CFTR staining (left), ZO1 staining (middle) and optical merge of CFTR and ZO1 staining (right). FIG. 11B depicts microscopic immunostaining images of lower airway epithelial cells from an animal treated with low dose CFTR mRNA (500 μg/kg); CFTR staining (left), ZO1staining (middle) and optical merge of CFTR and ZO1 staining (right). FIG. 11C shows representative microscopic immunostaining images of lower airway epithelial cells from an animal treated with high dose CFTR mRNA (1000 μg/kg); CFTR staining (left), ZO1staining (middle) and optical merge of CFTR and ZO1 staining (right). Arrows indicate positive staining for both CFTR and ZO1.

FIGS. 12A-12C depict expression of CFTR mRNA-derived protein in alveolar region of the non-human primate lung, and colocalization with ZO1 in alveolar membrane. FIG. 12A depicts microscopic immunostaining images from control animal treated with 10% trehalose; CFTR staining (left), ZO1 staining (middle) and optical merge of CFTR and ZO1 staining (right). FIG. 12B depicts microscopic immunostaining images from an animal treated with low dose CFTR mRNA (500 μg/kg); CFTR staining (left), ZO1staining (middle) and optical merge of CFTR and ZO1 staining (right). FIG. 12C shows representative microscopic immunostaining images from an animal treated with high dose CFTR mRNA (1000 μg/kg); CFTR staining (left), ZO1 staining (middle) and optical merge of CFTR and ZO1 staining (right). Arrows indicate positive staining for both CFTR and ZO1.

FIG. 13 depicts the percent change in LNP average size (Zₐᵥₑ) pre- and post-lyophilization.

FIG. 14 depicts the percent change in mRNA-LNP size dispersity (PDI) pre- and post-lyophilization.

FIG. 15 depicts the percent change in mRNA encapsulation pre- and post-lyophilization.

FIG. 16A depicts mRNA activity pre- and post-lyophilization for a LNP formulation comprising mRNA that encodes for ASSI protein. FIG. 16B depicts mRNA activity pre- and post-lyophilization for a LNP formulation comprising mRNA that encodes for CFTR.

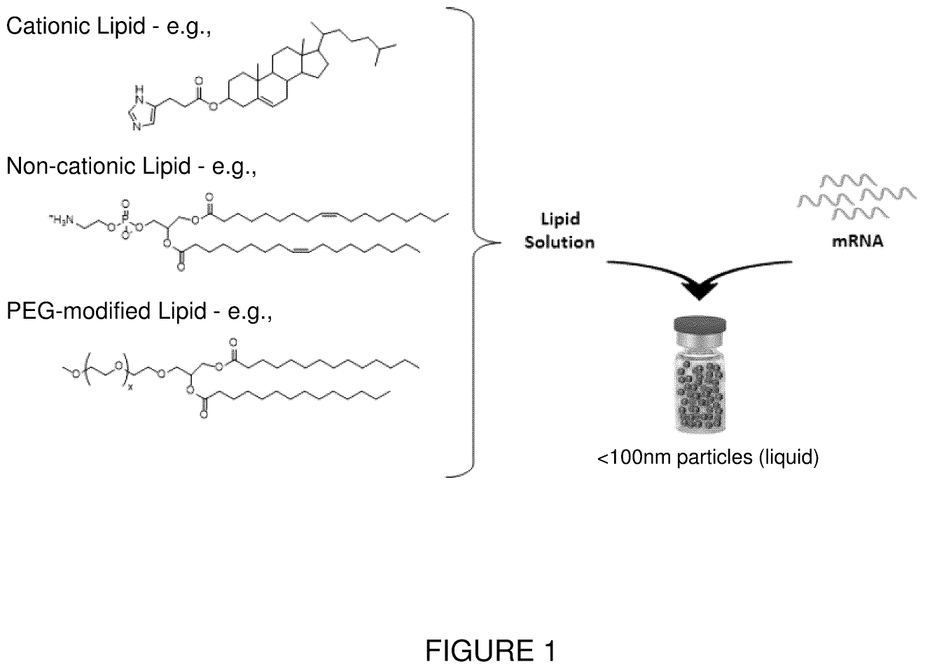

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Encapsulation: As used herein, the term “encapsulation,” or its grammatical equivalent, refers to the process of confining an mRNA molecule within a nanoparticle.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and their grammatical equivalents, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

Nominal dose: As used herein, the term “nominal dose” refers to a dose of a mRNA administered to a subject by nebulization. The nominal dose may not be identical to the dose actually delivered to the subject. For example, if a human subject is given a nominal dose of 8 μg of a CFTR mRNA composition disclosed herein, the actual dose that is delivered to the lungs of the subject may vary, e.g., depending on the nebulization parameters used to administer the composition. The actual dose cannot exceed the nominal dose, but typically the actual dose of mRNA delivered by nebulization to the lungs of the human subject is lower than the nominal dose that is administered via the nebulizer.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In specific embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, an improved method of treating cystic fibrosis (CF) in a human subject. In some embodiments, the method comprises administering a composition comprising an mRNA encoding a Cystic Fibrosis Transmembrane Conductance Regulator (CFTR) protein at a concentration of 0.5 mg/mL or greater to a human subject via nebulization. The composition is aerosolized using a nebulizer and a nominal dose of the mRNA is administered to the human subject via the nebulizer over a period of time, typically at least 30 minutes, at a suitable nebulization rate, e.g., at least 0.2 mL/minute.

Various aspects of the invention are described in detail in the following sections. The use of sections is not meant to limit the invention. Each section can apply to any aspect of the invention. In this application, the use of “or” means “and/or” unless stated otherwise.

### Cystic Fibrosis

Cystic fibrosis, also known as mucoviscidosis, is an autosomal recessive genetic disorder that affects most critically the lungs, and also the pancreas, liver, and intestine (Gibson et al., Am J Respir Crit Care Med. (2003) 168 (8): 918-951; Ratjen et al . . . , Lancet Lond Engl. (2003) 361 (9358): 681-689; O'Sullivan et al., Lancet Lond Engl. (2009) 373 (9678): 1891-1904). Cystic fibrosis is caused by mutations in the gene encoding for the cystic fibrosis transmembrane conductance regulator (CFTR) protein. This protein functions as a channel that transports chloride ions across the membrane of cells and is required to regulate the components of mucus, sweat, saliva, tears, and digestive enzymes. Disease-causing mutations in the CFTR protein cause dysfunction of its channel activity resulting in abnormal transport of chloride and sodium ions across the epithelium, leading to the thick, viscous secretions in the lung, pancreas and other organs characteristic of CF disease (O'Sulliven et al . . . , Lancet Lond Engl. (2009) 373 (9678): 1891-1904; Rowe et al., N Engl J Med. (2005) 352 (19): 1992-2001). Most CF patients develop severe, chronic lung disease related to airway obstruction partly due to increased levels of sulfated mucins, inflammation, and recurrent infections that are eventually lethal; the median predicted survival age in the US is 40.7 years. Cystic fibrosis is the most frequent lethal genetic disease in the white population.

Symptoms often appear in infancy and childhood, with respiratory symptoms the most frequent followed by failure to thrive, steatorrhea, and meconium ileus (Gibson et al., Am J Respir Crit Care Med. (2003) 168 (8): 918-951). The most common complications of CF are pulmonary related and include blockages of the narrow passages of affected organs with thickened secretions. These blockages lead to remodeling and infection in the lung, cause damage in the pancreas due to accumulated digestive enzymes, and blockages of the intestines. Diabetes is the most common non-pulmonary complication and is a distinct entity known as CF-related diabetes.

The lungs of individuals with CF are colonized and infected by bacteria from an early age. This leads to chronic airway infection and inflammation, progressing to bronchiectasis, gas trapping, hypoxemia, and hypercarbia. Pulmonary insufficiency is responsible for 68.1% of CF-related deaths in the US. In the initial stage, common bacteria such as Staphylococcus aureus and Hemophilus influenzae colonize and infect the lungs. Eventually, Pseudomonas aeruginosa (and sometimes Burkholderia cepacia) dominates. By 18 years of age, 80% of patients with classic CF harbor P, aeruginosa, and 3.5% harbor B. cepacia. Once within the lungs, these bacteria adapt to the environment and develop resistance to commonly used antibiotics.

The underlying defect causing CF is abnormal epithelial anion transport due to the lack of expression or dysfunction of the CFTR protein. The CFTR protein primarily functions as a chloride channel in epithelial cell membranes; however, it also involved in a number of other cellular membrane functions such as inhibition of sodium transport through the epithelial sodium channel, regulation of the outwardly rectifying chloride channel, and regulation of adenosine triphosphate (ATP) channels (O'Sullivan et al., Lancet Lond Engl. (2009) 373 (9678): 1891-1904). CF is caused by mutations in the gene encoding for the CFTR protein, of which more than 1,500 disease-causing mutations have been identified (O'Sullivan et al., Lancet Lond Engl. (2009) 373 (9678): 1891-1904). The more common gene mutations result in the lack of synthesis of the CFTR protein (class I), defective processing and maturation of the CFTR protein (class II), or the expression of a CFTR protein defective in regulation, e.g., diminished ATP binding and hydrolysis (class III) (Rowe et al., N Engl J Med. (2005) 352 (19): 1992-2001). A deletion of phenylalanine at position 508 (F508del) is the most common CFTR mutation worldwide and is a class II defect in which the misfolded protein is rapidly degraded by the cell soon after synthesis (Rowe et al., N Engl J Med. (2005) 352 (19): 1992-2001). The lack of a functional CFTR protein causes mucosal obstruction of exocrine glands in CF patients secondary to abnormal transport of chloride and sodium across the epithelium. In the lung, this leads to the development of thick, tenacious secretions that obstruct the airways and submucosal glands, which in turn leads to chronic bacterial infection and inflammation, as described above.

Respiratory symptoms of cystic fibrosis include: a persistent cough that produces thick mucus (sputum), wheezing, breathlessness, exercise intolerance, repeated lung infections and inflamed nasal passages or a stuffy nose. Digestive symptoms of cystic fibrosis include: foul-smelling, greasy stools, poor weight gain and growth, intestinal blockage, particularly in newborns (meconium ileus), and severe constipation.

There are several different methods for assessing symptoms of cystic fibrosis. In one embodiment, one or more symptoms of cystic fibrosis are assessed by forced expiratory volume (FEV), which measures how much air a person can exhale during a forced breath. In one embodiment, the amount of air exhaled in the first second of the forced breath is measured (FEV1). In one embodiment, the amount of air exhaled in the second of the forced breath is measured (FEV2). In one embodiment, the amount of air exhaled in the third second of the forced breath is measured (FEV3). In one embodiment, the forced vital capacity (FVC), which is the total amount of air exhaled during a FEV test, is measured. In one embodiment, one or more symptoms of cystic fibrosis are assessed by Cystic Fibrosis Questionnaire Revise (CFQ-R) respiratory domain score. CFQ-R respiratory domain score is a measure of respiratory symptoms relevant to patients with CF such as cough, sputum production, and difficulty breathing. In one embodiment, one or more symptoms of cystic fibrosis are assessed by relative risk of pulmonary exacerbation. In one embodiment, one or more symptoms of cystic fibrosis are assessed by change in body weight. In one embodiment, one or more symptoms of cystic fibrosis are assessed by change in sweat chloride (mmol/L).

### Patient Selection

The present invention is suitable for treatment of patients with various CFTR defects including, but not limited to, patients with different CFTR symptoms, mutations or classes described herein.

In some embodiments, the present invention may be used to treat patients carrying one or more, two or more, three or more, four or more, or five or more mutations from Class I (Defective Protein Synthesis) shown in Table 1. In some embodiments, the present invention may be used to treat patients carrying one or more, two or more, three or more, four or more, or five or more mutations from Class II (Abnormal Processing and Trafficking) shown in Table 1. In some embodiments, the present invention may be used to treat patients carrying one or more, two or more, three or more, four or more, or five or more mutations from Class III (Defective Chanel Regulation/Gating) shown in Table 1. In some embodiments, the present invention may be used to treat patients carrying one or more, two or more, three or more, four or more, or five or more mutations from Class IV (Decreased Channel Conductance) shown in Table 1. In some embodiments, the present invention may be used to treat patients carrying one or more, two or more, three or more, four or more, or five or more mutations from Class V (Reduced Synthesis and/or Trafficking) shown in Table 1. In some embodiments, the present invention may be used to treat patients carrying any combination of specific mutations selected from Table 1 (e.g., one or more, two or more, three or more, four or more, five or more, six or more, seven or more, eight or more, nine or more, or ten or more mutations from different classes shown in Table 1).

TABLE 1
Classification of CFTR Gene Mutations

| Category | Mutation | Specific mutations |
|---|---|---|
| Class I | Defective Protein Synthesis | 1078delT, 1154 insTC, 1525-2A > G, |
| | (nonsense, frameshift, aberrant | 1717-1G > A, 1898 + 1G > A, 2184delA, |
| | splicing) | 2184 insA, 3007delG, 3120 + 1G > A, |
| | | 3659delC, 3876delA, 3905insT, |
| | | 394delTT, 4010del4, 4016insT, |
| | | 4326delTC, 4374 + 1G > T, 441delA, |
| | | 556delA, 621 + 1G > T, 621 − 1G > T, |
| | | 711 + 1G > T, 875 + 1G > C, El 104X, |
| | | E585X, E60X, E822X, G542X, |
| | | G551D/R553X, Q493X, Q552X, Q814X, |
| | | R1066C, R1162X, R553X, V520F, |
| | | W1282X, Y1092X |
| Class II | Abnormal Processing and Trafficking | A559T, D979A, AF508, AI507, G480C, |
| | | G85E, N1303K, S549I, S549N, S549R |
| Class III | Defective Chanel Regulation/Gating | G1244E, G1349D, G551D, G551S, G85E, |
| | | H199R, I1072T, I48T, L1077P, R560T, |
| | | S1255P, S549N (R75Q) |
| Class IV | Decreased Channel Conductance | A800G, D1152H, D1154G, D614G, |
| | | delM1140, E822K, G314E, G576A, |
| | | G622D, G85E, H620Q, 11139V, 11234V, |
| | | L1335P, Ml 137V, P67L, RI 17C, R117P, |
| | | RI 17H, R334W, R347H, R347P, |
| | | R347P/R347H, R792G, S125 IN, V232D |
| Class V | Reduced Synthesis and/or Trafficking | 2789 + 5G > A, 3120G > A, 3272-26A > G, |
| | | 3849 + 10kbC > T, 5T variant, 621 + 3A > |
| | | G, 711 + 3A > G, A445E, A455E, IVS8 |
| | | poly T, P574H, 875 + 1G > C |

In some embodiments, a patient in need of treatment is a male or female of 2 years or older, or of 3 years or older, or of 6 years or older, or of 7 years or older, or of 12 years or older, or of 13 years or older, or of 18 years or older, or of 19 years or older, or of 25 years or older, or of 25 years or older, or of 30 years or older, or of 35 years or older, or of 40 years or older, or of 45 years or older, or of 50 years or older. In some embodiments, a patient in need of treatment is less than 50 years old, or less than 45 years old, or less than 40 years old, or less than 35 years old, or less than 30 years old, or less than 25 years old, or less than 20 years old, or less than 19 years old, or less than 18 years old, or less than 13 years old, or less than 12 years old, or less than 7 years old, or less than 6 years old, or less than 3 years old, or less than 2 years old. In some embodiments, a patient in need of treatment is a male or female from 2 to 18 years old, or from 2 to 12 years old, or from 2 to 6 years old, or from 6 to 12 years old, or from 6 to 18 years old, or from 12 to 16 years old, or from 2 to 50 years old, or from 6 to 50 years old, or from 12 to 50 years old, or from 18 to 50 years old. In some embodiments, a patient in need of treatment is a female who is pregnant or who may become pregnant.

In some embodiments, a patient is selected for treatment who has an F508del mutation. In some embodiments, the patient who is selected for treatment has a homozygous F508del mutation. In some embodiments, the patient who is selected for treatment has a heterozygous F508del mutation. In some embodiments, the patient who is selected for treatment does not have an F508del mutation.

In some embodiments, a patient in need of treatment has a sweat chloride value of ≥60 mmol/L, ≥65 mmol/L, ≥70 mmol/L, ≥75 mmol/L, ≥80 mmol/L, ≥85 mmol/L, ≥90 mmol/L, ≥95 mmol/L, ≥100 mmol/L, ≥110 mmol/L, ≥120 mmol/L, ≥130 mmol/L, ≥140 mmol/L or ≥150 mmol/L by quantitative pilocarpine iontophoresis (documented in the subject's medical record). In some embodiments, a patient in need of treatment has chronic sinopulmonary disease and/or gastrointestinal/nutritional abnormalities consistent with CF disease. In some embodiments, a patient in need of treatment has chronic sinopulmonary disease and/or gastrointestinal/nutritional abnormalities consistent with CF disease.

In some embodiments, a patient in need of treatment has FEV₁≥50% and ≤90% (e.g., ≤85%, ≤80%, ≤75%, ≤70%, ≤65%, <60%, or ≤55%) of the predicted normal (i.e., the average FEV of non-CF patients) based on the patient's age, gender, and height. In some embodiments, a patient in need of treatment has resting oxygen saturation ≥92% on room air (pulse oximetry). In some embodiments, a patient in need of treatment has a body mass index ≥17.5 kg/m² and weight ≥40 kg.

In some embodiments, a patient in need of treatment has received or is concurrently receiving other CF medications. For example, a patient in need of treatment may be receiving lumacaftor/ivacaftor combination drug (ORKAMBI®) or may have been on this treatment for at least 28 days prior to commencement of the treatment according to the present invention. Other CF medications may include, but are not limited to, routine inhaled therapies directed at airway clearance and management of respiratory infections, such as bronchodilators, rhDNase (PULMOZYME®), hypertonic saline, antibiotics, and steroids; and other routine CF-related therapies such as systemic antibiotics, pancreatic enzymes, multivitamins, and diabetes and liver medications.

In some embodiments, a patient in need of treatment has been a non-smoker for a minimum of 2 years. In some embodiments, a patient in need of treatment does not receive inhaled rhDNase (PULMOZYME®) treatment for 24 hours before and/or after administration of a composition comprising an mRNA encoding a CFTR protein according to the present invention.

In some embodiments, a patient in need of treatment has been treated or is currently being treated with hormone replacement therapies, thyroid hormone replacement therapy, non-steroidal inflammatory drugs, and prescription dronabinol (MARINOL®) during treatment.

In some embodiments, a patient in need of treatment has discontinued use of one or more other cystic fibrosis treatments described herein. In some embodiments, the patient has discontinued use of one or more other cystic fibrosis treatments for at least 12 hours, at least 24 hours, at least 36 hours, at least 48 hours, at least 72 hours, at least 1 week, at least 2 weeks, at least 3 weeks, at least 4 weeks, at least 5 weeks, at least 6 weeks, at least 7 weeks, or at least 8 weeks prior to administration of a CFTR mRNA according to the present invention. In some embodiments, the patient has discontinued use of one or more other cystic fibrosis treatments for less than 12 hours, less than 24 hours, less than 36 hours, less than 48 hours, less than 72 hours, less than 1 week, less than 2 weeks, less than 3 weeks, less than 4 weeks, less than 5 weeks, less than 6 weeks, less than 7 weeks, less than 8 weeks, less than 9 weeks, or less than 10 weeks prior to administration of a CFTR mRNA according to the present invention.

### Formulation and Administration

According to the present invention, a suitable formulation for the treatment contains an mRNA encoding any full length, fragment or portion of a CFTR protein which can be substituted for naturally-occurring CFTR protein activity and/or reduce the intensity, severity, and/or frequency of one or more symptoms associated with cystic fibrosis.

In some embodiments, a suitable mRNA sequence is an mRNA sequence encoding a human CFTR (hCFTR) protein. In some embodiments, a suitable mRNA sequence is codon optimized for efficient expression human cells. An exemplary codon-optimized CFTR mRNA coding sequence and the corresponding amino acid sequence are shown in Table 2:

TABLE 2
Exemplary CFTR mRNA and Protein Sequences

| | |
|---|---|
| Codon- | AUGCAACGCUCUCCUCUUGAAAAGGCCUCGGUGGUGUCCAAGCUCUU |
| Optimized | CUUCUCGUGGACUAGACCCAUCCUGAGAAAGGGGUACAGACAGCGCU |
| Human | UGGAGCUGUCCGAUAUCUAUCAAAUCCCUUCCGUGGACUCCGCGGAC |
| CFTR | AACCUGUCCGAGAAGCUCGAGAGAGAAUGGGACAGAGAACUCGCCUC |
| mRNA | AAAGAAGAACCCGAAGCUGAUUAAUGCGCUUAGGCGGUGCUUUUUC |
| coding | UGGCGGUUCAUGUUCUACGGCAUCUUCCUCUACCUGGGAGAGGUCAC |
| sequence | CAAGGCCGUGCAGCCCCUGUUGCUGGGACGGAUUAUUGCCUCCUACG |
| | ACCCCGACAACAAGGAAGAAAGAAGCAUCGCUAUCUACUUGGGCAUC |
| | GGUCUGUGCCUGCUUUUCAUCGUCCGGACCCUCUUGUUGCAUCCUGC |
| | UAUUUUCGGCCUGCAUCACAUUGGCAUGCAGAUGAGAAUUGCCAUG |
| | UUUUCCCUGAUCUACAAGAAAACUCUGAAGCUCUCGAGCCGCGUGCU |
| | UGACAAGAUUUCCAUCGGCCAGCUCGUGUCCCUGCUCUCCAACAAUC |
| | UGAACAAGUUCGACGAGGGCCUCGCCCUGGCCCACUUCGUGUGGAUC |
| | GCCCCUCUGCAAGUGGCGCUUCUGAUGGGCCUGAUCUGGGAGCUGCU |
| | GCAAGCCUCGGCAUUCUGUGGGCUUGGAUUCCUGAUCGUGCUGGCAC |
| | UGUUCCAGGCCGGACUGGGGCGGAUGAUGAUGAAGUACAGGGACCA |
| | GAGAGCCGGAAAGAUUUCCGAACGGCUGGUGAUCACUUCGGAAAUG |
| | AUCGAAAACAUCCAGUCAGUGAAGGCCUACUGCUGGGAAGAGGCCAU |
| | GGAAAAGAUGAUUGAAAACCUCCGGCAAACCGAGCUGAAGCUGACCC |
| | GCAAGGCCGCUUACGUGCGCUAUUUCAACUCGUCCGCUUUCUUCUUC |
| | UCCGGGUUCUUCGUGGUGUUUCUCUCCGUGCUCCCCUACGCCCUGAU |
| | UAAGGGAAUCAUCCUCAGGAAGAUCUUCACCACCAUUUCCUUCUGUA |
| | UCGUGCUCCGCAUGGCCGUGACCCGGCAGUUCCCAUGGGCCGUGCAG |
| | ACUUGGUACGACUCCCUGGGAGCCAUUAACAAGAUCCAGGACUUCCU |
| | UCAAAAGCAGGAGUACAAGACCCUCGAGUACAACCUGACUACUACCG |
| | AGGUCGUGAUGGAAAACGUCACCGCCUUUUGGGAGGAGGGAUUUGG |
| | CGAACUGUUCGAGAAGGCCAAGCAGAACAACAACAACCGCAAGACCU |
| | CGAACGGUGACGACUCCCUCUUCUUUUCAAACUUCAGCCUGCUCGGG |
| | ACGCCCGUGCUGAAGGACAUUAACUUCAAGAUCGAAAGAGGACAGCU |
| | CCUGGCGGUGGCCGGAUCGACCGGAGCCGGAAAGACUUCCCUGCUGA |
| | UGGUGAUCAUGGGAGAGCUUGAACCUAGCGAGGGAAAGAUCAAGCA |
| | CUCCGGCCGCAUCAGCUUCUGUAGCCAGUUUUCCUGGAUCAUGCCCG |
| | GAACCAUUAAGGAAAACAUCAUCUUCGGCGUGUCCUACGAUGAAUAC |
| | CGCUACCGGUCCGUGAUCAAAGCCUGCCAGCUGGAAGAGGAUAUUUC |
| | AAAGUUCGCGGAGAAAGAUAACAUCGUGCUGGGCGAAGGGGGUAUU |
| | ACCUUGUCGGGGGGCCAGCGGGCUAGAAUCUCGCUGGCCAGAGCCGU |
| | GUAUAAGGACGCCGACCUGUAUCUCCUGGACUCCCCCUUCGGAUACC |
| | UGGACGUCCUGACCGAAAAGGAGAUCUUCGAAUCGUGCGUGUGCAA |
| | GCUGAUGGCUAACAAGACUCGCAUCCUCGUGACCUCCAAAAUGGAGC |
| | ACCUGAAGAAGGCAGACAAGAUUCUGAUUCUGCAUGAGGGGUCCUCC |
| | UACUUUUACGGCACCUUCUCGGAGUUGCAGAACUUGCAGCCCGACUU |
| | CUCAUCGAAGCUGAUGGGUUGCGACAGCUUCGACCAGUUCUCCGCCG |
| | AAAGAAGGAACUCGAUCCUGACGGAAACCUUGCACCGCUUCUCUUUG |
| | GAAGGCGACGCCCCUGUGUCAUGGACCGAGACUAAGAAGCAGAGCUU |
| | CAAGCAGACCGGGGAAUUCGGCGAAAAGAGGAAGAACAGCAUCUUG |
| | AACCCCAUUAACUCCAUCCGCAAGUUCUCAAUCGUGCAAAAGACGCC |
| | ACUGCAGAUGAACGGCAUUGAGGAGGACUCCGACGAACCCCUUGAGA |
| | GGCGCCUGUCCCUGGUGCCGGACAGCGAGCAGGGAGAAGCCAUCCUG |
| | CCUCGGAUUUCCGUGAUCUCCACUGGUCCGACGCUCCAAGCCCGGCG |
| | GCGGCAGUCCGUGCUGAACCUGAUGACCCACAGCGUGAACCAGGGCC |
| | AAAACAUUCACCGCAAGACUACCGCAUCCACCCGGAAAGUGUCCCUG |
| | GCACCUCAAGCGAAUCUUACCGAGCUCGACAUCUACUCCCGGAGACU |
| | GUCGCAGGAAACCGGGCUCGAAAUUUCCGAAGAAAUCAACGAGGAG |
| | GAUCUGAAAGAGUGCUUCUUCGACGAUAUGGAGUCGAUACCCGCCGU |
| | GACGACUUGGAACACUUAUCUGCGGUACAUCACUGUGCACAAGUCAU |
| | UGAUCUUCGUGCUGAUUUGGUGCCUGGUGAUUUUCCUGGCCGAGGU |
| | CGCGGCCUCACUGGUGGUGCUCUGGCUGUUGGGAAACACGCCUCUGC |
| | AAGACAAGGGAAACUCCACGCACUCGAGAAACAACAGCUAUGCCGUG |
| | AUUAUCACUUCCACCUCCUCUUAUUACGUGUUCUACAUCUACGUCGG |
| | AGUGGCGGAUACCCUGCUCGCGAUGGGUUUCUUCAGAGGACUGCCGC |
| | UGGUCCACACCUUGAUCACCGUCAGCAAGAUUCUUCACCACAAGAUG |
| | UUGCAUAGCGUGCUGCAGGCCCCCAUGUCCACCCUCAACACUCUGAA |
| | GGCCGGAGGCAUUCUGAACAGAUUCUCCAAGGACAUCGCUAUCCUGG |
| | ACGAUCUCCUGCCGCUUACCAUCUUUGACUUCAUCCAGCUGCUGCUG |
| | AUCGUGAUUGGAGCAAUCGCAGUGGUGGCGGUGCUGCAGCCUUACA |
| | UUUUCGUGGCCACUGUGCCGGUCAUUGUGGCGUUCAUCAUGCUGCGG |
| | GCCUACUUCCUCCAAACCAGCCAGCAGCUGAAGCAACUGGAAUCCGA |
| | GGGACGAUCCCCCAUCUUCACUCACCUUGUGACGUCGUUGAAGGGAC |
| | UGUGGACCCUCCGGGCUUUCGGACGGCAGCCCUACUUCGAAACCCUC |
| | UUCCACAAGGCCCUGAACCUCCACACCGCCAAUUGGUUCCUGUACCU |
| | GUCCACCCUGCGGUGGUUCCAGAUGCGCAUCGAGAUGAUUUUCGUCA |
| | UCUUCUUCAUCGCGGUCACAUUCAUCAGCAUCCUGACUACCGGAGAG |
| | GGAGAGGGACGGGUCGGAAUAAUCCUGACCCUCGCCAUGAACAUUAU |
| | GAGCACCCUGCAGUGGGCAGUGAACAGCUCGAUCGACGUGGACAGCC |
| | UGAUGCGAAGCGUCAGCCGCGUGUUCAAGUUCAUCGACAUGCCUACU |
| | GAGGGAAAACCCACUAAGUCCACUAAGCCCUACAAAAAUGGCCAGCU |
| | GAGCAAGGUCAUGAUCAUCGAAAACUCCCACGUGAAGAAGGACGAU |
| | AUUUGGCCCUCCGGAGGUCAAAUGACCGUGAAGGACCUGACCGCAAA |
| | GUACACCGAGGGAGGAAACGCCAUUCUCGAAAACAUCAGCUUCUCCA |
| | UUUCGCCGGGACAGCGGGUCGGCCUUCUCGGGCGGACCGGUUCCGGG |
| | AAGUCAACUCUGCUGUCGGCUUUCCUCCGGCUGCUGAAUACCGAGGG |
| | GGAAAUCCAAAUUGACGGCGUGUCUUGGGAUUCCAUUACUCUGCAGC |
| | AGUGGCGGAAGGCCUUCGGCGUGAUCCCCCAGAAGGUGUUCAUCUUC |
| | UCGGGUACCUUCCGGAAGAACCUGGAUCCUUACGAGCAGUGGAGCGA |
| | CCAAGAAAUCUGGAAGGUCGCCGACGAGGUCGGCCUGCGCUCCGUGA |
| | UUGAACAAUUUCCUGGAAAGCUGGACUUCGUGCUCGUCGACGGGGG |
| | AUGUGUCCUGUCGCACGGACAUAAGCAGCUCAUGUGCCUCGCACGGU |
| | CCGUGCUCUCCAAGGCCAAGAUUCUGCUGCUGGACGAACCUUCGGCC |
| | CACCUGGAUCCGGUCACCUACCAGAUCAUCAGGAGGACCCUGAAGCA |
| | GGCCUUUGCCGAUUGCACCGUGAUUCUCUGCGAGCACCGCAUCGAGG |
| | CCAUGCUGGAGUGCCAGCAGUUCCUGGUCAUCGAGGAGAACAAGGUC |
| | CGCCAAUACGACUCCAUUCAAAAGCUCCUCAACGAGCGGUCGCUGUU |
| | CAGACAAGCUAUUUCACCGUCCGAUAGAGUGAAGCUCUUCCCGCAUC |
| | GGAACAGCUCAAAGUGCAAAUCGAAGCCGCAGAUCGCAGCCUUGAAG |
| | GAAGAGACUGAGGAAGAGGUGCAGGACACCCGGCUUUAA (SEQ ID |
| | NO: 1) |
| Human | MQRSPLEKASVVSKLFFSWTRPILRKGYRQRLELSDIYQIPSVDSADNLSEK |
| CFTR | LEREWDRELASKKNPKLINALRRCFFWRFMFYGIFLYLGEVTKAVQPLLL |
| Protein | GRIIASYDPDNKEERSIAIYLGIGLCLLFIVRTLLLHPAIFGLHHIGMQMRIA |
| Sequence | MFSLIYKKTLKLSSRVLDKISIGQLVSLLSNNLNKFDEGLALAHFVWIAPLQ |
| | VALLMGLIWELLQASAFCGLGFLIVLALFQAGLGRMMMKYRDQRAGKIS |
| | ERLVITSEMIENIQSVKAYCWEEAMEKMIENLRQTELKLTRKAAYVRYFN |
| | SSAFFFSGFFVVFLSVLPYALIKGIILRKIFTTISFCIVLRMAVTRQFPWAVQT |
| | WYDSLGAINKIQDFLQKQEYKTLEYNLTTTEVVMENVTAFWEEGFGELFE |
| | KAKQNNNNRKTSNGDDSLFFSNFSLLGTPVLKDINFKIERGQLLAVAGSTG |
| | AGKTSLLMVIMGELEPSEGKIKHSGRISFCSQFSWIMPGTIKENIIFGVSYDE |
| | YRYRSVIKACQLEEDISKFAEKDNIVLGEGGITLSGGQRARISLARAVYKD |
| | ADLYLLDSPFGYLDVLTEKEIFESCVCKLMANKTRILVTSKMEHLKKADKI |
| | LILHEGSSYFYGTFSELQNLQPDFSSKLMGCDSFDQFSAERRNSILTETLHR |
| | FSLEGDAPVSWTETKKQSFKQTGEFGEKRKNSILNPINSIRKFSIVQKTPLQ |
| | MNGIEEDSDEPLERRLSLVPDSEQGEAILPRISVISTGPTLQARRRQSVLNL |
| | MTHSVNQGQNIHRKTTASTRKVSLAPQANLTELDIYSRRLSQETGLEISEEI |
| | NEEDLKECFFDDMESIPAVTTWNTYLRYITVHKSLIFVLIWCLVIFLAEVAA |
| | SLVVLWLLGNTPLQDKGNSTHSRNNSYAVIITSTSSYYVFYIYVGVADTLL |
| | AMGFFRGLPLVHTLITVSKILHHKMLHSVLQAPMSTLNTLKAGGILNRFSK |
| | DIAILDDLLPLTIFDFIQLLLIVIGAIAVVAVLQPYIFVATVPVIVAFIMLRAY |
| | FLQTSQQLKQLESEGRSPIFTHLVTSLKGLWTLRAFGRQPYFETLFHKALN |
| | LHTANWFLYLSTLRWFQMRIEMIFVIFFIAVTFISILTTGEGEGRVGIILTLA |
| | MNIMSTLQWAVNSSIDVDSLMRSVSRVFKFIDMPTEGKPTKSTKPYKNGQ |
| | LSKVMIIENSHVKKDDIWPSGGQMTVKDLTAKYTEGGNAILENISFSISPGQ |
| | RVGLLGRTGSGKSTLLSAFLRLLNTEGEIQIDGVSWDSITLQQWRKAFGVIP |
| | QKVFIFSGTFRKNLDPYEQWSDQEIWKVADEVGLRSVIEQFPGKLDFVLVD |
| | GGCVLSHGHKQLMCLARSVLSKAKILLLDEPSAHLDPVTYQIIRRTLKQAF |
| | ADCTVILCEHRIEAMLECQQFLVIEENKVRQYDSIQKLLNERSLFRQAISPS |
| | DRVKLFPHRNSSKCKSKPQIAALKEETEEEVQDTRL (SEQ ID NO: 2) |

In one embodiment, a codon-optimized CFTR mRNA sequence includes SEQ ID NO: 1. In some embodiments, a codon-optimized CFTR mRNA sequence suitable for the present invention shares at least 60%, 65%, 70%, 75%, 80%, 85%, 90%, 95%, 96%, 97%, 98%, or 99% identity to SEQ ID NO:1 and encodes a CFTR protein having an amino acid sequence of SEQ ID NO: 2.

In some embodiments, a CFTR mRNA suitable for the invention also contains 5′ and 3′ UTR sequences. Exemplary 5′ and 3′ UTR sequences are shown below:

Exemplary 5′ UTR Sequence
(SEQ ID NO: 3)
GGACAGAUCGCCUGGAGACGCCAUCCACGCUGUUUUGACCUCCAUAGAAGA
CACCGGGACCGAUCCAGCCUCCGCGGCCGGGAACGGUGCAUUGGAACGCGG
AUUCCCCGUGCCAAGAGUGACUCACCGUCCUUGACACG
Exemplary 3′ UTR Sequence
(SEQ ID NO: 4)
CGGGUGGCAUCCCUGUGACCCCUCCCCAGUGCCUCUCCUGGCCCUGGAAGU
UGCCACUCCAGUGCCCACCAGCCUUGUCCUAAUAAAAUUAAGUUGCAUCAA
GCU
or
(SEQ ID NO: 5)
GGGUGGCAUCCCUGUGACCCCUCCCCAGUGCCUCUCCUGGCCCUGGAAGUU
GCCACUCCAGUGCCCACCAGCCUUGUCCUAAUAAAAUUAAGUUGCAUCAAA
GCU

Thus, in one embodiment, an exemplary full-length codon-optimized CFTR mRNA sequence suitable for the invention is:

(SEQ ID NO: 6)
GGACAGAUCGCCUGGAGACGCCAUCCACGCUGUUUUGACCUCCAUAGAAGACACCGG
GACCGAUCCAGCCUCCGCGGCCGGGAACGGUGCAUUGGAACGCGGAUUCCCCGUGCC
AAGAGUGACUCACCGUCCUUGACACGAUGCAACGCUCUCCUCUUGAAAAGGCCUCGG
UGGUGUCCAAGCUCUUCUUCUCGUGGACUAGACCCAUCCUGAGAAAGGGGUACAGAC
AGCGCUUGGAGCUGUCCGAUAUCUAUCAAAUCCCUUCCGUGGACUCCGCGGACAACC
UGUCCGAGAAGCUCGAGAGAGAAUGGGACAGAGAACUCGCCUCAAAGAAGAACCCGA
AGCUGAUUAAUGCGCUUAGGCGGUGCUUUUUCUGGCGGUUCAUGUUCUACGGCAUC
UUCCUCUACCUGGGAGAGGUCACCAAGGCCGUGCAGCCCCUGUUGCUGGGACGGAUU
AUUGCCUCCUACGACCCCGACAACAAGGAAGAAAGAAGCAUCGCUAUCUACUUGGGC
AUCGGUCUGUGCCUGCUUUUCAUCGUCCGGACCCUCUUGUUGCAUCCUGCUAUUUUC
GGCCUGCAUCACAUUGGCAUGCAGAUGAGAAUUGCCAUGUUUUCCCUGAUCUACAAG
AAAACUCUGAAGCUCUCGAGCCGCGUGCUUGACAAGAUUUCCAUCGGCCAGCUCGUG
UCCCUGCUCUCCAACAAUCUGAACAAGUUCGACGAGGGCCUCGCCCUGGCCCACUUC
GUGUGGAUCGCCCCUCUGCAAGUGGCGCUUCUGAUGGGCCUGAUCUGGGAGCUGCUG
CAAGCCUCGGCAUUCUGUGGGCUUGGAUUCCUGAUCGUGCUGGCACUGUUCCAGGCC
GGACUGGGGCGGAUGAUGAUGAAGUACAGGGACCAGAGAGCCGGAAAGAUUUCCGA
ACGGCUGGUGAUCACUUCGGAAAUGAUCGAAAACAUCCAGUCAGUGAAGGCCUACUG
CUGGGAAGAGGCCAUGGAAAAGAUGAUUGAAAACCUCCGGCAAACCGAGCUGAAGC
UGACCCGCAAGGCCGCUUACGUGCGCUAUUUCAACUCGUCCGCUUUCUUCUUCUCCG
GGUUCUUCGUGGUGUUUCUCUCCGUGCUCCCCUACGCCCUGAUUAAGGGAAUCAUCC
UCAGGAAGAUCUUCACCACCAUUUCCUUCUGUAUCGUGCUCCGCAUGGCCGUGACCC
GGCAGUUCCCAUGGGCCGUGCAGACUUGGUACGACUCCCUGGGAGCCAUUAACAAGA
UCCAGGACUUCCUUCAAAAGCAGGAGUACAAGACCCUCGAGUACAACCUGACUACUA
CCGAGGUCGUGAUGGAAAACGUCACCGCCUUUUGGGAGGAGGGAUUUGGCGAACUG
UUCGAGAAGGCCAAGCAGAACAACAACAACCGCAAGACCUCGAACGGUGACGACUCC
CUCUUCUUUUCAAACUUCAGCCUGCUCGGGACGCCCGUGCUGAAGGACAUUAACUUC
AAGAUCGAAAGAGGACAGCUCCUGGCGGUGGCCGGAUCGACCGGAGCCGGAAAGACU
UCCCUGCUGAUGGUGAUCAUGGGAGAGCUUGAACCUAGCGAGGGAAAGAUCAAGCA
CUCCGGCCGCAUCAGCUUCUGUAGCCAGUUUUCCUGGAUCAUGCCCGGAACCAUUAA
GGAAAACAUCAUCUUCGGCGUGUCCUACGAUGAAUACCGCUACCGGUCCGUGAUCAA
AGCCUGCCAGCUGGAAGAGGAUAUUUCAAAGUUCGCGGAGAAAGAUAACAUCGUGC
UGGGCGAAGGGGGUAUUACCUUGUCGGGGGGCCAGCGGGCUAGAAUCUCGCUGGCCA
GAGCCGUGUAUAAGGACGCCGACCUGUAUCUCCUGGACUCCCCCUUCGGAUACCUGG
ACGUCCUGACCGAAAAGGAGAUCUUCGAAUCGUGCGUGUGCAAGCUGAUGGCUAACA
AGACUCGCAUCCUCGUGACCUCCAAAAUGGAGCACCUGAAGAAGGCAGACAAGAUUC
UGAUUCUGCAUGAGGGGUCCUCCUACUUUUACGGCACCUUCUCGGAGUUGCAGAACU
UGCAGCCCGACUUCUCAUCGAAGCUGAUGGGUUGCGACAGCUUCGACCAGUUCUCCG
CCGAAAGAAGGAACUCGAUCCUGACGGAAACCUUGCACCGCUUCUCUUUGGAAGGCG
ACGCCCCUGUGUCAUGGACCGAGACUAAGAAGCAGAGCUUCAAGCAGACCGGGGAAU
UCGGCGAAAAGAGGAAGAACAGCAUCUUGAACCCCAUUAACUCCAUCCGCAAGUUCU
CAAUCGUGCAAAAGACGCCACUGCAGAUGAACGGCAUUGAGGAGGACUCCGACGAAC
CCCUUGAGAGGCGCCUGUCCCUGGUGCCGGACAGCGAGCAGGGAGAAGCCAUCCUGC
CUCGGAUUUCCGUGAUCUCCACUGGUCCGACGCUCCAAGCCCGGCGGCGGCAGUCCG
UGCUGAACCUGAUGACCCACAGCGUGAACCAGGGCCAAAACAUUCACCGCAAGACUA
CCGCAUCCACCCGGAAAGUGUCCCUGGCACCUCAAGCGAAUCUUACCGAGCUCGACA
UCUACUCCCGGAGACUGUCGCAGGAAACCGGGCUCGAAAUUUCCGAAGAAAUCAACG
AGGAGGAUCUGAAAGAGUGCUUCUUCGACGAUAUGGAGUCGAUACCCGCCGUGACG
ACUUGGAACACUUAUCUGCGGUACAUCACUGUGCACAAGUCAUUGAUCUUCGUGCUG
AUUUGGUGCCUGGUGAUUUUCCUGGCCGAGGUCGCGGCCUCACUGGUGGUGCUCUGG
CUGUUGGGAAACACGCCUCUGCAAGACAAGGGAAACUCCACGCACUCGAGAAACAAC
AGCUAUGCCGUGAUUAUCACUUCCACCUCCUCUUAUUACGUGUUCUACAUCUACGUC
GGAGUGGCGGAUACCCUGCUCGCGAUGGGUUUCUUCAGAGGACUGCCGCUGGUCCAC
ACCUUGAUCACCGUCAGCAAGAUUCUUCACCACAAGAUGUUGCAUAGCGUGCUGCAG
GCCCCCAUGUCCACCCUCAACACUCUGAAGGCCGGAGGCAUUCUGAACAGAUUCUCC
AAGGACAUCGCUAUCCUGGACGAUCUCCUGCCGCUUACCAUCUUUGACUUCAUCCAG
CUGCUGCUGAUCGUGAUUGGAGCAAUCGCAGUGGUGGCGGUGCUGCAGCCUUACAUU
UUCGUGGCCACUGUGCCGGUCAUUGUGGCGUUCAUCAUGCUGCGGGCCUACUUCCUC
CAAACCAGCCAGCAGCUGAAGCAACUGGAAUCCGAGGGACGAUCCCCCAUCUUCACU
CACCUUGUGACGUCGUUGAAGGGACUGUGGACCCUCCGGGCUUUCGGACGGCAGCCC
UACUUCGAAACCCUCUUCCACAAGGCCCUGAACCUCCACACCGCCAAUUGGUUCCUG
UACCUGUCCACCCUGCGGUGGUUCCAGAUGCGCAUCGAGAUGAUUUUCGUCAUCUUC
UUCAUCGCGGUCACAUUCAUCAGCAUCCUGACUACCGGAGAGGGAGAGGGACGGGUC
GGAAUAAUCCUGACCCUCGCCAUGAACAUUAUGAGCACCCUGCAGUGGGCAGUGAAC
AGCUCGAUCGACGUGGACAGCCUGAUGCGAAGCGUCAGCCGCGUGUUCAAGUUCAUC
GACAUGCCUACUGAGGGAAAACCCACUAAGUCCACUAAGCCCUACAAAAAUGGCCAG
CUGAGCAAGGUCAUGAUCAUCGAAAACUCCCACGUGAAGAAGGACGAUAUUUGGCCC
UCCGGAGGUCAAAUGACCGUGAAGGACCUGACCGCAAAGUACACCGAGGGAGGAAAC
GCCAUUCUCGAAAACAUCAGCUUCUCCAUUUCGCCGGGACAGCGGGUCGGCCUUCUC
GGGCGGACCGGUUCCGGGAAGUCAACUCUGCUGUCGGCUUUCCUCCGGCUGCUGAAU
ACCGAGGGGGAAAUCCAAAUUGACGGCGUGUCUUGGGAUUCCAUUACUCUGCAGCAG
UGGCGGAAGGCCUUCGGCGUGAUCCCCCAGAAGGUGUUCAUCUUCUCGGGUACCUUC
CGGAAGAACCUGGAUCCUUACGAGCAGUGGAGCGACCAAGAAAUCUGGAAGGUCGCC
GACGAGGUCGGCCUGCGCUCCGUGAUUGAACAAUUUCCUGGAAAGCUGGACUUCGUG
CUCGUCGACGGGGGAUGUGUCCUGUCGCACGGACAUAAGCAGCUCAUGUGCCUCGCA
CGGUCCGUGCUCUCCAAGGCCAAGAUUCUGCUGCUGGACGAACCUUCGGCCCACCUG
GAUCCGGUCACCUACCAGAUCAUCAGGAGGACCCUGAAGCAGGCCUUUGCCGAUUGC
ACCGUGAUUCUCUGCGAGCACCGCAUCGAGGCCAUGCUGGAGUGCCAGCAGUUCCUG
GUCAUCGAGGAGAACAAGGUCCGCCAAUACGACUCCAUUCAAAAGCUCCUCAACGAG
CGGUCGCUGUUCAGACAAGCUAUUUCACCGUCCGAUAGAGUGAAGCUCUUCCCGCAU
CGGAACAGCUCAAAGUGCAAAUCGAAGCCGCAGAUCGCAGCCUUGAAGGAAGAGACU
GAGGAAGAGGUGCAGGACACCCGGCUUUAACGGGUGGCAUCCCUGUGACCCCUCCCC
AGUGCCUCUCCUGGCCCUGGAAGUUGCCACUCCAGUGCCCACCAGCCUUGUCCUAAU
AAAAUUAAGUUGCAUCAAGCU

In another embodiment, an exemplary full-length codon-optimized CFTR mRNA sequence is:

(SEQ ID NO: 7)
GGACAGAUCGCCUGGAGACGCCAUCCACGCUGUUUUGACCUCCAUAGAAGACACCGG
GACCGAUCCAGCCUCCGCGGCCGGGAACGGUGCAUUGGAACGCGGAUUCCCCGUGCC
AAGAGUGACUCACCGUCCUUGACACGAUGCAACGCUCUCCUCUUGAAAAGGCCUCGG
UGGUGUCCAAGCUCUUCUUCUCGUGGACUAGACCCAUCCUGAGAAAGGGGUACAGAC
AGCGCUUGGAGCUGUCCGAUAUCUAUCAAAUCCCUUCCGUGGACUCCGCGGACAACC
UGUCCGAGAAGCUCGAGAGAGAAUGGGACAGAGAACUCGCCUCAAAGAAGAACCCGA
AGCUGAUUAAUGCGCUUAGGCGGUGCUUUUUCUGGCGGUUCAUGUUCUACGGCAUC
UUCCUCUACCUGGGAGAGGUCACCAAGGCCGUGCAGCCCCUGUUGCUGGGACGGAUU
AUUGCCUCCUACGACCCCGACAACAAGGAAGAAAGAAGCAUCGCUAUCUACUUGGGC
AUCGGUCUGUGCCUGCUUUUCAUCGUCCGGACCCUCUUGUUGCAUCCUGCUAUUUUC
GGCCUGCAUCACAUUGGCAUGCAGAUGAGAAUUGCCAUGUUUUCCCUGAUCUACAAG
AAAACUCUGAAGCUCUCGAGCCGCGUGCUUGACAAGAUUUCCAUCGGCCAGCUCGUG
UCCCUGCUCUCCAACAAUCUGAACAAGUUCGACGAGGGCCUCGCCCUGGCCCACUUC
GUGUGGAUCGCCCCUCUGCAAGUGGCGCUUCUGAUGGGCCUGAUCUGGGAGCUGCUG
CAAGCCUCGGCAUUCUGUGGGCUUGGAUUCCUGAUCGUGCUGGCACUGUUCCAGGCC
GGACUGGGGCGGAUGAUGAUGAAGUACAGGGACCAGAGAGCCGGAAAGAUUUCCGA
ACGGCUGGUGAUCACUUCGGAAAUGAUCGAAAACAUCCAGUCAGUGAAGGCCUACUG
CUGGGAAGAGGCCAUGGAAAAGAUGAUUGAAAACCUCCGGCAAACCGAGCUGAAGC
UGACCCGCAAGGCCGCUUACGUGCGCUAUUUCAACUCGUCCGCUUUCUUCUUCUCCG
GGUUCUUCGUGGUGUUUCUCUCCGUGCUCCCCUACGCCCUGAUUAAGGGAAUCAUCC
UCAGGAAGAUCUUCACCACCAUUUCCUUCUGUAUCGUGCUCCGCAUGGCCGUGACCC
GGCAGUUCCCAUGGGCCGUGCAGACUUGGUACGACUCCCUGGGAGCCAUUAACAAGA
UCCAGGACUUCCUUCAAAAGCAGGAGUACAAGACCCUCGAGUACAACCUGACUACUA
CCGAGGUCGUGAUGGAAAACGUCACCGCCUUUUGGGAGGAGGGAUUUGGCGAACUG
UUCGAGAAGGCCAAGCAGAACAACAACAACCGCAAGACCUCGAACGGUGACGACUCC
CUCUUCUUUUCAAACUUCAGCCUGCUCGGGACGCCCGUGCUGAAGGACAUUAACUUC
AAGAUCGAAAGAGGACAGCUCCUGGCGGUGGCCGGAUCGACCGGAGCCGGAAAGACU
UCCCUGCUGAUGGUGAUCAUGGGAGAGCUUGAACCUAGCGAGGGAAAGAUCAAGCA
CUCCGGCCGCAUCAGCUUCUGUAGCCAGUUUUCCUGGAUCAUGCCCGGAACCAUUAA
GGAAAACAUCAUCUUCGGCGUGUCCUACGAUGAAUACCGCUACCGGUCCGUGAUCAA
AGCCUGCCAGCUGGAAGAGGAUAUUUCAAAGUUCGCGGAGAAAGAUAACAUCGUGC
UGGGCGAAGGGGGUAUUACCUUGUCGGGGGGCCAGCGGGCUAGAAUCUCGCUGGCCA
GAGCCGUGUAUAAGGACGCCGACCUGUAUCUCCUGGACUCCCCCUUCGGAUACCUGG
ACGUCCUGACCGAAAAGGAGAUCUUCGAAUCGUGCGUGUGCAAGCUGAUGGCUAACA
AGACUCGCAUCCUCGUGACCUCCAAAAUGGAGCACCUGAAGAAGGCAGACAAGAUUC
UGAUUCUGCAUGAGGGGUCCUCCUACUUUUACGGCACCUUCUCGGAGUUGCAGAACU
UGCAGCCCGACUUCUCAUCGAAGCUGAUGGGUUGCGACAGCUUCGACCAGUUCUCCG
CCGAAAGAAGGAACUCGAUCCUGACGGAAACCUUGCACCGCUUCUCUUUGGAAGGCG
ACGCCCCUGUGUCAUGGACCGAGACUAAGAAGCAGAGCUUCAAGCAGACCGGGGAAU
UCGGCGAAAAGAGGAAGAACAGCAUCUUGAACCCCAUUAACUCCAUCCGCAAGUUCU
CAAUCGUGCAAAAGACGCCACUGCAGAUGAACGGCAUUGAGGAGGACUCCGACGAAC
CCCUUGAGAGGCGCCUGUCCCUGGUGCCGGACAGCGAGCAGGGAGAAGCCAUCCUGC
CUCGGAUUUCCGUGAUCUCCACUGGUCCGACGCUCCAAGCCCGGCGGCGGCAGUCCG
UGCUGAACCUGAUGACCCACAGCGUGAACCAGGGCCAAAACAUUCACCGCAAGACUA
CCGCAUCCACCCGGAAAGUGUCCCUGGCACCUCAAGCGAAUCUUACCGAGCUCGACA
UCUACUCCCGGAGACUGUCGCAGGAAACCGGGCUCGAAAUUUCCGAAGAAAUCAACG
AGGAGGAUCUGAAAGAGUGCUUCUUCGACGAUAUGGAGUCGAUACCCGCCGUGACG
ACUUGGAACACUUAUCUGCGGUACAUCACUGUGCACAAGUCAUUGAUCUUCGUGCUG
AUUUGGUGCCUGGUGAUUUUCCUGGCCGAGGUCGCGGCCUCACUGGUGGUGCUCUGG
CUGUUGGGAAACACGCCUCUGCAAGACAAGGGAAACUCCACGCACUCGAGAAACAAC
AGCUAUGCCGUGAUUAUCACUUCCACCUCCUCUUAUUACGUGUUCUACAUCUACGUC
GGAGUGGCGGAUACCCUGCUCGCGAUGGGUUUCUUCAGAGGACUGCCGCUGGUCCAC
ACCUUGAUCACCGUCAGCAAGAUUCUUCACCACAAGAUGUUGCAUAGCGUGCUGCAG
GCCCCCAUGUCCACCCUCAACACUCUGAAGGCCGGAGGCAUUCUGAACAGAUUCUCC
AAGGACAUCGCUAUCCUGGACGAUCUCCUGCCGCUUACCAUCUUUGACUUCAUCCAG
CUGCUGCUGAUCGUGAUUGGAGCAAUCGCAGUGGUGGCGGUGCUGCAGCCUUACAUU
UUCGUGGCCACUGUGCCGGUCAUUGUGGCGUUCAUCAUGCUGCGGGCCUACUUCCUC
CAAACCAGCCAGCAGCUGAAGCAACUGGAAUCCGAGGGACGAUCCCCCAUCUUCACU
CACCUUGUGACGUCGUUGAAGGGACUGUGGACCCUCCGGGCUUUCGGACGGCAGCCC
UACUUCGAAACCCUCUUCCACAAGGCCCUGAACCUCCACACCGCCAAUUGGUUCCUG
UACCUGUCCACCCUGCGGUGGUUCCAGAUGCGCAUCGAGAUGAUUUUCGUCAUCUUC
UUCAUCGCGGUCACAUUCAUCAGCAUCCUGACUACCGGAGAGGGAGAGGGACGGGUC
GGAAUAAUCCUGACCCUCGCCAUGAACAUUAUGAGCACCCUGCAGUGGGCAGUGAAC
AGCUCGAUCGACGUGGACAGCCUGAUGCGAAGCGUCAGCCGCGUGUUCAAGUUCAUC
GACAUGCCUACUGAGGGAAAACCCACUAAGUCCACUAAGCCCUACAAAAAUGGCCAG
CUGAGCAAGGUCAUGAUCAUCGAAAACUCCCACGUGAAGAAGGACGAUAUUUGGCCC
UCCGGAGGUCAAAUGACCGUGAAGGACCUGACCGCAAAGUACACCGAGGGAGGAAAC
GCCAUUCUCGAAAACAUCAGCUUCUCCAUUUCGCCGGGACAGCGGGUCGGCCUUCUC
GGGCGGACCGGUUCCGGGAAGUCAACUCUGCUGUCGGCUUUCCUCCGGCUGCUGAAU
ACCGAGGGGGAAAUCCAAAUUGACGGCGUGUCUUGGGAUUCCAUUACUCUGCAGCAG
UGGCGGAAGGCCUUCGGCGUGAUCCCCCAGAAGGUGUUCAUCUUCUCGGGUACCUUC
CGGAAGAACCUGGAUCCUUACGAGCAGUGGAGCGACCAAGAAAUCUGGAAGGUCGCC
GACGAGGUCGGCCUGCGCUCCGUGAUUGAACAAUUUCCUGGAAAGCUGGACUUCGUG
CUCGUCGACGGGGGAUGUGUCCUGUCGCACGGACAUAAGCAGCUCAUGUGCCUCGCA
CGGUCCGUGCUCUCCAAGGCCAAGAUUCUGCUGCUGGACGAACCUUCGGCCCACCUG
GAUCCGGUCACCUACCAGAUCAUCAGGAGGACCCUGAAGCAGGCCUUUGCCGAUUGC
ACCGUGAUUCUCUGCGAGCACCGCAUCGAGGCCAUGCUGGAGUGCCAGCAGUUCCUG
GUCAUCGAGGAGAACAAGGUCCGCCAAUACGACUCCAUUCAAAAGCUCCUCAACGAG
CGGUCGCUGUUCAGACAAGCUAUUUCACCGUCCGAUAGAGUGAAGCUCUUCCCGCAU
CGGAACAGCUCAAAGUGCAAAUCGAAGCCGCAGAUCGCAGCCUUGAAGGAAGAGACU
GAGGAAGAGGUGCAGGACACCCGGCUUUAAGGGUGGCAUCCCUGUGACCCCUCCCCA
GUGCCUCUCCUGGCCCUGGAAGUUGCCACUCCAGUGCCCACCAGCCUUGUCCUAAUA
AAAUUAAGUUGCAUCAAAGCU

(SEQ ID NO: 8)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCAATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTGGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACTCACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA

(SEQ ID NO: 9)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCTGATATCTACCAGA
TTCCTTCTGTGGACTCAGCTGACAATTTGAGTGAGAAGCTGGAGCGGGAGTGGGATAGA
GAGCTGGCGAGCAAAAAAAACCCCAAGCTTATCAATGCTCTGCGCCGCTGCTTTTTCTG
GAGGTTCATGTTTTATGGGATCTTCCTGTACCTGGGGGAGGTCACCAAAGCTGTTCAGC
CGCTCCTTCTTGGCCGCATCATCGCCAGCTATGACCCTGATAATAAAGAAGAAAGGTCT
ATTGCTATTTATCTGGGAATTGGCCTCTGCTTGCTCTTCATCGTCCGCACCCTTCTGCTGC
ACCCTGCCATTTTTGGCCTTCACCACATCGGCATGCAAATGAGAATTGCCATGTTCTCCC
TCATTTACAAAAAGACCCTGAAACTTTCCTCAAGAGTGTTAGATAAAATATCCATTGGT
CAGCTGGTCAGCCTGCTGTCCAACAATCTTAACAAATTTGATGAAGGCTTGGCGCTGGC
CCACTTCGTGTGGATTGCACCTCTGCAGGTGGCCCTGTTGATGGGACTTATATGGGAGC
TGCTTCAAGCCTCTGCTTTCTGTGGGCTGGGCTTTTTGATTGTACTGGCACTTTTTCAGGC
TGGGCTCGGAAGAATGATGATGAAATACAGAGATCAGCGGGCCGGGAAGATTTCAGAG
CGACTTGTGATCACCAGTGAAATGATTGAAAATATTCAGAGCGTGAAAGCCTACTGCTG
GGAAGAAGCCATGGAGAAGATGATTGAGAACCTGAGGCAGACAGAGCTCAAGCTCACT
CGGAAGGCTGCTTATGTTCGCTATTTCAACAGCAGCGCCTTCTTCTTCAGTGGCTTCTTT
GTTGTCTTCCTGTCTGTTCTGCCATATGCACTGATAAAAGGCATTATTTTACGAAAGATC
TTCACCACCATCAGTTTTTGCATCGTTCTCAGGATGGCCGTCACAAGACAGTTCCCCTGG
GCTGTGCAGACCTGGTACGATTCCTTGGGGGCCATCAACAAGATTCAAGATTTCTTGCA
AAAACAAGAATATAAAACTTTAGAATACAACCTCACCACCACTGAAGTGGTCATGGAA
AATGTGACAGCCTTTTGGGAGGAGGGTTTTGGAGAATTGTTCGAGAAGGCAAAGCAGA
ATAACAACAACAGGAAGACGAGCAATGGGGACGACTCTCTCTTCTTCAGCAACTTTTCA
CTGCTCGGGACCCCTGTGTTGAAAGATATAAACTTCAAGATCGAGAGGGGCCAGCTCTT
GGCTGTGGCAGGCTCCACTGGAGCTGGTAAAACATCTCTTCTCATGGTGATCATGGGGG
AACTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTATG
ATGAGTACCGCTACCGGTCAGTCATCAAAGCCTGTCAGTTGGAGGAGGACATCTCCAAG
TTTGCAGAGAAAGACAACATTGTGCTTGGAGAGGGGGGTATCACTCTTTCTGGAGGACA
AAGAGCCAGGATCTCTTTGGCCCGGGCAGTCTACAAGGATGCAGACCTCTACTTGTTGG
ACAGTCCCTTCGGCTACCTCGACGTGCTGACTGAAAAAGAAATTTTTGAAAGCTGTGTG
TGCAAACTGATGGCAAACAAGACCAGGATTCTTGTCACCAGCAAGATGGAACATCTGA
AGAAAGCGGACAAAATTCTGATTCTGCATGAAGGGAGCTCCTACTTCTATGGAACATTT
AGCGAGCTTCAGAACCTACAGCCAGACTTCTCCTCCAAATTAATGGGCTGTGACTCCTT
CGACCAGTTCTCTGCAGAAAGAAGAAACTCTATACTCACAGAGACCCTCCACCGCTTCT
CCCTTGAGGGAGATGCCCCAGTTTCTTGGACAGAAACCAAGAAGCAGTCCTTTAAGCAG
ACTGGCGAGTTTGGTGAAAAGAGGAAAAATTCAATTCTCAATCCAATTAACAGTATTCG
CAAGTTCAGCATTGTCCAGAAGACACCCCTCCAGATGAATGGCATCGAAGAAGATAGT
GACGAGCCGCTGGAGAGACGGCTGAGTCTGGTGCCAGATTCAGAACAGGGGGAGGCCA
TCCTGCCCCGGATCAGCGTCATTTCCACAGGCCCCACATTACAAGCACGGCGCCGGCAG
AGTGTTTTAAATCTCATGACCCATTCAGTGAACCAGGGCCAAAATATCCACAGGAAGAC
TACAGCTTCTACCCGGAAAGTGTCTCTGGCCCCTCAGGCCAATCTGACCGAGCTGGACA
TCTACAGCAGGAGGCTCTCCCAGGAAACAGGGCTGGAAATATCTGAAGAGATTAATGA
AGAGGATCTTAAAGAGTGCTTCTTTGATGACATGGAGAGCATCCCCGCGGTGACCACAT
GGAACACCTACCTTAGATATATTACTGTCCACAAGAGCCTCATATTTGTCCTCATCTGGT
GCCTGGTTATTTTCCTCGCTGAGGTGGCGGCCAGTCTTGTTGTGCTCTGGCTGCTGGGCA
ACACTCCTCTCCAGGACAAGGGCAATAGTACTCACAGCAGAAATAATTCTTATGCCGTC
ATCATTACAAGCACCTCCAGCTACTACGTGTTCTACATCTATGTGGGCGTGGCTGACAC
CCTCCTGGCCATGGGTTTCTTCCGGGGCCTGCCTTTGGTGCACACCCTCATCACAGTGTC
AAAAATTCTGCACCATAAAATGCTTCATTCTGTCCTGCAGGCACCCATGAGCACTTTGA
ACACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAGCC
ATAGCAGTGGTTGCTGTCCTGCAGCCCTACATTTTTGTGGCCACCGTGCCCGTGATTGTT
GCCTTTATTATGCTCAGAGCTTACTTCCTGCAAACTTCTCAACAGCTCAAACAGCTAGAA
TCTGAGGGCCGGAGCCCCATTTTTACCCACCTGGTGACTTCCCTGAAGGGACTGTGGAC
TCTGAGAGCATTCGGGCGACAGCCTTACTTTGAGACACTGTTCCACAAGGCCCTGAACT
TGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTTGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA

In yet another embodiment, an exemplary codon-optimized CFTR mRNA sequence is:

(SEQ ID NO: 10)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCAATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTTGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACACACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTTGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

In yet another embodiment, an exemplary codon-optimized CFTR mRNA sequence is:

(SEQ ID NO: 11)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCAATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTGGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACTCACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCAACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

In yet another embodiment, an exemplary codon-optimized CFTR mRNA sequence is:

(SEQ ID NO: 12)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCTATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTTGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACTCACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

(SEQ ID NO: 13)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATTTCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCAATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTGGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACTCACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCAACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

In another embodiment, an exemplary codon-optimized CFTR mRNA sequence is:

(SEQ ID NO: 14)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCTGATATCTACCAGA
TTCCTTCTGTGGACTCAGCTGACAATTTGAGTGAGAAGCTGGAGCGGGAGTGGGATAGA
GAGCTGGCGAGCAAAAAAAACCCCAAGCTTATCAATGCTCTGCGCCGCTGCTTTTTCTG
GAGGTTCATGTTTTATGGGATCTTCCTGTACCTGGGGGAGGTCACCAAAGCTGTTCAGC
CGCTCCTTCTTGGCCGCATCATCGCCAGCTATGACCCTGATAATAAAGAAGAAAGGTCT
ATTGCTATTTATCTGGGAATTGGCCTCTGCTTGCTCTTCATCGTCCGCACCCTTCTGCTGC
ACCCTGCCATTTTTGGCCTTCACCACATCGGCATGCAAATGAGAATTGCCATGTTCTCCC
TCATTTACAAAAAGACCCTGAAACTTTCCTCAAGAGTGTTAGATAAAATATCCATTGGT
CAGCTGGTCAGCCTGCTGTCCAACAATCTTAACAAATTTGATGAAGGCTTGGCGCTGGC
CCACTTCGTGTGGATTGCACCTCTGCAGGTGGCCCTGTTGATGGGACTTATATGGGAGC
TGCTTCAAGCCTCTGCTTTCTGTGGGCTGGGCTTTTTGATTGTACTGGCACTTTTTCAGGC
TGGGCTCGGAAGAATGATGATGAAATACAGAGATCAGCGGGCCGGGAAGATATCAGAG
CGACTTGTGATCACCAGTGAAATGATTGAAAATATTCAGAGCGTGAAAGCCTACTGCTG
GGAAGAAGCCATGGAGAAGATGATTGAGAACCTGAGGCAGACAGAGCTCAAGCTCACT
CGGAAGGCTGCTTATGTTCGCTATTTCAACAGCAGCGCCTTCTTCTTCAGTGGCTTCTTT
GTTGTCTTCCTGTCTGTTCTGCCATATGCACTGATAAAAGGCATTATTTTACGAAAGATC
TTCACCACCATCAGTTTTTGCATCGTTCTCAGGATGGCCGTCACAAGACAGTTCCCCTGG
GCTGTGCAGACCTGGTACGATTCCTTGGGGGCCATCAACAAGATTCAAGATTTCTTGCA
AAAACAAGAATATAAAACTTTAGAATACAACCTCACCACCACTGAAGTGGTCATGGAA
AATGTGACAGCCTTTTGGGAGGAGGGTTTTGGAGAATTGTTCGAGAAGGCAAAGCAGA
ATAACAACAACAGGAAGACGAGCAATGGGGACGACTCTCTCTTCTTCAGCAACTTTTCA
CTGCTCGGGACCCCTGTGTTGAAAGATATAAACTTCAAGATCGAGAGGGGCCAGCTCTT
GGCTGTGGCAGGCTCCACTGGAGCTGGTAAAACATCTCTTCTCATGGTGATCATGGGGG
AACTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTATG
ATGAGTACCGCTACCGGTCCGTCATCAAAGCCTGTCAGTTGGAGGAGGACATCTCCAAG
TTTGCAGAGAAAGACAACATTGTGCTTGGAGAGGGGGGTATCACTCTTTCTGGAGGACA
AAGAGCCAGGATCTCTTTGGCCCGGGCAGTCTACAAGGATGCAGACCTCTACTTGTTGG
ACAGTCCCTTCGGCTACCTCGACGTGCTGACTGAAAAAGAAATTTTTGAAAGCTGTGTG
TGCAAACTGATGGCAAACAAGACCAGGATTCTTGTCACCAGCAAGATGGAACATCTGA
AGAAAGCGGACAAAATTCTGATTCTGCATGAAGGGAGCTCCTACTTCTATGGAACATTT
AGCGAGCTTCAGAACCTACAGCCAGACTTCTCCTCCAAATTAATGGGCTGTGACTCCTT
CGACCAGTTCTCTGCAGAAAGAAGAAACTCTATACTCACAGAGACCCTCCACCGCTTCT
CCCTTGAGGGAGATGCCCCAGTTTCTTGGACAGAAACCAAGAAGCAGTCCTTTAAGCAG
ACTGGCGAGTTTGGTGAAAAGAGGAAAAATTCAATTCTCAATCCAATTAACAGTATTCG
CAAGTTCAGCATTGTCCAGAAGACACCCCTCCAGATGAATGGCATCGAAGAAGATAGT
GACGAGCCGCTGGAGAGACGGCTGAGTCTGGTGCCAGATTCAGAACAGGGGGAGGCCA
TCCTGCCCCGGATCAGCGTCATTTCCACAGGCCCCACATTACAAGCACGGCGCCGGCAG
AGTGTTTTAAATCTCATGACCCATTCAGTGAACCAGGGCCAAAATATCCACAGGAAGAC
TACAGCTTCTACCCGGAAAGTGTCTCTGGCCCCTCAGGCCAATCTGACCGAGCTGGACA
TCTACAGCAGGAGGCTCTCCCAGGAAACAGGGCTTGAAATATCTGAAGAGATTAATGA
AGAGGATCTTAAAGAGTGCTTCTTTGATGACATGGAGAGCATCCCCGCGGTGACCACAT
GGAACACCTACCTTAGATATATTACTGTCCACAAGAGCCTCATATTTGTCCTCATCTGGT
GCCTGGTTATTTTCCTCGCTGAGGTGGCGGCCAGTCTTGTTGTGCTCTGGCTGCTGGGCA
ACACTCCTCTCCAGGACAAGGGCAATAGTACTCACAGCAGAAATAATTCTTATGCCGTC
ATCATTACAAGCACCTCCAGCTACTACGTGTTCTACATCTATGTGGGCGTGGCTGACAC
CCTCCTGGCCATGGGTTTCTTCCGGGGCCTGCCTTTGGTGCACACCCTCATCACAGTGTC
AAAAATTCTGCACCATAAAATGCTTCATTCTGTCCTGCAGGCACCCATGAGCACTTTGA
ACACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAGCC
ATAGCAGTGGTTGCTGTCCTGCAGCCCTACATTTTTGTGGCCACCGTGCCCGTGATTGTT
GCCTTTATTATGCTCAGAGCTTACTTCCTGCAAACTTCTCAACAGCTCAAACAGCTAGAA
TCTGAGGGCCGGAGCCCCATTTTTACCCACCTGGTGACTTCCCTGAAGGGACTGTGGAC
TCTGAGAGCATTCGGGCGACAGCCTTACTTTGAGACACTGTTCCACAAGGCCCTGAACT
TGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAGCCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

(SEQ ID NO: 15)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCTATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTTGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACACACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

In yet another embodiment, an exemplary codon-optimized CFTR mRNA sequence is:

(SEQ ID NO: 16)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCCGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GAC TGGCGAGTTTGGTGAAAAGAGGAAAAATTCAATTCTCAATCCAATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTGGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACTCACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
GTCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

In another embodiment, an exemplary codon-optimized CFTR mRNA sequence is:

(SEQ ID NO: 17)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCTGATATCTACCAGA
TTCCTTCTGTGGACTCAGCTGACAATTTGAGTGAGAAGCTGGAGCGGGAGTGGGATAGA
GAGCTGGCGAGCAAAAAAAACCCCAAGCTTATCAATGCTCTGCGCCGCTGCTTTTTCTG
GAGGTTCATGTTTTATGGGATCTTCCTGTACCTGGGGGAGGTCACCAAAGCTGTTCAGC
CGCTCCTTCTTGGCCGCATCATCGCCAGCTATGACCCTGATAATAAAGAAGAAAGGTCT
ATTGCTATTTATCTGGGAATTGGCCTCTGCTTGCTCTTCATCGTCCGCACCCTTCTGCTGC
ACCCTGCCATTTTTGGCCTTCACCACATCGGCATGCAAATGAGAATTGCCATGTTCTCCC
TCATTTACAAAAAGACCCTGAAACTTTCCTCAAGAGTGTTAGATAAAATATCCATTGGT
CAGCTGGTCAGCCTGCTGTCCAACAATCTTAACAAATTTGATGAAGGCTTGGCGCTGGC
CCACTTCGTGTGGATTGCACCTCTGCAGGTGGCCCTGTTGATGGGACTTATATGGGAGC
TGCTTCAAGCCTCTGCTTTCTGTGGGCTGGGCTTTTTGATTGTACTGGCACTTTTTCAGGC
TGGGCTCGGAAGAATGATGATGAAATACAGAGATCAGCGGGCCGGGAAGATTTCAGAG
CGACTTGTGATCACCAGTGAAATGATTGAAAATATTCAGAGCGTGAAAGCCTACTGCTG
GGAAGAAGCCATGGAGAAGATGATTGAGAACCTGAGGCAGACAGAGCTCAAGCTCACT
CGGAAGGCTGCTTATGTTCGCTATTTCAACAGCAGCGCCTTCTTCTTCAGTGGCTTCTTT
GTTGTCTTCCTGTCTGTTCTGCCATATGCACTGATAAAAGGCATTATTTTACGAAAGATC
TTCACCACCATCAGTTTTTGCATCGTTCTCAGGATGGCCGTCACAAGACAGTTCCCCTGG
GCTGTGCAGACCTGGTACGATTCCTTGGGGGCCATCAACAAGATTCAAGATTTCTTGCA
AAAACAAGAATATAAAACTTTAGAATACAACCTCACCACCACTGAAGTGGTCATGGAA
AATGTGACAGCCTTTTGGGAGGAGGGTTTTGGAGAATTGTTCGAGAAGGCAAAGCAGA
ATAACAACAACAGGAAGACGAGCAATGGGGACGACTCTCTCTTCTTCAGCAACTTTTCA
CTGCTCGGGACCCCTGTGTTGAAAGATATAAACTTCAAGATCGAGAGGGGCCAGCTCTT
GGCTGTGGCAGGCTCCACTGGAGCTGGTAAAACATCTCTTCTCATGGTGATCATGGGGG
AACTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTATG
ATGAGTACCGCTACCGGTCAGTCATCAAAGCCTGTCAGTTGGAGGAGGACATCTCCAAG
TTTGCAGAGAAAGACAACATTGTGCTTGGAGAGGGGGGTATCACTCTTTCTGGAGGACA
AAGAGCCAGGATCTCTTTGGCCCGGGCAGTCTACAAGGATGCAGACCTCTACTTGTTGG
ACAGTCCCTTCGGCTACCTCGACGTGCTGACTGAAAAAGAAATTTTTGAAAGCTGTGTG
TGCAAACTGATGGCAAACAAGACCAGGATTCTTGTCACCAGCAAGATGGAACATCTGA
AGAAAGCGGACAAAATTCTGATTCTGCATGAAGGGAGCTCCTACTTCTATGGAACATTT
AGCGAGCTTCAGAACCTACAGCCAGACTTCTCCTCCAAATTAATGGGCTGTGACTCCTT
CGACCAGTTCTCTGCAGAAAGAAGAAACTCTATACTCACAGAGACCCTCCACCGCTTCT
CCCTTGAGGGAGATGCCCCAGTTTCTTGGACAGAAACCAAGAAGCAGTCCTTTAAGCAG
ACTGGCGAGTTTGGTGAAAAGAGGAAAAATTCAATTCTCAATCCTATTAACAGTATTCG
CAAGTTCAGCATTGTCCAGAAGACACCCCTCCAGATGAATGGCATCGAAGAAGATAGT
GACGAGCCGCTGGAGAGACGGCTGAGTCTGGTGCCAGATTCAGAACAGGGGGAGGCCA
TCCTGCCCCGGATCAGCGTCATTTCCACAGGCCCCACATTACAAGCACGGCGCCGGCAG
AGTGTTTTAAATCTCATGACCCATTCAGTGAACCAGGGCCAAAATATCCACAGGAAGAC
TACAGCTTCTACCCGGAAAGTGTCTCTGGCCCCTCAGGCCAATCTGACCGAGCTGGACA
TCTACAGCAGGAGGCTCTCCCAGGAAACAGGGCTGGAAATATCTGAAGAGATTAATGA
AGAGGATCTTAAAGAGTGCTTCTTTGATGACATGGAGAGCATCCCCGCGGTGACCACAT
GGAACACCTACCTTAGATATATTACTGTCCACAAGAGCCTCATATTTGTCCTCATCTGGT
GCCTGGTTATTTTCCTCGCTGAGGTGGCGGCCAGTCTTGTTGTGCTCTGGCTGCTGGGCA
ACACTCCTCTCCAGGACAAGGGCAATAGTACACACAGCAGAAATAATTCTTATGCCGTC
ATCATTACAAGCACCTCCAGCTACTACGTGTTCTACATCTATGTGGGCGTGGCTGACAC
CCTCCTGGCCATGGGTTTCTTCCGGGGCCTGCCTTTGGTGCACACCCTCATCACAGTGTC
AAAAATTCTGCACCATAAAATGCTTCATTCTGTCCTGCAGGCACCCATGAGCACTTTGA
ACACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAGCC
ATAGCAGTGGTTGCTGTCCTGCAGCCCTACATTTTTGTGGCCACCGTGCCCGTGATTGTT
GCCTTTATTATGCTCAGAGCTTACTTCCTGCAAACTTCTCAACAGCTCAAACAGCTAGAA
TCTGAGGGCCGGAGCCCCATTTTTACCCACCTGGTGACTTCCCTGAAGGGACTGTGGAC
TCTGAGAGCATTCGGGCGACAGCCTTACTTTGAGACACTGTTCCACAAGGCCCTGAACT
TGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

In another embodiment, an exemplary codon-optimized CFTR mRNA sequence is:

(SEQ ID NO: 18)
ATGCAGAGAAGCCCCCTGGAGAAGGCCTCTGTGGTGAGCAAGCTGTTCTTCAGCTGGAC
CAGACCCATCCTGAGAAAGGGCTACAGACAGAGACTGGAGCTGTCTGACATCTACCAG
ATCCCCTCTGTGGACTCTGCCGACAACCTGTCTGAGAAGCTGGAGAGAGAGTGGGACA
GAGAGCTGGCCAGCAAGAAGAACCCCAAGCTGATCAATGCCCTGAGAAGATGCTTCTT
CTGGAGATTCATGTTCTATGGCATCTTCCTGTACCTGGGAGAGGTGACCAAGGCCGTGC
AGCCCCTGCTGCTGGGCAGGATCATTGCCAGCTATGACCCTGACAACAAGGAGGAGAG
AAGCATTGCCATCTACCTGGGCATTGGCCTGTGCCTGCTGTTCATTGTGAGAACCCTGCT
GCTGCACCCTGCCATCTTTGGCCTGCACCACATTGGCATGCAGATGAGAATTGCCATGT
TCAGCCTGATCTACAAGAAGACCCTGAAGCTGAGCAGCAGAGTGCTGGACAAGATCAG
CATTGGCCAGCTGGTGAGCCTGCTGAGCAACAACCTGAACAAGTTTGATGAGGGCCTGG
CCCTGGCCCACTTTGTGTGGATTGCCCCCCTGCAGGTGGCCCTGCTGATGGGCCTGATCT
GGGAGCTGCTGCAGGCCTCTGCCTTCTGTGGCCTGGGCTTCCTGATTGTGCTGGCCCTGT
TCCAGGCCGGCCTGGGCAGAATGATGATGAAGTACAGAGACCAGAGAGCCGGCAAGAT
CTCTGAGAGACTGGTGATCACCTCTGAGATGATTGAGAACATCCAGTCTGTGAAGGCCT
ACTGCTGGGAGGAGGCCATGGAGAAGATGATTGAGAACCTGAGACAGACAGAGCTGAA
GCTGACCAGGAAGGCCGCCTATGTGAGATACTTCAACAGCTCTGCCTTCTTCTTCTCTGG
CTTCTTTGTGGTGTTCCTGTCTGTGCTGCCCTATGCCCTGATCAAGGGCATCATCCTGAG
GAAGATCTTCACCACCATCAGCTTCTGCATTGTGCTGAGGATGGCCGTGACCAGGCAGT
TCCCCTGGGCCGTGCAGACCTGGTATGACAGCCTGGGGGCCATCAACAAGATCCAGGA
CTTCCTGCAGAAGCAGGAGTACAAGACCCTGGAGTACAACCTGACCACCACAGAGGTG
GTGATGGAGAATGTGACAGCCTTCTGGGAGGAGGGCTTTGGAGAGCTGTTTGAGAAGG
CCAAGCAGAACAACAACAACAGAAAGACCAGCAATGGAGATGACAGCCTGTTCTTCAG
CAACTTCAGCCTGCTGGGCACCCCTGTGCTGAAGGACATCAACTTCAAGATTGAGAGGG
GCCAGCTGCTGGCCGTGGCCGGCAGCACAGGAGCCGGCAAGACCAGCCTGCTGATGGT
GATCATGGGAGAGCTGGAGCCCTCTGAGGGCAAGATCAAGCACTCTGGCAGAATCAGC
TTCTGCAGCCAGTTCAGCTGGATCATGCCTGGCACCATCAAGGAGAACATCATCTTTGG
GGTGAGCTATGATGAGTACAGGTACAGATCTGTGATCAAGGCCTGCCAGCTGGAGGAG
GACATCTCCAAGTTTGCCGAGAAGGACAACATTGTGCTGGGGGAGGGAGGCATCACCC
TGTCTGGGGGCCAGAGAGCCAGAATCAGCCTGGCCAGAGCCGTGTACAAGGATGCCGA
CCTGTACCTGCTGGACAGCCCCTTTGGCTACCTGGATGTGCTGACAGAGAAGGAGATCT
TTGAGAGCTGTGTGTGCAAGCTGATGGCCAACAAGACCAGGATCCTGGTGACCAGCAA
GATGGAGCACCTGAAGAAGGCCGACAAGATCCTGATCCTGCATGAGGGCAGCAGCTAC
TTCTATGGCACCTTCTCTGAGCTGCAGAACCTGCAGCCTGACTTCAGCAGCAAGCTGAT
GGGCTGTGACAGCTTTGACCAGTTCTCTGCTGAGAGAAGAAACAGCATCCTGACAGAG
ACCCTGCACAGGTTCAGCCTGGAGGGGGATGCCCCTGTGAGCTGGACAGAGACCAAGA
AGCAGAGCTTCAAGCAGACAGGAGAGTTTGGGGAGAAGAGGAAGAACAGCATCCTGA
ACCCCATCAACAGCATCAGGAAGTTCAGCATTGTGCAGAAGACCCCCCTGCAGATGAA
TGGCATTGAGGAGGACTCTGATGAGCCCCTGGAGAGAAGACTGAGCCTGGTGCCAGAC
TCTGAGCAGGGAGAGGCCATCCTGCCCAGGATCTCTGTGATCAGCACAGGCCCCACCCT
GCAGGCCAGAAGAAGACAGTCTGTGCTGAACCTGATGACCCACTCTGTGAACCAGGGC
CAGAATATCCACAGAAAGACCACAGCCAGCACCAGAAAGGTGAGCCTGGCCCCCCAGG
CCAACCTGACAGAGCTGGACATCTACAGCAGAAGGCTGAGCCAGGAGACAGGCCTGGA
GATCTCTGAGGAGATCAATGAGGAGGACCTGAAGGAGTGCTTCTTTGATGACATGGAG
AGCATCCCTGCCGTGACCACCTGGAACACCTACCTGAGATACATCACAGTGCACAAGA
GCCTGATCTTTGTGCTGATCTGGTGCCTGGTGATCTTCCTGGCCGAGGTGGCCGCCAGCC
TGGTGGTGCTGTGGCTGCTGGGCAACACCCCCCTGCAGGACAAGGGCAACAGCACCCA
CAGCAGAAACAACAGCTATGCTGTGATCATCACCAGCACCAGCAGCTACTATGTGTTCT
ACATCTATGTGGGAGTGGCTGACACCCTGCTGGCCATGGGCTTCTTCAGAGGCCTGCCC
CTGGTGCACACCCTGATCACAGTGAGCAAGATCCTGCACCACAAGATGCTGCACTCTGT
GCTGCAGGCCCCCATGAGCACCCTGAACACCCTGAAGGCTGGAGGCATCCTGAACAGA
TTCAGCAAGGACATTGCCATCCTGGATGACCTGCTGCCCCTGACCATCTTTGACTTCATC
CAGCTGCTGCTGATTGTGATTGGAGCCATTGCCGTGGTGGCCGTGCTGCAGCCCTACAT
CTTTGTGGCCACAGTGCCTGTGATTGTGGCCTTCATCATGCTGAGGGCCTACTTCCTGCA
GACCAGCCAGCAGCTGAAGCAGCTGGAGTCTGAGGGCAGAAGCCCCATCTTCACCCAC
CTGGTGACCAGCCTGAAGGGCCTGTGGACCCTGAGGGCCTTTGGCAGACAGCCCTACTT
TGAGACCCTGTTCCACAAGGCCCTGAACCTGCACACAGCCAACTGGTTCCTGTACCTGA
GCACCCTGAGATGGTTCCAGATGAGGATTGAGATGATCTTTGTGATCTTCTTCATTGCCG
TGACCTTCATCAGCATCCTGACCACAGGGGAGGGCGAGGGCAGAGTGGGCATCATCCT
GACCCTGGCCATGAACATCATGAGCACCCTGCAGTGGGCCGTGAACAGCAGCATTGAT
GTGGACAGCCTGATGAGATCTGTGAGCAGAGTGTTCAAGTTCATTGACATGCCCACAGA
GGGCAAGCCCACCAAGAGCACCAAGCCCTACAAGAATGGCCAGCTGAGCAAGGTGATG
ATCATTGAGAACAGCCATGTGAAGAAGGATGACATCTGGCCCTCTGGAGGCCAGATGA
CAGTGAAGGACCTGACAGCCAAGTACACAGAGGGGGGCAATGCCATCCTGGAGAACAT
CAGCTTCAGCATCAGCCCTGGCCAGAGGGTGGGCCTGCTGGGCAGAACAGGCTCTGGC
AAGAGCACCCTGCTGTCTGCCTTCCTGAGGCTGCTGAACACAGAGGGAGAGATCCAGA
TTGATGGGGTGAGCTGGGACAGCATCACCCTGCAGCAGTGGAGGAAGGCCTTTGGGGT
GATCCCCCAGAAGGTGTTCATCTTCTCTGGCACCTTCAGGAAGAACCTGGACCCCTATG
AGCAGTGGTCTGACCAGGAGATCTGGAAGGTGGCCGATGAGGTGGGCCTGAGATCTGT
GATTGAGCAGTTCCCTGGCAAGCTGGACTTTGTGCTGGTGGATGGAGGCTGTGTGCTGA
GCCATGGCCACAAGCAGCTGATGTGCCTGGCCAGATCTGTGCTGAGCAAGGCCAAGAT
CCTGCTGCTGGATGAGCCCTCTGCCCACCTGGACCCTGTGACCTACCAGATCATCAGAA
GAACCCTGAAGCAGGCCTTTGCCGACTGCACAGTGATCCTGTGTGAGCACAGAATTGAG
GCCATGCTGGAGTGCCAGCAGTTCCTGGTGATTGAGGAGAACAAGGTGAGGCAGTATG
ACAGCATCCAGAAGCTGCTGAATGAGAGAAGCCTGTTCAGACAGGCCATCAGCCCCTC
TGACAGAGTGAAGCTGTTCCCCCACAGGAACAGCAGCAAGTGCAAGAGCAAGCCCCAG
ATTGCCGCCCTGAAGGAGGAGACAGAGGAGGAGGTGCAGGACACCAGACTGTGA.

In yet another embodiment, an exemplary codon-optimized CFTR mRNA sequence is:

(SEQ ID NO: 19)
ATGCAGAGGAGCCCCCTGGAGAAGGCCAGCGTGGTGAGCAAGCTGTTCTTCAGCTGGA
CCAGGCCCATCCTGAGGAAGGGCTACAGGCAGAGGCTGGAGCTGAGCGACATCTACCA
GATCCCCAGCGTGGACAGCGCCGACAACCTGAGCGAGAAGCTGGAGAGGGAGTGGGA
CAGGGAGCTGGCCAGCAAGAAGAACCCCAAGCTGATCAACGCCCTGAGGAGGTGCTTC
TTCTGGAGGTTCATGTTCTACGGCATCTTCCTGTACCTGGGCGAGGTGACCAAGGCCGT
GCAGCCCCTGCTGCTGGGCAGGATCATCGCCAGCTACGACCCCGACAACAAGGAGGAG
AGGAGCATCGCCATCTACCTGGGCATCGGCCTGTGCCTGCTGTTCATCGTGAGGACCCT
GCTGCTGCACCCCGCCATCTTCGGCCTGCACCACATCGGCATGCAGATGAGGATCGCCA
TGTTCAGCCTGATCTACAAGAAGACCCTGAAGCTGAGCAGCAGGGTGCTGGACAAGAT
CAGCATCGGCCAGCTGGTGAGCCTGCTGAGCAACAACCTGAACAAGTTCGACGAGGGC
CTGGCCCTGGCCCACTTCGTGTGGATCGCCCCCCTGCAGGTGGCCCTGCTGATGGGCCT
GATCTGGGAGCTGCTGCAGGCCAGCGCCTTCTGCGGCCTGGGCTTCCTGATCGTGCTGG
CCCTGTTCCAGGCCGGCCTGGGCAGGATGATGATGAAGTACAGGGACCAGAGGGCCGG
CAAGATCAGCGAGAGGCTGGTGATCACCAGCGAGATGATCGAGAACATCCAGAGCGTG
AAGGCCTACTGCTGGGAGGAGGCCATGGAGAAGATGATCGAGAACCTGAGGCAGACCG
AGCTGAAGCTGACCAGGAAGGCCGCCTACGTGAGGTACTTCAACAGCAGCGCCTTCTTC
TTCAGCGGCTTCTTCGTGGTGTTCCTGAGCGTGCTGCCCTACGCCCTGATCAAGGGCATC
ATCCTGAGGAAGATCTTCACCACCATCAGCTTCTGCATCGTGCTGAGGATGGCCGTGAC
CAGGCAGTTCCCCTGGGCCGTGCAGACCTGGTACGACAGCCTGGGCGCCATCAACAAG
ATCCAGGACTTCCTGCAGAAGCAGGAGTACAAGACCCTGGAGTACAACCTGACCACCA
CCGAGGTGGTGATGGAGAACGTGACCGCCTTCTGGGAGGAGGGCTTCGGCGAGCTGTT
CGAGAAGGCCAAGCAGAACAACAACAACAGGAAGACCAGCAACGGCGACGACAGCCT
GTTCTTCAGCAACTTCAGCCTGCTGGGCACCCCCGTGCTGAAGGACATCAACTTCAAGA
TCGAGAGGGGCCAGCTGCTGGCCGTGGCCGGCAGCACCGGCGCCGGCAAGACCAGCCT
GCTGATGGTGATCATGGGCGAGCTGGAGCCCAGCGAGGGCAAGATCAAGCACAGCGGC
AGGATCAGCTTCTGCAGCCAGTTCAGCTGGATCATGCCCGGCACCATCAAGGAGAACAT
CATCTTCGGCGTGAGCTACGACGAGTACAGGTACAGGAGCGTGATCAAGGCCTGCCAG
CTGGAGGAGGACATCAGCAAGTTCGCCGAGAAGGACAACATCGTGCTGGGCGAGGGCG
GCATCACCCTGAGCGGCGGCCAGAGGGCCAGGATCAGCCTGGCCAGGGCCGTGTACAA
GGACGCCGACCTGTACCTGCTGGACAGCCCCTTCGGCTACCTGGACGTGCTGACCGAGA
AGGAGATCTTCGAGAGCTGCGTGTGCAAGCTGATGGCCAACAAGACCAGGATCCTGGT
GACCAGCAAGATGGAGCACCTGAAGAAGGCCGACAAGATCCTGATCCTGCACGAGGGC
AGCAGCTACTTCTACGGCACCTTCAGCGAGCTGCAGAACCTGCAGCCCGACTTCAGCAG
CAAGCTGATGGGCTGCGACAGCTTCGACCAGTTCAGCGCCGAGAGGAGGAACAGCATC
CTGACCGAGACCCTGCACAGGTTCAGCCTGGAGGGCGACGCCCCCGTGAGCTGGACCG
AGACCAAGAAGCAGAGCTTCAAGCAGACCGGCGAGTTCGGCGAGAAGAGGAAGAACA
GCATCCTGAACCCCATCAACAGCATCAGGAAGTTCAGCATCGTGCAGAAGACCCCCCTG
CAGATGAACGGCATCGAGGAGGACAGCGACGAGCCCCTGGAGAGGAGGCTGAGCCTG
GTGCCCGACAGCGAGCAGGGCGAGGCCATCCTGCCCAGGATCAGCGTGATCAGCACCG
GCCCCACCCTGCAGGCCAGGAGGAGGCAGAGCGTGCTGAACCTGATGACCCACAGCGT
GAACCAGGGCCAGAACATCCACAGGAAGACCACCGCCAGCACCAGGAAGGTGAGCCT
GGCCCCCCAGGCCAACCTGACCGAGCTGGACATCTACAGCAGGAGGCTGAGCCAGGAG
ACCGGCCTGGAGATCAGCGAGGAGATCAACGAGGAGGACCTGAAGGAGTGCTTCTTCG
ACGACATGGAGAGCATCCCCGCCGTGACCACCTGGAACACCTACCTGAGGTACATCAC
CGTGCACAAGAGCCTGATCTTCGTGCTGATCTGGTGCCTGGTGATCTTCCTGGCCGAGG
TGGCCGCCAGCCTGGTGGTGCTGTGGCTGCTGGGCAACACCCCCCTGCAGGACAAGGG
CAACAGCACCCACAGCAGGAACAACAGCTACGCCGTGATCATCACCAGCACCAGCAGC
TACTACGTGTTCTACATCTACGTGGGCGTGGCCGACACCCTGCTGGCCATGGGCTTCTTC
AGGGGCCTGCCCCTGGTGCACACCCTGATCACCGTGAGCAAGATCCTGCACCACAAGAT
GCTGCACAGCGTGCTGCAGGCCCCCATGAGCACCCTGAACACCCTGAAGGCCGGCGGC
ATCCTGAACAGGTTCAGCAAGGACATCGCCATCCTGGACGACCTGCTGCCCCTGACCAT
CTTCGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAGGG
CCTACTTCCTGCAGACCAGCCAGCAGCTGAAGCAGCTGGAGAGCGAGGGCAGGAGCCC
CATCTTCACCCACCTGGTGACCAGCCTGAAGGGCCTGTGGACCCTGAGGGCCTTCGGCA
GGCAGCCCTACTTCGAGACCCTGTTCCACAAGGCCCTGAACCTGCACACCGCCAACTGG
TTCCTGTACCTGAGCACCCTGAGGTGGTTCCAGATGAGGATCGAGATGATCTTCGTGAT
CTTCTTCATCGCCGTGACCTTCATCAGCATCCTGACCACCGGCGAGGGCGAGGGCAGGG
TGGGCATCATCCTGACCCTGGCCATGAACATCATGAGCACCCTGCAGTGGGCCGTGAAC
AGCAGCATCGACGTGGACAGCCTGATGAGGAGCGTGAGCAGGGTGTTCAAGTTCATCG
ACATGCCCACCGAGGGCAAGCCCACCAAGAGCACCAAGCCCTACAAGAACGGCCAGCT
GAGCAAGGTGATGATCATCGAGAACAGCCACGTGAAGAAGGACGACATCTGGCCCAGC
GGCGGCCAGATGACCGTGAAGGACCTGACCGCCAAGTACACCGAGGGCGGCAACGCCA
TCCTGGAGAACATCAGCTTCAGCATCAGCCCCGGCCAGAGGGTGGGCCTGCTGGGCAG
GACCGGCAGCGGCAAGAGCACCCTGCTGAGCGCCTTCCTGAGGCTGCTGAACACCGAG
GGCGAGATCCAGATCGACGGCGTGAGCTGGGACAGCATCACCCTGCAGCAGTGGAGGA
AGGCCTTCGGCGTGATCCCCCAGAAGGTGTTCATCTTCAGCGGCACCTTCAGGAAGAAC
CTGGACCCCTACGAGCAGTGGAGCGACCAGGAGATCTGGAAGGTGGCCGACGAGGTGG
GCCTGAGGAGCGTGATCGAGCAGTTCCCCGGCAAGCTGGACTTCGTGCTGGTGGACGG
CGGCTGCGTGCTGAGCCACGGCCACAAGCAGCTGATGTGCCTGGCCAGGAGCGTGCTG
AGCAAGGCCAAGATCCTGCTGCTGGACGAGCCCAGCGCCCACCTGGACCCCGTGACCT
ACCAGATCATCAGGAGGACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACA
AGGTGAGGCAGTACGACAGCATCCAGAAGCTGCTGAACGAGAGGAGCCTGTTCAGGCA
GGCCATCAGCCCCAGCGACAGGGTGAAGCTGTTCCCCCACAGGAACAGCAGCAAGTGC
AAGAGCAAGCCCCAGATCGCCGCCCTGAAGGAGGAGACCGAGGAGGAGGTGCAGGAC
ACCAGGCTGTGA.

In another embodiment, an exemplary codon-optimized CFTR mRNA sequence is:

(SEQ ID NO: 20)
ATGCAGAGATCCCCTCTGGAGAAGGCCTCAGTGGTGTCCAAGCTTTTCTTCTCCTGGAC
CAGGCCCATTTTAAGAAAGGGCTACAGGCAGAGACTTGAGCTGTCTGACATCTATCAGA
TCCCTTCTGTGGATTCTGCTGACAATCTTAGTGAAAAATTGGAAAGGGAGTGGGACAGA
GAGCTGGCAAGTAAAAAGAACCCCAAGCTGATTAATGCCCTGAGGCGCTGCTTTTTTTG
GAGATTCATGTTCTATGGCATATTCCTCTACCTTGGAGAAGTAACCAAAGCTGTACAGC
CTCTCCTCCTTGGCAGAATCATTGCCTCCTATGATCCTGATAACAAGGAGGAGAGAAGC
ATAGCCATCTACCTGGGCATTGGGCTGTGCCTCTTGTTTATTGTGAGGACCCTTCTCTTG
CACCCTGCCATCTTTGGCCTTCATCACATTGGCATGCAAATGAGAATAGCAATGTTTAGT
CTTATTTACAAAAAAACATTAAAACTCTCTTCCAGGGTGTTGGACAAGATCAGTATTGG
ACAACTGGTCAGCCTGCTGAGCAACAACCTGAACAAGTTTGATGAAGGACTGGCCCTG
GCCCACTTTGTCTGGATTGCCCCCCTTCAGGTGGCTCTTTTGATGGGCCTGATCTGGGAA
CTCCTGCAGGCCTCTGCCTTCTGTGGGTTAGGCTTCCTGATAGTGCTAGCTCTCTTTCAG
GCAGGGTTGGGTAGAATGATGATGAAGTACAGAGACCAGAGGGCTGGGAAGATATCTG
AGAGGCTGGTCATTACTTCTGAAATGATAGAAAACATCCAGTCTGTTAAAGCTTACTGC
TGGGAGGAGGCTATGGAAAAGATGATTGAGAACTTGAGGCAAACAGAGCTCAAGCTGA
CTAGGAAGGCAGCCTATGTCAGGTATTTCAACAGCAGTGCTTTCTTCTTCTCAGGCTTTT
TCGTGGTCTTCTTGAGTGTTCTGCCCTATGCCCTCATCAAGGGGATAATTTTGAGAAAGA
TTTTCACCACTATTTCCTTTTGCATTGTCCTGAGGATGGCTGTCACCAGGCAATTCCCCT
GGGCTGTGCAGACATGGTATGACTCTCTGGGGGCCATCAACAAAATCCAAGATTTCCTG
CAGAAGCAGGAGTACAAGACCCTGGAATACAACCTCACCACCACAGAAGTTGTGATGG
AGAATGTGACTGCATTCTGGGAGGAAGGATTTGGGGAGCTGTTTGAGAAAGCAAAACA
AAACAATAATAACAGGAAAACCAGCAATGGAGATGACTCCCTGTTCTTTTCCAACTTCT
CTTTGTTGGGCACCCCTGTCCTGAAAGATATAAACTTTAAAATTGAAAGAGGGCAGCTG
TTGGCAGTTGCTGGCTCCACAGGAGCTGGAAAAACTTCACTACTGATGGTGATCATGGG
GGAGTTAGAACCCTCTGAAGGGAAAATAAAACATTCTGGGAGGATTAGTTTCTGCAGCC
AGTTCAGCTGGATCATGCCTGGGACCATTAAAGAAAATATTATATTTGGAGTGAGCTAT
GATGAATATAGATATAGGAGTGTCATCAAAGCCTGTCAGTTGGAGGAAGACATCAGCA
AATTTGCAGAGAAAGACAACATTGTTCTGGGTGAAGGTGGCATCACCCTGTCAGGAGG
GCAAAGGGCCAGGATCAGCTTGGCCAGAGCAGTCTATAAAGATGCTGATCTGTACCTCC
TGGATAGCCCTTTTGGCTATCTGGATGTTTTGACAGAGAAGGAAATTTTTGAGTCCTGTG
TCTGCAAGTTAATGGCAAATAAAACAAGGATACTTGTGACCTCAAAAATGGAACACCT
GAAGAAGGCTGACAAAATTCTGATCCTGCATGAGGGCAGCAGCTACTTTTATGGAACAT
TTTCTGAACTGCAGAATTTGCAACCAGACTTTTCATCAAAGCTCATGGGATGTGACAGT
TTTGATCAGTTTTCTGCAGAAAGGAGAAACTCCATTTTGACTGAGACCCTGCACAGGTT
CAGTCTGGAGGGGGATGCCCCAGTGAGTTGGACTGAGACAAAGAAACAGAGCTTCAAG
CAGACTGGAGAGTTTGGAGAAAAGAGGAAAAACTCAATTCTCAATCCCATCAATAGCA
TCAGGAAGTTCAGCATAGTTCAGAAGACTCCTTTGCAGATGAATGGGATTGAAGAGGA
CTCAGATGAGCCCCTGGAAAGGAGACTCTCCTTGGTGCCAGATTCAGAGCAGGGGGAA
GCCATACTGCCAAGGATCTCTGTGATTTCTACAGGGCCCACCCTCCAAGCAAGAAGGAG
ACAGTCAGTTTTAAACCTGATGACCCACTCTGTCAACCAGGGACAGAACATTCATAGAA
AGACAACAGCATCTACAAGAAAAGTTTCACTGGCCCCTCAAGCCAATTTAACTGAACTA
GATATCTACAGCAGGAGGCTCAGCCAAGAAACAGGCCTGGAGATCTCAGAAGAAATAA
ATGAGGAGGATTTGAAGGAATGCTTCTTTGATGATATGGAGAGCATCCCAGCTGTCACA
ACCTGGAACACCTACCTGAGATACATCACAGTGCACAAATCCCTCATCTTTGTACTTAT
ATGGTGCCTTGTCATCTTCTTAGCTGAGGTGGCTGCTTCCCTGGTGGTGCTGTGGCTGCT
GGGAAACACACCCCTCCAGGATAAAGGGAACTCTACTCACAGCAGGAACAACAGTTAT
GCTGTGATCATCACCAGTACCTCCTCCTACTATGTGTTCTACATTTATGTTGGAGTTGCA
GACACATTGCTTGCCATGGGTTTTTTTAGAGGACTCCCCCTGGTGCATACTCTCATCACT
GTTTCCAAAATCCTTCACCACAAGATGCTGCACAGTGTACTACAGGCTCCCATGAGCAC
CCTCAACACTCTTAAAGCAGGAGGAATCTTGAACAGATTTAGCAAGGACATTGCAATTC
TTGATGACCTGCTTCCACTGACCATCTTTGACTTCATCCAGCTTCTGCTCATTGTAATTG
GTGCCATTGCTGTGGTAGCAGTGCTCCAGCCATATATTTTTGTGGCCACTGTGCCTGTTA
TTGTGGCCTTCATTATGTTGAGAGCCTACTTCCTGCAGACCTCTCAGCAGCTCAAGCAAC
TTGAAAGTGAGGGCAGGAGCCCCATATTTACACACTTGGTCACTTCCCTCAAAGGCCTC
TGGACACTCAGAGCTTTTGGAAGACAACCTTATTTTGAAACTCTCTTCCACAAGGCTCTG
AATCTCCACACAGCCAACTGGTTTCTGTATCTTTCAACACTGCGCTGGTTCCAGATGAGG
ATTGAGATGATCTTTGTTATCTTCTTCATAGCTGTTACCTTCATCTCTATTCTGACAACTG
GTGAGGGGGAAGGGAGAGTAGGCATCATCCTCACACTAGCCATGAACATAATGTCTAC
CTTACAATGGGCCGTGAACAGCTCCATAGATGTGGACAGCCTCATGAGAAGTGTGTCAA
GAGTTTTCAAATTCATTGACATGCCCACAGAAGGCAAACCAACCAAGAGCACAAAACC
CTACAAGAATGGCCAGCTGAGTAAGGTCATGATCATTGAAAATTCTCATGTGAAGAAG
GATGATATTTGGCCCAGTGGGGGCCAGATGACAGTCAAGGACCTCACTGCCAAATACA
CAGAGGGTGGAAATGCTATCCTAGAGAACATCTCCTTCTCCATCTCCCCAGGCCAAAGA
GTTGGCTTGCTGGGCAGGACTGGCAGTGGCAAGTCCACCTTGCTCTCAGCATTTCTCAG
GCTTTTAAATACAGAGGGAGAGATTCAAATTGATGGGGTGTCTTGGGATAGTATAACAC
TTCAACAGTGGAGGAAAGCCTTTGGTGTGATTCCTCAGAAAGTGTTTATCTTCTCTGGCA
CTTTCAGAAAAAATCTGGACCCCTATGAACAGTGGAGTGACCAGGAAATCTGGAAGGT
GGCAGATGAAGTGGGCCTAAGATCAGTCATAGAGCAGTTTCCTGGAAAGTTGGATTTTG
TGCTTGTAGATGGAGGCTGTGTGCTGTCCCATGGCCATAAACAGCTAATGTGCCTGGCT
AGGTCAGTGCTGAGCAAGGCCAAGATCCTGCTGTTAGATGAGCCTTCAGCCCATCTGGA
CCCTGTGACATACCAGATTATCAGAAGAACTCTGAAGCAGGCCTTTGCTGACTGCACTG
TCATCCTGTGTGAGCACAGAATTGAGGCCATGCTGGAGTGCCAGCAGTTCCTTGTTATA
GAAGAGAATAAGGTTAGGCAGTATGACAGCATTCAGAAACTGCTAAATGAAAGATCTC
TCTTCAGGCAAGCTATTTCACCATCTGATAGAGTGAAACTTTTTCCCCACAGAAATTCCT
CTAAATGTAAATCTAAGCCCCAGATAGCTGCCTTGAAAGAGGAGACTGAAGAAGAAGT
CCAGGACACCAGACTGTGA.

(SEQ ID NO: 21)
ATGCAGAGATCCCCGCTGGAGAAGGCATCTGTGGTGTCAAAACTGTTCTTTAGCTGGAC
AAGGCCCATCCTTAGGAAAGGGTACAGACAGAGGTTGGAGCTGTCAGACATATATCAG
ATCCCTTCAGTGGACTCTGCAGACAACCTCTCTGAAAAGCTGGAGAGGGAATGGGACA
GGGAACTGGCCAGCAAAAAAAACCCTAAACTGATTAATGCCCTGAGGAGGTGCTTCTTT
TGGAGATTCATGTTCTATGGGATCTTCCTTTACCTGGGGGAGGTGACTAAAGCTGTTCA
GCCTCTTCTTCTGGGGAGGATTATTGCCTCCTATGACCCAGACAACAAAGAAGAAAGAA
GCATAGCCATTTACTTAGGCATAGGCCTCTGCTTGCTCTTCATAGTTAGAACCCTCCTAC
TCCACCCAGCCATCTTTGGTCTCCACCACATAGGTATGCAGATGAGAATAGCAATGTTC
TCCTTGATCTACAAGAAGACCCTCAAGCTGTCCAGCAGGGTGCTGGACAAGATCTCCAT
AGGCCAGTTAGTCAGTCTACTGTCCAATAACTTAAATAAGTTTGATGAGGGACTGGCAC
TGGCACATTTTGTGTGGATTGCCCCCCTCCAAGTGGCCCTTCTTATGGGCCTTATCTGGG
AGCTGTTGCAGGCCTCTGCTTTCTGTGGCCTGGGTTTCCTCATAGTCCTAGCCTTATTCC
AGGCTGGACTGGGCAGAATGATGATGAAGTATAGGGACCAAAGAGCAGGGAAGATTTC
TGAAAGGCTGGTTATAACTTCTGAGATGATTGAGAACATTCAGTCAGTGAAAGCTTACT
GCTGGGAAGAAGCTATGGAAAAAATGATTGAAAATCTCAGACAGACTGAATTAAAGTT
GACCAGGAAAGCTGCTTATGTCAGATACTTCAACTCCTCAGCCTTCTTTTTTTCTGGCTT
CTTTGTTGTATTCCTTTCAGTCCTCCCCTATGCCCTGATTAAGGGCATTATCTTGAGGAA
AATTTTCACAACCATCTCCTTTTGTATTGTCCTCAGGATGGCTGTTACAAGGCAATTTCC
TTGGGCTGTGCAAACTTGGTATGATAGCCTTGGAGCAATCAACAAGATCCAGGATTTCC
TGCAAAAGCAGGAGTACAAGACATTGGAATACAACCTTACCACCACTGAGGTGGTGAT
GGAAAATGTGACTGCCTTCTGGGAGGAGGGGTTTGGAGAGCTGTTTGAGAAAGCCAAA
CAGAACAACAACAATAGAAAGACCTCTAATGGTGATGATTCCCTGTTCTTTTCTAACTTT
AGTCTTCTGGGGACCCCAGTTCTGAAAGATATTAACTTTAAAATTGAAAGGGGACAGTT
GCTGGCTGTGGCTGGGTCCACTGGGGCTGGGAAGACAAGCCTGCTCATGGTGATCATGG
GAGAGCTGGAACCCAGTGAAGGAAAGATCAAACACTCAGGCAGGATCTCCTTCTGCAG
CCAGTTCTCATGGATTATGCCAGGCACTATTAAAGAAAATATCATCTTTGGTGTAAGCT
ATGATGAGTACAGGTATAGATCTGTAATTAAAGCCTGCCAGCTGGAGGAAGACATCTCT
AAGTTTGCTGAGAAGGATAACATTGTGTTGGGGGAAGGGGGCATCACCCTTTCTGGTGG
GCAGAGGGCTAGGATCTCCCTTGCTAGGGCAGTATACAAGGATGCTGACTTGTACCTCT
TGGATAGTCCTTTTGGCTACCTAGATGTGCTGACAGAGAAAGAAATATTTGAAAGCTGT
GTGTGTAAGCTCATGGCTAACAAGACCAGGATCCTGGTCACCAGTAAAATGGAACACCT
CAAAAAAGCAGACAAGATCCTTATTCTCCATGAGGGCTCCTCCTACTTCTATGGGACCT
TCAGTGAGCTGCAGAATCTGCAGCCAGACTTCTCCTCAAAACTTATGGGCTGTGACTCC
TTTGACCAATTCTCTGCAGAAAGAAGGAATAGCATACTGACAGAAACACTGCATAGATT
CTCCCTGGAAGGAGATGCCCCAGTGAGTTGGACAGAAACCAAAAAGCAGAGCTTCAAG
CAGACTGGTGAGTTTGGTGAAAAGAGGAAGAATTCTATCCTGAACCCCATCAATAGCAT
CAGGAAATTTAGCATAGTCCAAAAGACCCCCCTCCAGATGAATGGAATAGAGGAGGAT
AGTGATGAGCCTCTTGAGAGAAGGCTGTCCCTGGTTCCAGACAGTGAACAGGGTGAAG
CCATTCTTCCGAGGATCAGTGTCATCTCCACTGGGCCCACATTGCAGGCCAGAAGAAGA
CAGTCTGTTCTGAATTTGATGACACATTCTGTGAATCAAGGCCAGAATATCCATAGAAA
AACCACTGCCAGCACCAGAAAAGTTTCTCTAGCCCCCCAGGCTAACCTGACTGAGTTAG
ACATCTACAGCAGAAGGCTGAGCCAAGAGACTGGCTTGGAAATATCTGAGGAGATCAA
TGAGGAGGACCTCAAGGAGTGCTTCTTTGATGACATGGAGTCAATCCCTGCAGTCACTA
CATGGAACACTTACCTAAGGTACATCACAGTTCATAAGAGCCTCATCTTTGTCCTCATAT
GGTGTCTGGTCATCTTTTTAGCAGAAGTGGCTGCCAGCCTAGTTGTGCTGTGGTTACTGG
GCAATACACCTCTTCAGGACAAAGGCAATAGCACACACAGCAGAAACAACTCCTATGC
AGTGATCATCACCTCTACAAGCTCTTACTATGTATTCTATATATATGTGGGAGTGGCAGA
TACTCTCCTGGCCATGGGATTCTTCAGGGGATTACCTCTAGTTCACACATTGATCACAGT
GTCAAAAATTCTCCACCACAAGATGTTACACAGTGTCCTGCAAGCCCCAATGTCTACTC
TGAACACACTTAAGGCAGGTGGAATTTTGAATAGGTTTAGCAAGGACATAGCTATCCTG
GATGATCTCCTCCCTCTGACCATCTTTGACTTCATCCAGTTACTGCTCATTGTAATTGGA
GCCATTGCAGTGGTAGCAGTCCTACAGCCTTACATTTTTGTGGCTACTGTTCCTGTTATT
GTGGCCTTCATTATGCTAAGAGCTTACTTCCTGCAAACAAGCCAACAGTTGAAACAGCT
AGAAAGTGAGGGAAGGTCCCCCATCTTCACCCACCTGGTGACATCACTCAAGGGGCTAT
GGACTCTTAGGGCTTTTGGGAGACAGCCGTACTTTGAGACCTTATTCCATAAGGCCCTT
AACCTCCATACAGCAAACTGGTTCTTATACCTGAGTACTCTGAGGTGGTTTCAAATGAG
GATTGAAATGATTTTTGTGATCTTCTTCATTGCTGTGACCTTCATCTCAATCTTGACCAC
AGGAGAGGGGGAGGGCAGGGTGGGCATCATACTGACCTTGGCCATGAACATTATGTCA
ACCCTGCAGTGGGCTGTCAATAGCTCCATTGATGTGGACAGTCTGATGAGGAGTGTCTC
CAGGGTCTTCAAGTTTATTGACATGCCAACTGAGGGCAAACCCACCAAAAGCACTAAG
CCATATAAAAATGGCCAACTGTCCAAAGTGATGATCATTGAAAATTCACATGTAAAGAA
GGATGATATCTGGCCCTCTGGAGGACAGATGACAGTGAAAGACCTGACTGCCAAGTAC
ACAGAGGGTGGTAATGCCATTCTTGAGAACATTAGTTTCAGTATTTCCCCGGGGCAAAG
GGTGGGCCTCCTTGGCAGAACAGGCTCTGGCAAGAGTACCCTGCTGTCAGCCTTTTTAA
GACTGTTGAACACTGAGGGAGAAATTCAGATTGATGGTGTCTCCTGGGATAGCATCACC
CTCCAGCAGTGGAGAAAAGCTTTTGGAGTGATCCCGCAAAAGGTTTTCATCTTTTCAGG
CACCTTCCGGAAGAACCTGGACCCCTATGAGCAGTGGTCTGACCAGGAAATATGGAAG
GTAGCTGATGAAGTTGGGCTTAGGTCAGTCATAGAGCAGTTCCCAGGCAAACTGGACTT
TGTCCTGGTGGATGGTGGATGTGTACTGAGTCATGGGCACAAACAGCTGATGTGCCTAG
CCAGGTCTGTGCTCAGCAAGGCAAAGATATTGCTGCTTGATGAACCCAGTGCCCATCTG
GACCCAGTCACATATCAGATCATCAGAAGAACATTGAAGCAGGCCTTTGCTGATTGCAC
AGTTATCCTCTGTGAGCACAGGATTGAGGCCATGCTGGAGTGCCAGCAGTTTCTGGTGA
TTGAGGAGAATAAAGTAAGGCAGTATGACTCCATCCAGAAGCTGCTCAATGAAAGAAG
CCTCTTTAGACAAGCTATCTCCCCCTCAGACAGGGTCAAATTGTTCCCTCACAGAAACA
GCAGCAAGTGCAAGAGCAAGCCCCAAATTGCAGCCTTGAAAGAGGAGACAGAGGAAG
AGGTGCAGGACACCAGACTCTGA.

(SEQ ID NO: 22)
ATGCAGAGAAGCCCCCTGGAGAAGGCCAGCGTGGTGAGCAAGCTGTTCTTCAGCTGGA
CCAGACCCATCCTGAGAAAGGGCTACAGACAGAGACTGGAGCTGAGCGACATCTACCA
GATCCCCAGCGTGGACAGCGCCGACAACCTGAGCGAGAAGCTGGAGAGAGAGTGGGA
CAGAGAGCTGGCCAGCAAGAAGAACCCCAAGCTGATCAACGCCCTGAGAAGATGCTTC
TTCTGGAGATTCATGTTCTACGGCATCTTCCTGTACCTGGGCGAGGTGACCAAGGCCGT
GCAGCCCCTGCTGCTGGGCAGAATCATCGCCAGCTACGACCCCGACAACAAGGAGGAG
AGAAGCATCGCCATCTACCTGGGCATCGGCCTGTGCCTGCTGTTCATCGTGAGAACCCT
GCTGCTGCACCCCGCCATCTTCGGCCTGCACCACATCGGCATGCAGATGAGAATCGCCA
TGTTCAGCCTGATCTACAAGAAGACCCTGAAGCTGAGCAGCAGAGTGCTGGACAAGAT
CAGCATCGGCCAGCTGGTGAGCCTGCTGAGCAACAACCTGAACAAGTTCGACGAGGGC
CTGGCCCTGGCCCACTTCGTGTGGATCGCCCCCCTGCAGGTGGCCCTGCTGATGGGCCT
GATCTGGGAGCTGCTGCAGGCCAGCGCCTTCTGCGGCCTGGGCTTCCTGATCGTGCTGG
CCCTGTTCCAGGCCGGCCTGGGCAGAATGATGATGAAGTACAGAGACCAGAGAGCCGG
CAAGATCAGCGAGAGACTGGTGATCACCAGCGAGATGATCGAGAACATCCAGAGCGTG
AAGGCCTACTGCTGGGAGGAGGCCATGGAGAAGATGATCGAGAACCTGAGACAGACCG
AGCTGAAGCTGACCAGAAAGGCCGCCTACGTGAGATACTTCAACAGCAGCGCCTTCTTC
TTCAGCGGCTTCTTCGTGGTGTTCCTGAGCGTGCTGCCCTACGCCCTGATCAAGGGCATC
ATCCTGAGAAAGATCTTCACCACCATCAGCTTCTGCATCGTGCTGAGAATGGCCGTGAC
CAGACAGTTCCCCTGGGCCGTGCAGACCTGGTACGACAGCCTGGGCGCCATCAACAAG
ATCCAGGACTTCCTGCAGAAGCAGGAGTACAAGACCCTGGAGTACAACCTGACCACCA
CCGAGGTGGTGATGGAGAACGTGACCGCCTTCTGGGAGGAGGGCTTCGGCGAGCTGTT
CGAGAAGGCCAAGCAGAACAACAACAACAGAAAGACCAGCAACGGCGACGACAGCCT
GTTCTTCAGCAACTTCAGCCTGCTGGGCACCCCCGTGCTGAAGGACATCAACTTCAAGA
TCGAGAGAGGCCAGCTGCTGGCCGTGGCCGGCAGCACCGGCGCCGGCAAGACCAGCCT
GCTGATGGTGATCATGGGCGAGCTGGAGCCCAGCGAGGGCAAGATCAAGCACAGCGGC
AGAATCAGCTTCTGCAGCCAGTTCAGCTGGATCATGCCCGGCACCATCAAGGAGAACAT
CATCTTCGGCGTGAGCTACGACGAGTACAGATACAGAAGCGTGATCAAGGCCTGCCAG
CTGGAGGAGGACATCAGCAAGTTCGCCGAGAAGGACAACATCGTGCTGGGCGAGGGCG
GCATCACCCTGAGCGGCGGCCAGAGAGCCAGAATCAGCCTGGCCAGAGCCGTGTACAA
GGACGCCGACCTGTACCTGCTGGACAGCCCCTTCGGCTACCTGGACGTGCTGACCGAGA
AGGAGATCTTCGAGAGCTGCGTGTGCAAGCTGATGGCCAACAAGACCAGAATCCTGGT
GACCAGCAAGATGGAGCACCTGAAGAAGGCCGACAAGATCCTGATCCTGCACGAGGGC
AGCAGCTACTTCTACGGCACCTTCAGCGAGCTGCAGAACCTGCAGCCCGACTTCAGCAG
CAAGCTGATGGGCTGCGACAGCTTCGACCAGTTCAGCGCCGAGAGAAGAAACAGCATC
CTGACCGAGACCCTGCACAGATTCAGCCTGGAGGGCGACGCCCCCGTGAGCTGGACCG
AGACCAAGAAGCAGAGCTTCAAGCAGACCGGCGAGTTCGGCGAGAAGAGAAAGAACA
GCATCCTGAACCCCATCAACAGCATCAGAAAGTTCAGCATCGTGCAGAAGACCCCCCTG
CAGATGAACGGCATCGAGGAGGACAGCGACGAGCCCCTGGAGAGAAGACTGAGCCTG
GTGCCCGACAGCGAGCAGGGCGAGGCCATCCTGCCCAGAATCAGCGTGATCAGCACCG
GCCCCACCCTGCAGGCCAGAAGAAGACAGAGCGTGCTGAACCTGATGACCCACAGCGT
GAACCAGGGCCAGAACATCCACAGAAAGACCACCGCCAGCACCAGAAAGGTGAGCCT
GGCCCCCCAGGCCAACCTGACCGAGCTGGACATCTACAGCAGAAGACTGAGCCAGGAG
ACCGGCCTGGAGATCAGCGAGGAGATCAACGAGGAGGACCTGAAGGAGTGCTTCTTCG
ACGACATGGAGAGCATCCCCGCCGTGACCACCTGGAACACCTACCTGAGATACATCAC
CGTGCACAAGAGCCTGATCTTCGTGCTGATCTGGTGCCTGGTGATCTTCCTGGCCGAGG
TGGCCGCCAGCCTGGTGGTGCTGTGGCTGCTGGGCAACACCCCCCTGCAGGACAAGGG
CAACAGCACCCACAGCAGAAACAACAGCTACGCCGTGATCATCACCAGCACCAGCAGC
TACTACGTGTTCTACATCTACGTGGGCGTGGCCGACACCCTGCTGGCCATGGGCTTCTTC
AGAGGCCTGCCCCTGGTGCACACCCTGATCACCGTGAGCAAGATCCTGCACCACAAGAT
GCTGCACAGCGTGCTGCAGGCCCCCATGAGCACCCTGAACACCCTGAAGGCCGGCGGC
ATCCTGAACAGATTCAGCAAGGACATCGCCATCCTGGACGACCTGCTGCCCCTGACCAT
CTTCGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAGAG
CCTACTTCCTGCAGACCAGCCAGCAGCTGAAGCAGCTGGAGAGCGAGGGCAGAAGCCC
CATCTTCACCCACCTGGTGACCAGCCTGAAGGGCCTGTGGACCCTGAGAGCCTTCGGCA
GACAGCCCTACTTCGAGACCCTGTTCCACAAGGCCCTGAACCTGCACACCGCCAACTGG
TTCCTGTACCTGAGCACCCTGAGATGGTTCCAGATGAGAATCGAGATGATCTTCGTGAT
CTTCTTCATCGCCGTGACCTTCATCAGCATCCTGACCACCGGCGAGGGCGAGGGCAGAG
TGGGCATCATCCTGACCCTGGCCATGAACATCATGAGCACCCTGCAGTGGGCCGTGAAC
AGCAGCATCGACGTGGACAGCCTGATGAGAAGCGTGAGCAGAGTGTTCAAGTTCATCG
ACATGCCCACCGAGGGCAAGCCCACCAAGAGCACCAAGCCCTACAAGAACGGCCAGCT
GAGCAAGGTGATGATCATCGAGAACAGCCACGTGAAGAAGGACGACATCTGGCCCAGC
GGCGGCCAGATGACCGTGAAGGACCTGACCGCCAAGTACACCGAGGGCGGCAACGCCA
TCCTGGAGAACATCAGCTTCAGCATCAGCCCCGGCCAGAGAGTGGGCCTGCTGGGCAG
AACCGGCAGCGGCAAGAGCACCCTGCTGAGCGCCTTCCTGAGACTGCTGAACACCGAG
GGCGAGATCCAGATCGACGGCGTGAGCTGGGACAGCATCACCCTGCAGCAGTGGAGAA
AGGCCTTCGGCGTGATCCCCCAGAAGGTGTTCATCTTCAGCGGCACCTTCAGAAAGAAC
CTGGACCCCTACGAGCAGTGGAGCGACCAGGAGATCTGGAAGGTGGCCGACGAGGTGG
GCCTGAGAAGCGTGATCGAGCAGTTCCCCGGCAAGCTGGACTTCGTGCTGGTGGACGG
CGGCTGCGTGCTGAGCCACGGCCACAAGCAGCTGATGTGCCTGGCCAGAAGCGTGCTG
AGCAAGGCCAAGATCCTGCTGCTGGACGAGCCCAGCGCCCACCTGGACCCCGTGACCT
ACCAGATCATCAGAAGAACCCTGAAGCAGGCCTTCGCCGACTGCACCGTGATCCTGTGC
GAGCACAGAATCGAGGCCATGCTGGAGTGCCAGCAGTTCCTGGTGATCGAGGAGAACA
AGGTGAGACAGTACGACAGCATCCAGAAGCTGCTGAACGAGAGAAGCCTGTTCAGACA
GGCCATCAGCCCCAGCGACAGAGTGAAGCTGTTCCCCCACAGAAACAGCAGCAAGTGC
AAGAGCAAGCCCCAGATCGCCGCCCTGAAGGAGGAGACCGAGGAGGAGGTGCAGGAC
ACCAGACTGTGA.

(SEQ ID NO: 23)
ATGCAGCGCAGCCCCCTGGAGAAGGCCAGCGTGGTGAGCAAGCTGTTCTTCAGCTGGA
CCCGCCCCATCCTGCGCAAGGGCTACCGCCAGCGCCTGGAGCTGAGCGACATCTACCAG
ATCCCCAGCGTGGACAGCGCCGACAACCTGAGCGAGAAGCTGGAGCGCGAGTGGGACC
GCGAGCTGGCCAGCAAGAAGAACCCCAAGCTGATCAACGCCCTGCGCCGCTGCTTCTTC
TGGCGCTTCATGTTCTACGGCATCTTCCTGTACCTGGGCGAGGTGACCAAGGCCGTGCA
GCCCCTGCTGCTGGGCCGCATCATCGCCAGCTACGACCCCGACAACAAGGAGGAGCGC
AGCATCGCCATCTACCTGGGCATCGGCCTGTGCCTGCTGTTCATCGTGCGCACCCTGCTG
CTGCACCCCGCCATCTTCGGCCTGCACCACATCGGCATGCAGATGCGCATCGCCATGTT
CAGCCTGATCTACAAGAAGACCCTGAAGCTGAGCAGCCGCGTGCTGGACAAGATCAGC
ATCGGCCAGCTGGTGAGCCTGCTGAGCAACAACCTGAACAAGTTCGACGAGGGCCTGG
CCCTGGCCCACTTCGTGTGGATCGCCCCCCTGCAGGTGGCCCTGCTGATGGGCCTGATC
TGGGAGCTGCTGCAGGCCAGCGCCTTCTGCGGCCTGGGCTTCCTGATCGTGCTGGCCCT
GTTCCAGGCCGGCCTGGGCCGCATGATGATGAAGTACCGCGACCAGCGCGCCGGCAAG
ATCAGCGAGCGCCTGGTGATCACCAGCGAGATGATCGAGAACATCCAGAGCGTGAAGG
CCTACTGCTGGGAGGAGGCCATGGAGAAGATGATCGAGAACCTGCGCCAGACCGAGCT
GAAGCTGACCCGCAAGGCCGCCTACGTGCGCTACTTCAACAGCAGCGCCTTCTTCTTCA
GCGGCTTCTTCGTGGTGTTCCTGAGCGTGCTGCCCTACGCCCTGATCAAGGGCATCATCC
TGCGCAAGATCTTCACCACCATCAGCTTCTGCATCGTGCTGCGCATGGCCGTGACCCGC
CAGTTCCCCTGGGCCGTGCAGACCTGGTACGACAGCCTGGGCGCCATCAACAAGATCCA
GGACTTCCTGCAGAAGCAGGAGTACAAGACCCTGGAGTACAACCTGACCACCACCGAG
GTGGTGATGGAGAACGTGACCGCCTTCTGGGAGGAGGGCTTCGGCGAGCTGTTCGAGA
AGGCCAAGCAGAACAACAACAACCGCAAGACCAGCAACGGCGACGACAGCCTGTTCTT
CAGCAACTTCAGCCTGCTGGGCACCCCCGTGCTGAAGGACATCAACTTCAAGATCGAGC
GCGGCCAGCTGCTGGCCGTGGCCGGCAGCACCGGCGCCGGCAAGACCAGCCTGCTGAT
GGTGATCATGGGCGAGCTGGAGCCCAGCGAGGGCAAGATCAAGCACAGCGGCCGCATC
AGCTTCTGCAGCCAGTTCAGCTGGATCATGCCCGGCACCATCAAGGAGAACATCATCTT
CGGCGTGAGCTACGACGAGTACCGCTACCGCAGCGTGATCAAGGCCTGCCAGCTGGAG
GAGGACATCAGCAAGTTCGCCGAGAAGGACAACATCGTGCTGGGCGAGGGCGGCATCA
CCCTGAGCGGCGGCCAGCGCGCCCGCATCAGCCTGGCCCGCGCCGTGTACAAGGACGC
CGACCTGTACCTGCTGGACAGCCCCTTCGGCTACCTGGACGTGCTGACCGAGAAGGAGA
TCTTCGAGAGCTGCGTGTGCAAGCTGATGGCCAACAAGACCCGCATCCTGGTGACCAGC
AAGATGGAGCACCTGAAGAAGGCCGACAAGATCCTGATCCTGCACGAGGGCAGCAGCT
ACTTCTACGGCACCTTCAGCGAGCTGCAGAACCTGCAGCCCGACTTCAGCAGCAAGCTG
ATGGGCTGCGACAGCTTCGACCAGTTCAGCGCCGAGCGCCGCAACAGCATCCTGACCG
AGACCCTGCACCGCTTCAGCCTGGAGGGCGACGCCCCCGTGAGCTGGACCGAGACCAA
GAAGCAGAGCTTCAAGCAGACCGGCGAGTTCGGCGAGAAGCGCAAGAACAGCATCCTG
AACCCCATCAACAGCATCCGCAAGTTCAGCATCGTGCAGAAGACCCCCCTGCAGATGA
ACGGCATCGAGGAGGACAGCGACGAGCCCCTGGAGCGCCGCCTGAGCCTGGTGCCCGA
CAGCGAGCAGGGCGAGGCCATCCTGCCCCGCATCAGCGTGATCAGCACCGGCCCCACC
CTGCAGGCCCGCCGCCGCCAGAGCGTGCTGAACCTGATGACCCACAGCGTGAACCAGG
GCCAGAACATCCACCGCAAGACCACCGCCAGCACCCGCAAGGTGAGCCTGGCCCCCCA
GGCCAACCTGACCGAGCTGGACATCTACAGCCGCCGCCTGAGCCAGGAGACCGGCCTG
GAGATCAGCGAGGAGATCAACGAGGAGGACCTGAAGGAGTGCTTCTTCGACGACATGG
AGAGCATCCCCGCCGTGACCACCTGGAACACCTACCTGCGCTACATCACCGTGCACAAG
AGCCTGATCTTCGTGCTGATCTGGTGCCTGGTGATCTTCCTGGCCGAGGTGGCCGCCAG
CCTGGTGGTGCTGTGGCTGCTGGGCAACACCCCCCTGCAGGACAAGGGCAACAGCACC
CACAGCCGCAACAACAGCTACGCCGTGATCATCACCAGCACCAGCAGCTACTACGTGTT
CTACATCTACGTGGGCGTGGCCGACACCCTGCTGGCCATGGGCTTCTTCCGCGGCCTGC
CCCTGGTGCACACCCTGATCACCGTGAGCAAGATCCTGCACCACAAGATGCTGCACAGC
GTGCTGCAGGCCCCCATGAGCACCCTGAACACCCTGAAGGCCGGCGGCATCCTGAACC
GCTTCAGCAAGGACATCGCCATCCTGGACGACCTGCTGCCCCTGACCATCTTCGACTTC
ATCCAGCTGCTGCTGATCGTGATCGGCGCCATCGCCGTGGTGGCCGTGCTGCAGCCCTA
CATCTTCGTGGCCACCGTGCCCGTGATCGTGGCCTTCATCATGCTGCGCGCCTACTTCCT
GCAGACCAGCCAGCAGCTGAAGCAGCTGGAGAGCGAGGGCCGCAGCCCCATCTTCACC
CACCTGGTGACCAGCCTGAAGGGCCTGTGGACCCTGCGCGCCTTCGGCCGCCAGCCCTA
CTTCGAGACCCTGTTCCACAAGGCCCTGAACCTGCACACCGCCAACTGGTTCCTGTACC
TGAGCACCCTGCGCTGGTTCCAGATGCGCATCGAGATGATCTTCGTGATCTTCTTCATCG
CCGTGACCTTCATCAGCATCCTGACCACCGGCGAGGGCGAGGGCCGCGTGGGCATCATC
CTGACCCTGGCCATGAACATCATGAGCACCCTGCAGTGGGCCGTGAACAGCAGCATCG
ACGTGGACAGCCTGATGCGCAGCGTGAGCCGCGTGTTCAAGTTCATCGACATGCCCACC
GAGGGCAAGCCCACCAAGAGCACCAAGCCCTACAAGAACGGCCAGCTGAGCAAGGTG
ATGATCATCGAGAACAGCCACGTGAAGAAGGACGACATCTGGCCCAGCGGCGGCCAGA
TGACCGTGAAGGACCTGACCGCCAAGTACACCGAGGGCGGCAACGCCATCCTGGAGAA
CATCAGCTTCAGCATCAGCCCCGGCCAGCGCGTGGGCCTGCTGGGCCGCACCGGCAGC
GGCAAGAGCACCCTGCTGAGCGCCTTCCTGCGCCTGCTGAACACCGAGGGCGAGATCC
AGATCGACGGCGTGAGCTGGGACAGCATCACCCTGCAGCAGTGGCGCAAGGCCTTCGG
CGTGATCCCCCAGAAGGTGTTCATCTTCAGCGGCACCTTCCGCAAGAACCTGGACCCCT
ACGAGCAGTGGAGCGACCAGGAGATCTGGAAGGTGGCCGACGAGGTGGGCCTGCGCA
GCGTGATCGAGCAGTTCCCCGGCAAGCTGGACTTCGTGCTGGTGGACGGCGGCTGCGTG
CTGAGCCACGGCCACAAGCAGCTGATGTGCCTGGCCCGCAGCGTGCTGAGCAAGGCCA
AGATCCTGCTGCTGGACGAGCCCAGCGCCCACCTGGACCCCGTGACCTACCAGATCATC
CGCCGCACCCTGAAGCAGGCCTTCGCCGACTGCACCGTGATCCTGTGCGAGCACCGCAT
CGAGGCCATGCTGGAGTGCCAGCAGTTCCTGGTGATCGAGGAGAACAAGGTGCGCCAG
TACGACAGCATCCAGAAGCTGCTGAACGAGCGCAGCCTGTTCCGCCAGGCCATCAGCC
CCAGCGACCGCGTGAAGCTGTTCCCCCACCGCAACAGCAGCAAGTGCAAGAGCAAGCC
CCAGATCGCCGCCCTGAAGGAGGAGACCGAGGAGGAGGTGCAGGACACCCGCCTGTAA.

In yet another embodiment, an exemplary codon-optimized CFTR mRNA sequence is:

(SEQ ID NO: 24)
ATGCAGAGAAGCCCCCTGGAGAAGGCCAGCGTGGTGAGCAAGCTGTTCTTCAGCTGGA
CCAGACCCATCCTGAGAAAGGGCTACAGACAGAGACTGGAGCTGAGCGACATCTACCA
GATCCCCAGCGTGGACAGCGCCGACAACCTGAGCGAGAAGCTGGAGAGAGAGTGGGA
CAGAGAGCTGGCCAGCAAGAAGAACCCCAAGCTGATCAACGCCCTGAGAAGATGCTTC
TTCTGGAGATTCATGTTCTACGGCATCTTCCTGTACCTGGGCGAGGTGACCAAGGCCGT
GCAGCCCCTGCTGCTGGGCAGAATCATCGCCAGCTACGACCCCGACAACAAGGAGGAG
AGAAGCATCGCCATCTACCTGGGCATCGGCCTGTGCCTGCTGTTCATCGTGAGAACCCT
GCTGCTGCACCCCGCCATCTTCGGCCTGCACCACATCGGCATGCAGATGAGAATCGCCA
TGTTCAGCCTGATCTACAAGAAGACCCTGAAGCTGAGCAGCAGAGTGCTGGACAAGAT
CAGCATCGGCCAGCTGGTGAGCCTGCTGAGCAACAACCTGAACAAGTTCGACGAGGGC
CTGGCCCTGGCCCACTTCGTGTGGATCGCCCCCCTGCAGGTGGCCCTGCTGATGGGCCT
GATCTGGGAGCTGCTGCAGGCCAGCGCCTTCTGCGGCCTGGGCTTCCTGATCGTGCTGG
CCCTGTTCCAGGCCGGCCTGGGCAGAATGATGATGAAGTACAGGGACCAGAGAGCCGG
CAAGATCAGCGAGAGACTGGTGATCACCAGCGAGATGATCGAGAACATCCAGAGCGTG
AAGGCCTACTGCTGGGAGGAGGCCATGGAGAAGATGATCGAGAACCTGAGACAGACCG
AGCTGAAGCTGACCAGAAAGGCCGCCTACGTGAGATACTTCAACAGCAGCGCCTTCTTC
TTCAGCGGCTTCTTCGTGGTGTTCCTGAGCGTGCTGCCCTACGCCCTGATCAAGGGCATC
ATCCTGAGAAAGATCTTCACCACCATCAGCTTCTGCATCGTGCTGAGAATGGCCGTGAC
CAGACAGTTCCCCTGGGCCGTGCAGACCTGGTACGACAGCCTGGGCGCCATCAACAAG
ATCCAGGACTTCCTGCAGAAGCAGGAGTACAAGACCCTGGAGTACAACCTGACCACCA
CCGAGGTGGTGATGGAGAACGTGACCGCCTTCTGGGAGGAGGGCTTCGGCGAGCTGTT
CGAGAAGGCCAAGCAGAACAACAACAACAGAAAGACCAGCAACGGCGACGACAGCCT
GTTCTTCAGCAACTTCAGCCTGCTGGGCACCCCCGTGCTGAAGGACATCAACTTCAAGA
TCGAGAGAGGCCAGCTGCTGGCCGTGGCCGGCAGCACCGGCGCCGGCAAGACCAGCCT
GCTGATGGTGATCATGGGCGAGCTGGAGCCCAGCGAGGGCAAGATCAAGCACAGCGGC
AGAATCAGCTTCTGCAGCCAGTTCAGCTGGATCATGCCCGGCACCATCAAGGAGAACAT
CATCTTCGGCGTGAGCTACGACGAGTACAGATACAGAAGCGTGATCAAGGCCTGCCAG
CTGGAGGAGGACATCAGCAAGTTCGCCGAGAAGGACAACATCGTGCTGGGCGAGGGCG
GCATCACCCTGAGCGGCGGCCAGAGAGCCAGAATCAGCCTGGCCAGAGCCGTGTACAA
GGACGCCGACCTGTACCTGCTGGACAGCCCCTTCGGCTACCTGGACGTGCTGACCGAGA
AGGAGATCTTCGAGAGCTGCGTGTGCAAGCTGATGGCCAACAAGACCAGAATCCTGGT
GACCAGCAAGATGGAGCACCTGAAGAAGGCCGACAAGATCCTGATCCTGCACGAGGGC
AGCAGCTACTTCTACGGCACCTTCAGCGAGCTGCAGAACCTGCAGCCCGACTTCAGCAG
CAAGCTGATGGGCTGCGACAGCTTCGACCAGTTCAGCGCCGAGAGAAGAAACAGCATC
CTGACCGAGACCCTGCACAGATTCAGCCTGGAGGGCGACGCCCCCGTGAGCTGGACCG
AGACCAAGAAGCAGAGCTTCAAGCAGACCGGCGAGTTCGGCGAGAAGAGAAAGAACA
GCATCCTGAACCCCATCAACAGCATCAGAAAGTTCAGCATCGTGCAGAAGACCCCCCTG
CAGATGAACGGCATCGAGGAGGACAGCGACGAGCCCCTGGAGAGAAGACTGAGCCTG
GTGCCCGACAGCGAGCAGGGCGAGGCCATCCTGCCCAGAATCAGCGTGATCAGCACCG
GCCCCACCCTGCAGGCCAGAAGAAGACAGAGCGTGCTGAACCTGATGACCCACAGCGT
GAACCAGGGCCAGAACATCCACAGAAAGACCACCGCCAGCACCAGAAAGGTGAGCCT
GGCCCCCCAGGCCAACCTGACCGAGCTGGACATCTACAGCAGAAGACTGAGCCAGGAG
ACCGGCCTGGAGATCAGCGAGGAGATCAACGAGGAGGACCTGAAGGAGTGCTTCTTCG
ACGACATGGAGAGCATCCCCGCCGTGACCACCTGGAACACCTACCTGAGATACATCAC
CGTGCACAAGAGCCTGATCTTCGTGCTGATCTGGTGCCTGGTGATCTTCCTGGCCGAGG
TGGCCGCCAGCCTGGTGGTGCTGTGGCTGCTGGGCAACACCCCCCTGCAGGACAAGGG
CAACAGCACCCACAGCAGAAACAACAGCTACGCCGTGATCATCACCAGCACCAGCAGC
TACTACGTGTTCTACATCTACGTGGGCGTGGCCGACACCCTGCTGGCCATGGGCTTCTTC
AGAGGCCTGCCCCTGGTGCACACCCTGATCACCGTGAGCAAGATCCTGCACCACAAGAT
GCTGCACAGCGTGCTGCAGGCCCCCATGAGCACCCTGAACACCCTGAAGGCCGGCGGC
ATCCTGAACAGATTCAGCAAGGACATCGCCATCCTGGACGACCTGCTGCCCCTGACCAT
CTTCGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAGAG
CCTACTTCCTGCAGACCAGCCAGCAGCTGAAGCAGCTGGAGAGCGAGGGCAGGAGCCC
CATCTTCACCCACCTGGTGACCAGCCTGAAGGGCCTGTGGACCCTGAGAGCCTTCGGCA
GACAGCCCTACTTCGAGACCCTGTTCCACAAGGCCCTGAACCTGCACACCGCCAACTGG
TTCCTGTACCTGAGCACCCTGAGATGGTTCCAGATGAGAATCGAGATGATCTTCGTGAT
CTTCTTCATCGCCGTGACCTTCATCAGCATCCTGACCACCGGCGAGGGCGAGGGCAGAG
TGGGCATCATCCTGACCCTGGCCATGAACATCATGAGCACCCTGCAGTGGGCCGTGAAC
AGCAGCATCGACGTGGACAGCCTGATGAGAAGCGTGAGCAGAGTGTTCAAGTTCATCG
ACATGCCCACCGAGGGCAAGCCCACCAAGAGCACCAAGCCCTACAAGAACGGCCAGCT
GAGCAAGGTGATGATCATCGAGAACAGCCACGTGAAGAAGGACGACATCTGGCCCAGC
GGCGGCCAGATGACCGTGAAGGACCTGACCGCCAAGTACACCGAGGGCGGCAACGCCA
TCCTGGAGAACATCAGCTTCAGCATCAGCCCCGGCCAGAGAGTGGGCCTGCTGGGCAG
AACCGGCAGCGGCAAGAGCACCCTGCTGAGCGCCTTCCTGAGACTGCTGAACACCGAG
GGCGAGATCCAGATCGACGGCGTGAGCTGGGACAGCATCACCCTGCAGCAGTGGAGAA
AGGCCTTCGGCGTGATCCCCCAGAAGGTGTTCATCTTCAGCGGCACCTTCAGAAAGAAC
CTGGACCCCTACGAGCAGTGGAGCGACCAGGAGATCTGGAAGGTGGCCGACGAGGTGG
GCCTGAGAAGCGTGATCGAGCAGTTCCCCGGCAAGCTGGACTTCGTGCTGGTGGACGG
CGGCTGCGTGCTGAGCCACGGCCACAAGCAGCTGATGTGCCTGGCCAGAAGCGTGCTG
AGCAAGGCCAAGATCCTGCTGCTGGACGAGCCCAGCGCCCACCTGGACCCCGTGACCT
ACCAGATCATCAGAAGAACCCTGAAGCAGGCCTTCGCCGACTGCACCGTGATCCTGTGC
GAGCACAGAATCGAGGCCATGCTGGAGTGCCAGCAGTTCCTGGTGATCGAGGAGAACA
AGGTGAGACAGTACGACAGCATCCAGAAGCTGCTGAACGAGAGAAGCCTGTTCAGACA
GGCCATCAGCCCCAGCGACAGAGTGAAGCTGTTCCCCCACAGAAACAGCAGCAAGTGC
AAGAGCAAGCCCCAGATCGCCGCCCTGAAGGAGGAGACCGAGGAGGAGGTGCAGGAC
ACCAGACTGTGA.

(SEQ ID NO: 25)
ATGCAGAGGTCACCTCTGGAAAAGGCTAGCGTGGTCAGCAAGCTATTTTTTTCCTGGAC
CCGCCCGATACTCAGGAAGGGCTACCGACAGCGGCTGGAGCTGAGTGACATTTATCAG
ATTCCCTCCGTCGATTCCGCTGACAACCTGTCTGAGAAACTGGAGCGGGAATGGGATAG
GGAACTGGCGTCCAAAAAAAACCCCAAACTCATCAATGCACTCCGCAGATGCTTCTTCT
GGCGGTTTATGTTTTATGGCATATTCCTGTATCTGGGGGAGGTGACGAAAGCCGTGCAG
CCGCTGCTGCTTGGTCGCATTATCGCGTCATACGATCCAGATAACAAGGAGGAAAGAAG
TATCGCTATCTATCTCGGGATAGGGCTGTGCCTGCTCTTCATTGTGCGGACTCTTCTCTT
GCACCCCGCCATTTTCGGTCTGCATCATATAGGTATGCAGATGAGAATTGCGATGTTCTC
ATTGATTTACAAAAAAACGCTTAAGCTAAGTTCAAGGGTGCTAGATAAGATATCGATCG
GCCAGCTGGTGTCTCTGCTTAGCAACAACCTCAATAAATTCGACGAAGGCCTTGCACTG
GCCCACTTCGTGTGGATCGCCCCTCTGCAGGTGGCTCTGCTGATGGGGTTAATATGGGA
GCTGTTGCAGGCCTCCGCTTTTTGTGGCCTGGGGTTTCTCATCGTGTTGGCCTTGTTTCA
GGCAGGGCTGGGACGTATGATGATGAAATATAGGGATCAGAGGGCTGGCAAAATCTCT
GAGCGCCTGGTTATTACGAGTGAAATGATTGAGAACATCCAGTCAGTGAAGGCCTATTG
CTGGGAGGAGGCCATGGAAAAAATGATTGAGAACCTACGCCAGACTGAGCTGAAGTTA
ACCAGAAAAGCCGCCTATGTGCGCTACTTTAACAGTAGCGCATTTTTCTTCTCCGGTTTT
TTCGTGGTGTTTCTTAGTGTGTTGCCGTATGCCTTAATCAAGGGAATAATACTCCGGAAG
ATTTTCACTACCATCAGCTTCTGTATCGTGTTGCGGATGGCCGTCACCCGGCAGTTTCCC
TGGGCAGTACAGACTTGGTACGATTCTCTCGGAGCAATTAACAAAATCCAAGACTTTCT
ACAAAAGCAGGAGTACAAGACCCTGGAGTACAATCTGACCACCACAGAAGTCGTAATG
GAGAATGTAACTGCCTTCTGGGAAGAGGGCTTTGGCGAACTCTTTGAAAAGGCCAAGC
AGAACAATAACAACCGGAAGACCTCCAACGGGGACGACAGCTTATTTTTCAGCAATTTT
TCTTTGCTCGGGACCCCTGTACTGAAAGATATTAACTTTAAGATCGAGCGCGGACAACT
CCTGGCTGTCGCCGGCAGCACTGGAGCTGGAAAAACATCACTGCTTATGGTGATAATGG
GAGAACTCGAACCAAGCGAGGGAAAAATAAAGCACTCTGGACGGATTAGTTTTTGCTC
CCAGTTCTCGTGGATAATGCCTGGCACCATTAAGGAGAATATCATCTTTGGAGTGAGTT
ACGACGAATACCGGTACCGGTCCGTTATCAAGGCTTGTCAACTCGAGGAGGACATTTCT
AAATTCGCCGAAAAAGATAATATAGTGCTGGGCGAAGGAGGCATTACACTGAGCGGGG
GTCAGAGAGCTCGAATTAGCCTCGCCCGAGCAGTCTATAAAGACGCCGATCTTTACCTG
CTGGATTCCCCTTTTGGGTATTTGGATGTTCTGACAGAGAAGGAAATCTTTGAATCATGT
GTCTGTAAACTGATGGCCAATAAGACTAGGATTCTAGTGACTTCGAAAATGGAGCACCT
GAAAAAAGCGGACAAAATTCTGATACTCCATGAAGGGTCTTCCTACTTCTACGGCACCT
TCTCAGAGTTGCAGAACTTACAACCTGATTTTTCATCTAAGCTTATGGGGTGCGACTCGT
TTGACCAGTTCTCCGCTGAAAGACGAAACAGCATCTTAACGGAAACTCTTCACAGGTTC
TCATTAGAGGGAGATGCGCCGGTGTCCTGGACAGAGACAAAAAAACAGTCTTTCAAAC
AGACAGGAGAGTTTGGCGAGAAGAGAAAAAACTCAATCCTCAATCCCATCAATTCTATT
AGAAAGTTTAGCATCGTCCAAAAAACACCATTGCAGATGAATGGGATTGAGGAGGACA
GTGATGAGCCTTTGGAACGAAGACTGTCCCTGGTACCCGATAGCGAACAGGGTGAGGC
CATCCTTCCTAGGATCTCGGTCATAAGTACAGGGCCCACACTGCAGGCCAGGCGACGTC
AAAGTGTCCTCAATCTTATGACGCACAGTGTGAATCAGGGGCAGAACATCCATCGTAAG
ACGACAGCTTCAACTCGAAAGGTCAGTCTAGCTCCACAAGCCAATCTTACAGAGCTGGA
CATTTATTCCCGCCGCCTCAGTCAGGAGACCGGATTGGAAATATCAGAGGAAATTAATG
AAGAGGATCTGAAGGAATGCTTCTTTGATGACATGGAATCGATCCCCGCTGTTACTACC
TGGAACACATATCTGAGATATATTACCGTCCATAAGAGCTTAATCTTTGTACTGATATG
GTGCTTGGTGATTTTCCTGGCAGAGGTTGCGGCGAGTTTGGTCGTGCTATGGCTCCTTGG
AAACACTCCCCTGCAGGATAAGGGGAACTCCACTCATAGCAGGAATAACAGCTATGCC
GTGATCATCACCTCTACCTCCTCTTATTACGTGTTTTACATATACGTCGGTGTTGCGGAT
ACCCTGTTGGCAATGGGGTTCTTTAGAGGACTACCCCTAGTTCACACCCTGATCACCGTT
TCGAAGATCTTGCACCACAAGATGCTTCATAGCGTTCTCCAAGCTCCTATGAGCACCCT
TAATACACTGAAAGCAGGAGGTATCCTTAACCGCTTTTCCAAAGACATCGCTATACTCG
ACGATTTGCTCCCATTGACCATCTTCGACTTCATTCAGCTGCTCCTCATTGTGATCGGCG
CCATTGCCGTGGTCGCAGTGTTACAGCCATATATTTTCGTAGCCACCGTGCCCGTCATCG
TGGCATTTATCATGCTGCGCGCATATTTCTTACAGACATCTCAGCAACTGAAGCAGCTG
GAATCTGAGGGCAGATCTCCTATTTTTACACACCTGGTTACCAGCCTGAAGGGCCTGTG
GACCCTGCGTGCTTTCGGTCGCCAACCCTACTTTGAGACTCTCTTCCATAAGGCTCTGAA
TTTACATACTGCCAATTGGTTCCTATACCTTAGTACCCTTCGGTGGTTCCAGATGCGGAT
AGAAATGATCTTCGTGATTTTCTTCATCGCAGTCACTTTCATCTCTATTTTGACGACCGG
TGAGGGCGAGGGCAGGGTGGGCATCATTCTGACTTTGGCCATGAACATTATGTCAACAC
TCCAGTGGGCCGTTAATTCAAGCATTGATGTGGATTCCTTGATGCGTTCCGTCAGCAGG
GTATTTAAATTCATAGACATGCCCACCGAGGGCAAGCCAACAAAATCTACCAAGCCAT
ACAAAAATGGCCAACTAAGCAAGGTCATGATTATCGAGAATTCTCATGTGAAAAAGGA
CGACATTTGGCCTTCCGGGGGTCAAATGACTGTAAAGGACCTGACGGCTAAATACACTG
AGGGCGGTAATGCTATCTTGGAGAACATCTCTTTCAGCATCTCCCCTGGCCAGAGAGTG
GGACTGCTCGGGCGGACAGGCTCCGGAAAGTCTACGCTCCTTTCAGCATTCCTTAGACT
TCTGAACACCGAAGGTGAGATTCAGATTGACGGGGTCTCTTGGGACTCCATCACACTTC
AGCAATGGAGGAAGGCATTCGGTGTAATCCCCCAAAAGGTTTTTATCTTCTCCGGAACA
TTTCGTAAGAATCTGGACCCGTACGAGCAGTGGTCAGATCAGGAGATCTGGAAAGTAG
CAGACGAGGTCGGGCTACGGAGCGTTATTGAACAGTTTCCTGGCAAACTGGACTTCGTT
TTGGTGGACGGAGGCTGTGTGCTGAGTCACGGCCATAAACAACTGATGTGCTTAGCTAG
GTCTGTTCTCAGCAAGGCAAAGATTTTACTGCTGGATGAACCAAGCGCCCACCTTGATC
CAGTGACATATCAAATCATCAGAAGAACTCTTAAACAGGCGTTCGCCGACTGCACAGTG
ATCCTGTGTGAGCACAGAATAGAAGCCATGCTGGAATGTCAACAGTTTCTCGTGATTGA
GGAGAACAAGGTGCGCCAGTACGATAGCATCCAGAAGTTACTCAATGAAAGGTCACTC
TTCAGGCAGGCCATCTCACCCAGCGACCGCGTTAAGCTGTTTCCACACCGAAACAGTTC
CAAGTGCAAAAGTAAGCCACAGATTGCTGCACTGAAGGAAGAGACAGAAGAAGAAGTT
CAGGACACTCGGCTCTGA.

In another embodiment, an exemplary codon-optimized CFTR mRNA sequence is:

(SEQ ID NO: 26)
ATGCAGAGGAGCCCACTGGAGAAAGCCTCCGTGGTGAGTAAACTCTTTTTTAGTTGGAC
CAGACCCATCCTGCGAAAAGGATACAGGCAGCGCCTCGAGTTGTCAGATATCTACCAG
ATTCCTTCTGTGGACTCAGCTGACAATTTGAGTGAGAAGCTGGAGCGGGAGTGGGATAG
AGAGCTGGCGAGCAAAAAAAACCCCAAGCTTATCAATGCTCTGCGCCGCTGCTTTTTCT
GGAGGTTCATGTTTTATGGGATCTTCCTGTACCTGGGGGAGGTCACCAAAGCTGTTCAG
CCGCTCCTTCTTGGCCGCATCATCGCCAGCTATGACCCTGATAATAAAGAAGAAAGGTC
TATTGCTATTTATCTGGGAATTGGCCTCTGCTTGCTCTTCATCGTCCGCACCCTTCTGCTG
CACCCTGCCATTTTTGGCCTTCACCACATCGGCATGCAAATGAGAATTGCCATGTTCTCC
CTCATTTACAAAAAGACCCTGAAACTTTCCTCAAGAGTGTTAGATAAAATATCCATTGG
TCAGCTGGTCAGCCTGCTGTCCAACAATCTTAACAAATTTGATGAAGGCTTGGCGCTGG
CCCACTTCGTGTGGATTGCACCTCTGCAGGTGGCCCTGTTGATGGGACTTATATGGGAG
CTGCTTCAAGCCTCTGCTTTCTGTGGGCTGGGCTTTTTGATTGTACTGGCACTTTTTCAGG
CTGGGCTCGGAAGAATGATGATGAAATACAGAGATCAGCGGGCCGGGAAGATATCAGA
GCGACTTGTGATCACCAGTGAAATGATTGAAAATATTCAGAGCGTGAAAGCCTACTGCT
GGGAAGAAGCCATGGAGAAGATGATTGAGAACCTGAGGCAGACAGAGCTCAAGCTCAC
TCGGAAGGCTGCTTATGTTCGCTATTTCAACAGCAGCGCCTTCTTCTTCAGTGGCTTCTT
TGTTGTCTTCCTGTCTGTTCTGCCATATGCACTGATAAAAGGCATTATTTTACGAAAGAT
CTTCACCACCATCAGTTTTTGCATCGTTCTCAGGATGGCCGTCACAAGACAGTTCCCCTG
GGCTGTGCAGACCTGGTACGATTCCTTGGGGGCCATCAACAAGATTCAAGATTTCTTGC
AAAAACAAGAATATAAAACTTTAGAATACAACCTCACCACCACTGAAGTGGTCATGGA
AAATGTGACAGCCTTTTGGGAGGAGGGTTTTGGAGAATTGTTCGAGAAGGCAAAGCAG
AATAACAACAACAGGAAGACGAGCAATGGGGACGACTCTCTCTTCTTCAGCAACTTTTC
ACTGCTCGGGACCCCTGTGTTGAAAGATATAAACTTCAAGATCGAGAGGGGCCAGCTCT
TGGCTGTGGCAGGCTCCACTGGAGCTGGTAAAACATCTCTTCTCATGGTGATCATGGGG
GAACTGGAGCCTTCCGAAGGAAAAATCAAGCACAGTGGGAGAATCTCATTCTGCAGCC
AGTTTTCCTGGATCATGCCCGGCACCATTAAGGAAAACATCATATTTGGAGTGTCCTAT
GATGAGTACCGCTACCGGTCAGTCATCAAAGCCTGTCAGTTGGAGGAGGACATCTCCAA
GTTTGCAGAGAAAGACAACATTGTGCTTGGAGAGGGGGGTATCACTCTTTCTGGAGGAC
AAAGAGCCAGGATCTCTTTGGCCCGGGCAGTCTACAAGGATGCAGACCTCTACTTGTTG
GACAGTCCCTTCGGCTACCTCGACGTGCTGACTGAAAAAGAAATTTTTGAAAGCTGTGT
GTGCAAACTGATGGCAAACAAGACCAGGATTCTTGTCACCAGCAAGATGGAACATCTG
AAGAAAGCGGACAAAATTCTGATTCTGCATGAAGGGAGCTCCTACTTCTATGGAACATT
TAGCGAGCTTCAGAACCTACAGCCAGACTTCTCCTCCAAATTAATGGGCTGTGACTCCT
TCGACCAGTTCTCTGCAGAAAGAAGAAACTCTATACTCACAGAGACCCTCCACCGCTTC
TCCCTTGAGGGAGATGCCCCAGTTTCTTGGACAGAAACCAAGAAGCAGTCCTTTAAGCA
GACTGGCGAGTTTGGTGAAAAGAGGAAAAATTCAATTCTCAATCCAATTAACAGTATTC
GCAAGTTCAGCATTGTCCAGAAGACACCCCTCCAGATGAATGGCATCGAAGAAGATAG
TGACGAGCCGCTGGAGAGACGGCTGAGTCTGGTGCCAGATTCAGAACAGGGGGAGGCC
ATCCTGCCCCGGATCAGCGTCATTTCCACAGGCCCCACATTACAAGCACGGCGCCGGCA
GAGTGTTTTAAATCTCATGACCCATTCAGTGAACCAGGGCCAAAATATCCACAGGAAGA
CTACAGCTTCTACCCGGAAAGTGTCTCTGGCCCCTCAGGCCAATCTGACCGAGCTGGAC
ATCTACAGCAGGAGGCTCTCCCAGGAAACAGGGCTGGAAATATCTGAAGAGATTAATG
AAGAGGATCTTAAAGAGTGCTTCTTTGATGACATGGAGAGCATCCCCGCGGTGACCACA
TGGAACACCTACCTTAGATATATTACTGTCCACAAGAGCCTCATATTTGTCCTCATCTGG
TGCCTGGTTATTTTCCTCGCTGAGGTGGCGGCCAGTCTTGTTGTGCTCTGGCTGCTGGGC
AACACTCCTCTCCAGGACAAGGGCAATAGTACTCACAGCAGAAATAATTCTTATGCCGT
CATCATTACAAGCACCTCCAGCTACTACGTGTTCTACATCTATGTGGGCGTGGCTGACA
CCCTCCTGGCCATGGGTTTCTTCCGGGGCCTGCCTTTGGTGCACACCCTCATCACAGTGT
CAAAAATTCTGCACCATAAAATGCTTCATTCTGTCCTGCAGGCACCCATGAGCACTTTG
AACACATTGAAGGCTGGCGGCATCCTCAACAGATTTTCTAAAGATATTGCTATCCTGGA
TGATCTCCTCCCCCTGACAATCTTTGACTTTATCCAGCTTCTGCTGATCGTGATTGGAGC
CATAGCAGTGGTTGCTGTCCTGCAGCCCTACATTTTTGTGGCCACCGTGCCCGTGATTGT
TGCCTTTATTATGCTCAGAGCTTACTTCCTGCAAACTTCTCAACAGCTCAAACAGCTAGA
ATCTGAGGGCCGGAGCCCCATTTTTACCCACCTGGTGACTTCCCTGAAGGGACTGTGGA
CTCTGAGAGCATTCGGGCGACAGCCTTACTTTGAGACACTGTTCCACAAGGCCCTGAAC
TTGCACACTGCCAACTGGTTTCTTTACCTGAGCACACTCCGCTGGTTCCAGATGCGGATA
GAGATGATCTTCGTCATCTTTTTTATAGCTGTAACCTTCATTTCTATCCTTACAACAGGA
GAAGGAGAGGGCAGGGTGGGAATCATCCTCACGCTGGCTATGAACATAATGTCCACCT
TGCAGTGGGCCGTGAATTCCAGTATAGATGTGGATTCTCTAATGAGGAGTGTCTCCCGG
GTGTTTAAATTCATTGATATGCCTACTGAGGGGAAACCCACCAAGTCAACAAAACCTTA
TAAGAATGGACAGCTGAGCAAGGTGATGATAATTGAGAACAGCCACGTGAAGAAGGAT
GACATTTGGCCCAGCGGGGGCCAGATGACTGTGAAGGACCTGACGGCCAAGTACACCG
AAGGTGGAAATGCCATTTTGGAAAACATCAGCTTCTCAATCTCTCCTGGGCAGAGAGTT
GGATTGCTGGGTCGCACGGGCAGCGGCAAATCAACCCTGCTCAGTGCCTTCCTTCGGCT
CCTGAATACAGAAGGCGAAATCCAAATTGACGGGGTGAGCTGGGACAGCATCACCCTG
CAGCAGTGGAGAAAAGCATTTGGGGTCATTCCACAGAAAGTTTTCATCTTCTCTGGCAC
TTTCAGAAAGAACCTGGACCCCTATGAGCAGTGGAGCGACCAGGAGATCTGGAAGGTT
GCAGATGAAGTTGGCCTGCGGAGTGTGATAGAACAATTTCCTGGCAAGCTGGATTTTGT
GCTGGTAGATGGAGGCTGCGTGCTGTCCCACGGCCACAAACAGCTGATGTGCCTCGCCC
GCTCCGTTCTTTCAAAGGCCAAAATCTTGCTTTTGGATGAGCCCAGTGCTCACCTCGACC
CAGTGACCTATCAGATAATCCGCAGGACCTTAAAGCAAGCTTTTGCCGACTGCACCGTC
ATACTGTGTGAGCACCGGATTGAAGCAATGCTGGAATGCCAGCAGTTTCTGGTGATCGA
GGAGAATAAGGTCCGGCAGTACGACAGCATCCAGAAGTTGTTGAATGAGCGCAGCCTT
TTCCGCCAGGCCATCTCCCCATCTGACAGAGTCAAGCTGTTTCCACATAGGAACTCCTCT
AAGTGCAAGTCCAAGCCCCAGATCGCTGCCCTCAAGGAGGAAACTGAGGAAGAGGTGC
AGGATACCCGCCTGTGA.

(SEQ ID NO: 27)
ATGCAACGGAGTCCTCTGGAAAAAGCCTCTGTCGTATCTAAGCTTTTCTTCAGTTGGAC
ACGCCCGATTTTGAGAAAGGGTTATCGGCAACGCTTGGAACTTAGTGACATCTACCAAA
TTCCAAGTGTAGACTCAGCCGATAACTTGAGCGAAAAGCTCGAACGAGAGTGGGATCG
AGAACTGGCTAGCAAAAAAAATCCCAAACTCATAAATGCCCTGCGACGCTGTTTCTTTT
GGCGATTTATGTTTTACGGTATTTTCCTTTATTTGGGTGAGGTCACGAAGGCTGTACAGC
CACTGCTGCTGGGTCGCATCATTGCCTCTTACGACCCTGACAACAAAGAGGAGCGGTCA
ATAGCTATCTACCTTGGTATAGGACTTTGCTTGCTCTTCATAGTCCGCACGTTGCTTCTC
CACCCTGCTATATTTGGTCTCCATCACATTGGGATGCAAATGCGGATCGCGATGTTCAGT
CTTATATATAAAAAGACTCTTAAACTTTCCAGCCGGGTTCTGGATAAGATCTCTATTGGT
CAACTGGTATCTCTTTTGTCTAACAACCTGAATAAGTTCGACGAGGGCCTTGCATTGGCC
CATTTTGTATGGATTGCCCCTTTGCAAGTCGCCCTCCTGATGGGATTGATCTGGGAACTC
CTGCAAGCTAGTGCTTTTTGCGGATTGGGATTCCTCATAGTCCTTGCGCTCTTTCAGGCG
GGACTTGGACGCATGATGATGAAGTATCGCGACCAACGAGCTGGCAAGATCAGTGAAC
GGCTTGTAATAACCAGTGAAATGATAGAGAACATCCAGAGCGTAAAAGCTTACTGTTG
GGAAGAAGCGATGGAAAAGATGATTGAGAACCTTCGCCAGACAGAACTTAAACTTACA
CGAAAGGCCGCTTATGTCCGGTACTTCAACTCTTCAGCATTTTTTTTTAGTGGCTTCTTTG
TAGTGTTCCTGTCCGTCCTTCCGTATGCACTTATCAAGGGTATAATACTTAGGAAAATCT
TCACAACAATCAGTTTTTGCATAGTCCTTCGCATGGCAGTAACTCGCCAATTTCCCTGGG
CAGTTCAGACGTGGTACGACTCACTTGGCGCAATTAACAAAATTCAAGATTTCCTCCAA
AAGCAAGAGTATAAAACCTTGGAATACAACCTTACCACCACAGAAGTTGTAATGGAAA
ATGTCACAGCCTTCTGGGAGGAAGGTTTCGGCGAACTTTTTGAGAAGGCGAAGCAAAAT
AACAATAATCGGAAAACATCAAACGGTGACGATTCACTGTTCTTTTCTAACTTTAGCCTT
CTTGGGACGCCCGTCCTGAAGGACATAAACTTTAAGATTGAACGGGGTCAACTTCTCGC
GGTCGCAGGGAGTACTGGAGCGGGGAAAACGAGCCTGCTGATGGTGATAATGGGGGAG
TTGGAGCCCTCAGAAGGCAAGATCAAGCATAGTGGTAGAATTAGCTTCTGCAGTCAATT
TAGTTGGATTATGCCGGGCACGATCAAAGAAAATATAATCTTTGGGGTATCCTACGATG
AATACAGGTACCGATCAGTGATAAAAGCGTGCCAGCTTGAAGAAGACATTTCAAAGTTT
GCTGAGAAGGATAATATCGTACTTGGAGAAGGAGGTATCACCCTGTCTGGGGGTCAAC
GAGCGAGGATCTCCCTGGCACGCGCCGTCTACAAGGACGCGGACCTCTATCTGTTGGAT
TCACCGTTCGGATATTTGGACGTGCTTACGGAGAAAGAAATATTTGAGAGCTGTGTTTG
CAAGCTCATGGCAAATAAAACCAGAATATTGGTTACAAGCAAGATGGAGCATCTTAAG
AAAGCAGATAAAATCCTGATATTGCACGAGGGCTCTTCATACTTCTACGGGACGTTTTC
TGAGTTGCAGAACCTCCAGCCGGATTTCAGCTCTAAGCTGATGGGCTGTGATTCCTTTG
ATCAGTTTAGTGCGGAAAGACGAAACAGTATACTCACCGAAACACTGCACAGGTTCTCT
CTGGAGGGCGACGCCCCGGTTTCCTGGACAGAGACGAAGAAGCAGTCCTTCAAACAGA
CAGGCGAGTTTGGGGAGAAAAGGAAAAATAGCATACTCAACCCGATTAACAGCATTCG
CAAGTTCAGTATAGTACAAAAGACCCCGTTGCAGATGAACGGTATAGAGGAAGATTCT
GATGAGCCACTGGAAAGACGGCTTTCTCTCGTTCCGGACAGTGAACAGGGAGAGGCAA
TACTGCCTCGGATCAGCGTTATCTCTACAGGACCTACTTTGCAAGCTCGGCGCCGACAG
TCAGTCTTGAATCTTATGACTCATAGTGTTAATCAAGGCCAGAATATCCATCGCAAGAC
CACCGCAAGTACAAGGAAAGTGAGCTTGGCACCTCAAGCAAACCTTACTGAACTTGAT
ATCTACTCACGGCGACTTTCACAGGAGACCGGACTTGAAATTAGTGAAGAAATTAACGA
GGAGGACCTCAAGGAGTGCTTCTTCGATGACATGGAATCAATCCCCGCAGTCACAACCT
GGAACACTTATCTGAGGTATATAACAGTTCACAAGAGCCTCATTTTTGTACTTATTTGGT
GTTTGGTAATTTTCCTGGCGGAGGTTGCTGCTTCTTTGGTCGTCCTTTGGCTCCTCGGGA
ATACACCGCTCCAAGACAAAGGCAACTCTACCCATAGTAGGAACAATTCATATGCAGT
GATTATAACCAGTACATCATCTTATTACGTTTTCTATATTTATGTCGGGGTAGCTGACAC
GCTGTTGGCGATGGGCTTCTTTAGGGGCCTCCCCTTGGTACACACCCTTATCACGGTGAG
TAAAATCCTGCATCACAAAATGCTTCATTCTGTACTCCAAGCGCCGATGAGTACGCTTA
ATACGCTGAAAGCAGGAGGGATACTGAATCGGTTCAGCAAGGACATCGCCATTCTGGA
TGACCTGCTTCCATTGACAATATTTGATTTCATTCAGCTCCTTCTCATAGTTATTGGAGC
CATAGCGGTGGTGGCTGTGCTTCAGCCTTATATATTCGTTGCCACAGTTCCCGTTATAGT
GGCATTTATAATGCTCAGGGCCTACTTTCTCCAGACTTCCCAGCAGTTGAAGCAACTCG
AATCAGAAGGAAGGTCACCTATTTTCACACATCTTGTGACTTCCTTGAAGGGCTTGTGG
ACGCTGCGGGCCTTCGGAAGACAACCATATTTTGAAACTCTCTTCCACAAAGCTTTGAA
TCTTCATACTGCGAACTGGTTCCTGTATTTGAGTACTTTGCGCTGGTTCCAGATGAGGAT
AGAAATGATATTCGTTATCTTCTTTATCGCGGTTACGTTCATAAGTATCCTCACTACGGG
GGAGGGTGAGGGTAGAGTGGGCATAATACTGACCCTCGCCATGAACATTATGTCCACCC
TGCAGTGGGCGGTAAACAGCAGCATAGATGTGGATTCTTTGATGCGCAGTGTGAGCAG
GGTTTTTAAGTTTATCGATATGCCGACGGAAGGAAAGCCCACTAAAAGCACGAAACCCT
ATAAAAATGGACAGCTTAGCAAAGTAATGATAATCGAGAATAGCCATGTGAAAAAGGA
TGACATATGGCCTTCCGGAGGCCAAATGACTGTTAAAGATCTGACCGCTAAATATACCG
AGGGCGGCAACGCAATACTCGAAAACATAAGCTTTTCCATAAGCCCCGGCCAACGCGT
GGGTCTTCTGGGGAGGACTGGCTCCGGAAAATCAACGTTGCTTAGCGCGTTTTTGCGGC
TCCTTAACACTGAAGGTGAGATCCAAATAGATGGCGTTAGTTGGGACTCTATAACACTG
CAACAATGGCGGAAAGCTTTCGGCGTCATACCTCAGAAGGTGTTCATCTTTAGCGGAAC
GTTCAGGAAGAACTTGGATCCCTACGAACAATGGAGTGATCAAGAAATATGGAAAGTG
GCAGATGAGGTAGGCTTGCGCAGTGTCATTGAACAATTCCCAGGGAAACTCGACTTTGT
ACTGGTGGACGGCGGTTGCGTCTTGTCACACGGGCACAAACAGTTGATGTGTTTGGCCC
GCAGTGTTTTGTCTAAGGCGAAGATTCTGTTGCTCGACGAACCGAGTGCTCATCTTGATC
CCGTCACCTACCAAATCATCAGAAGGACGTTGAAGCAAGCTTTCGCCGACTGCACTGTA
ATCCTTTGTGAGCATAGGATCGAAGCAATGCTCGAGTGCCAACAGTTCTTGGTTATAGA
GGAGAATAAGGTTCGGCAATACGACTCAATACAGAAACTGCTTAATGAGCGGTCACTCT
TTCGACAAGCTATCTCTCCTAGTGACAGGGTAAAGCTTTTTCCTCATCGGAATTCCAGCA
AGTGTAAGAGTAAACCACAGATCGCCGCCCTTAAAGAGGAGACCGAAGAAGAGGTGCA
GGATACGAGACTTTAG.

In some embodiments, a codon-optimized CFTR mRNA sequence suitable for the present invention shares at least 60%, 65%, 70%, 75%, 80%, 85%, 90%, 95%, 96%, 97%, 98%, or 99% identity to SEQ ID NO:6 or SEQ ID NO:7 and encodes a CFTR protein having an amino acid sequence of SEQ ID NO:2. In a specific embodiment, a codon-optimized CFTR mRNA sequence suitable for the present invention has the nucleotide sequence of SEQ ID NO:6.

In some embodiments, a suitable mRNA sequence may be an mRNA sequence encoding a homolog or an analog of human CFTR (hCFTR) protein. For example, a homolog or an analog of hCFTR protein may be a modified hCFTR protein containing one or more amino acid substitutions, deletions, and/or insertions as compared to a wild-type or naturally-occurring hCFTR protein while retaining substantial hCFTR protein activity. In some embodiments, an mRNA suitable for the present invention encodes an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 2. In some embodiments, an mRNA suitable for the present invention encodes a protein substantially identical to hCFTR protein. In some embodiments, an mRNA suitable for the present invention encodes an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO: 2. In some embodiments, an mRNA suitable for the present invention encodes a fragment or a portion of hCFTR protein. In some embodiments, an mRNA suitable for the present invention encodes a fragment or a portion of hCFTR protein, wherein the fragment or portion of the protein still maintains CFTR activity similar to that of the wild-type protein. Thus, in some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ ID NO: 1, SEQ ID NO: 6 or SEQ ID NO: 7.

In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical to any one of SEQ ID NO: 8, SEQ ID NO: 29, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

In some embodiments, a suitable mRNA encodes a fusion protein comprising a full length, fragment or portion of an hCFTR protein fused to another protein (e.g., an N or C terminal fusion). In some embodiments, the protein fused to the mRNA encoding a full length, fragment or portion of an hCFTR protein encodes a signal or a cellular targeting sequence.

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs (e.g., mRNAs encoding CFTR) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′-5′ triphosphate linkage; and the 7-nitrogen of guanine is then methylated by a methyltransferase. In some embodiments, the nucleotide forming the cap is further methylated at the 3′position. In some embodiments, the nucleotide directly adjacent to the cap is further methylated at the 2′ position. Examples of cap structures include, but are not limited to, m⁷G(5′)ppp(5′) (2′OMeG), m⁷G(5′)ppp(5′) (2′OMeA), m⁷ (3′OMeG) (5′)ppp(5′) (2′OMeG), m⁷ (3′OMeG) (5′)ppp(5′) (2′OMeA), m⁷G(5′)ppp(5′ (A,G(5′)ppp(5′) A and G(5′)ppp(5′) G. In a specific embodiment, the cap structure is m⁷G(5′)ppp(5′) (2′OMeG). Additional cap structures are described in published US Application No. US 2016/0032356 and U.S. Provisional Application 62/464,327, filed Feb. 27, 2017, which are incorporated herein by reference.

In some embodiments, mRNAs (e.g., mRNAs encoding CFTR) include a 3′ tail structure. Typically, a tail structure includes a poly(A) and/or poly(C) tail. A poly-A or poly-C tail on the 3′ terminus of mRNA typically includes at least 50 adenosine or cytosine nucleotides, at least 100 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly-A or poly-C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly(A) and poly(C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

In a specific embodiment, an mRNA encoding CFTR that has a poly(A) tail of between 200 and 1000 adenosine nucleotides (e.g., as determined using agarose gel electrophoresis) is particularly suitable for practicing the invention. Typically, an mRNA encoding CFTR for use with the invention has a poly(A) tail that is between 400 and 700 adenosine nucleotides (e.g., as determined using agarose gel electrophoresis).

In a specific embodiment, the mRNA encoding CFTR has the following sequence and structural elements:

(SEQ ID NO: 28)
GGACAGAUCGCCUGGAGACGCCAUCCACGCUGUUUUGACCUCCAUAGAAGACACCGG
GACCGAUCCAGCCUCCGCGGCCGGGAACGGUGCAUUGGAACGCGGAUUCCCCGUGCC
AAGAGUGACUCACCGUCCUUGACACGAUGCAACGCUCUCCUCUUGAAAAGGCCUCGG
UGGUGUCCAAGCUCUUCUUCUCGUGGACUAGACCCAUCCUGAGAAAGGGGUACAGAC
AGCGCUUGGAGCUGUCCGAUAUCUAUCAAAUCCCUUCCGUGGACUCCGCGGACAACC
UGUCCGAGAAGCUCGAGAGAGAAUGGGACAGAGAACUCGCCUCAAAGAAGAACCCGA
AGCUGAUUAAUGCGCUUAGGCGGUGCUUUUUCUGGCGGUUCAUGUUCUACGGCAUC
UUCCUCUACCUGGGAGAGGUCACCAAGGCCGUGCAGCCCCUGUUGCUGGGACGGAUU
AUUGCCUCCUACGACCCCGACAACAAGGAAGAAAGAAGCAUCGCUAUCUACUUGGGC
AUCGGUCUGUGCCUGCUUUUCAUCGUCCGGACCCUCUUGUUGCAUCCUGCUAUUUUC
GGCCUGCAUCACAUUGGCAUGCAGAUGAGAAUUGCCAUGUUUUCCCUGAUCUACAAG
AAAACUCUGAAGCUCUCGAGCCGCGUGCUUGACAAGAUUUCCAUCGGCCAGCUCGUG
UCCCUGCUCUCCAACAAUCUGAACAAGUUCGACGAGGGCCUCGCCCUGGCCCACUUC
GUGUGGAUCGCCCCUCUGCAAGUGGCGCUUCUGAUGGGCCUGAUCUGGGAGCUGCUG
CAAGCCUCGGCAUUCUGUGGGCUUGGAUUCCUGAUCGUGCUGGCACUGUUCCAGGCC
GGACUGGGGCGGAUGAUGAUGAAGUACAGGGACCAGAGAGCCGGAAAGAUUUCCGA
ACGGCUGGUGAUCACUUCGGAAAUGAUCGAAAACAUCCAGUCAGUGAAGGCCUACUG
CUGGGAAGAGGCCAUGGAAAAGAUGAUUGAAAACCUCCGGCAAACCGAGCUGAAGC
UGACCCGCAAGGCCGCUUACGUGCGCUAUUUCAACUCGUCCGCUUUCUUCUUCUCCG
GGUUCUUCGUGGUGUUUCUCUCCGUGCUCCCCUACGCCCUGAUUAAGGGAAUCAUCC
UCAGGAAGAUCUUCACCACCAUUUCCUUCUGUAUCGUGCUCCGCAUGGCCGUGACCC
GGCAGUUCCCAUGGGCCGUGCAGACUUGGUACGACUCCCUGGGAGCCAUUAACAAGA
UCCAGGACUUCCUUCAAAAGCAGGAGUACAAGACCCUCGAGUACAACCUGACUACUA
CCGAGGUCGUGAUGGAAAACGUCACCGCCUUUUGGGAGGAGGGAUUUGGCGAACUG
UUCGAGAAGGCCAAGCAGAACAACAACAACCGCAAGACCUCGAACGGUGACGACUCC
CUCUUCUUUUCAAACUUCAGCCUGCUCGGGACGCCCGUGCUGAAGGACAUUAACUUC
AAGAUCGAAAGAGGACAGCUCCUGGCGGUGGCCGGAUCGACCGGAGCCGGAAAGACU
UCCCUGCUGAUGGUGAUCAUGGGAGAGCUUGAACCUAGCGAGGGAAAGAUCAAGCA
CUCCGGCCGCAUCAGCUUCUGUAGCCAGUUUUCCUGGAUCAUGCCCGGAACCAUUAA
GGAAAACAUCAUCUUCGGCGUGUCCUACGAUGAAUACCGCUACCGGUCCGUGAUCAA
AGCCUGCCAGCUGGAAGAGGAUAUUUCAAAGUUCGCGGAGAAAGAUAACAUCGUGC
UGGGCGAAGGGGGUAUUACCUUGUCGGGGGGCCAGCGGGCUAGAAUCUCGCUGGCCA
GAGCCGUGUAUAAGGACGCCGACCUGUAUCUCCUGGACUCCCCCUUCGGAUACCUGG
ACGUCCUGACCGAAAAGGAGAUCUUCGAAUCGUGCGUGUGCAAGCUGAUGGCUAACA
AGACUCGCAUCCUCGUGACCUCCAAAAUGGAGCACCUGAAGAAGGCAGACAAGAUUC
UGAUUCUGCAUGAGGGGUCCUCCUACUUUUACGGCACCUUCUCGGAGUUGCAGAACU
UGCAGCCCGACUUCUCAUCGAAGCUGAUGGGUUGCGACAGCUUCGACCAGUUCUCCG
CCGAAAGAAGGAACUCGAUCCUGACGGAAACCUUGCACCGCUUCUCUUUGGAAGGCG
ACGCCCCUGUGUCAUGGACCGAGACUAAGAAGCAGAGCUUCAAGCAGACCGGGGAAU
UCGGCGAAAAGAGGAAGAACAGCAUCUUGAACCCCAUUAACUCCAUCCGCAAGUUCU
CAAUCGUGCAAAAGACGCCACUGCAGAUGAACGGCAUUGAGGAGGACUCCGACGAAC
CCCUUGAGAGGCGCCUGUCCCUGGUGCCGGACAGCGAGCAGGGAGAAGCCAUCCUGC
CUCGGAUUUCCGUGAUCUCCACUGGUCCGACGCUCCAAGCCCGGCGGCGGCAGUCCG
UGCUGAACCUGAUGACCCACAGCGUGAACCAGGGCCAAAACAUUCACCGCAAGACUA
CCGCAUCCACCCGGAAAGUGUCCCUGGCACCUCAAGCGAAUCUUACCGAGCUCGACA
UCUACUCCCGGAGACUGUCGCAGGAAACCGGGCUCGAAAUUUCCGAAGAAAUCAACG
AGGAGGAUCUGAAAGAGUGCUUCUUCGACGAUAUGGAGUCGAUACCCGCCGUGACG
ACUUGGAACACUUAUCUGCGGUACAUCACUGUGCACAAGUCAUUGAUCUUCGUGCUG
AUUUGGUGCCUGGUGAUUUUCCUGGCCGAGGUCGCGGCCUCACUGGUGGUGCUCUGG
CUGUUGGGAAACACGCCUCUGCAAGACAAGGGAAACUCCACGCACUCGAGAAACAAC
AGCUAUGCCGUGAUUAUCACUUCCACCUCCUCUUAUUACGUGUUCUACAUCUACGUC
GGAGUGGCGGAUACCCUGCUCGCGAUGGGUUUCUUCAGAGGACUGCCGCUGGUCCAC
ACCUUGAUCACCGUCAGCAAGAUUCUUCACCACAAGAUGUUGCAUAGCGUGCUGCAG
GCCCCCAUGUCCACCCUCAACACUCUGAAGGCCGGAGGCAUUCUGAACAGAUUCUCC
AAGGACAUCGCUAUCCUGGACGAUCUCCUGCCGCUUACCAUCUUUGACUUCAUCCAG
CUGCUGCUGAUCGUGAUUGGAGCAAUCGCAGUGGUGGCGGUGCUGCAGCCUUACAUU
UUCGUGGCCACUGUGCCGGUCAUUGUGGCGUUCAUCAUGCUGCGGGCCUACUUCCUC
CAAACCAGCCAGCAGCUGAAGCAACUGGAAUCCGAGGGACGAUCCCCCAUCUUCACU
CACCUUGUGACGUCGUUGAAGGGACUGUGGACCCUCCGGGCUUUCGGACGGCAGCCC
UACUUCGAAACCCUCUUCCACAAGGCCCUGAACCUCCACACCGCCAAUUGGUUCCUG
UACCUGUCCACCCUGCGGUGGUUCCAGAUGCGCAUCGAGAUGAUUUUCGUCAUCUUC
UUCAUCGCGGUCACAUUCAUCAGCAUCCUGACUACCGGAGAGGGAGAGGGACGGGUC
GGAAUAAUCCUGACCCUCGCCAUGAACAUUAUGAGCACCCUGCAGUGGGCAGUGAAC
AGCUCGAUCGACGUGGACAGCCUGAUGCGAAGCGUCAGCCGCGUGUUCAAGUUCAUC
GACAUGCCUACUGAGGGAAAACCCACUAAGUCCACUAAGCCCUACAAAAAUGGCCAG
CUGAGCAAGGUCAUGAUCAUCGAAAACUCCCACGUGAAGAAGGACGAUAUUUGGCCC
UCCGGAGGUCAAAUGACCGUGAAGGACCUGACCGCAAAGUACACCGAGGGAGGAAAC
GCCAUUCUCGAAAACAUCAGCUUCUCCAUUUCGCCGGGACAGCGGGUCGGCCUUCUC
GGGCGGACCGGUUCCGGGAAGUCAACUCUGCUGUCGGCUUUCCUCCGGCUGCUGAAU
ACCGAGGGGGAAAUCCAAAUUGACGGCGUGUCUUGGGAUUCCAUUACUCUGCAGCAG
UGGCGGAAGGCCUUCGGCGUGAUCCCCCAGAAGGUGUUCAUCUUCUCGGGUACCUUC
CGGAAGAACCUGGAUCCUUACGAGCAGUGGAGCGACCAAGAAAUCUGGAAGGUCGCC
GACGAGGUCGGCCUGCGCUCCGUGAUUGAACAAUUUCCUGGAAAGCUGGACUUCGUG
CUCGUCGACGGGGGAUGUGUCCUGUCGCACGGACAUAAGCAGCUCAUGUGCCUCGCA
CGGUCCGUGCUCUCCAAGGCCAAGAUUCUGCUGCUGGACGAACCUUCGGCCCACCUG
GAUCCGGUCACCUACCAGAUCAUCAGGAGGACCCUGAAGCAGGCCUUUGCCGAUUGC
ACCGUGAUUCUCUGCGAGCACCGCAUCGAGGCCAUGCUGGAGUGCCAGCAGUUCCUG
GUCAUCGAGGAGAACAAGGUCCGCCAAUACGACUCCAUUCAAAAGCUCCUCAACGAG
CGGUCGCUGUUCAGACAAGCUAUUUCACCGUCCGAUAGAGUGAAGCUCUUCCCGCAU
CGGAACAGCUCAAAGUGCAAAUCGAAGCCGCAGAUCGCAGCCUUGAAGGAAGAGACU
GAGGAAGAGGUGCAGGACACCCGGCUUUAACGGGUGGCAUCCCUGUGACCCCUCCCC
AGUGCCUCUCCUGGCCCUGGAAGUUGCCACUCCAGUGCCCACCAGCCUUGUCCUAAU
AAAAUUAAGUUGCAUCAAGCU

TABLE A
mRNA Structural Elements

| Structural | | Sequence |
|---|---|---|
| Element | Description | Coordinates |
| Cap Structure | 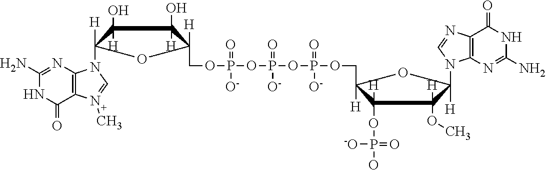 | 7 mG is attached to the nucleotide in position 1 of the CFTR mRNA where the first nucleotide (underlined) is methylated at the 2’position |
| 5’ UTR | GGAC . . . CACG | 1-140 |
| Start Codon hCFTR | AUG (Bold) | 141-143 |
| Stop Codon hCFTR | UAA (Bold) | 4581-4583 |
| 3’ UTR | CGGG . . . AGCU | 4584-4688 |
| Poly A tail | (A)ₓ, x = 200-1000* | 4689-ff |

In another embodiment, the mRNA encoding CFTR has the following sequence and structural elements:

(SEQ ID NO: 29)
GGACAGAUCGCCUGGAGACGCCAUCCACGCUGUUUUGACCUCCAUAGA
AGACACCGGGACCGAUCCAGCCUCCGCGGCCGGGAACGGUGCAUUGGAACGCGGAUU
CCCCGUGCCAAGAGUGACUCACCGUCCUUGACACGAUGCAGCGGUCCCCGCUCGAAA
AGGCCAGUGUCGUGUCCAAACUCUUCUUCUCAUGGACUCGGCCUAUCCUUAGAAAGG
GGUAUCGGCAGAGGCUUGAGUUGUCUGACAUCUACCAGAUCCCCUCGGUAGAUUCGG
CGGAUAACCUCUCGGAGAAGCUCGAACGGGAAUGGGACCGCGAACUCGCGUCUAAGA
AAAACCCGAAGCUCAUCAACGCACUGAGAAGGUGCUUCUUCUGGCGGUUCAUGUUCU
ACGGUAUCUUCUUGUAUCUCGGGGAGGUCACAAAAGCAGUCCAACCCCUGUUGUUGG
GUCGCAUUAUCGCCUCGUACGACCCCGAUAACAAAGAAGAACGGAGCAUCGCGAUCU
ACCUCGGGAUCGGACUGUGUUUGCUUUUCAUCGUCAGAACACUUUUGUUGCAUCCAG
CAAUCUUCGGCCUCCAUCACAUCGGUAUGCAGAUGCGAAUCGCUAUGUUUAGCUUGA
UCUACAAAAAGACACUGAAACUCUCGUCGCGGGUGUUGGAUAAGAUUUCCAUCGGUC
AGUUGGUGUCCCUGCUUAGUAAUAACCUCAACAAAUUCGAUGAGGGACUGGCGCUG
GCACAUUUCGUGUGGAUUGCCCCGUUGCAAGUCGCCCUUUUGAUGGGCCUUAUUUGG
GAGCUGUUGCAGGCAUCUGCCUUUUGUGGCCUGGGAUUUCUGAUUGUGUUGGCAUU
GUUUCAGGCUGGGCUUGGGCGGAUGAUGAUGAAGUAUCGCGACCAGAGAGCGGGUA
AAAUCUCGGAAAGACUCGUCAUCACUUCGGAAAUGAUCGAAAACAUCCAGUCGGUCA
AAGCCUAUUGCUGGGAAGAAGCUAUGGAGAAGAUGAUUGAAAACCUCCGCCAAACU
GAGCUGAAACUGACCCGCAAGGCGGCGUAUGUCCGGUAUUUCAAUUCGUCAGCGUUC
UUCUUUUCCGGGUUCUUCGUUGUCUUUCUCUCGGUUUUGCCUUAUGCCUUGAUUAAG
GGGAUUAUCCUCCGCAAGAUUUUCACCACGAUUUCGUUCUGCAUUGUAUUGCGCAUG
GCAGUGACACGGCAAUUUCCGUGGGCCGUGCAGACAUGGUAUGACUCGCUUGGAGCG
AUCAACAAAAUCCAAGACUUCUUGCAAAAGCAAGAGUACAAGACCCUGGAGUACAAU
CUUACUACUACGGAGGUAGUAAUGGAGAAUGUGACGGCUUUUUGGGAAGAGGGUUU
UGGAGAACUGUUUGAGAAAGCAAAGCAGAAUAACAACAACCGCAAGACCUCAAAUG
GGGACGAUUCCCUGUUUUUCUCGAACUUCUCCCUGCUCGGAACACCCGUGUUGAAGG
ACAUCAAUUUCAAGAUUGAGAGGGGACAGCUUCUCGCGGUAGCGGGAAGCACUGGU
GCGGGAAAAACUAGCCUCUUGAUGGUGAUUAUGGGGGAGCUUGAGCCCAGCGAGGG
GAAGAUUAAACACUCCGGGCGUAUCUCAUUCUGUAGCCAGUUUUCAUGGAUCAUGCC
CGGAACCAUUAAAGAGAACAUCAUUUUCGGAGUAUCCUAUGAUGAGUACCGAUACA
GAUCGGUCAUUAAGGCGUGCCAGUUGGAAGAGGACAUUUCUAAGUUCGCCGAGAAG
GAUAACAUCGUCUUGGGAGAAGGGGGUAUUACAUUGUCGGGAGGGCAGCGAGCGCG
GAUCAGCCUCGCGAGAGCGGUAUACAAAGAUGCAGAUUUGUAUCUGCUUGAUUCACC
GUUUGGAUACCUCGACGUAUUGACAGAAAAAGAAAUCUUCGAGUCGUGCGUGUGUA
AACUUAUGGCUAAUAAGACGAGAAUCCUGGUGACAUCAAAAAUGGAACACCUUAAG
AAGGCGGACAAGAUCCUGAUCCUCCACGAAGGAUCGUCCUACUUUUACGGCACUUUC
UCAGAGUUGCAAAACUUGCAGCCGGACUUCUCAAGCAAACUCAUGGGGUGUGACUCA
UUCGACCAGUUCAGCGCGGAACGGCGGAACUCGAUCUUGACGGAAACGCUGCACCGA
UUCUCGCUUGAGGGUGAUGCCCCGGUAUCGUGGACCGAGACAAAGAAGCAGUCGUUU
AAGCAGACAGGAGAAUUUGGUGAGAAAAGAAAGAACAGUAUCUUGAAUCCUAUUAA
CUCAAUUCGCAAGUUCUCAAUCGUCCAGAAAACUCCACUGCAGAUGAAUGGAAUUGA
AGAGGAUUCGGACGAACCCCUGGAGCGCAGGCUUAGCCUCGUGCCGGAUUCAGAGCA
AGGGGAGGCCAUUCUUCCCCGGAUUUCGGUGAUUUCAACCGGACCUACACUUCAGGC
GAGGCGAAGGCAAUCCGUGCUCAACCUCAUGACGCAUUCGGUAAACCAGGGGCAAAA
CAUUCACCGCAAAACGACGGCCUCAACGAGAAAAGUGUCACUUGCACCCCAGGCGAA
UUUGACUGAACUCGACAUCUACAGCCGUAGGCUUUCGCAAGAAACCGGACUUGAGAU
CAGCGAAGAAAUCAAUGAAGAAGAUUUGAAAGAGUGUUUCUUUGAUGACAUGGAAU
CAAUCCCAGCGGUGACAACGUGGAACACAUACUUGCGUUACAUCACGGUGCACAAGU
CCUUGAUUUUCGUCCUCAUCUGGUGUCUCGUGAUCUUUCUCGCUGAGGUCGCAGCGU
CACUUGUGGUCCUCUGGCUGCUUGGUAAUACGCCCUUGCAAGACAAAGGCAAUUCUA
CACACUCAAGAAACAAUUCCUAUGCCGUGAUUAUCACUUCUACAAGCUCGUAUUACG
UGUUUUACAUCUACGUAGGAGUGGCCGACACUCUGCUCGCGAUGGGUUUCUUCCGAG
GACUCCCACUCGUUCACACGCUUAUCACUGUCUCCAAGAUUCUCCACCAUAAGAUGC
UUCAUAGCGUACUGCAGGCUCCCAUGUCCACCUUGAAUACGCUCAAGGCGGGAGGUA
UUUUGAAUCGCUUCUCAAAAGAUAUUGCAAUUUUGGAUGACCUUCUGCCCCUGACGA
UCUUCGACUUCAUCCAGUUGUUGCUGAUCGUGAUUGGGGCUAUUGCAGUAGUCGCU
GUCCUCCAGCCUUACAUUUUUGUCGCGACCGUUCCGGUGAUCGUGGCGUUUAUCAUG
CUGCGGGCCUAUUUCUUGCAGACGUCACAGCAGCUUAAGCAACUGGAGUCUGAAGGG
AGGUCGCCUAUCUUUACGCAUCUUGUGACCAGUUUGAAGGGAUUGUGGACGUUGCG
CGCCUUUGGCAGGCAGCCCUACUUUGAAACACUGUUCCACAAAGCGCUGAAUCUCCA
UACGGCAAAUUGGUUUUUGUAUUUGAGUACCCUCCGAUGGUUUCAGAUGCGCAUUG
AGAUGAUUUUUGUGAUCUUCUUUAUCGCGGUGACUUUUAUCUCCAUCUUGACCACG
GGAGAGGGCGAGGGACGGGUCGGUAUUAUCCUGACACUCGCCAUGAACAUUAUGAG
CACUUUGCAGUGGGCAGUGAACAGCUCGAUUGAUGUGGAUAGCCUGAUGAGGUCCG
UUUCGAGGGUCUUUAAGUUCAUCGACAUGCCGACGGAGGGAAAGCCCACAAAAAGU
ACGAAACCCUAUAAGAAUGGGCAAUUGAGUAAGGUAAUGAUCAUCGAGAACAGUCA
CGUGAAGAAGGAUGACAUCUGGCCUAGCGGGGGUCAGAUGACCGUGAAGGACCUGA
CGGCAAAAUACACCGAGGGAGGGAACGCAAUCCUUGAAAACAUCUCGUUCAGCAUUA
GCCCCGGUCAGCGUGUGGGGUUGCUCGGGAGGACCGGGUCAGGAAAAUCGACGUUGC
UGUCGGCCUUCUUGAGACUUCUGAAUACAGAGGGUGAGAUCCAGAUCGACGGCGUU
UCGUGGGAUAGCAUCACCUUGCAGCAGUGGCGGAAAGCGUUUGGAGUAAUCCCCCAA
AAGGUCUUUAUCUUUAGCGGAACCUUCCGAAAGAAUCUCGAUCCUUAUGAACAGUG
GUCAGAUCAAGAGAUUUGGAAAGUCGCGGACGAGGUUGGCCUUCGGAGUGUAAUCG
AGCAGUUUCCGGGAAAACUCGACUUUGUCCUUGUAGAUGGGGGAUGCGUCCUGUCGC
AUGGGCACAAGCAGCUCAUGUGCCUGGCGCGAUCCGUCCUCUCUAAAGCGAAAAUUC
UUCUCUUGGAUGAACCUUCGGCCCAUCUGGACCCGGUAACGUAUCAGAUCAUCAGAA
GGACACUUAAGCAGGCGUUUGCCGACUGCACGGUGAUUCUCUGUGAGCAUCGUAUCG
AGGCCAUGCUCGAAUGCCAGCAAUUUCUUGUCAUCGAAGAGAAUAAGGUCCGCCAGU
ACGACUCCAUCCAGAAGCUGCUUAAUGAGAGAUCAUUGUUCCGGCAGGCGAUUUCAC
CAUCCGAUAGGGUGAAACUUUUUCCACACAGAAAUUCGUCGAAGUGCAAGUCCAAAC
CGCAGAUCGCGGCCUUGAAAGAAGAGACUGAAGAAGAAGUUCAAGACACGCGUCUU
UAACGGGUGGCAUCCCUGUGACCCCUCCCCAGUGCCUCUCCUGGCCCUGGAAGUUGC
CACUCCAGUGCCCACCAGCCUUGUCCUAAUAAAAUUAAGUUGCAUCAAGCU

TABLE B
mRNA Structural Elements

| Structural | | Sequence |
|---|---|---|
| Element | Description | Coordinates |
| Cap Structure | 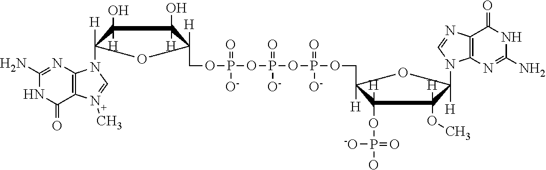 | 7 mG is attached to the nucleotide in position 1 of the CFTR mRNA where the first nucleotide (underlined) is methylated at the 2’ position |
| 5’ UTR | GGAC . . . CACG | 1-140 |
| Start Codon hCFTR | AUG (Bold) | 141-143 |
| Stop Codon hCFTR | UAA (Bold) | 4581-4583 |
| 3’ UTR | CGGG . . . AGCU | 4584-4689 |
| Poly A tail | (A)ₓ, x = 200-1000* | 4690-ff |

In another embodiment, the mRNA encoding CFTR has the following sequence and structural elements:

(SEQ ID NO: 30)
GGACAGAUCGCCUGGAGACGCCAUCCACGCUGUUUUGACCUCCAUAGA
AGACACCGGGACCGAUCCAGCCUCCGCGGCCGGGAACGGUGCAUUGGAACGCGGAUU
CCCCGUGCCAAGAGUGACUCACCGUCCUUGACACGAUGCAGAGGAGCCCACUGGAGA
AAGCCUCCGUGGUGAGUAAACUCUUUUUUAGUUGGACCAGACCCAUCCUGCGAAAAG
GAUACAGGCAGCGCCUCGAGUUGUCUGAUAUCUACCAGAUUCCUUCUGUGGACUCAG
CUGACAAUUUGAGUGAGAAGCUGGAGCGGGAGUGGGAUAGAGAGCUGGCGAGCAAA
AAAAACCCCAAGCUUAUCAAUGCUCUGCGCCGCUGCUUUUUCUGGAGGUUCAUGUUU
UAUGGGAUCUUCCUGUACCUGGGGGAGGUCACCAAAGCUGUUCAGCCGCUCCUUCUU
GGCCGCAUCAUCGCCAGCUAUGACCCUGAUAAUAAAGAAGAAAGGUCUAUUGCUAUU
UAUCUGGGAAUUGGCCUCUGCUUGCUCUUCAUCGUCCGCACCCUUCUGCUGCACCCU
GCCAUUUUUGGCCUUCACCACAUCGGCAUGCAAAUGAGAAUUGCCAUGUUCUCCCUC
AUUUACAAAAAGACCCUGAAACUUUCCUCAAGAGUGUUAGAUAAAAUAUCCAUUGG
UCAGCUGGUCAGCCUGCUGUCCAACAAUCUUAACAAAUUUGAUGAAGGCUUGGCGCU
GGCCCACUUCGUGUGGAUUGCACCUCUGCAGGUGGCCCUGUUGAUGGGACUUAUAUG
GGAGCUGCUUCAAGCCUCUGCUUUCUGUGGGCUGGGCUUUUUGAUUGUACUGGCACU
UUUUCAGGCUGGGCUCGGAAGAAUGAUGAUGAAAUACAGAGAUCAGCGGGCCGGGA
AGAUUUCAGAGCGACUUGUGAUCACCAGUGAAAUGAUUGAAAAUAUUCAGAGCGUG
AAAGCCUACUGCUGGGAAGAAGCCAUGGAGAAGAUGAUUGAGAACCUGAGGCAGAC
AGAGCUCAAGCUCACUCGGAAGGCUGCUUAUGUUCGCUAUUUCAACAGCAGCGCCUU
CUUCUUCAGUGGCUUCUUUGUUGUCUUCCUGUCUGUUCUGCCAUAUGCACUGAUAAA
AGGCAUUAUUUUACGAAAGAUCUUCACCACCAUCAGUUUUUGCAUCGUUCUCAGGAU
GGCCGUCACAAGACAGUUCCCCUGGGCUGUGCAGACCUGGUACGAUUCCUUGGGGGC
CAUCAACAAGAUUCAAGAUUUCUUGCAAAAACAAGAAUAUAAAACUUUAGAAUACA
ACCUCACCACCACUGAAGUGGUCAUGGAAAAUGUGACAGCCUUUUGGGAGGAGGGU
UUUGGAGAAUUGUUCGAGAAGGCAAAGCAGAAUAACAACAACAGGAAGACGAGCAA
UGGGGACGACUCUCUCUUCUUCAGCAACUUUUCACUGCUCGGGACCCCUGUGUUGAA
AGAUAUAAACUUCAAGAUCGAGAGGGGCCAGCUCUUGGCUGUGGCAGGCUCCACUGG
AGCUGGUAAAACAUCUCUUCUCAUGGUGAUCAUGGGGGAACUGGAGCCUUCCGAAG
GAAAAAUCAAGCACAGUGGGAGAAUCUCAUUCUGCAGCCAGUUUUCCUGGAUCAUGC
CCGGCACCAUUAAGGAAAACAUCAUAUUUGGAGUGUCCUAUGAUGAGUACCGCUACC
GGUCAGUCAUCAAAGCCUGUCAGUUGGAGGAGGACAUCUCCAAGUUUGCAGAGAAA
GACAACAUUGUGCUUGGAGAGGGGGGUAUCACUCUUUCUGGAGGACAAAGAGCCAG
GAUCUCUUUGGCCCGGGCAGUCUACAAGGAUGCAGACCUCUACUUGUUGGACAGUCC
CUUCGGCUACCUCGACGUGCUGACUGAAAAAGAAAUUUUUGAAAGCUGUGUGUGCA
AACUGAUGGCAAACAAGACCAGGAUUCUUGUCACCAGCAAGAUGGAACAUCUGAAG
AAAGCGGACAAAAUUCUGAUUCUGCAUGAAGGGAGCUCCUACUUCUAUGGAACAUU
UAGCGAGCUUCAGAACCUACAGCCAGACUUCUCCUCCAAAUUAAUGGGCUGUGACUC
CUUCGACCAGUUCUCUGCAGAAAGAAGAAACUCUAUACUCACAGAGACCCUCCACCG
CUUCUCCCUUGAGGGAGAUGCCCCAGUUUCUUGGACAGAAACCAAGAAGCAGUCCUU
UAAGCAGACUGGCGAGUUUGGUGAAAAGAGGAAAAAUUCAAUUCUCAAUCCAAUUA
ACAGUAUUCGCAAGUUCAGCAUUGUCCAGAAGACACCCCUCCAGAUGAAUGGCAUCG
AAGAAGAUAGUGACGAGCCGCUGGAGAGACGGCUGAGUCUGGUGCCAGAUUCAGAA
CAGGGGGAGGCCAUCCUGCCCCGGAUCAGCGUCAUUUCCACAGGCCCCACAUUACAA
GCACGGCGCCGGCAGAGUGUUUUAAAUCUCAUGACCCAUUCAGUGAACCAGGGCCAA
AAUAUCCACAGGAAGACUACAGCUUCUACCCGGAAAGUGUCUCUGGCCCCUCAGGCC
AAUCUGACCGAGCUGGACAUCUACAGCAGGAGGCUCUCCCAGGAAACAGGGCUGGAA
AUAUCUGAAGAGAUUAAUGAAGAGGAUCUUAAAGAGUGCUUCUUUGAUGACAUGGA
GAGCAUCCCCGCGGUGACCACAUGGAACACCUACCUUAGAUAUAUUACUGUCCACAA
GAGCCUCAUAUUUGUCCUCAUCUGGUGCCUGGUUAUUUUCCUCGCUGAGGUGGCGGC
CAGUCUUGUUGUGCUCUGGCUGCUGGGCAACACUCCUCUCCAGGACAAGGGCAAUAG
UACUCACAGCAGAAAUAAUUCUUAUGCCGUCAUCAUUACAAGCACCUCCAGCUACUA
CGUGUUCUACAUCUAUGUGGGCGUGGCUGACACCCUCCUGGCCAUGGGUUUCUUCCG
GGGCCUGCCUUUGGUGCACACCCUCAUCACAGUGUCAAAAAUUCUGCACCAUAAAAU
GCUUCAUUCUGUCCUGCAGGCACCCAUGAGCACUUUGAACACAUUGAAGGCUGGCGG
CAUCCUCAACAGAUUUUCUAAAGAUAUUGCUAUCCUGGAUGAUCUCCUCCCCCUGAC
AAUCUUUGACUUUAUCCAGCUUCUGCUGAUCGUGAUUGGAGCCAUAGCAGUGGUUG
CUGUCCUGCAGCCCUACAUUUUUGUGGCCACCGUGCCCGUGAUUGUUGCCUUUAUUA
UGCUCAGAGCUUACUUCCUGCAAACUUCUCAACAGCUCAAACAGCUAGAAUCUGAGG
GCCGGAGCCCCAUUUUUACCCACCUGGUGACUUCCCUGAAGGGACUGUGGACUCUGA
GAGCAUUCGGGCGACAGCCUUACUUUGAGACACUGUUCCACAAGGCCCUGAACUUGC
ACACUGCCAACUGGUUUCUUUACCUGAGCACACUCCGCUGGUUCCAGAUGCGGAUAG
AGAUGAUCUUCGUCAUCUUUUUUAUAGCUGUAACCUUCAUUUCUAUCCUUACAACAG
GAGAAGGAGAGGGCAGGGUGGGAAUCAUCCUCACGCUGGCUAUGAACAUAAUGUCC
ACCUUGCAGUGGGCCGUGAAUUCCAGUAUAGAUGUGGAUUCUCUAAUGAGGAGUGU
CUCCCGGGUGUUUAAAUUCAUUGAUAUGCCUACUGAGGGGAAACCCACCAAGUCAAC
AAAACCUUAUAAGAAUGGACAGCUGAGCAAGGUGAUGAUAAUUGAGAACAGCCACG
UGAAGAAGGAUGACAUUUGGCCCAGCGGGGGCCAGAUGACUGUGAAGGACCUGACG
GCCAAGUACACCGAAGGUGGAAAUGCCAUUUUGGAAAACAUCAGCUUCUCAAUCUCU
CCUGGGCAGAGAGUUGGAUUGCUGGGUCGCACGGGCAGCGGCAAAUCAACCCUGCUC
AGUGCCUUCCUUCGGCUCCUGAAUACAGAAGGCGAAAUCCAAAUUGACGGGGUGAGC
UGGGACAGCAUCACCCUGCAGCAGUGGAGAAAAGCAUUUGGGGUCAUUCCACAGAAA
GUUUUCAUCUUCUCUGGCACUUUCAGAAAGAACCUGGACCCCUAUGAGCAGUGGAGC
GACCAGGAGAUCUGGAAGGUUGCAGAUGAAGUUGGCCUGCGGAGUGUGAUAGAACA
AUUUCCUGGCAAGCUGGAUUUUGUGCUGGUAGAUGGAGGCUGCGUGCUGUCCCACG
GCCACAAACAGCUGAUGUGCCUCGCCCGCUCCGUUCUUUCAAAGGCCAAAAUCUUGC
UUUUGGAUGAGCCCAGUGCUCACCUUGACCCAGUGACCUAUCAGAUAAUCCGCAGGA
CCUUAAAGCAAGCUUUUGCCGACUGCACCGUCAUACUGUGUGAGCACCGGAUUGAAG
CAAUGCUGGAAUGCCAGCAGUUUCUGGUGAUCGAGGAGAAUAAGGUCCGGCAGUAC
GACAGCAUCCAGAAGUUGUUGAAUGAGCGCAGCCUUUUCCGCCAGGCCAUCUCCCCA
UCUGACAGAGUCAAGCUGUUUCCACAUAGGAACUCCUCUAAGUGCAAGUCCAAGCCC
CAGAUCGCUGCCCUCAAGGAGGAAACUGAGGAAGAGGUGCAGGAUACCCGCCUGUGA
CGGGUGGCAUCCCUGUGACCCCUCCCCAGUGCCUCUCCUGGCCCUGGAAGUUGCCAC
UCCAGUGCCCACCAGCCUUGUCCUAAUAAAAUUAAGUUGCAUCAAGCU

TABLE C
mRNA Structural Elements

| Structural | | Sequence |
|---|---|---|
| Element | Description | Coordinates |
| Cap Structure | 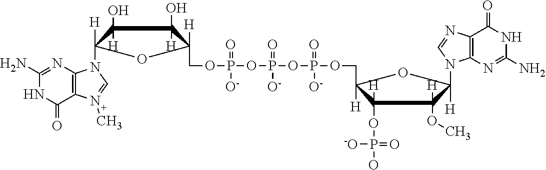 | 7 mG is attached to the nucleotide in position 1 of the CFTR mRNA where the first nucleotide (underlined) is methylated at the 2’ position |
| 5’ UTR | GGAC . . . CACG | 1-140 |
| Start Codon hCFTR | AUG (Bold) | 141-143 |
| Stop Codon hCFTR | UGA (Bold) | 4581-4583 |
| 3’ UTR | CGGG . . . AGCU | 4584-4688 |
| Poly A tail | (A)ₓ, x = 200-1000* | 4689-ff |

Modified mRNA

A CFTR mRNA may contain only naturally-occurring nucleotides (or unmodified nucleotides). In some embodiments, however, a suitable CFTR mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, mRNAs (e.g., mRNAs encoding CFTR) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, mRNAs (e.g., mRNAs encoding CFTR) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In a specific embodiment of the invention, mRNAs encoding CFTR are unmodified.

### Delivery Vehicles

According to the present invention, mRNA encoding a CFTR protein (e.g., a full length, fragment, or portion of a CFTR protein) as described herein may be delivered as naked mRNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

According to various embodiments, suitable delivery vehicles include, but are not limited to polymer based carriers, such as polyethyleneimine (PEI), lipid nanoparticles (LNPs) and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags.

### Liposomal Delivery Vehicles

In some embodiments, a suitable delivery vehicle is a liposomal delivery vehicle, e.g., a lipid nanoparticle (LNP) or liposome. In some embodiments, liposomes may comprise one or more cationic lipids. In some embodiments, a liposome comprises one or more cationic lipids, one or more non-cationic lipids, one or more cholesterol-based lipids and one or more PEG-modified lipids. In some embodiments, a liposome comprises one or more cationic lipids, one or more non-cationic lipids, and one or more PEG-modified lipids. In some embodiments, a liposome comprises no more than four distinct lipid components. In some embodiments, a liposome comprises no more than three distinct lipid components. In some embodiments, one distinct lipid component is a sterol-based cationic lipid.

As used herein, the phrase “cationic lipid” refers to any of a number of lipid species that have a net positive charge at a selected pH, such as physiological pH. Several cationic lipids have been described in the literature, many of which are commercially available. An example of suitable cationic lipids for use in the compositions and methods of the invention include those described in international patent publications WO 2010/053572 (for example, C12-200 described at paragraph [00225]) and WO 2012/170930, both of which are incorporated herein by reference. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g., (15Z, 18Z)—N,N-dimethyl-6-(9Z, 12Z)-octadeca-9, 12-dien-1-yl)tetracosa-15,18-dien-1-amine (HGT5000), (15Z, 18Z)—N,N-dimethyl-6-((9Z, 12Z)-octadeca-9, 12-dien-1-yl)tetracosa-4, 15,18-trien-1-amine (HGT5001), and (15Z, 18Z)—N,N-dimethyl-6-((9Z, 12Z)-octadeca-9, 12-dien-1-yl)tetracosa-5, 15, 18-trien-1-amine (HGT5002).

In some embodiments, provided liposomes include a cationic lipid described in international patent publications WO 2013/063468 and WO 2015/061467 both of which are incorporated by reference herein.

In particular embodiments, provided liposomes include a cationic lipid cKK-E12, or (3,6-bis(4-(bis(2-hydroxydodecyl)amino)butyl)piperazine-2,5-dione, OF-00, OF-01, OF-02, or OF-03 (see, e.g., Fenton, Owen S., et al. “Bioinspired Alkenyl Amino Alcohol Ionizable Lipid Materials for Highly Potent In Vivo mRNA Delivery.” Advanced materials (2016)).

In some embodiments, suitable cationic lipids may be N-[1-(2,3-diolcyloxy)propyl]-N,N,N-trimethylammonium chloride or “DOTMA” (Feigner et al. (Proc. Nat'l Acad. Sci. 84, 7413 (1987); U.S. Pat. No. 4,897,355). DOTMA can be formulated alone or can be combined with the neutral lipid, diolcoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2 (spermine-carboxamido)ethyl]-N,N-dimethyl-1-propanaminium or “DOSPA” (Behr et al. Proc. Nat.′l Acad. Sci. 86, 6982 (1989); U.S. Pat. Nos. 5,171,678; 5,334,761), and 1,2-Diolcoyl-3-Dimethylammonium-Propane or “DODAP”, 1,2-Dioleoyl-3-Trimethylammonium-Propane or “DOTAP”.

Additional exemplary cationic lipids also include 1,2-distearyloxy-N,N-dimethyl-3-aminopropane or “DSDMA”, 1,2-dioleyloxy-N,N-dimethyl-3-aminopropane or “DODMA,” 1,2-dilinoleyloxy-N,N-dimethyl-3-aminopropane or “DLinDMA,” 1,2-dilinolenyloxy-N,N-dimethyl-3-aminopropane or “DLenDMA,” N-dioleyl-N,N-dimethylammonium chloride or “DODAC,” N,N-distearyl-N,N-dimethylarnrnonium bromide or “DDAB,” N-(1,2-dimyristyloxyprop-3-yl)-N,N-dimethyl-N-hydroxyethyl ammonium bromide or “DMRIE,” 3-dimethylamino-2-(cholest-5-en-3-beta-oxybutan-4-oxy)-1-(cis,cis-9,12-octadecadienoxy)propane or “CLinDMA,” 2-[5′-(cholest-5-en-3-beta-oxy)-3′-oxapentoxy)-3-dimethyl-1-(cis,cis-9′,1-2′-octadecadienoxy)propane or “CpLinDMA,” N,N-dimethyl-3,4-dioleyloxybenzylamine or “DMOBA,” 1,2-N,N′-diolcylcarbamyl-3-dimethylaminopropane or “DOcarbDAP,” 2,3-Dilinoleoyloxy-N,N-dimethylpropylamine or “DLinDAP,” 1,2-N,N′-Dilinoleylcarbamyl-3-dimethylaminopropane or “DLincarbDAP,” 1,2-Dilinoleoylcarbamyl-3-dimethylaminopropane or “DLinCDAP,” 2,2-dilinoleyl-4-dimethylaminomethyl-[1,3]-dioxolane or “DLinDMA,” 2,2-dilinoleyl-4-dimethylaminoethyl-[1,3]-dioxolane or “DLin-K-XTC2-DMA,” and 2-(2,2-di((9Z,12Z)-octadeca-9,1 2-dien-1-yl)-1,3-dioxolan-4-yl)-N,N-dimethylethanamine (DLin-KC2-DMA)) (See WO 2010/042877; Semple et al., Nature Biotech. 28:172-176 (2010)), or mixtures thereof. (Heyes, J., et al . . . , J Controlled Release 107:276-287 (2005); Morrissey, D V., et al., Nat. Biotechnol. 23 (8): 1003-1007 (2005); PCT Publication WO2005/121348A1). In some embodiments, one or more of the cationic lipids comprise at least one of an imidazole, dialkylamino, or guanidinium moiety.

In some embodiments, the one or more cationic lipids may be chosen from XTC (2,2-Dilinoleyl-4-dimethylaminocthyl-[1,3]-dioxolane), MC3 (((6Z,9Z,28Z,31Z)-heptatriaconta-6,9,28,31-tetraen-19-yl 4-(dimethylamino) butanoate), ALNY-100 ((3aR,5s,6aS)—N,N-dimethyl-2,2-di((9Z, 12Z)-octadeca-9,12-dienyl)tetrahydro-3aH-cyclopenta [d][1,3]dioxol-5-amine)), NC98-5 (4,7, 13-tris(3-oxo-3-(undecylamino)propyl)-N1,N16-diundecyl-4,7,10, 13-tetraazahexadecane-1,16-diamide), HGT4003 (WO 2012/170889, the teachings of which are incorporated herein by reference in their entirety), ICE (WO 2011/068810, the teachings of which are incorporated herein by reference in their entirety), and aminoalcohol lipidoids such as those disclosed in WO2010/053572.

In some embodiments, sterol-based cationic lipids may be use instead or in addition to cationic lipids described herein. Suitable sterol-based cationic lipids are dialkylamino-, imidazole-, and guanidinium-containing sterol-based cationic lipids. For example, certain embodiments are directed to a composition comprising one or more sterol-based cationic lipids comprising an imidazole, for example, the imidazole cholesterol ester or “ICE” lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by structure (I) below. In certain embodiments, a lipid nanoparticle for delivery of RNA (e.g., mRNA) encoding a functional protein may comprise one or more imidazole-based cationic lipids, for example, the imidazole cholesterol ester or “ICE” lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by structure (I).

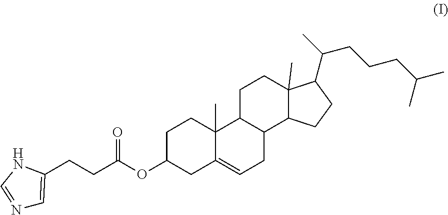 [SMILES: CC(C)CCCC(C)C1CCC2C3CC=C4CC(OC(=O)CCc5cnc[nH]5)CCC4(C)C3CCC12C]

In some embodiments, the percentage of cationic lipid in a liposome may be greater than 10%, greater than 20%, greater than 30%, greater than 40%, greater than 50%, greater than 60%, or greater than 70%. In some embodiments, cationic lipid(s) constitute(s) about 30-50% (e.g., about 30-45%, about 30-40%, about 35-50%, about 35-45%, or about 35-40%) of the liposome by weight. In some embodiments, the cationic lipid (e.g., ICE lipid) constitutes about 30%, about 35%, about 40%, about 45%, or about 50% of the liposome by molar ratio.

As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoylolcoylphosphatidylcholine (POPC), palmitoyloleoyl-phosphatidylethanolamine (POPE), diolcoyl-phosphatidylethanolamine 4-(N-malcimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-olcoyl-phosphatidyethanolamine (SOPE), or a mixture thereof.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

Suitable cholesterol-based cationic lipids include, for example, DC-Chol (N,N-dimethyl-N-cthylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

The use of polyethylene glycol (PEG)-modified phospholipids and derivatized lipids such as derivatized cerarmides (PEG-CER), including N-Octanoyl-Sphingosine-1-[Succinyl(Methoxy Polyethylene Glycol)-2000](C8 PEG-2000 ceramide) is also contemplated by the present invention, cither alone or preferably in combination with other lipid formulations together which comprise the transfer vehicle (e.g., a lipid nanoparticle). Contemplated PEG-modified lipids include, but are not limited to, a polyethylene glycol chain of up to S kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

A suitable liposome for the present invention may include one or more of any of the cationic lipids, non-cationic lipids, cholesterol lipids, PEG-modified lipids and/or polymers described herein at various ratios. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5. In some embodiments, the ratio of sterol lipid(s) to non-cationic lipid(s) to PEG-modified lipid(s) is 50:45:5. In some embodiments, the ratio of sterol lipid(s) to non-cationic lipid(s) to PEG-modified lipid(s) is 50:40:10. In some embodiments, the ratio of sterol lipid(s) to non-cationic lipid(s) to PEG-modified lipid(s) is 55:40:5. In some embodiments, the ratio of sterol lipid(s) to non-cationic lipid(s) to PEG-modified lipid(s) is 55:35:10. In some embodiments, the ratio of sterol lipid(s) to non-cationic lipid(s) to PEG-modified lipid(s) is 60:35:5. In some embodiments, the ratio of sterol lipid(s) to non-cationic lipid(s) to PEG-modified lipid(s) is 60:30:10.

In some embodiments, the nominal nitrogen/phosphorus (N/P) charge ratio which refers to the positively charged nitrogens in the cationic lipid and the negatively charged phosphodiester linkages within mRNA is about between 1 and 10. In some embodiments, the N/P is about 1. In some embodiments, the N/P is about 2. In some embodiments, the N/P is about 3. In some embodiments, the N/P is about 4. In some embodiments, the N/P is about 5. In some embodiments, the N/P is about 6. In some embodiments, the N/P is about 7. In some embodiments, the N/P is about 8. In some embodiments, the N/P is about 9. In some embodiments, the N/P is about 10.

A liposome comprising ICE, DOPE, and DMG-PEG2K is particularly suitable for use with the present invention. In some embodiments, a suitable liposome for the present invention comprises ICE and DOPE at an ICE: DOPE molar ratio of >1:1. In some embodiments, the ICE: DOPE molar ratio is <2.5:1. In some embodiments, the ICE: DOPE molar ratio is between 1:1 and 2.5:1. In some embodiments, the ICE: DOPE molar ratio is approximately 1.5:1. In some embodiments, the ICE: DOPE molar ratio is approximately 1.7:1. In some embodiments, the ICE: DOPE molar ratio is approximately 2:1. In some embodiments, a suitable liposome for the present invention comprises ICE and DMG-PEG-2K at an ICE: DMG-PEG-2K molar ratio of >10:1. In some embodiments, the ICE: DMG-PEG-2K molar ratio is <16:1. In some embodiments, the ICE: DMG-PEG-2K molar ratio is approximately 12:1. In some embodiments, the ICE: DMG-PEG-2K molar ratio is approximately 14:1. In some embodiments, a suitable liposome for the present invention comprises DOPE and DMG-PEG-2K at a DOPE: DMG-PEG-2K molar ratio of >5:1. In some embodiments, the DOPE: DMG-PEG-2K molar ratio is <11:1. In some embodiments, the DOPE: DMG-PEG-2K molar ratio is approximately 7:1. In some embodiments, the DOPE: DMG-PEG-2K molar ratio is approximately 10:1. In some embodiments, a suitable liposome for the present invention comprises ICE, DOPE and DMG-PEG-2K at a molar ratio of 50%-60% ICE, 30%-40% DOPE and 5%-10% DMG-PEG-2K. In some embodiments, a suitable liposome for the present invention comprises ICE, DOPE and DMG-PEG-2K at an ICE: DOPE: DMG-PEG-2K molar ratio of 50:45:5. In some embodiments, a suitable liposome for the present invention comprises ICE, DOPE and DMG-PEG-2K at an ICE: DOPE: DMG-PEG-2K molar ratio of 50:40:10. In some embodiments, a suitable liposome for the present invention comprises ICE, DOPE and DMG-PEG-2K at an ICE: DOPE: DMG-PEG-2K molar ratio of 55: 40: 5. In some embodiments, a suitable liposome for the present invention comprises ICE, DOPE and DMG-PEG-2K at an ICE: DOPE: DMG-PEG-2K molar ratio of 55:35:10. In some embodiments, a suitable liposome for the present invention comprises ICE, DOPE and DMG-PEG-2K at an ICE: DOPE: DMG-PEG-2K molar ratio of 60:35:5. In some embodiments, a suitable liposome for the present invention comprises ICE, DOPE and DMG-PEG-2K at an ICE: DOPE: DMG-PEG-2K molar ratio of 60:30:10.

The liposomal transfer vehicles for use in the compositions of the invention can be prepared by various techniques which are presently known in the art. Various methods are described in published U.S. Application No. US 2011/0244026, published U.S. Application No. US 2016/0038432 and provisional U.S. Application No. 62/580,155, filed Nov. 1, 2017 and can be used to practice the present invention, all of which are incorporated herein by reference.

Briefly, the process of preparing improved CFTR-mRNA lipid liposomes includes a step of heating one or more of the solutions (i.e., applying heat from a heat source to the solution) to a temperature (or to maintain at a temperature) greater than ambient temperature, the one more solutions being the solution comprising the pre-formed lipid nanoparticles, the solution comprising the mRNA and the mixed solution comprising the lipid nanoparticle encapsulated mRNA. In some embodiments, the process includes the step of heating one or both of the mRNA solution and the pre-formed lipid nanoparticle solution, prior to the mixing step. In some embodiments, the process includes heating one or more one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

To facilitate expression of mRNA in vivo, delivery vehicles such as liposomes can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating cystic fibrosis). For example, a therapeutically effective amount may be an amount sufficient to achieve a desired therapeutic and/or prophylactic effect.

In some embodiments, the composition comprising an mRNA encoding CFTR comprises mRNA at a concentration of at least 0.1 mg/mL. In some embodiments, the composition comprising an mRNA encoding CFTR comprises mRNA at a concentration of at least 0.2 mg/mL. In some embodiments, the composition comprising an mRNA encoding CFTR comprises mRNA at a concentration of at least 0.3 mg/mL. In some embodiments, the composition comprising an mRNA encoding CFTR comprises mRNA at a concentration of at least 0.4 mg/mL. In some embodiments, the mRNA encoding a CFTR protein is at a concentration of at least 0.5 mg/mL. In some embodiments, the mRNA encoding a CFTR protein is at a concentration of at least 0.6 mg/mL. In some embodiments, the mRNA encoding a CFTR protein is at a concentration of at least 0.7 mg/mL. In some embodiments, the mRNA encoding a CFTR protein is at a concentration of at least 0.8 mg/mL. In some embodiments, the mRNA encoding a CFTR protein is at a concentration of at least 0.9 mg/mL. In some embodiments, the mRNA encoding a CFTR protein is at a concentration of at least 1.0 mg/mL. In some embodiments, the mRNA encoding a CFTR protein is at a concentration of at least 2.0 mg/mL. In some embodiments, the mRNA encoding a CFTR protein is at a concentration of at least 3.0 mg/mL. In some embodiments, the mRNA encoding a CFTR protein is at a concentration of at least 4.0 mg/mL. In some embodiments, the mRNA encoding a CFTR protein is at a concentration of at least 5.0 mg/mL. In some embodiments, the mRNA encoding a CFTR protein is at a concentration of at least 6.0 mg/mL. In some embodiments, the mRNA encoding a CFTR protein is at a concentration of at least 7.0 mg/mL. In some embodiments, the mRNA encoding a CFTR protein is at a concentration of at least 8.0 mg/mL. In some embodiments, the mRNA encoding a CFTR protein is at a concentration of at least 9.0 mg/mL. In some embodiments, the mRNA encoding a CFTR protein is at a concentration of at least 10.0 mg/mL. In some embodiments, the mRNA encoding a CFTR protein is at a concentration ranging from 0.1 mg/mL to 10.0 mg/mL. Typically, the mRNA encoding a CFTR protein is at a concentration ranging from 0.5 mg/mL to 0.8 mg/mL, e.g., 0.6 mg/mL.

In some embodiments, the composition comprising an mRNA encoding CFTR is formulated with a diluent. In some embodiments, the diluent is selected from a group consisting of DMSO, ethylene glycol, glycerol, 2-Methyl-2,4-pentanediol (MPD), propylene glycol, sucrose, and trehalose. In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent.

Trehalose as a diluent has been shown to be particularly effective in providing a stable composition comprising liposome-encapsulated mRNA encoding CFTR. A suitable trehalose concentration is between about 5% and about 15% (w/v), e.g., about 10% (w/v).

### Lyophilization

The liposomal CFTR mRNA compositions of the invention may be provided in form of a dry powder. In some embodiments, CFTR mRNA dry powder is formed by lyophilization of the mRNA-lipid complex. Applicant hereby fully incorporates by reference their earlier patent application U.S. Ser. No. 14/124,615 filed on Jun. 8, 2012, which was granted a U.S. Pat. No. 9,717,690 on 8 Jan. 2017. The lyophilized dry powder is suitable for long term storage. It can be reconstituted with purified water for administration to a subject in need thereof. In certain embodiments, upon reconstitution with an appropriate rehydration media (e.g., purified water, deionized water, 5% dextrose (w/v), 10% trehalose (w/v) and/or normal saline, the reconstituted composition demonstrates pharmacological or biological activity comparable with that observed prior to lyophilization. For example, in certain embodiments, the pharmacological and biological activity of an encapsulated polynucleotide is equivalent to that observed prior to lyophilization of the composition; or alternatively demonstrates a negligible reduction in pharmacological and biological activity (e.g., less than about a 1%, 2%, 2.5%, 3%, 4%, 5%, 6%, 7%, 8% 9% or 10% reduction in the biological or pharmacological activity of an encapsulated polynucleotide).

In certain embodiments, the pharmaceutical compositions comprising lyophilized nanoparticles or liposomal delivery vehicles are characterized as being stable (e.g., as stable as pharmaceutical compositions comprising an equivalent unlyophilized vehicles). Lyophilization of the lipid nanoparticles does not appreciably change or alter the particle size of the lipid nanoparticles following lyophilizaiton and/or reconstitution. For example, disclosed herein are pharmaceutical compositions comprising lyophilized lipid delivery vehicles, wherein upon reconstitution (e.g., with purified water) the lipid nanoparticles do not flocculate or aggregate, or alternatively demonstrated limited or negligible flocculation or aggregation (e.g., as determined by the particle size of the reconstituted lipid nanoparticles).

Accordingly, in certain embodiments, upon reconstitution of a lyophilized lipid nanoparticle the lipid nanoparticles have a Dv₅₀ of less than about 500 nm (e.g., less than about 300 nm, 200 nm, 150 nm, 125 nm, 120 nm, 100 nm, 75 nm, 50 nm, 25 nm, or smaller). Similarly, in certain embodiments, upon reconstitution of a lyophilized lipid nanoparticle the lipid nanoparticles have a Dv₉₀ of less than about 750 nm (e.g., less than about 700 nm, 500 nm, 300 nm, 200 nm, 150 nm, 125 nm, 100 nm, 75 nm, 50 nm, 25 nm, or smaller).

In other embodiments, the pharmaceutical compositions comprising lyophilized lipid delivery vehicles are characterized as having a polydispersion index of less than about 1 (e.g., less than 0.95, 0.9, 0.8, 0.75, 0.7, 0.6, 0.5, 0.4, 0.3, 0.25, 0.2, 0.1, 0.05, or less). In some embodiments, the pharmaceutical compositions comprising lyophilized lipid delivery vehicles demonstrate a reduced tendency to flocculate or otherwise aggregate (e.g., during lyophilization or upon reconstitution). For example, upon reconstitution the lipid delivery vehicles may have an average particle size (Zₐᵥₑ) of less than 500 nm (e.g., less than about 400 nm, 300 nm, 200 nm, 175 nm, 150 nm, 125 nm, 100 nm, 75 nm, 50 nm, 25 nm, or smaller in a PBS solution). Typically, the average particle size (Zₐᵥₑ) of lipid delivery vehicles for use with the invention is between 40 nm and 60 nm.

In some embodiments, the lyophilized lipid delivery vehicles (e.g., lyophilized lipid nanoparticles) further comprise or are alternatively prepared using one or more lyoprotectants (e.g., sugars and/or carbohydrates). In certain embodiments, the inclusion of one or more lyoprotectants in the lipid nanoparticle may improve or otherwise enhance the stability of the lyophilized lipid delivery vehicles (e.g., under normal storage conditions) and/or facilitate reconstitution of the lyophilized lipid delivery vehicles using a rehydration media, thereby preparing an aqueous formulation. For example, in certain embodiments the lipid nanoparticles are prepared and prior to lyophilization the buffer present in the liposomal formulation may be replaced (e.g., via centrifugation) with a lyoprotectant such as a sucrose solution or suspension (e.g., an aqueous solution comprising between about 1-50% (w/v) or 10-25% (w/v) sucrose). In some embodiments, the lyoprotectant in trehalose. In some embodiments, the lyoprotectant comprises 10-50% (w/v), or 10-25% (w/v) or 10-20% (w/v) or 10-15% (w/v) trehalose. Other lyoprotectants that may be used to prepare the lyophilized compositions described herein include, for example, dextran (e.g., 1.5 kDa, 5 kDa and/or 40 kDa) and inulin (e.g., 1.8 kDa and/or 4 kDa). The lyophilized lipid delivery vehicles have an encapsulation efficiency of greater than about 80%.

A pharmaceutical composition comprising a lyophilized lipid nanoparticle comprising CFTR-encoding mRNA is stable at 4° C. for at least 1 month, at least 2 months, at least 3 months, at least 4 months, at least 5 months, at least 6 months, or for at least 1 year. In some embodiments, the lyophilized lipid delivery vehicles may be stored under refrigeration and remain stable (e.g., as demonstrated by minimal or no losses in their intended pharmaceutical or biological activity) for extended periods of time (e.g., stable for at least about 1, 2, 3, 4, 5, 6, 9, 12, 18, 24, 36 months or longer upon storage at about 4° C.). In other embodiments, the lyophilized lipid delivery vehicles may be stored without refrigeration and remain stable for extended periods of time (e.g., stable for at least about 1, 2, 3, 4, 5, 6, 9, 12, 18, 24, 36 months or longer upon storage at about 25° C.).

The pharmaceutical composition in lyophilized form can be stored in frozen condition for 1, 2, 3, 4, 5 or 10 years without loss of pharmacological or biological activity.

Accordingly, also provided herein are methods for treating disease in a subject by administering an effective amount of pharmaceutical compositions comprising lyophilized CFTR mRNA-lipid delivery vehicles to a subject (e.g., upon reconstitution with a rehydrating media such as sterile water for injection).

In some embodiments, the formulation is administered by a metered-dose inhaler.

In some embodiments, the formulation is administered by a nebulizer.

Suitable CFTR mRNA formulation for nebulization may be stored as a frozen liquid, or sterile liquid, or lyophilized or dry powder and reconstituted prior to nebulization. In some embodiments, the composition is stored in a single-use vial prior to nebulization. In some embodiments, the single-use vial comprises 50 mL or less of the composition. In some embodiments, the single-use vial comprises 40 mL or less of the composition. In some embodiments, the single-use vial comprises 30 mL or less of the composition. In some embodiments, the single-use vial comprises 20 mL or less of the composition. In some embodiments, the single-use vial comprises 10 mL or less of the composition. In some embodiments, the single-use vial comprises 9.0 mL or less of the composition. In some embodiments, the single-use vial comprises 8.0 mL or less of the composition. In some embodiments, the single-use vial comprises 7.0 mL or less of the composition. In some embodiments, the single-use vial comprises 6.0 mL or less of the composition. In some embodiments, the single-use vial comprises 5.0 mL or less of the composition. In some embodiments, the single-use vial comprises between 4.0 mL and 5.0 mL of the composition. More typically, the single-use vial comprises between 3.0 and 4.0 mL of the composition. In a specific embodiment, the single-use vial comprises 3.2 mL of the composition.

### Exemplary Formulations

### Compositions Comprising SEQ ID NO: 28

In embodiments, a composition comprises:
a. an mRNA encoding the CFTR protein, wherein the mRNA comprises:
   i. the sequence as set out in SEQ ID NO: 28;
   ii. a 5′ cap structure, wherein the 5′ cap structure is

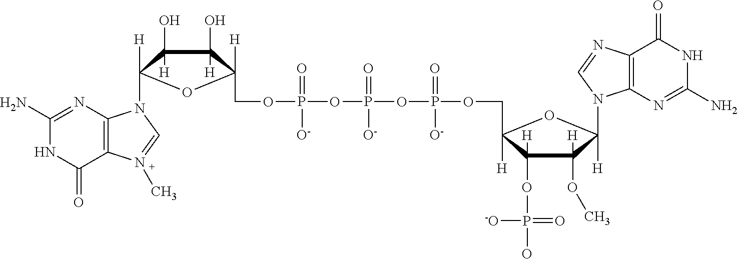

(“m7G(5′)ppp(5′) (2′OMeG)”); and
iii. a poly A tail of between 200 and 1000 adenosine nucleotides;
b. imidazole cholesterol ester (ICE);
c. 1,2-dioleoyl-SN-glycero-3-phosphoethanolamine (DOPE)
d. 1,2-dimyristoyl-rac-glycero-3-methylpolyoxyethylene (DMG-PEG-2K); and
e. trehalose.

In embodiments, the mRNA of SEQ ID NO: 28 has an average molecular weight of about 1.63 megadaltons. In embodiments, the 5′ UTR, hCFTR start codon, hCFTR stop codon, and 3′ UTR of the mRNA of SEQ ID NO: 28 are as set forth in Table A. In embodiments, the concentration of mRNA is about 0.6 mg/mL.

In embodiments, the nitrogen/phosphorus (N/P) ratio (i.e., the ratio of positively-charged nitrogens within ICE and the negatively charged phosphodiester lipids with the mRNA) is about 4. In embodiments, the average particle size range for the LNP formulation is about 40-60 nm.

Exemplary compositions comprising the mRNA of SEQ ID NO: 28 also include those described in Table D.

TABLE D
Exemplary Formulations of SEQ ID NO: 28

| | Formulation 1 | Formulation 2 | Formulation 3 | Formulation 4 |
|---|---|---|---|---|
| CFTR mRNA | SEQ ID NO: 28 | SEQ ID NO: 28 | SEQ ID NO: 28 | SEQ ID NO: 28 |
| 5′Cap | m7G(5′)ppp(5′) | m7G(5′)ppp(5′) | m7G(5′)ppp(5′) | m7G(5′)ppp(5′) |
| | (2′OMeG) | (2′OMeG) | (2′OMeG) | (2′OMeG) |
| 5′UTR | as described | as described | as described | as described |
| | in Table A | in Table A | in Table A | in Table A |
| Start Codon | AUG | AUG | AUG | AUG |
| hCFTR | | | | |
| Stop Codon | UAA | UAA | UAA | UAA |
| hCFTR | | | | |
| 3′UTR | as described | as described | as described | as described |
| | in Table A | in Table A | in Table A | in Table A |
| PolyA Tail | ~200-1000 | ~200-1000 | ~200-1000 | ~200-1000 |
| | adenosine | adenosine | adenosine | adenosine |
| | nucleotides | nucleotides | nucleotides | nucleotides |
| ICE:DOPE: | 60:35:5 | 60:30:10 | 60:35:5 | 60:35:5 |
| DMG-PEG-2K | | | | |
| Diluent | 10% trehalose | 10% trehalose | 10% sucrose | 10% glucose |
| Average | 40-60 nM | 40-60 nM | 40-60 nM | 40-60 nM |
| particle | | | | |
| size | | | | |
| N/P charge | 4 | 4 | 4 | 4 |
| ratio | | | | |

In embodiments, a formulation is Formulation 1. In embodiments, Formulation 1 is further characterized by a concentration of the mRNA that is about 0.6 mg/ml.

In embodiments, a formulation is Formulation 2. In embodiments, Formulation 2 is further characterized by a concentration of the mRNA that is about 0.6 mg/ml.

In embodiments, a formulation is Formulation 3. In embodiments, Formulation 3 is further characterized by a concentration of the mRNA that is about 0.6 mg/ml.

In embodiments, a formulation is Formulation 4. In embodiments, Formulation 4 is further characterized by a concentration of the mRNA that is about 0.6 mg/ml.

### Compositions Comprising SEQ ID NO: 29

In embodiments, a composition comprises:
a. an mRNA encoding the CFTR protein, wherein the mRNA comprises:
   i. the sequence as set out in SEQ ID NO: 29;
   ii. a 5′ cap structure, wherein the 5′ cap structure is

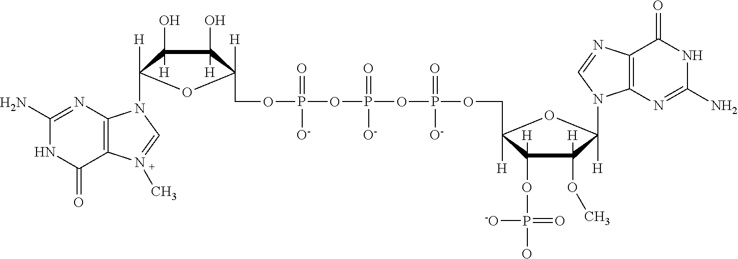

(“m7G(5′)ppp(5′) (2′OMeG)”); and
iii. a poly A tail of between 200 and 1000 adenosine nucleotides;
b. imidazole cholesterol ester (ICE);
c. 1,2-dioleoyl-SN-glycero-3-phosphoethanolamine (DOPE)
d. 1,2-dimyristoyl-rac-glycero-3-methylpolyoxyethylene (DMG-PEG-2K); and
e. trehalose.

In embodiments, the mRNA of SEQ ID NO: 29 has an average molecular weight of about 1.63 megadaltons. In embodiments, the 5′ UTR, hCFTR start codon, hCFTR stop codon, and 3′ UTR of the mRNA of SEQ ID NO: 29 are as set forth in Table B. In embodiments, the concentration of mRNA is about 0.6 mg/mL.

In embodiments, the nitrogen/phosphorus (N/P) ratio (i.e., the ratio of positively-charged nitrogens within ICE and the negatively charged phosphodiester lipids with the mRNA) is about 4. In embodiments, the average particle size range for the LNP formulation is about 40-60 nm.

Exemplary compositions comprising the mRNA of SEQ ID NO: 29 also include those described in Table E.

TABLE E
Exemplary Formulations of SEQ ID NO: 29

| | Formulation 5 | Formulation 6 | Formulation 7 | Formulation 8 |
|---|---|---|---|---|
| CFTR mRNA | SEQ ID NO: 29 | SEQ ID NO: 29 | SEQ ID NO: 29 | SEQ ID NO: 29 |
| 5′Cap | m7G(5′)ppp(5′) | m7G(5′)ppp(5′) | m7G(5′)ppp(5′) | m7G(5′)ppp(5′) |
| | (2′OMeG) | (2′OMeG) | (2′OMeG) | (2′OMeG) |
| 5′UTR | as described | as described | as described | as described |
| | in Table B | in Table B | in Table B | in Table B |
| Start Codon | AUG | AUG | AUG | AUG |
| hCFTR | | | | |
| Stop Codon | UAA | UAA | UAA | UAA |
| hCFTR | | | | |
| 3′UTR | as described | as described | as described | as described |
| | in Table B | in Table B | in Table B | in |
| PolyA Tail | ~200-1000 | ~200-1000 | ~200-1000 | ~200-1000 |
| | adenosine | adenosine | adenosine | adenosine |
| | nucleotides | nucleotides | nucleotides | nucleotides |
| ICE:DOPE: | 60:35:5 | 60:30:10 | 60:35:5 | 60:35:5 |
| DMG-PEG-2K | | | | |
| Diluent | 10% trehalose | 10% trehalose | 10% sucrose | 10% glucose |
| Average | 40-60 nM | 40-60 nM | 40-60 nM | 40-60 nM |
| particle | | | | |
| size | | | | |
| N/P charge | 4 | 4 | 4 | 4 |
| ratio | | | | |

In embodiments, a formulation is Formulation 5. In embodiments, Formulation 5 is further characterized by a concentration of the mRNA that is about 0.6 mg/ml.

In embodiments, a formulation is Formulation 6. In embodiments, Formulation 6 is further characterized by a concentration of the mRNA that is about 0.6 mg/ml.

In embodiments, a formulation is Formulation 7. In embodiments, Formulation 7 is further characterized by a concentration of the mRNA that is about 0.6 mg/ml.

In embodiments, a formulation is Formulation 8. In embodiments, Formulation 8 is further characterized by a concentration of the mRNA that is about 0.6 mg/ml.

### Compositions Comprising SEQ ID NO: 30

In embodiments, a composition comprises:
a. an mRNA encoding the CFTR protein, wherein the mRNA comprises:
   i. the sequence as set out in SEQ ID NO: 30;
   ii. a 5′ cap structure, wherein the 5′ cap structure is

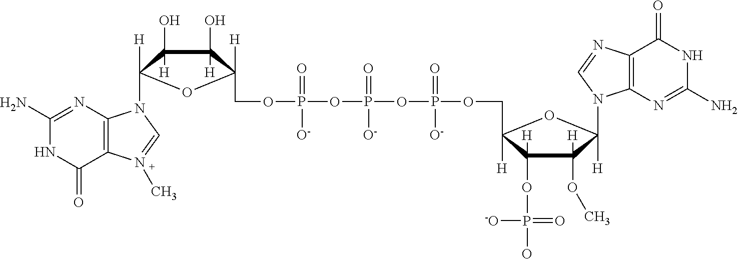

(“m7G(5′)ppp(5′) (2′OMeG)”); and
iii. a poly A tail of between 200 and 1000 adenosine nucleotides;
b. imidazole cholesterol ester (ICE);
c. 1,2-dioleoyl-SN-glycero-3-phosphoethanolamine (DOPE)
d. 1,2-dimyristoyl-rac-glycero-3-methylpolyoxyethylene (DMG-PEG-2K); and
e. trehalose.

In embodiments, the mRNA of SEQ ID NO: 30 has an average molecular weight of about 1.63 megadaltons. In embodiments, the 5′ UTR, hCFTR start codon, hCFTR stop codon, and 3′ UTR of the mRNA of SEQ ID NO: 30 are as set forth in Table C. In embodiments, the concentration of mRNA is about 0.6 mg/mL.

In embodiments, the nitrogen/phosphorus (N/P) ratio (i.e., the ratio of positively-charged nitrogens within ICE and the negatively charged phosphodiester lipids with the mRNA) is about 4. In embodiments, the average particle size range for the LNP formulation is about 40-60 nm.

Exemplary compositions comprising the mRNA of SEQ ID NO: 30 also include those described in Table F.

TABLE F
Exemplary Formulations of SEQ ID NO: 30

| | Formulation 9 | Formulation 10 | Formulation 11 | Formulation 12 |
|---|---|---|---|---|
| CFTR mRNA | SEQ ID NO: 30 | SEQ ID NO: 30 | SEQ ID NO: 30 | SEQ ID NO: 30 |
| 5′Cap | m7G(5′)ppp(5′) | m7G(5′)ppp(5′) | m7G(5′)ppp(5′) | m7G(5′)ppp(5′) |
| | (2′OMeG) | (2′OMeG) | (2′OMeG) | (2′OMeG) |
| 5′UTR | as described | as described | as described | as described |
| | in Table C | in Table C | in Table C | in Table C |
| Start Codon | AUG | AUG | AUG | AUG |
| hCFTR | | | | |
| Stop Codon | UGA | UGA | UGA | UGA |
| hCFTR | | | | |
| 3′UTR | as described | as described | as described | as described |
| | in Table C | in Table C | in Table C | in Table C |
| PolyA Tail | ~200-1000 | ~200-1000 | ~200-1000 | ~200-1000 |
| | adenosine | adenosine | adenosine | adenosine |
| | nucleotides | nucleotides | nucleotides | nucleotides |
| ICE:DOPE: | 60:35:5 | 60:30:10 | 60:35:5 | 60:35:5 |
| DMG-PEG-2K | | | | |
| Diluent | 10% trehalose | 10% trehalose | 10% sucrose | 10% glucose |
| Average | 40-60 nM | 40-60 nM | 40-60 nM | 40-60 nM |
| particle | | | | |
| size | | | | |
| N/P charge | 4 | 4 | 4 | 4 |
| ratio | | | | |

In embodiments, a formulation is Formulation 9. In embodiments, Formulation 9 is further characterized by a concentration of the mRNA that is about 0.6 mg/ml.

In embodiments, a formulation is Formulation 10. In embodiments, Formulation 10 is further characterized by a concentration of the mRNA that is about 0.6 mg/ml.

In embodiments, a formulation is Formulation 11. In embodiments, Formulation 11 is further characterized by a concentration of the mRNA that is about 0.6 mg/ml.

In embodiments, a formulation is Formulation 12. In embodiments, Formulation 12 is further characterized by a concentration of the mRNA that is about 0.6 mg/ml.

### Assessment of Formulation Characteristics

The formulation may be assessed for one or more of the following characteristics: appearance, identity, quantity, concentration, presence of impurities, microbiological assessment, pH level and activity.

In some embodiments, acceptable appearance of the formulation includes a clear, colorless solution, essentially free of visible particulates.

In some embodiments, the identity of the CFTR mRNA is assessed by sequencing methods. The sequencing methods are performed to confirm the correct sequence of the desired CFTR mRNA.

In some embodiments, the concentration of the CFTR mRNA is assessed by a suitable method, such as UV spectrophotometry. In some embodiments, a suitable concentration is between about 90% and 110% nominal (0.9-1.1 mg/mL). Accordingly, in some embodiments, a suitable concentration is about 90% nominal (0.9 mg/mL). In some embodiments, a suitable concentration is about 91% nominal (0.91 mg/mL). In some embodiments, a suitable concentration is about 92% nominal (0.92 mg/mL). In some embodiments, a suitable concentration is about 93% nominal (0.93 mg/mL). In some embodiments, a suitable concentration is about 94% nominal (0.94 mg/mL). In some embodiments, a suitable concentration is about 95% nominal (0.95 mg/mL). In some embodiments, a suitable concentration is about 96% nominal (0.96 mg/mL). In some embodiments, a suitable concentration is about 97% nominal (0.97 mg/mL). In some embodiments, a suitable concentration is about 98% nominal (0.98 mg/mL). In some embodiments, a suitable concentration is about 99% nominal (0.99 mg/mL). In some embodiments, a suitable concentration is about 100% nominal (1.0 mg/mL). In some embodiments, a suitable concentration is about 101% nominal (1.01 mg/mL). In some embodiments, a suitable concentration is about 102% nominal (1.02 mg/mL). In some embodiments, a suitable concentration is about 103% nominal (1.03 mg/mL). In some embodiments, a suitable concentration is about 104% nominal (1.04 mg/mL). In some embodiments, a suitable concentration is about 105% nominal (1.05 mg/mL). In some embodiments, a suitable concentration is about 106% nominal (1.06 mg/mL). In some embodiments, a suitable concentration is about 107% nominal (1.07 mg/mL). In some embodiments, a suitable concentration is about 108% nominal (1.08 mg/mL). In some embodiments, a suitable concentration is about 109% nominal (1.09 mg/mL). In some embodiments, a suitable concentration is about 110% nominal (1.10 mg/mL).

In some embodiments, the formulation is assessed to determine CFTR mRNA integrity, to determine whether there is residual plasmid DNA, and to determine the presence of residual solvent. In some embodiments, CFTR mRNA integrity is assessed by agarose gel electrophoresis. The gels are analyzed to determine whether the banding pattern and apparent nucleotide length is consistent with an analytical reference standard. For example, gels are assessed to determine whether banding pattern and apparent nucleotide length is consistent with an analytical reference standard and is oriented between the 7,000 nt and 3,000 nt bands. Additional methods to assess CFTR mRNA integrity include, for example, assessment of the purified mRNA using capillary gel electrophoresis (CGE). In some embodiments, acceptable purity of the CFTR mRNA in the formulation as determined by CGE is that the main peak is not less than about 55%, 50%, 45%, 40%, 35%, or 30%. Accordingly, in some embodiments, acceptable purity of the CFTR mRNA in the formulation is a CGE with a main peak not less than about 55%. In some embodiments, acceptable purity of the CFTR mRNA in the formulation is a CGE with a main peak not less than about 50%. In some embodiments, acceptable purity of the CFTR mRNA in the formulation is a CGE with a main peak not less than about 45%. In some embodiments, acceptable purity of the CFTR mRNA in the formulation is a CGE with a main peak not less than about 40%. In some embodiments, acceptable purity of the CFTR mRNA in the formulation is a CGE with a main peak not less than about 35%. In some embodiments, acceptable purity of the CFTR mRNA in the formulation is a CGE with a main peak not less than about 30%.

The formulation can also be assessed for the presence of any residual plasmid DNA. Various methods can be used to assess the presence of residual plasmid DNA, for example qPCR. In some embodiments, less than 10 pg/mg (e.g., less than 10 pg/mg, less than 9 pg/mg, less than 8 pg/mg, less than 7 pg/mg, less than 6 pg/mg, less than 5 pg/mg, less than 4 pg/mg, less than 3 pg/mg, less than 2 pg/mg, or less than 1 pg/mg) is an acceptable level of residual plasmid DNA. Accordingly, in some embodiments, the formulation has less than 10 pg/mg of residual plasmid DNA. In some embodiments, the formulation has less than 9 pg/mg of residual plasmid DNA. In some embodiments, the formulation has less than 8 pg/mg of residual plasmid DNA. In some embodiments, the formulation has less than 7 pg/mg of residual plasmid DNA. In some embodiments, the formulation has less than 6 pg/mg of residual plasmid DNA. In some embodiments, the formulation has less than 5 pg/mg of residual plasmid DNA. In some embodiments, the formulation has less than 4 pg/mg of residual plasmid DNA. In some embodiments, the formulation has less than 3 pg/mg of residual plasmid DNA. In some embodiments, the formulation has less than 2 pg/mg of residual plasmid DNA. In some embodiments, the formulation has less than 1 pg/mg of residual plasmid DNA.

The formulation can also be assessed for the presence of any residual solvents. Various methods can be used to determine the presence of residual solvent. In some embodiments, acceptable residual solvent levels are not more than 10,000 ppm. In some embodiments, acceptable residual solvent levels are not more than 9,000 ppm. In some embodiments, acceptable residual solvent levels are not more than 8,000 ppm. In some embodiments, acceptable residual solvent levels are not more than 7,000 ppm. In some embodiments, acceptable residual solvent levels are not more than 6,000 ppm. In some embodiments, acceptable residual solvent levels are not more than 5,000 ppm. In some embodiments, acceptable residual solvent levels are not more than 4,000 ppm. In some embodiments, acceptable residual solvent levels are not more than 3,000 ppm. In some embodiments, acceptable residual solvent levels are not more than 2,000 ppm. In some embodiments, acceptable residual solvent levels are not more than 1,000 ppm. In some embodiments, the residual solvent is, for example, ethanol.

The formulation can also be assessed for the presence of bacterial endotoxins. In some embodiments, bacterial endotoxins are <0.5 EU/mL, <0.4 EU/mL, <0.3 EU/mL, <0.2 EU/mL or <0.1 EU/mL. Accordingly, in some embodiments, bacterial endotoxins in the purified mRNA are <0.5 EU/mL. In some embodiments, bacterial endotoxins in the purified mRNA are <0.4 EU/mL. In some embodiments, bacterial endotoxins in the purified mRNA are <0.3 EU/mL. In some embodiments, bacterial endotoxins in the purified mRNA are <0.2 EU/mL. In some embodiments, bacterial endotoxins in the purified mRNA are <0.2 EU/mL. In some embodiments, bacterial endotoxins in the purified mRNA are <0.1 EU/mL.

The formulation can also be assessed for microbial contaminants (e.g., “bioburden testing”). The tests can include for example an assessment of total aerobic microbial count (“TAMC”) and/or an assessment of total yeast/mold count (“TYMC”). In some embodiments, the purified mRNA has not more than 1 CFU/10 mL, 1 CFU/25 mL, 1CFU/50 mL, 1CFU/75 mL, or not more than 1 CFU/100 mL. Accordingly, in some embodiments, the purified mRNA has not more than 1 CFU/10 mL. In some embodiments, the purified mRNA has not more than 1 CFU/25 mL. In some embodiments, the purified mRNA has not more than 1 CFU/50 mL. In some embodiments, the purified mRNA has not more than 1 CFR/75 mL. In some embodiments, the purified mRNA has 1 CFU/100 mL.

The pH of the formulation can also be assessed. In some embodiments, acceptable pH of the formulation is between 5 and 8. Accordingly, in some embodiments, the formulation has a pH of about 5. In some embodiments, the formulation has a pH of about 6. In some embodiments, the formulation has a pH of about 7. In some embodiments, the formulation has a pH of about 7. In some embodiments, the formulation has a pH of about 8.

The formulation can also be assessed for translational fidelity of the CFTR mRNA. The translational fidelity can be assessed by various methods such as, for example, transfection and Western blot analysis. Acceptable characteristics of the purified mRNA includes banding pattern on a Western blot that migrates at a similar molecular weight as a reference standard. For example, the sample main band migrates at a similar apparent molecular weight as the reference standard and is oriented between the 100 kDa and 250 kDa markers.

The formulation can also be assessed for conductance. In some embodiments, acceptable characteristics of the purified mRNA include a conductance of between about 50% and 150% of a reference standard. Accordingly, in some embodiments, the formulation has a conductance of about 50% of a reference standard. In some embodiments, the formulation has a conductance of about 55% of a reference standard. In some embodiments, the formulation has a conductance of about 60% of a reference standard. In some embodiments, the formulation has a conductance of about 65% of a reference standard. In some embodiments, the formulation has a conductance of about 70% of a reference standard. In some embodiments, the formulation has a conductance of about 75% of a reference standard. In some embodiments, the formulation has a conductance of about 80% of a reference standard. In some embodiments, the formulation has a conductance of about 85% of a reference standard. In some embodiments, the formulation has a conductance of about 90% of a reference standard. In some embodiments, the formulation has a conductance of about 95% of a reference standard. In some embodiments, the formulation has a conductance of about 100% of a reference standard. In some embodiments, the formulation has a conductance of about 105% of a reference standard. In some embodiments, the formulation has a conductance of about 110% of a reference standard. In some embodiments, the formulation has a conductance of about 115% of a reference standard. In some embodiments, the formulation has a conductance of about 120% of a reference standard. In some embodiments, the formulation has a conductance of about 125% of a reference standard. In some embodiments, the formulation has a conductance of about 130% of a reference standard. In some embodiments, the formulation has a conductance of about 135% of a reference standard. In some embodiments, the formulation has a conductance of about 140% of a reference standard. In some embodiments, the formulation has a conductance of about 145% of a reference standard. In some embodiments, the formulation has a conductance of about 150% of a reference standard.

The CFTR mRNA in the formulation can also be assessed for Cap percentage.

Various methods can be used to assess Cap percentage, for example Ultra Performance Liquid Chromatography (“UPLC”). In some embodiments, an acceptable Cap percentage includes Cap1, % area of not less than about 80%, 85%, 90%, or 95%. Accordingly, in some embodiments, an acceptable Cap percentage includes Cap1, % area of not less than about 80%. In some embodiments, an acceptable Cap percentage includes Cap1, % area of not less than about 85%. In some embodiments, an acceptable Cap percentage includes Cap1, % area of not less than about 90%. In some embodiments, an acceptable Cap percentage includes Cap1, % area of not less than about 95%.

Furthermore, the CFTR mRNA in the formulation can be assessed for PolyA tail length. Various methods can be used to assess PolyA tail length, for example capillary electrophoresis. In some embodiments, an acceptable PolyA tail length is about 100-1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides). Accordingly, in some embodiments an acceptable PolyA tail length is about 100 nucleotides. In some embodiments, an acceptable PolyA tail length is about 200 nucleotides. In some embodiments, an acceptable PolyA tail length is about 250 nucleotides. In some embodiments, an acceptable PolyA tail length is about 300 nucleotides. In some embodiments, an acceptable PolyA tail length is about 350 nucleotides. In some embodiments, an acceptable PolyA tail length is about 400 nucleotides. In some embodiments, an acceptable PolyA tail length is about 450 nucleotides. In some embodiments, an acceptable PolyA tail length is about 500 nucleotides. In some embodiments, an acceptable PolyA tail length is about 550 nucleotides. In some embodiments, an acceptable PolyA tail length is about 600 nucleotides. In some embodiments, an acceptable PolyA tail length is about 650 nucleotides. In some embodiments, an acceptable PolyA tail length is about 700 nucleotides. In some embodiments, an acceptable PolyA tail length is about 750 nucleotides. In some embodiments, an acceptable PolyA tail length is about 800 nucleotides. In some embodiments, an acceptable PolyA tail length is about 850 nucleotides. In some embodiments, an acceptable PolyA tail length is about 900 nucleotides. In some embodiments, an acceptable PolyA tail length is about 950 nucleotides. In some embodiments, an acceptable PolyA tail length is about 1000 nucleotides. In some embodiments, an acceptable PolyA tail length is about 1100 nucleotides. In some embodiments, an acceptable PolyA tail length is about 1200 nucleotides. In some embodiments, an acceptable PolyA tail length is about 1300 nucleotides. In some embodiments, an acceptable PolyA tail length is about 1400 nucleotides. In some embodiments, an acceptable PolyA tail length is about 1500 nucleotides. In some embodiments, an acceptable PolyA tail length is between about 200-1000 nt. In some embodiments, an acceptable PolyA tail length is between about 300-900 nt. In some embodiments, an acceptable PolyA tail length is between about 400 and 800 nt.

### Pulmonary Delivery

A CFTR mRNA may be formulated for delivery via different administration routes including, but not limited to, oral, rectal, vaginal, transmucosal, or intestinal administration; parenteral delivery, including intradermal, transdermal (topical), intramuscular, subcutaneous, intramedullary injections, as well as intrathecal, direct intraventricular, intravenous, intraperitoneal, and/or intranasal administration.

In some embodiments, a CFTR mRNA is formulated for pulmonary delivery. As used herein, pulmonary delivery refers to delivery to lung via, e.g., nasal cavity, trachea, bronchi, bronchioles, and/or other pulmonary system. In particular embodiments, a CFTR mRNA is formulated for nebulization. In these embodiments, the delivery vehicle may be in an aerosolized composition which can be inhaled. In some embodiments, pulmonary delivery involves inhalation (e.g., for nasal, tracheal, or bronchial delivery). In some embodiments, the CFTR mRNA formulation is nebulized prior to inhalation.

### Nebulization

Inhaled aerosol droplets of a particle size of 1-5 um can penetrate into the narrow branches of the lower airways. Aerosol droplets with a larger diameter are typically absorbed by the epithelia cells lining the oral cavity, and are unlikely to reach the lower airway epithelium and the deep alveolar lung tissue.

Particle size in an aerosol is commonly described in reference to the Mass Median Aerodynamic Diameter (MMAD). MMAD, together with the geometric standard deviation (GSD), describes the particle size distribution of any aerosol statistically, based on the weight and size of the particles. Means of calculating the MMAD of an aerosol are well known in the art.

A specific method of calculating the MMAD using a cascade impactor was first described in 1959 by Mitchell et al. The cascade impactor for measuring particle sizes is constructed of a succession of jets, each followed by an impaction slide, and is based on the principle that particles in a moving air stream impact on a slide placed in their path, if their momentum is sufficient to overcome the drag exerted by the air stream as it moves around the slide. As each jet is smaller than the preceding one, the velocity of the air stream and therefore that of the dispersed particles are increased as the aerosol advances through the impactor. Consequently, smaller particles eventually acquire enough momentum to impact on a slide, and a complete particle size classification of the aerosol is achieved. The improved Next Generation Impactor, used herein to measure the MMAD of the pharmaceutical composition of the invention, was first described by Marple et al. in 2003 and has been widely used in the pharmacopocia since.

Another parameter to describe particle size in an aerosol is the Volume Median Diameter (VMD). VMD also describes the particle size distribution of an aerosol based on the volume of the particles. Means of calculating the VMD of an aerosol are well known in the art. A specific method used for determining the VMD is laser diffraction, which is used herein to measure the VMD of the pharmaceutical composition of the invention (see, e.g., Clark, 1995, Int J Pharm. 115:69-78).

In some embodiments, the mean particle size of the nebulized CFTR mRNA formulation of the invention is between about 4 μm and 6 μm, e.g., about 4 μm, about 4.5 μm, about 5 μm, about 5.5 μm, or about 6 μm.

The Fine Particle Fraction (FPF) is defined as the proportion of particles in an aerosol which have an MMAD or a VMD smaller than a specified value. In some embodiments, the FPF of the nebulized CFTR mRNA formulation of the invention with a particle size <5 μm is at least about 30%, more typically at least about 40%, e.g., at least about 50%, more typically at least about 60%.

In some embodiments, nebulization is performed in such a manner that the mean respirable emitted dose (i.e., the percentage of FPF with a particle size <5 μm; e.g., as determined by next generation impactor with 15 L/min extraction) is at least about 30% of the emitted dose, e.g., at least about 31%, at least about 32%, at least about 33%, at least about 34%, or at least about 35% the emitted dose. In some embodiments, nebulization is performed in such a manner that the mean respirable delivered dose (i.e., the percentage of FPF with a particle size <5 μm; e.g., as determined by next generation impactor with 15 L/min extraction) is at least about 15% of the emitted dose, e.g., at least 16% or 16.5% of the emitted dose.

### Nebulizer

Nebulization can be achieved by any nebulizer known in the art. A nebulizer transforms a liquid to a mist so that it can be inhaled more easily into the lungs. Nebulizers are effective for infants, children and adults. Nebulizers are able to nebulize large doses of inhaled medications. Typically, a nebulizer for use with the invention comprises a mouthpiece that is detachable. This is important because only clean mouthpieces that are RNase free should be used when administering the CFTR mRNA formulation of the invention.

In some embodiments, the reservoir volume of the nebulizer ranges from about 5.0 mL to about 8.0 mL. In some embodiments, the reservoir volume of the nebulizer is about 5.0 mL. In some embodiments, the reservoir volume of the nebulizer is about 6.0 mL. In some embodiments, the reservoir volume of the nebulizer is about 7.0 mL. In some embodiments, the reservoir volume of the nebulizer is about 8.0 mL.

One type of nebulizer is a jet nebulizer, which comprises tubing connected to a compressor, which causes compressed air or oxygen to flow at a high velocity through a liquid medicine to turn it into an aerosol, which is then inhaled by the patient.

Another type of nebulizer is the ultrasonic wave nebulizer, which comprises an electronic oscillator that generates a high frequency ultrasonic wave, which causes the mechanical vibration of a piezoelectric element, which is in contact with a liquid reservoir. The high frequency vibration of the liquid is sufficient to produce a vapor mist. Exemplary ultrasonic wave nebulizers are the Omron NE-U17 and the Beurer Nebulizer IH30.

A third type of nebulizer comprises vibrating mesh technology (VMT). A VMT nebulizer typically comprises a mesh/membrane with 1000-7000 holes that vibrates at the top of a liquid reservoir and thereby pressures out a mist of very fine aerosol droplets through the holes in the mesh/membrane. VMT nebulizers suitable for delivery of the CFTR mRNA formulation include any of the following: cFlow (PARI Medical Ltd.), i-Neb (Respironics Respiratory Drug Delivery Ltd), Nebulizer IH50 (Beurer Ltd.), AcroNeb Go (Acrogen Ltd.), InnoSpire Go (Respironics Respiratory Drug Delivery Ltd), Mesh Nebulizer (Shenzhen Homed Medical Device Co, Ltd.), Portable Nebulizer (Microbase Technology Corporation) and Airworks (Convexity Scientific LLC). In some embodiments, the mesh or membrane of the VMT nebulizer is made to vibrate by a piezoelectric element. In some embodiments, the mesh or membrane of the VMT nebulizer is made to vibrate by ultrasound.

VMT nebulizers have been found to be particularly suitable for practicing the invention because they do not affect the mRNA integrity of the CFTR mRNA formulation of the invention. Typically, at least about 60%, e.g., at least about 65% or at least about 70%, of the mRNA in the CFTR mRNA formulation of the invention maintains its integrity after nebulization.

In some embodiments, nebulization is continuous during inhalation and exhalation. More typically, nebulization is breath-actuated. Suitable nebulizers for use with the invention have nebulization rate of >0.2 mL/min. In some embodiments, the nebulization rate is >0.25 mL/min. In other embodiment, the nebulization rate is >0.3 mL/min. In certain embodiments, the nebulization rate is >0.45 mL/min. In a typical embodiment, the nebulization rate ranges between 0.2 mL/minute and 0.5 mL/minute.

In some embodiments, the nebulization volume is at a volume ranging from 13.0 mL to 42.0 mL, e.g., between 14 mL and 28 mL. In some embodiments, the nebulization volume is at a volume less than or equal to 13.9 mL. In some embodiments, the nebulization volume is at a volume less than or equal to 16.0 mL. In some embodiments, the nebulization volume is at a volume less than or equal to 18.0 mL. In some embodiments, the nebulization volume is at a volume less than or equal to 20.0 mL. In some embodiments, the nebulization volume is at a volume less than or equal to 22.0 mL. In some embodiments, the nebulization volume is at a volume less than or equal to 24.0 mL. In some embodiments, the nebulization volume is at a volume less than or equal to 26.0 mL. In some embodiments, the nebulization volume is at a volume less than or equal to 27.9 mL. In some embodiments, the nebulization volume is at a volume less than or equal to 30.0 mL. In some embodiments, the nebulization volume is at a volume less than or equal to 32.0 mL. In some embodiments, the nebulization volume is at a volume less than or equal to 34.0 mL. In some embodiments, the nebulization volume is at a volume less than or equal to 36.0 mL. In some embodiments, the nebulization volume is at a volume less than or equal to 38.0 mL. In some embodiments, the nebulization volume is at a volume less than or equal to 40.0 mL. In some embodiments, the nebulization volume is at a volume less than or equal to 41.8 mL.

A human subject may display adverse effects during treatment, when the nebulization volume exceeds 10 mL. In particular, such adverse effects may be more common when volumes greater than 20 mL are administered. In some embodiments, the nebulization volume does not exceed 20 mL.

In some embodiments, a single dose of the CO-hCFTR mRNA composition of the invention can be administered with only a one or two refills per nebulization treatment. For example, if the total volume of the CO-hCFTR mRNA composition that is to be administered to the patient is 13 mL, then only a single refill is required to administer the entire volume when using a nebulizer with an 8 mL reservoir, but two refills are required to administer the same volume when using a nebulizer with a 5 mL reservoir. In another embodiment, at least three refills are required per nebulization treatment, e.g., to administer a volume of 26 mL, at least three refills are required when using a nebulizer with an 8 mL reservoir. In yet a further embodiment, at least four refills are required. For example, to deliver 42 mL with a nebulizer having a 5 mL reservoir, at least eight refills are required. Typically, no more than 1-3 refills will be required to administer the CO-hCFTR mRNA composition of the invention.

Typically, the duration of nebulization is between 30 and 300 minutes. An average nebulization session may exceed 30 minutes, e.g., it may last for at least 35 minutes or more, at least 45 minutes or more, or at least 1 hour or more. For example, most patients are treated with a nebulization session that last between about 45 minutes to about 90 minutes, although some patients may require nebulization sessions that may last from about 100 minutes to about 180 minutes. Longer treatment may last for 1 hour, 1.5 hours, or 2 hours. Accordingly, in some embodiments, the nebulization session is about 45 minutes, about 60 minutes, about 75 minutes, about 90 minutes, about 105 minutes, about 120 minutes, about 135 minutes, about 150 minutes, about 165 minutes, or about 180 minutes. In some embodiments, the nebulization session is about 45 minutes. In some embodiments, nebulization is about 2 hours and 25 minutes. In some embodiments, patients may require nebulization sessions that may last from about 150 minutes to about 300 minutes, e.g., between 3 hours and 4.5 hours.

In some embodiments, the number of nebulizers used during a single nebulization session ranges from 2-8. In some embodiments, 1 nebulizer is used during a single nebulization session. In some embodiments, 2 nebulizers are used during a single nebulization session. In some embodiments, 3 nebulizers are used during a single nebulization session. In some embodiments, 4 nebulizers are used during a single nebulization session. In some embodiments, 5 nebulizers are used during a single nebulization session. In some embodiments, 6 nebulizers are used during a single nebulization session. In some embodiments, 7 nebulizers are used during a single nebulization session. In some embodiments, 8 nebulizers are used during a single nebulization session.

### Pharmacokinetics and Tissue Distribution

According to the present invention, administration of a formulation comprising a CFTR mRNA results in delivery of the mRNA and encoded CFTR protein in various targets tissues described herein. In particular, administration of a formulation comprising a CFTR mRNA according to the present invention results in a therapeutically or clinically effective level or activity of CFTR in the target tissue. In various embodiments, a target tissue includes lung, pancreas, kidney, liver, spleen, testes/ovaries, salivary glands, sweat glands, heart and brain. In some embodiments, a target tissue is lung. In some embodiments, a target tissue is the upper (i.e., superior) lobe of the right or left lung. In some embodiments, a target tissue is the lower (i.e., inferior) lobe of the right or left lung. In some embodiments, a target tissue is the middle lobe of the right lung.

In some embodiments, a target tissue is the apical segment of the right lung or the apicoposterior segment of the left lung. In some embodiments, a target tissue is the posterior segment of the right lung. In some embodiments, a target tissue is the anterior segment of the right or left lung. In some embodiments, a target tissue is the superior segment of the right or left lung. In some embodiments, a target tissue is the lateral basal segment of the right or left lung. In some embodiments, a target tissue is the anterior basal segment of the right lung. In some embodiments, a target tissue is the anteromedial basal segment of the left lung. In some embodiments, a target tissue is the lateral segment of the right lung. In some embodiments, a target tissue is the medial segment of the right lung. In some embodiments, a target tissue is the superior lingular segment of the left lung. In some embodiments, a target tissue is the inferior lingular segment of the left lung. In some embodiments, a target tissue is the posterior basal segment of the right or left lung. In some embodiments, a target tissue is the medial basal segment of the right lung.

In particular embodiments, a target tissue is epithelial cells in the lung. In some embodiments, a target tissue is smooth muscle cells in the lung. In some embodiment, a target tissue is pancreatic duct epithelial cells. In some embodiment, a target tissue is bile-duct epithelial cells. In some embodiment, a target tissue is epithelial cells of the salivary glands. In some embodiment, a target tissue is renal epithelial cells. In some embodiment, a target tissue is beta-S cells in sweat gland secretory coils of sweat glands. In some embodiment, a target tissue is epithelial cells of the reproductive tract.

In some embodiments, a CFTR mRNA delivered according to the present invention achieves a level of CFTR protein expression or activity that is at least 5%, 10%, 20%, 30%, 40%, 50%, 60%, 70%, 80%, 90%, or 95% of the normal level of CFTR protein expression or activity in a target tissue described herein. In some embodiments, a CFTR mRNA delivered according to the present invention achieves a level of CFTR protein expression or activity that is increased by at least 1-fold, 2-fold, 3-fold, 4-fold, 5-fold, 6-fold, 7-fold, 8-fold, 9-fold or 10-fold as compared to a control (e.g., endogenous level of protein or activity without or before the treatment according to the invention, or a historical reference level) in a target tissue described herein.

In general, a CFTR mRNA delivered according to the present invention have sufficiently long half time in a target tissue described herein. In some embodiments, a CFTR mRNA delivered according to the present invention has a half-life of at least approximately 30 minutes, 45 minutes, 60 minutes, 90 minutes, 2 hours, 3 hours, 4 hours, 5 hours, 6 hours, 7 hours, 8 hours, 9 hours, 10 hours, 12 hours, 16 hours, 18 hours, 20 hours, 25 hours, 30 hours, 35 hours, 40 hours, 3 days, 7 days, 14 days, 21 days, or a month. In some embodiments, a CFTR mRNA delivered according to the present invention results in detectable CFTR protein level or activity in a target tissue (e.g., the lung) or bloodstream after 12 hours, 24 hours, 30 hours, 36 hours, 42 hours, 48 hours, 54 hours, 60 hours, 66 hours, 72 hours, 78 hours, 84 hours, 90 hours, 96 hours, 102 hours, a week, two weeks, three weeks, or a month following administration. Detectable level or activity may be determined using various methods known in the art.

In some embodiments, a CFTR mRNA delivered according to the present invention results in increased CFTR protein level or activity in upper lobe lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in lower lobe lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in middle lobe lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in distal lung tissues by, e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 200-fold, 300-fold, 400-fold, or 500-fold as compared to a control (e.g., endogenous level of protein or activity without or before the treatment according to the invention, or a historical reference level).

In some embodiments, a CFTR mRNA delivered according to the present invention results in increased CFTR protein level or activity in distal peripheral lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 200-fold, or 300-fold as compared to a control (e.g., endogenous level of protein or activity without or before the treatment according to the invention, or a historical reference level).

In some embodiments, a CFTR mRNA delivered according to the present invention results in increased CFTR protein level or activity in lateral peripheral lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in medial peripheral lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, or 1000-fold as compared to a control (e.g., endogenous level of protein or activity without or before the treatment according to the invention, or a historical reference level).

In some embodiments, a CFTR mRNA delivered according to the present invention results in increased CFTR protein level or activity in middle lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 150-fold, 200-fold, 250-fold, 300-fold, 350-fold, 400-fold, 450-fold, or 500-fold as compared to a control (e.g., endogenous level of protein or activity without or before the treatment according to the invention, or a historical reference level).

In some embodiments, a CFTR mRNA delivered according to the present invention results in increased CFTR protein level or activity in proximal lung tissue by, e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in detectable CFTR protein or activity in the larynx, trachea, nasal turbinate, and/or bronchoalveolar lavage fluid (BALF). In some embodiments, a CFTR mRNA delivered according to the present invention results in detectable CFTR protein or activity in blood. In some embodiments, a CFTR mRNA delivered according to the present invention results in detectable CFTR protein or activity in lung, pancreas, kidney, liver, spleen, testes/ovaries, salivary glands, sweat glands, heart and brain.

In some embodiments, a CFTR mRNA delivered according to the present invention results in increased CFTR protein level or activity in larynx, trachea, tracheobronchial lymph node, and/or blood by, e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

The CFTR mRNA expression may be detected or quantified by qPCR on RNA purified from tissue samples. The CFTR protein expression may be determined by measuring immune responses to CFTR protein. In some embodiments, IgG antibody to CFTR protein is measured by an enzyme-linked immunosorbent assay in collected serum samples. In some embodiments, CFTR-specific T cell responses are assessed using collected peripheral blood mononuclear cells. In some embodiments, T cell responses to CFTR are measured by a human interferon-γ enzyme-linked immunospot assay as described by Calcedo et al. (Calcedo et al., Hum Gene Ther Clin Dev. (2013) 24:108-15). Qualitative assessment of CFTR protein may also be performed by Western blot analysis. The CFTR protein activity may be measured by CFTR chloride channel activity in appropriate tissue cells. A stable potential with the mean value of a 10 second scoring interval after perfusion of solution is recorded. CFTR activity is estimated by the change in potential difference following perfusion with chloride-free isoproterenol. Various other methods are known in the art and may be used to determine the CFTR mRNA and CFTR protein expression or activity.

### Therapeutic Efficacy

According to the present invention, a CFTR mRNA is delivered to a CF patient in need of treatment at a therapeutically effective dose and an administration interval for a treatment period sufficient to improve, stabilize or reduce one or more symptoms of cystic fibrosis relative to a control. The terms “treat” or “treatment”, as used in the context of cystic fibrosis herein, refers to amelioration of one or more symptoms associated with cystic fibrosis, prevention or delay of the onset of one or more symptoms of cystic fibrosis, and/or lessening of the severity or frequency of one or more symptoms of cystic fibrosis.

In some embodiments, a therapeutically effective dose of a CFTR mRNA is or greater than about 2 mg, 4 mg, 6 mg, 8 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, or 40 mg per dose or equivalent thereof. In some embodiments, a therapeutically effective dose of a CFTR mRNA is or less than about 50 mg, 48 mg, 46 mg, 44 mg, 42 mg, 40 mg, 38 mg, 36 mg, 34 mg, 32 mg, 30 mg, 28 mg, 26 mg, 24 mg, 22 mg, 20 mg, 18 mg, 16 mg, 14 mg, 12 mg, 10 mg, 8 mg, 6 mg, or 4 mg per dose or equivalent thereof. In some embodiments, a therapeutically effective dose of a CFTR mRNA is about 2-50 mg, 4-45 mg, 4-40 mg, 6-40 mg, 6-38 mg, 6-36 mg, 6-34 mg, 6-32 mg, 6-30 mg, 6-28 mg, 6-26 mg, 6-24 mg, 6-22 mg, 6-20 mg, 6-18 mg, 6-16 mg, 8-50 mg, 8-45 mg, 8-40 mg, 8-38 mg, 8-36 mg, 8-34 mg, 8-32 mg, 8-30 mg, 8-28 mg, 8-26 mg, 8-24 mg, 8-22 mg, 8-20 mg per dose, or the equivalent thereof.

In some embodiments, a therapeutically effective dose of a CFTR mRNA is administered daily, twice a week, weekly, once every two weeks, once every three weeks, once every four weeks, monthly, once every two months, once every three months. Typically, weekly administration of a therapeutically effective dose of a CFTR mRNA in accordance with the invention is sufficient to effectively reduce the severity of one or more symptoms in a cystic fibrosis patient. For example, a nominal dose of 4-40 mg of a CFTR mRNA (e.g., a nominal dose of 6-30 mg, e.g., 8 mg, 16 mg, or 24 mg) administered weekly by nebulization is effective in reducing the severity of one or more symptoms in a cystic fibrosis patient.

In some embodiments, a therapeutically effective dose of a CFTR mRNA is administered for a period of at least two weeks, three weeks, four weeks, a month, two months, three months, four months, five months, six months, seven months, eight months, nine months, ten months, eleven months, twelve months, 1 year, 2 years, 3 years, 4 years, 5 years, or 10 years.

Typically, the therapeutic effect of administration of a CFTR mRNA on a cystic fibrosis patient is measured relative to a control. In some embodiments, a control is the severity of one or more symptoms in the same patient before the treatment. In some embodiments, a control is indicative of a historical reference level of one or more symptoms in CF patients. In some embodiments, a control is indicative of a normal level of ability, physical conditions or biomarker corresponding to the one or more symptoms being measured.

In some embodiments, the therapeutic effect of administration of a CFTR mRNA according to the present invention is measured by a score on a Cystic Fibrosis Questionnaire Revise (CFQ-R) respiratory domain. In some embodiments, the therapeutic effect of administration of a CFTR mRNA according to the present invention is measured by a sweat chloride value. In some embodiments, the therapeutic effect of administration of a CFTR mRNA according to the present invention is measured by a body mass index and/or body weight. In some embodiments, the therapeutic effect of administration of a CFTR mRNA according to the present invention is measured by onset or severity of pulmonary exacerbation.

In some embodiments, the therapeutic effect of administration of a CFTR mRNA according to the present invention is measured by minute volume, respiratory rate, and/or tidal volume. In some embodiments, the therapeutic effect of administration of a CFTR mRNA according to the present invention on the respiratory system is determined by performing spirometry and assessing the following parameters: forced expiratory volume in 1 second (FEV1): absolute volume (L) and percent based on the patient's age, gender, and height, forced vital capacity (FVC): absolute volume (L) and percent based on the patient's age, gender, and height, FEV/FVC: ratio and percent based on the patient's age, gender, and height, and/or forced expiratory flow over the middle one-half of the FVC (FEF25-75%): absolute volume (L) and percent based on the patient's age, gender, and height. In some embodiments, the parameters can be normalized using the ERS Global Lung Function Initiative (GLI) prediction equations. In some embodiments, the therapeutic effect of administration of a CFTR mRNA according to the present invention on the respiratory system is determined by chest x-ray.

In some embodiments, administration of a CFTR mRNA according to the present invention results in a change in the CFQ-R respiratory domain score by at least 1, at least 2, at least 3, at least 4, at least 5, at least 6, at least 7, at least 8, at least 9, at least 10, at least 11, at least 12, at least 13, at least 14, or at least 15 points relative to a control. In some embodiments, administration of a CFTR mRNA according to the present invention results in a change in the CFQ-R respiratory domain score by at least 1%, 2%, 3%, 4%, 5%, 6%, 7%, 8%, 9%, 10%, 11%, 12%, 13%, 14%, 15%, 16%, 17%, 18%, 19%, or 20% relative to a control.

In some embodiments, administration of a CFTR mRNA according to the present invention results in amelioration, prevention or delay in onset of pulmonary exacerbation. As used herein, pulmonary exacerbation refers to one or more of the following sino-pulmonary signs/symptoms: change in sputum, new or increased hemoptysis, increased cough, increased dyspnea, malaise/fatigue/lethargy, temperature >38° C. (˜100.4° F.), anorexia/weight loss, sinus pain/tenderness, change in sinus discharge, change in physical chest exam, decrease in pulmonary function and radiographic indication of pulmonary infection.

In some embodiments, administration of a CFTR mRNA according to the present invention results in prevention or reduced inflammation associated with pulmonary exacerbation. For example, administration of a CFTR mRNA according to the present invention results in reduced expression of markers of inflammation and/or lung damage, including but not limited to, C-reactive protein, white cell counts, interleukin-8, neutrophil elastase alpha 1-antiprotease complexes and matrix metalloproteins, in blood or serum as compared to a control indicative of the corresponding level of relevant markers in a CF patient without treatment. Additionally or alternatively, administration of a CFTR mRNA according to the present invention results in reduced sputum concentrations of bioactive lipid mediators, such as the cysteinyl leukotrienes and prostaglandin-E2, or sputum cell counts as compared to a control indicative of the corresponding level of relevant markers in a CF patient without treatment.

In some embodiments, administration of a CFTR mRNA according to the present invention results in a weight gain of at least 1 pound, at least 2 pounds, at least 3 pounds, at least 4 pounds, at least 5 pounds, at least 6 pounds, at least 7 pounds, at least 8 pounds, at least 9 pounds, at least 10 pounds, at least 11 pounds, at least 12 pounds, at least 13 pounds, at least 14 pounds or at least 15 pounds as compared to pre-treatment body weight.

In some embodiments, a CFTR mRNA is administered in combination with one or more CFTR potentiators and/or correctors. Suitable CFTR potentiators and/or correctors include ivacaftor (trade name Kalydeco®), lumacaftor (trade name Orkambi®) or the combination of ivacaftor and lumacaftor. In some embodiments, a CFTR mRNA is administered in combination with one or more other CF treatment such as hormone replacement therapies, thyroid hormone replacement therapy, non-steroidal inflammatory drugs, and prescription dronabinol (Marinol®) during treatment.

In some embodiments, the CF patient receives a concomitant CFTR modulator therapy. In some embodiments, the concomitant CFTR modulator therapy is given during the CFTR mRNA treatment regimen. In some embodiments, the concomitant CFTR modulator therapy is given before commencing the CFTR mRNA treatment regimen. In some embodiments, the concomitant CFTR modulator therapy is commenced after the CFTR mRNA treatment regimen.

Various CFTR modulator therapies are known in the art. For example, in some embodiments, the CF patient receives a CFTR modulator therapy comprising ivacaftor. In some embodiments, the CF patient receives a CFTR modulator therapy comprising lumacaftor. In some embodiments, the CF patient receives a CFTR modulator therapy comprising tezacaftor. In some embodiments, the CF patient receives a CFTR modulator therapy comprising ivacaftor/lumacaftor. In some embodiments, the CF patient receives a CFTR modulator therapy comprising tezacaftor/lumacaftor.

In some embodiments, CFTR potentiators and/or correctors and/or other cystic fibrosis treatments may be administered prior to, concurrently or subsequent to the administration of a CFTR mRNA according to the present invention. For example, CFTR potentiators and/or correctors and/or other cystic fibrosis treatments may be administered at 1 hour or longer, at 2 hours or longer, at 4 hours or longer, at 6 hours or longer, at 8 hours or longer, at 10 hours or longer, at 12 hours or longer, at 18 hours or longer, at 24 hours or longer, at 36 hours or longer, at 48 hours or longer, at 72 hours or longer, at 1 week or longer, at 2 weeks or longer, at 3 weeks or longer, or at 1 month or longer prior to or following administration of a CFTR mRNA according to the invention.

### Examples

While certain compounds, compositions and methods of the present invention have been described with specificity in accordance with certain embodiments, the following examples serve only to illustrate the compounds of the invention and are not intended to limit the same.

### Example 1. Formulation of mRNA/Liposome Composition

Codon-optimized Human Cystic Fibrosis Transmembrane Conductance Regulator (CFTR) messenger RNA comprising the sequence of SEQ ID NO: 1 was synthesized by in vitro transcription from a plasmid DNA template encoding the CFTR gene, which was followed by the addition of a 5′ cap structure (Cap 1) (Fechter, P.; Brownlee, G.G. “Recognition of mRNA cap structures by viral and cellular proteins” J. Gen. Virology 2005, 86, 1239-1249) and a 3′ poly(A) tail. The mRNA encoding CFTR protein also comprised 5′ and 3′ untranslated regions (UTRs). The final mRNA construct had the sequence of SEQ ID NO: 6 with a 3′ poly(A) tail of approximately 400 to 700 nucleotides in length, as determined by gel electrophoresis.

Three biodegradable lipid components were combined in solid form and then dissolved together in ethanol to provide an ethanol-based lipid solution. An aqueous-based solution comprising the exemplary mRNA encoding CFTR protein in a citrate buffer was combined with the ethanol-based lipid solution, isolated and dialyzed into the final formulation containing 10% (w/v) trehalose in water for injection, adjusted to an mRNA concentration of 0.6 mg/mL. For administration to human subjects, the final formulation was filled into single-use vials. The nominal fill volume of each vial was 3.2 mL.

The three biodegradable components all contributed to the final drug product characteristics. The first component was the ionizable lipid, imidazole cholesterol ester (ICE). This afforded a positively charged environment at low pH which facilitates efficient encapsulation of the negatively charged mRNA. It may also play a key role in cell surface interaction to allow for cellular uptake. The second component of the LNP was 1,2-dioleoyl-sn-glycero-3-phosphoethanolamine (DOPE). DOPE is a zwitterionic lipid that has been reported to have fusogenic properties to enhance uptake and release of the drug payload. The final component was a PEGylated (i.e., PEG-modified) lipid known as 1,2-dimyristoyl-sn-glycerol, methoxypolyethylene glycol (DMG-PEG-2K). The addition of this PEGylated lipid provided control over particle size and stability of the nanoparticle and may provide enhanced mucopentrating properties for lung uptake.

The nominal nitrogen/phosphorus (N/P) charge ratio of the LNP was 4 and the average particle size range for the mRNA encapsulated in the LNP was 40-60 nm.

### Example 2. Dosing of CFTR mRNA/Liposome Composition

These studies evaluated a CFTR mRNA/liposome composition in a Sprague-Dawley rat model. No adverse effects on the central nervous system (CNS), cardiovascular (CV) system or respiration were observed.

### CNS Evaluations

Neurobehavioral evaluations were performed on 6 males/group prior to dosing and on Day 1 (4 and 24 hours post-dosing). Temperature, humidity, noise level, and illumination of each room were measured and recorded to ensure that variations in environmental conditions were minimal during all evaluations. There were no effects on neurobehavior related to treatment with hCFTR mRNA-loaded ICE-based liposomes or ICE-based liposome vehicle control at inhaled doses up to 6.70 mg/kg hCFTR mRNA-loaded ICE-based liposomes.

### Cardiovascular Evaluations

Male Sprague-Dawley rats (n=5/group), which had previously been implanted with telemetry devices, were dosed via nose-only inhalation with hCFTR mRNA-loaded ICE-based liposomes for up to 6 hours at target doses of 0 (0.9% Saline control) or 0.7, 3.75 or 6.4 mg/kg of hCFTR mRNA-loaded ICE-based liposomes or ICE-based liposome vehicle control (non-mRNA containing LNP suspended in a solution containing 10% trehalose) (achieved doses of 0, 0.86, 3.52, 6.02 or 0 mg/kg, respectively). For target doses at 0.7 and 3.75 mg/kg, air was administered following the end of exposure to make the total restrained time equivalent among all doses. Each dose day was followed by a minimum 7-day washout period. All animals were returned to the colony upon completion of all evaluations.

Telemetry parameters included cardiovascular parameters (systolic, diastolic, mean blood pressure, pulse pressure, heart rate and electrocardiogramameters [PR, QRS, QT, QTc]), activity, and body temperature. Other parameters evaluated during the study were viability, clinical observations and body weight. Mean aerosol concentrations and estimated total delivered doses of hCFTR mRNA-loaded ICE-based liposomes and ICE-based liposome vehicle (total lipid) are summarized in Table 3.

TABLE 3
Mean hCFTR mRNA-Loaded Liposome and Liposome Aerosol
Concentrations and Estimated Total Delivered Doses

| hCFTR mRNA-loaded | | hCFTR mRNA-loaded Liposome | |
|---|---|---|---|
| Liposome Aerosol | | Delivered Dose (mg/kg) | |
| Concentration (μg/L) | | Exposure Duration | Mean |
| Treatment | Achieved | (mins) | Achieved^{a} |
| Control | 0 | 360 | 0 |
| Vehicle | 0 | 360 | 0 |
| Low | 30.5 | 40 | 0.86 |
| Mid | 23.7 | 210 | 3.52 |
| High | 23.7 | 360 | 6.02 |

^{a}Overall mean of each individual animal for each treatment group. This dose reflects the total delivered dose of hCFTR mRNA.

In addition to this study in rats, similar cardiovascular evaluations were also performed as repeat-dose studies in rats and monkeys. In those repeat-dose studies, no test article-related effects were observed on any CV parameters evaluated up to the highest doses evaluated (6.7 mg/kg in rats and 0.691 mg/kg in monkeys).

### Respiratory Evaluations

Respiratory effects of hCFTR mRNA-loaded ICE-based liposomes were evaluated as part of the single-dose and repeat-dose studies in rats and monkeys. An increase in minute volume was observed after inhalation administration of hCFTR mRNA-loaded ICE-based liposomes to Sprague Dawley rats, as well as respiratory rate and tidal volume, in all dose groups up to 6.4 mg/kg hCFTR mRNA-loaded ICE-based liposomes, as well as in 10% trehalose controls.

No effects were observed on respiratory parameters, including respiration rate, tidal volume and derived minute volume after inhalation administration of hCFTR mRNA-loaded ICE-based liposomes to Sprague-Dawley rats at repeat doses up to 6.7 mg/kg or cynomolgus monkeys at single or repeat doses up to 0.85 mg/kg or 0.691 mg/kg, respectively.

There is minimal toxicological concern regarding ICE, DOPE, and DMG-PEG 2000 as components of the composition developed for inhalation administration. In an in silico genotoxicity evaluation, ICE is predicted to be negative for bacterial mutagenicity. This is consistent with the negative mutagenicity/genotoxicity data that are available for imidazole and propionic acid, the 2 components of the imidazolepropionic acid moiety of ICE, and for cholesterol. DOPE is a variant of the glycerophospholipid, phosphatidylethanolamine, which is a component of lung surfactant. Degradation of DOPE would be expected to follow a similar path as for other glycerophospholipids, with the ultimate formation of ethanolamine and oleic acid, both of which are present in the circulation of infants and adults. DMG PEG 2000 is anticipated to have low toxicity based on information for the anticipated metabolic breakdown products PEG 2000 and myristic acid. There is minimal concern for local or systemic toxicity based on data from studies with PEGs of various sizes, while myristic acid is a fatty acid that is present in most animal and vegetable fats and is present in the circulation of infants and adults.

### Example 3. Pharmacokinetics

In the study in this Example, Sprague-Dawley rats or monkeys were dosed for up to 6 hours (for rats) or for up to 2 hours (for monkeys) via inhalation with hCFTR mRNA-loaded ICE-based liposomes or with the ICE-based liposomes alone, and then sacrificed 24 hours later to measure the levels of hCFTR in various tissues. The target doses for are shown in Tables 4-6. The actual doses measured were 420, 630 and 850 μg/kg for hCFTR mRNA-loaded ICE-based liposomes administered to monkeys (corresponding to the target doses in the header of Table 4); 0.77, 4.05 and 6.70 mg/kg for hCFTR mRNA-loaded ICE-based liposomes administered to rats (corresponding to the target doses in the header of Table 5); and 0.77, 4.05 and 6.70 mg/kg for ICE-based liposomes administered to rats (corresponding to the target doses in the header of Table 6). Detailed tissue distribution results are presented below in Tables 4, 5, and 6.

TABLE 4
Mean Concentrations of hCFTR mRNA in Monkey Tissues and Blood
24 Hours Post-Inhalation of hCFTR mRNA-Loaded Liposomes

| | Males | | | | Females | | | |
|---|---|---|---|---|---|---|---|---|
| | | Target Inhaled Dose | | | | Target Inhaled Dose | | |
| | | 500 | 750 | 1000 | | 500 | 750 | 1000 |
| Tissue ^{a} | Vehicle | μg/kg | μg/kg | μg/kg | Vehicle | μg/kg | μg/kg | μg/kg |
| Brain | BQL | 3.91 | 0.0968 | BQL | BQL | 2.05 | 0.110 | 1.18 |
| Heart | BQL | BQL | 0.165 | BQL | BQL | BQL | BQL | 0.537 |
| Kidney | BQL | BQL | BQL | 0.0523 | BQL | BQL | BQL | 0.231 |
| Larynx | 0.166 | BQL | 5.54 | 1.87 | BQL | 5.15 | 3.37 | 2.59 |
| Liver | BQL | BQL | 0.0670 | 1.27 | BQL | 0.917 | 0.147 | 8.48 |
| Lung (Average) | 0.110 | 208 | 67.2 | 82.1 | 0.426 | 819 | 1390 | 1880 |
| Spleen | BQL | 0.420 | BQL | BQL | BQL | BQL | 0.307 | 1.85 |
| Testis | BQL | BQL | BQL | 0.0912 | — | — | — | — |
| Ovary | — | — | — | — | BQL | 0.596 | BQL | 0.976 |
| Trachea | BQL | 0.176 | 0.993 | 1.65 | 0.0657 | 1.54 | 14.4 | 4.72 |
| Tracheobronchial LN | BQL | 30.9 | 0.284 | 70.3 | BQL | 0.147 | 468 | 5.28 |
| Blood | 0.0501 | BQL | 0.0433 | 0.0120 | 0.0188 | 0.00351 | 0.385 | 0.0121 |

BQL: Below limit of quantification
^{a} Levels in tissue expressed as 10⁶ × copies/gm and levels in blood as 10⁶ × copies/mL, to express levels in comparable masses since 1 mL of blood~1 gm.

TABLE 5
Mean hCFTR mRNA Concentrations in Rat Tissues and Blood
24 hours Post hCFTR mRNA-Loaded Liposome Doses of 0.7, 3.75 or 6.4 mg/kg

| | Males | | | | Females | | | |
|---|---|---|---|---|---|---|---|---|
| | | 0.7 | 3.75 | 6.4 | | 0.7 | 3.75 | 6.4 |
| Tissue | Vehicle | mg/kg | mg/kg | mg/kg | Vehicle | mg/kg | mg/kg | mg/kg |
| Brain | BLQ | BLQ | BLQ | 55.4 | BLQ | BLQ | BLQ | 32.4 |
| Heart | BLQ | BLQ | BLQ | 17.0 | BLQ | BLQ | BLQ | 41.1 |
| Kidney | BLQ | BLQ | BLQ | 0.37 | BLQ | BLQ | BLQ | 0.95 |
| Larynx | 0.20 | BLQ | BLQ | 4178 | BLQ | BLQ | BLQ | 1410 |
| Liver | NC | BLQ | BLQ | 2.3 | BLQ | BLQ | BLQ | 9.75 |
| Lung | .061 | 2057 | 59,094 | 156130 | BLQ | 1,361 | 33,649 | 180,000 |
| Nasal Turbinate | .12 | BLQ | BLQ | 792 | BLQ | BLQ | BLQ | 1450 |
| Spleen | BLQ | BLQ | BLQ | 3.8 | BLQ | BLQ | BLQ | 1.09 |
| Testis | 0.08 | BLQ | BLQ | 9.5 | BLQ | BLQ | BLQ | BLQ |
| Ovary | BLQ | BLQ | BLQ | BLQ | BLQ | BLQ | BLQ | 6.99 |
| Trachea | BLQ | BLQ | BLQ | 2980 | BLQ | BLQ | BLQ | 787 |
| Tracheobronchial | BLQ | BLQ | BLQ | 108 | BLQ | BLQ | BLQ | 1.48 |
| LN | | | | | | | | |
| Blood | 0.076 | 0.68 | 14.7 | 0.20 | .016 | 0.29 | 133 | 1.48 |

BLQ: below level of quantitation
Concentrations in tissues (copies × 106/g)/concentrations in blood (copies × 106/mL), with the assumption that 1 mL of blood ≈1 g.

TABLE 6
Mean ICE Concentrations in Rat Tissues (μg/g) and Blood (μg/mL) 24
hours Post-Inhalation Dosing with hCFTR mRNA-Loaded Liposomes
(Doses of 0.7, 3.75 or 6.4 mg/kg)

| | Males | | | | Females | | | |
|---|---|---|---|---|---|---|---|---|
| | | 0.7 | 3.75 | 6.4 | | 0.7 | 3.75 | 6.4 |
| Tissue | Vehicle | mg/kg | mg/kg | mg/kg | Vehicle | mg/kg | mg/kg | mg/kg |
| Brain | BLQ | BLQ | BLQ | BLQ | BLQ | BLQ | BLQ | BLQ |
| Heart | BLQ | BLQ | BLQ | BLQ | BLQ | BLQ | BLQ | BLQ |
| Kidney | BLQ | BLQ | BLQ | BLQ | BLQ | BLQ | BLQ | BLQ |
| Larynx | 1.57 | 2.61 | BLQ | 1.57 | BLQ | BLQ | BLQ | 1.41 |
| Liver | BLQ | BLQ | BLQ | BLQ | BLQ | BLQ | BLQ | BLQ |
| Lung | 317 | 20.3 | 139 | 245 | 293 | 22.5 | 147 | 317 |
| Nasal Turbinate | 0.290 | BLQ | BLQ | 0.796 | BLQ | BLQ | BLQ | 0.724 |
| Spleen | NA | BLQ | BLQ | BLQ | BLQ | BLQ | BLQ | BLQ |
| Testis | BLQ | BLQ | BLQ | BLQ | BLQ | BLQ | BLQ | BLQ |
| Ovary | BLQ | BLQ | BLQ | BLQ | BLQ | BLQ | BLQ | BLQ |
| Trachea | 6.27 | BLQ | BLQ | 3.65 | BLQ | BLQ | BLQ | 2.40 |
| Tracheobronchial | BLQ | BLQ | BLQ | BLQ | BLQ | BLQ | BLQ | BLQ |
| LN | | | | | | | | |
| Blood | BLQ | BLQ | BLQ | BLQ | BLQ | BLQ | BLQ | BLQ |

BLQ: below level of quantitation
Concentrations in tissues (copies × 106/g)/concentrations in blood (copies × 106/mL), with the assumption that 1 mL of blood ≈ 1 g.

These data show high levels of mRNA in lung tissue and associated respiratory tract issues such as larynx, trachea, tracheobronchial lymph nodes, and nasal turbinates with lower or background levels in heart, brain, liver, kidney, spleen, testis, and ovary, particularly at lower doses of administration, with the highest dose showing hCFTR mRNA distribution across various tissues. Lung levels were high and dose-responsive in both rats and NHP, with the highest levels seen at 6.4 mg/kg in rats.

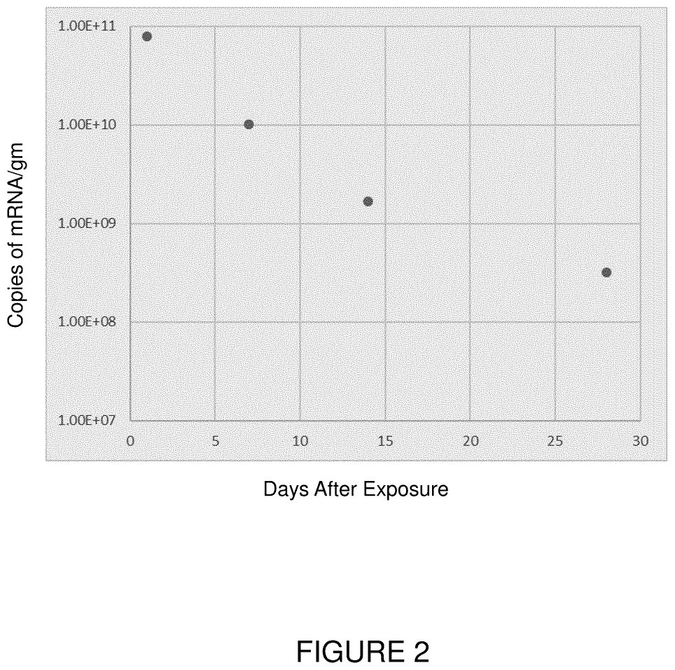

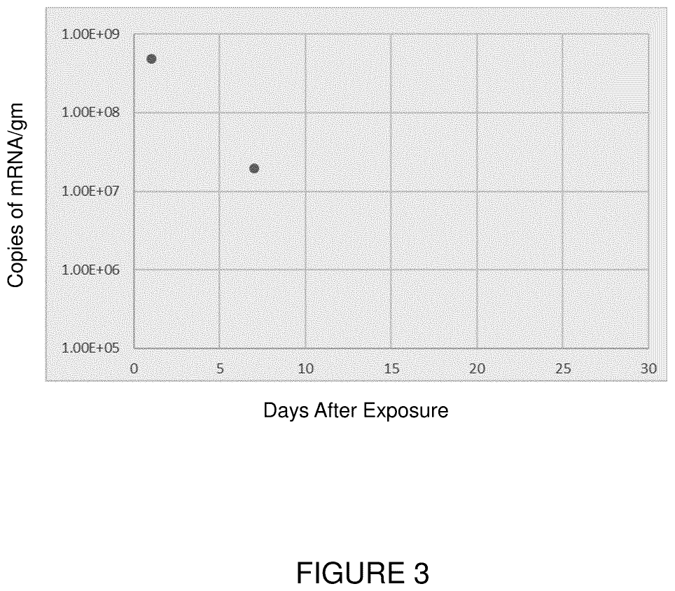

Kinetics of lung clearance of mRNA was measured reliably in rats since more sacrifice times could be used than for monkeys. FIG. 2 indicates a single component exponential decay with a half-life of approximately 2-3 days. Only two data points were available for NHP (FIG. 3) and these data appear consistent with the rat data in view of differences in dose.

As shown in Table 5, levels of mRNA in the lung were dose-dependent in a relatively linear manner. Lung tissue measurements made after a 28 day recovery period at the end of the 29-day study showed a decline in exposure of approximately 100-fold, similar to that seen 28 days after the single dose study.

The toxicokinetics of ICE liposomes were also examined. There were no measurable levels of ICE liposomes in whole blood. There were, however, measurable and dose-responsive levels of ICE liposomes in the lung tissue in rats (Table 6).

### Example 4. In vitro Activity of hCFTR in Human Bronchial Epithelial Cells

This Example illustrates a study where hCFTR mRNA was transfected into cultured human bronchial epithelial cells, whereupon the protein expressed from transfected hCFTR mRNA provided a significant increase in chloride transport across the bronchial epithelial cell membrane compared to buffer, thereby demonstrating the functional efficacy of the transfected mRNA.

The changes in chloride transport across the bronchial epithelial cell membrane was measured by short circuit current output in a Ussing epithelial voltage clamp apparatus (i.e., a Ussing chamber). Specifically, using an established Ussing Chamber procedure (Charles River Laboratories), hCFTR mRNA encapsulated in a liposome comprising ICE, DOPE, and a PEG-modified lipid was incubated for 2 or 4 hours on the apical (mucosal) or basolateral (serosal) sides, or both sides, of human bronchial epithelial cells. A buffer blank also was included as a control, for example, to assess chloride transport by endogenous CFTR in the cells. Next, Forskolin-induced chloride channel activity was measured using the Ussing chamber assay. Following the measure of the current change as indicative of chloride transport across the bronchial epithelial cell membrane, a CFTR inhibitor was added to the samples to show that current change was due to CFTR activity.

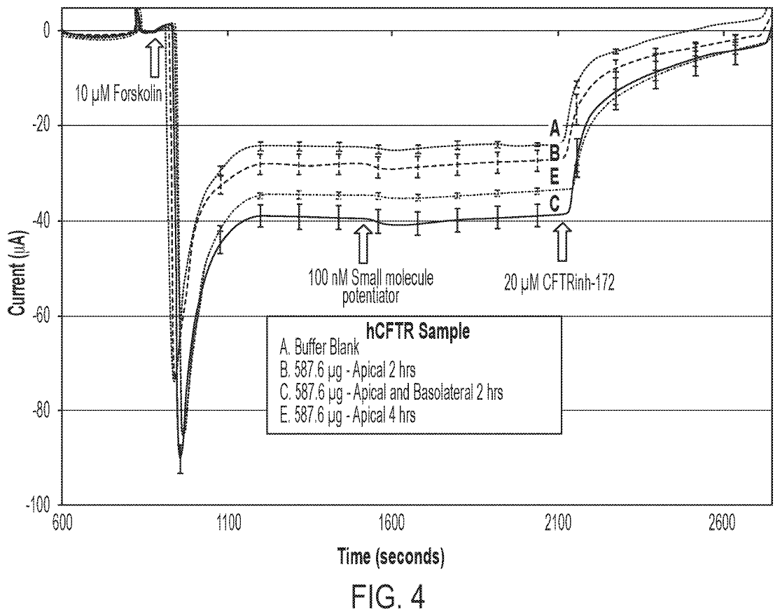

As shown in FIG. 4, compared to the control group(A. Buffer Blank), treatment of the apical (mucosal) epithelial surface for 2 or 4 hours (Samples B and E, respectively) and the apical and basolateral (serosal) epithelial surfaces for 2 hours (Sample C) with hCFTR mRNA provided a significant increase in chloride channel activity. Additionally, the chloride activity in all groups was inhibited by the CFTR inhibitor-172. The results of this study show that the hCFTR mRNA delivered in a liposome to human bronchial epithelial cells produced active CFTR protein in those cells. It also shows that the active CFTR protein produced from the hCFTR mRNA provided significantly increased chloride transport, compared to endogenous CFTR protein, across the cell membranes of the transfected human bronchial epithelial cells.

### Example 5. hCFTR Protein Expression in Mice after Single Dose of hCFTR mRNA/Liposome Composition

This Example illustrates that CFTR protein deficiency in lung cells of CFTR knockout (KO) mice can be addressed by pulmonary delivery of an hCFTR mRNA/liposome composition.

Delivery of an hCFTR mRNA/liposome composition to the respiratory tract of CFTR KO mice was achieved by nebulization of the composition via a vibrating mesh nebulizer. The dose of the composition delivered was a function of the concentration of the test article, the flow rate of the nebulized material, and the length of the nebulization period. Following dose administration, a human-specific antibody directed to the carboxy terminus of hCFTR was utilized to visualize protein expression and distribution using immunohistochemistry (IHC).

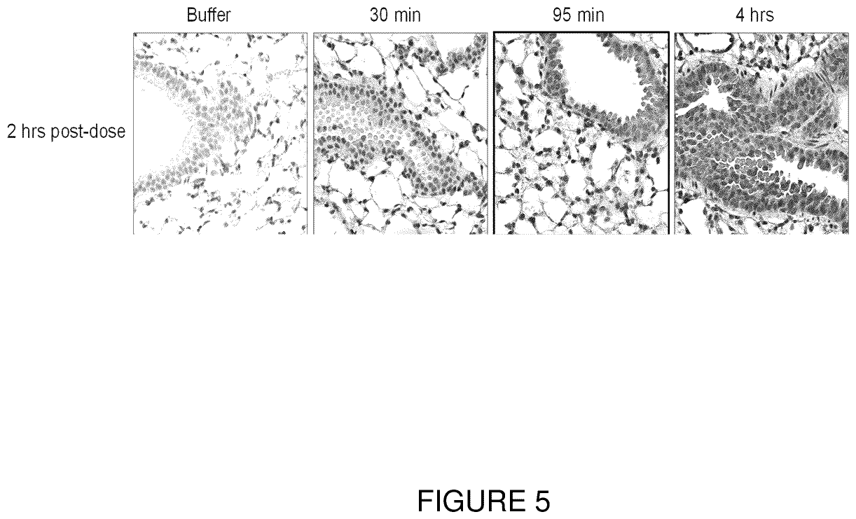

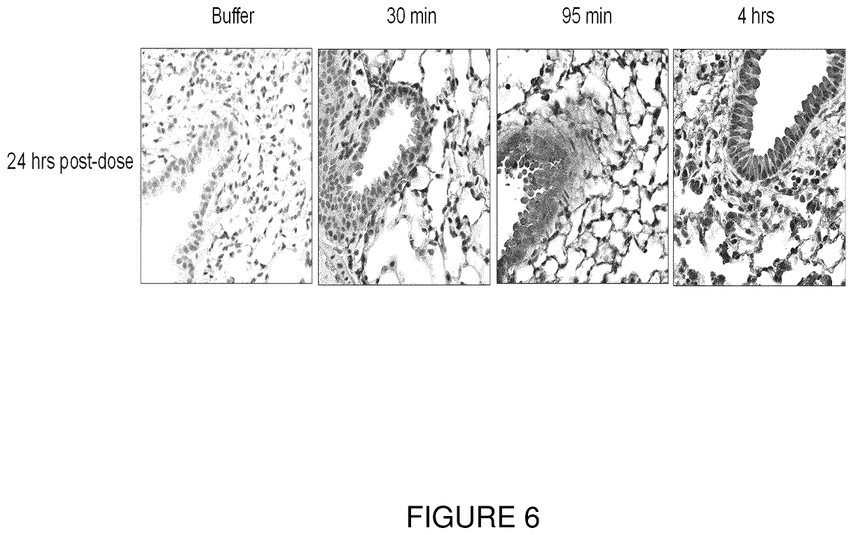

CFTR KO mice were given a single inhaled administration of the composition for nebulization periods of 30, 95 or 240 minutes. A control group received a 240-minute nebulization with buffer only. At 2 or 24 hours post administration, animals were sacrificed, and lung tissue isolated for IHC (FIGS. 5 and 6). Dose-dependent expression of hCFTR protein was observed, with minimal staining for hCFTR present after a 30-minute nebulization period at both time points. Increased staining was observed in the 90- and 240-minute nebulization periods. Furthermore, an increase in staining was also seen between the 2- and 24-hour time points. Administration of the hCFTR mRNA/liposome composition led to wide distribution of hCFTR protein throughout the lung, including the bronchial epithelium and alveoli of both the upper and lower airways.

### Example 6. Distribution of hCFTR after Single Dose of hCFTR mRNA/Liposome Composition in Non-Human Primates

This Example illustrates that administration of the hCFTR mRNA/liposome composition to non-human primates (NHPs) by aerosol results in widespread distribution of hCFTR mRNA in the lung and limited delivery thereof to the larynx, trachea and tracheobronchial lymph nodes.

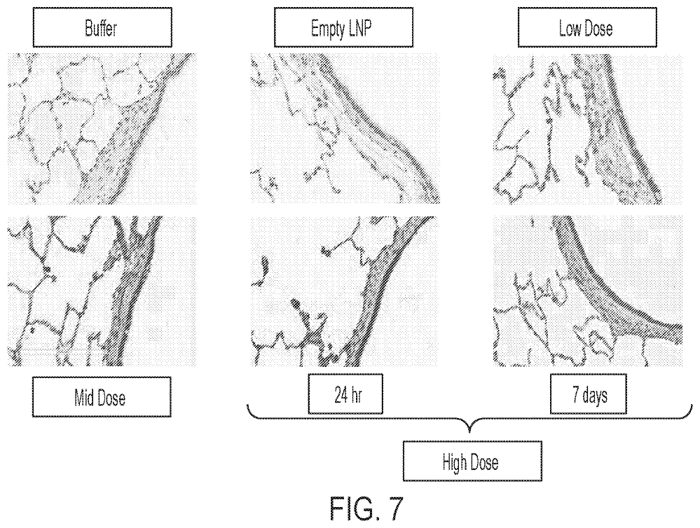

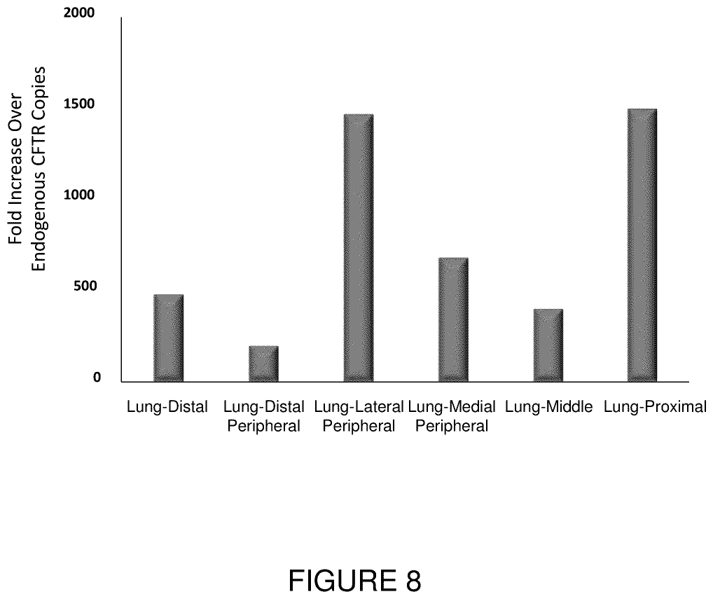

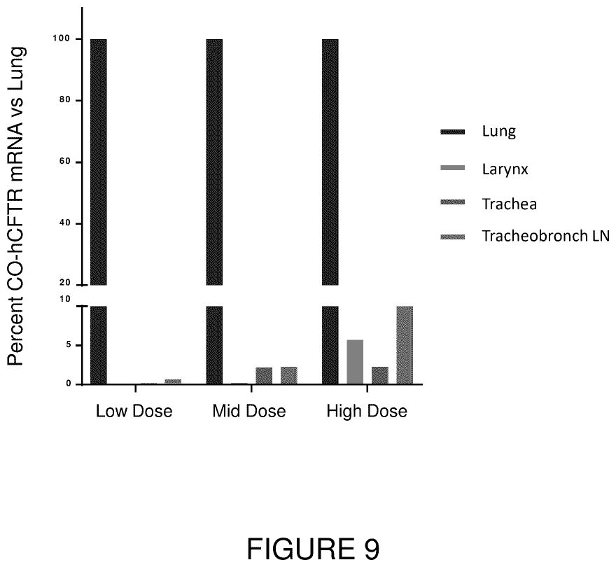

NHPs were treated with a single aerosol exposure of a CO-hCFTR mRNA/liposome composition. As shown in FIG. 7, immunohistochemistry (IHC) staining of lung cross-sections demonstrate a dose-dependent increase in intensity for positive hCFTR protein detection. Additionally, when hCFTR mRNA was detected and quantified in tissues from the NHPs, widespread distribution was found throughout the lungs of the monkeys, with CO-hCFTR levels orders of magnitude (>200-fold) over endogenous CFTR (FIG. 8). Additionally, the lungs of the monkeys, which were the target organs, received >90% of the CO-CFTR dose that entered the airway, when compared to other respiratory tract tissues that were tested (FIG. 9).

### Example 7. Systemic biodistribution of hCFTR after Single Dose of hCFTR mRNA/Liposome Composition in Non-Human Primates

This Example illustrates that administration of the hCFTR mRNA/liposome composition to non-human primates (NHPs) by aerosol results minimal systemic exposure.

NHPs were treated with a single aerosol exposure of a CO-hCFTR mRNA/liposome composition. As shown in Table 7, the biodistribution of hCFTR mRNA in other organs is several orders of magnitude lower than in the lung.

TABLE 7
Biodistribution of hCFTR mRNA in non-human primates after nebulization of
hCFTR mRNA-loaded liposomes.

| | Males | | | | Females | | | |
|---|---|---|---|---|---|---|---|---|
| | LNP | 0.191 | 0.329 | 0.691 | LNP | 0.191 | 0.329 | 0.691 |
| Tissue | Vehicle | mg/kg | mg/kg | mg/kg | Vehicle | mg/kg | mg/kg | mg/kg |
| Brain | BQL | — | — | 1.75E+06 | BQL | — | — | 6.17E+06 |
| Heart | 1.24E+04 | — | — | 3.02E+05 | 4.67E+05 | — | — | 2.22E+07 |
| Kidney | BQL | — | — | 3.23E+04 | 6.10E+04 | — | — | 1.06E+05 |
| Larynx | 1.26E+04 | — | — | 1.71E+07 | 8.50E+04 | — | — | 1.74E+08 |
| Liver | BQL | — | — | 6.37E+06 | 7.83E+04 | — | — | 4.25E+05 |
| Lung Distal | 4.48E+05 | 5.39E+08 | 1.69E+09 | 8.58E+09 | 1.56E+06 | 9.35E+08 | 3.61E+09 | 9.89E+09 |
| Lung Distal | 1.69E+05 | 2.88E+08 | 1.49E+09 | 4.31E+09 | 7.56E+05 | 1.43E+09 | 1.34E+09 | 3.04E+09 |
| Peripheral | | | | | | | | |
| Lung Lateral | 3.35E+05 | 5.79E+08 | 4.36E+09 | 1.61E+10 | 1.84E+06 | 1.53E+09 | 5.82E+09 | 2.12E+10 |
| Peripheral | | | | | | | | |
| Lung Medial | 3.89E+05 | 5.97E+08 | 4.91E+09 | 6.28E+09 | 1.86E+06 | 1.88E+09 | 3.17E+09 | 1.21E+10 |
| Peripheral | | | | | | | | |
| Lung Middle | 2.01E+05 | 4.88E+08 | 2.59E+09 | 1.49E+10 | 1.40E+06 | 2.12E+09 | 5.34E+09 | 2.45E+10 |
| Lung Proximal | 2.96E+05 | 5.60E+08 | 5.52E+09 | 1.59E+10 | 2.16E+06 | 2.89E+09 | 5.98E+09 | 2.49E+10 |
| Ovary | — | — | — | — | 1.45E+05 | — | — | 5.47E+05 |
| Spleen | 4.43E+04 | — | — | 2.98E+05 | BQL | — | — | 1.85E+07 |
| Testes | BQL | — | — | 1.38E+05 | — | — | — | — |
| Trachea | 2.11E+04 | — | — | 4.92E+06 | 9.34E+05 | — | — | 9.79E+08 |
| Tracheobronchial | 1.11E+06 | — | — | 1.95E+09 | 1.39E+05 | — | — | 7.13E+09 |
| LN | | | | | | | | |
| Blood (copies/mL) | 6.56E+04 | 5.96E+03 | 3.04E+03 | 9.83E+03 | 6.22E+03 | 8.06E+04 | 5.97E+05 | 1.30E+05 |

—: no sample collected, per study plan;
BQL: below quantifiable level
Lung and blood was collected from all groups, the other tissues were collected only from Groups 2 and 5. LNP vehicle is the ‘empty’ LNP, without hCFTR mRNA

### Example 8. Treatment of Cystic Fibrosis Subjects with CO-hCFTR mRNA/Liposome Composition

The study in this Example is designed to evaluate a CO-CFTR mRNA liposome composition in patients with cystic fibrosis.

A CO-hCFTR mRNA liposome composition (CO-hCFTR composition) is administered by nebulization to subjects with cystic fibrosis. The concentration of the CO-hCFTR mRNA is 0.6 mg/ml. A nebulizer is used to administer the CO-hCFTR composition by nebulization at a nebulisation rate of approximately 0.3 ml/minute. The CO-hCFTR composition will be administered to subjects at the following 3 dose levels: 8.0, 16.0, or 24.0 mg of CO-hCFTR mRNA (nominal dose levels) either once or once per week for five weeks. Other subjects will be dosed with placebo control.

In order to receive treatment with administration of the CO-hCFTR composition, patients will have a confirmed diagnosis of CF as defined by all of the following: a sweat chloride value of ≥60 mmol/L by quantitative pilocarpine iontophoresis (documented in the subject's medical record), a confirmed disease-causing CFTR mutation (genotype confirmed at the screening visit), and chronic sinopulmonary disease and/or gastrointestinal/nutritional abnormalities consistent with CF disease; clinically stable CF disease, e.g., FEV1 ≥50% and ≤90% of the predicted normal for age, gender, and height at screening, resting oxygen saturation ≥92% on room air (pulse oximetry), and body mass index ≥17.5 kg/m2 and weight >40 kg. Subjects who are receiving lumacaftor/ivacaftor combination drug (ORKAMBI) will remain on it for the duration of the study preferably at a stable dose.

Procedures and tests that will be conducted both for screening subjects and during the study to evaluate the biological activity of the CO-CFTR mRNA liposome composition include: vital signs, pulse oximetry, physical examination, spirometry, clinical laboratory tests (serum chemistry, hematology, coagulation, urinalysis, CRP), ECG, chest x-ray, Cystic Fibrosis Questionnaire-Revised (CFQ-R), serum pregnancy test, AE and concomitant medication reporting, weight measurement, blood sampling for CO-hCFTR mRNA and ICE assays and blood sampling for immune response assays. Subjects who were administered 24.0 mg of CO-hCFTR mRNA once per week for five weeks will additionally undergo bronchoscopy.

Bronchial epithelial cells obtained during bronchoscopies will be prepared for quantification of exogenous CO-hCFTR mRNA and endogenous CFTR mRNA by qPCR, and for a qualitative assessment of CFTR protein by Western blot analysis.

Additionally, during bronchoscopy, lower airway potential difference measurements will be performed to assess CFTR chloride channel activity in the bronchial epithelium. Potential difference measurements will be made at the lingula outlet of the left lung, as described by Dransfield et al. (Dransfield et al., Chest. (2013) 144:498-506). A stable potential with the mean value of a 10 second scoring interval after perfusion of each solution will recorded. CFTR activity will be estimated by the change in potential difference following perfusion with chloride-free isoproterenol.

The Cystic Fibrosis Questionnaire-Revised (CFQ-R; version for adolescents and adults [patients 14 years old and older]) will be completed in order to achieve both a baseline score and scores during the study for comparison. The results of the respiratory domain of the CFQ-R will be of primary interest; the minimal change from baseline representing a clinically important improvement in the respiratory domain was determined to be ≥4 (Quittner et al., Chest. (2009) 135:1610-8).

### Example 9. CFTR colocalization with membrane protein in upper airway bronchial cells

This Example demonstrates CFTR protein expression in the lung and colocalization with the membrane tight junctions (ZO-1) after administration of the codon-optimized human CFTR mRNA.

Colocalization study protocol: The immunohistochemistry and colocalization study method described in this paragraph is common for Examples 9, 10, and 11. Lung delivery of the CFTR mRNA in non-human primates was followed by immunohistochemistry to detect the protein expression and membrane colocalization in the upper airway bronchial cells and lower airway epithelial cells and deep alveolar lung. The non-human primates in this study were grouped into five categories and were administered the following: (1) Control, Trehalose 10%; (2) Control, LNP vehicle; (3) CO-hCFTR low dose, 500 μg/kg, (4) CO-hCFTR medium dose, 750 μg/kg, (5) CO-hCFTR high dose, 1000 μg/kg. The mRNA-LNP formulation or controls (without mRNA) were administered daily. Accordingly, the animals were exposed for 60 minutes to the aerosol composition (Group 3, low dose, 500 μg/kg), 90 minutes of aerosol (Group 4, medium dose, 750 μg/kg), or 120 minutes of aerosol (Group 5, high dose) as described in Table 4. At the end of the study the animals were sacrificed and tissues collected for immunohistochemistry to detect the expression and localization of human CFTR protein in the lungs. For immunohistochemical detection of codon-optimized human CFTR protein, lung sections were incubated overnight at 4° C. with a 1:250 dilution of each of mouse monoclonal anti-CFTR antibody (MAB 25031, R & D Systems), and rabbit anti-ZO1 antibody (Ab214228, Abcam); or with anti-CFTR alone; and anti-ZO1 alone. Following overnight incubation, the sections were blocked with first blocking agent containing hydrogen peroxide, incubated with the secondary antibody (anti-mouse DyLight 594 for CFTR antibody, and anti-rabbit DyLight 488 antibody) and followed by second blocking in a solution containing 3% horse serum and 3% BSA and subjected to confocal microscopy for colocalization. DyLight 594 emits red signal, and DyLight 488 emits green, and upon colocalization of the two signals (and therefore the proteins each bind to), the optical merge yields yellow signal.

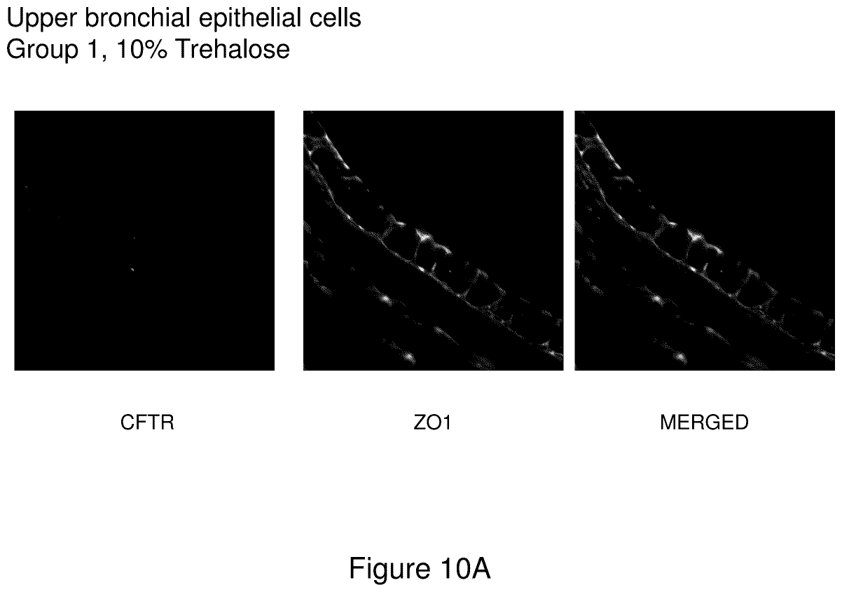

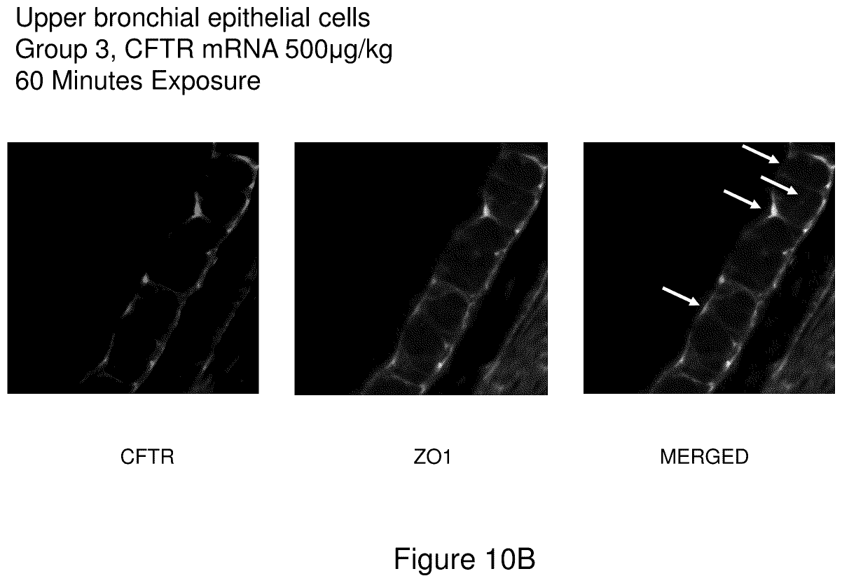

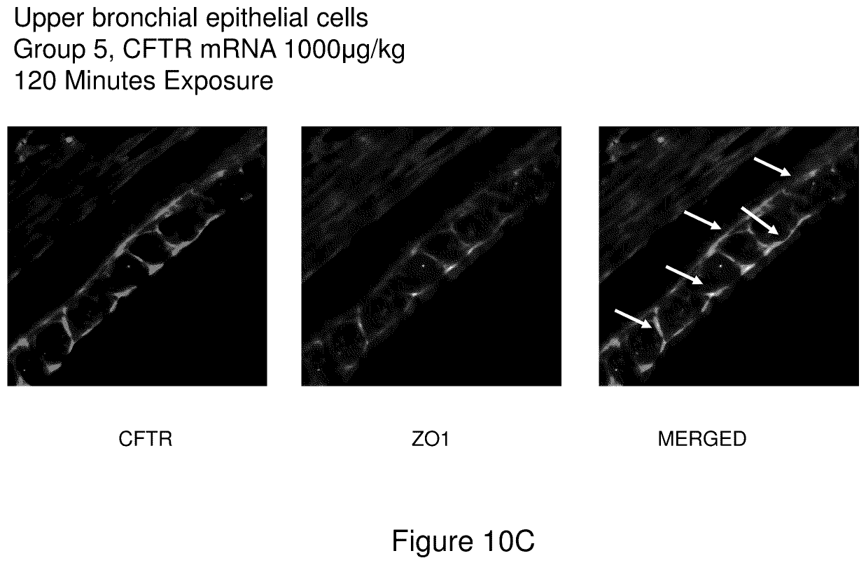

As shown in FIGS. 10A-C, CFTR protein translated from administered CFTR mRNA is expressed in the upper respiratory tract in bronchial epithelial cells. In the exemplary study, CFTR mRNA-derived protein exhibited a dose-dependent expression and colocalized with endogenous membrane tight junction protein ZO1 in upper bronchial epithelial cell membrane. FIG. 10A depicts representative microscopic images of bronchial epithelial cells from a control animal that received 10% treahalose alone, visualized for CFTR staining (left), ZO1staining (middle) and optical merge of CFTR and ZO1 staining (right). FIG. 10B depicts representative microscopic images of bronchial epithelial cells of an animal that received low dose CFTR mRNA (500 μg/kg), visualized for CFTR staining (left), ZO1staining (middle) and optical merge of CFTR and ZO1 staining (right). FIG. 10C depicts representative microscopic images of bronchial epithelial cells of an animal that received high dose CFTR mRNA (1000 μg/kg), visualized for CFTR staining (left), ZO1 staining (middle) and optical merge of CFTR and ZO1 staining (right). As indicated in the figure, the expression of CFTR was dose-dependent, that is, the immunostaining signal was stronger at the higher dose (FIG. 10C left image) than that of the low dose (FIG. 10B, left image). Also, as evident from the positioning of the CFTR and ZO1 signals, CFTR and ZO1 are expressed in the membrane of the epithelial cells. The two proteins colocalized yielding an intensified signal, which was yellow upon visual observation (FIGS. 10B and 10C right image). No red signal corresponding to CFTR was observed in FIG. 10A indicating the specificity of the staining. Also the merged optical signal was not intensified and monochromatic for ZO-1 signal alonc.

### Example 10. CFTR colocalization with membrane protein in lower airway epithelial cells

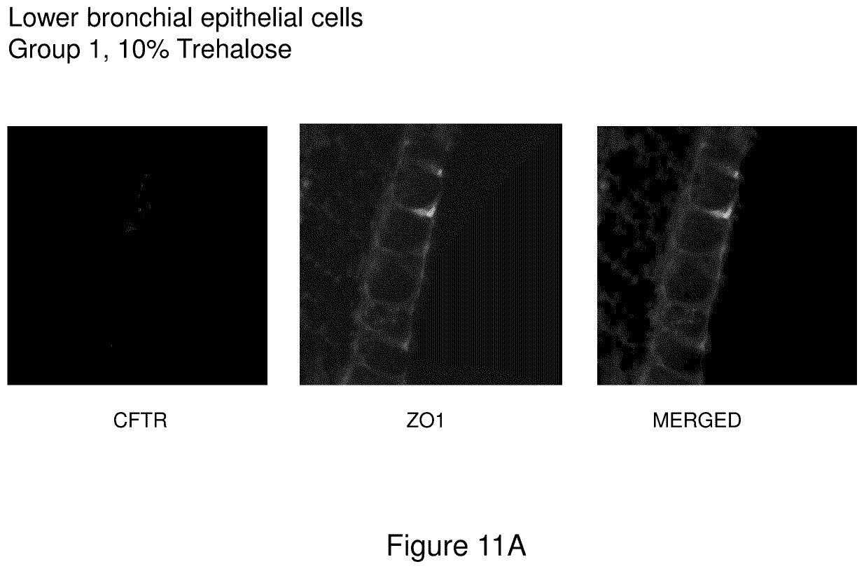

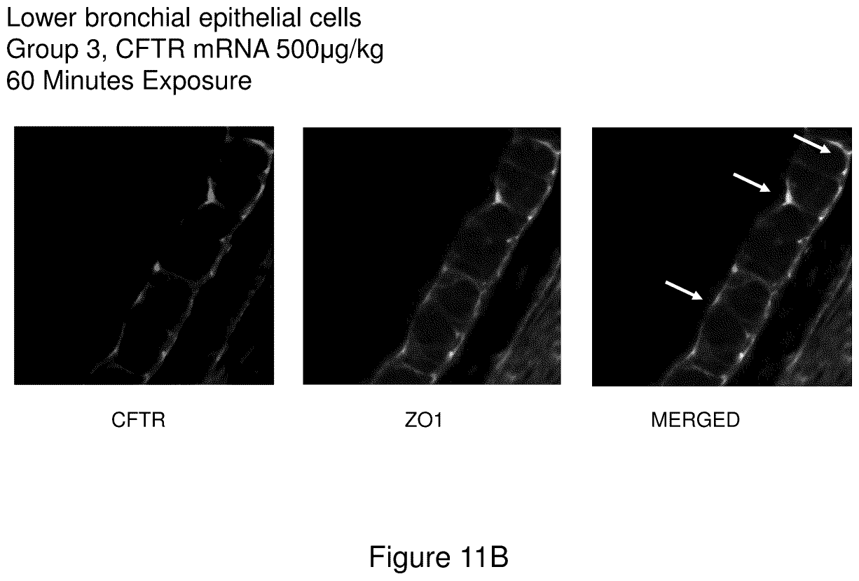

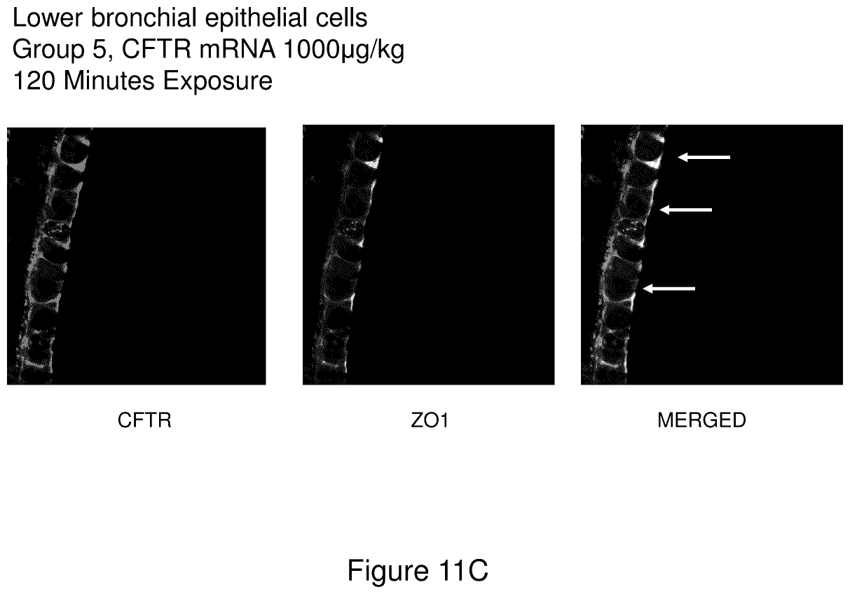

For successful therapeutic effect, the administered CFTR mRNA has to be delivered to the lower lung. As shown in FIGS. 11A-C, CFTR protein translated from administered CFTR mRNA was expressed in lower respiratory tract epithelial cells. CFTR mRNA-derived protein exhibited a dose-dependent expression and colocalization with endogenous membrane tight junction protein ZO1 protein in lower airway epithelial cell membrane. FIG. 11A shows representative microscopic images of a tissue section from the lower airway epithelial cell membrane from a control animal that received 10% trehalose alone, visualized for CFTR staining (left), ZO1staining (middle) and optical merge of CFTR and ZO1 staining (right). FIG. 11B shows representative microscopic images of a tissue section from the lower airway epithelial cell membrane of an animal that received low dose CFTR mRNA (500 μg/kg), visualized for CFTR staining (left), ZO1staining (middle) and optical merge of CFTR and ZO1 staining (right). FIG. 11C shows representative microscopic images of a tissue section from the lower airway of an animal that received high dose CFTR mRNA (1000 μg/kg), visualized for CFTR staining (left), ZO1staining (middle) and optical merge of CFTR and ZO1 staining (right). The intensity of the CFTR expression was higher in the high dose (FIG. 11C, left image) compared to low dose (FIG. 11B, left image). The merged image in FIG. 11C was yellow indicating that the proteins colocalized, whereas FIG. 11A had no signal for CFTR (left) as was expected of the control.

### Example 11. CFTR mRNA Derived Protein Expression in Alveolar Lung

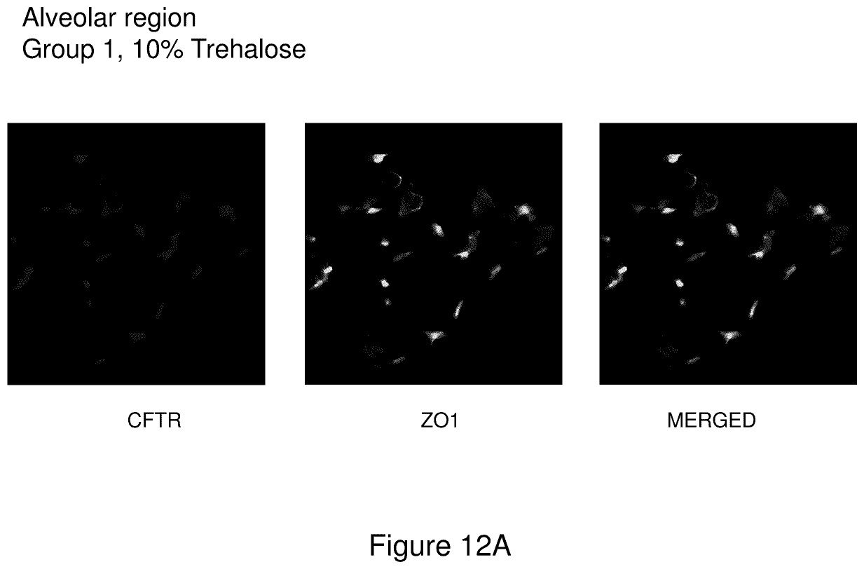

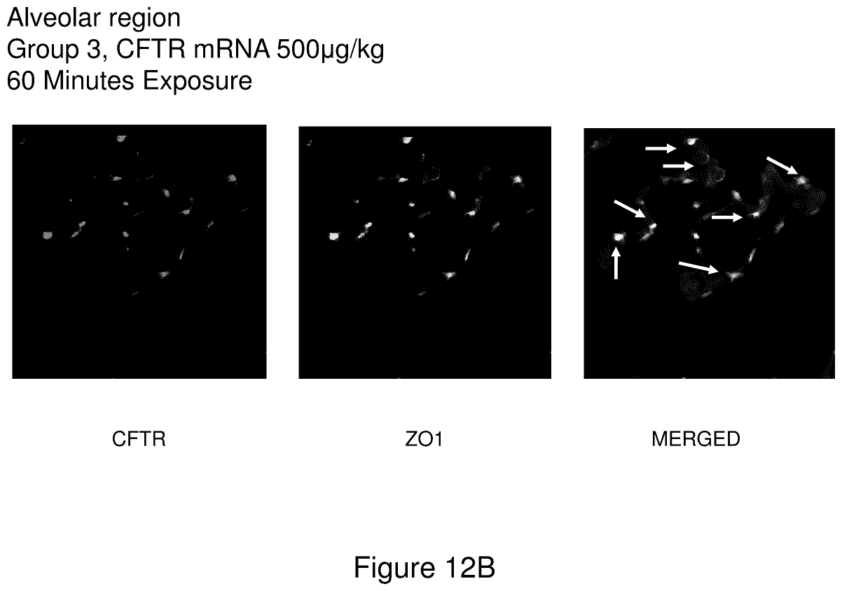

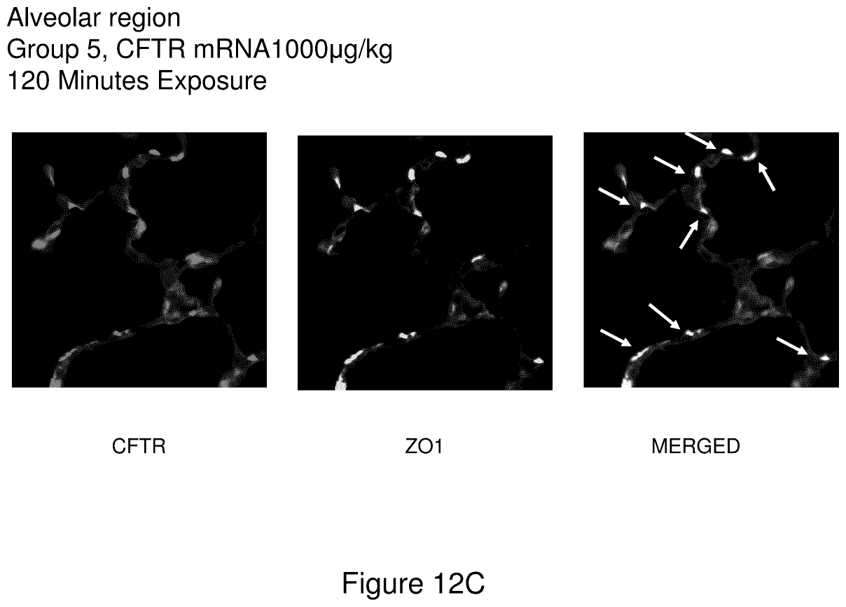

CFTR mRNA was successfully taken up by alveolar cells in the deep lung. As shown in FIGS. 12A-C, CFTR protein translated from administered CFTR mRNA was expressed in alveolar cell membranes and colocalized with endogenous membrane tight junction protein ZO1 protein in the alveolar cell membrane. FIG. 12A shows representative microscopic images of a tissue section from the lower airway epithelial cell membrane from a control animal that received 10% trehalose alone, visualized for CFTR staining (left), ZO1staining (middle) and optical merge of CFTR and ZO1 staining (right). FIG. 12B shows representative microscopic images of a tissue section from the lower airway epithelial cell membrane of an animal that received low dose CFTR mRNA (500 μg/kg), visualized for CFTR staining (left), ZO1 staining (middle) and optical merge of CFTR and ZO1 staining (right). FIG. 12C shows representative microscopic images of a tissue section from the lower airway of an animal that received high dose CFTR mRNA (1000 μg/kg), visualized for CFTR staining (left), ZO1 staining (middle) and optical merge of CFTR and ZO1 staining (right). The intensity of the CFTR expression was higher in the high dose (FIG. 12C, left image) compared to low dose (FIG. 12B, left image). The merged image in FIG. 12C was yellow indicating that the proteins colocalized, whereas FIG. 12A had no signal for CFTR (left) as was expected for the control.

### Example 12. Particle Size Characterization with Simultaneous Emitted Dose and Output Rate Determination Study (VMT nebulizer 1)

The studies described in Examples 12-15 evaluated nebulized particles of a CFTR mRNA/liposome composition as described in Example 1. The composition was nebulized using three devices of VMT nebulizer 1, a commercially available VMT nebulizer, and the resulting aerosol was examined in triplicate to determine the following parameters:
Volume median diameter (VMD)
Fine particle fraction (FPF); specifically, the percentage of the aerosol that has a particle size
   <5.0 μm
   <3.3 μm
   <2.0 μm
Span

Simultaneous gravimetric measurements (8 mL fill) were made using continuous 15 L/min extraction. Each nebulizer was run in triplicate to determine:
Delivery efficiency (emitted mass over whole treatment as a percentage of the fill mass [%])
Emitted mass over the first minute of nebulization (gravimetric weight loss [mg/min])
Emitted mass rate over the entire treatment (total output rate [mg dosing solution/min]).Total emitted mass (delivered dose over the entire treatment [mg])
Residual drug solution (mg and uL equivalent of dosing solution)
Treatment time (time to end of aerosol generation [s])

The results demonstrate that the CFTR mRNA/liposome composition described in Example 1 can be delivered from VMT nebulizer 1 with a mean volume median diameter (VMD) of 4.35 μm (n=9, standard deviation 0.46 μm) and a mean treatment time of 38 minutes 49 seconds (standard deviation 12 minutes 35 seconds) for an 8-mL fill. The specific gravity of the composition was determined to be 1.03 g/mL. A data summary is provided in Table 8.

As the VMD of the generated particles is <5 μm, the majority of the nebulized composition is capable of penetrating into the lower airways of a patient and therefore reaching a large proportion of the relevant population of epithelial cells. At a CO-hCFTR mRNA concentration of 0.6 mg/mL, a single 8 mL fill can theoretically administer a total dose of 4.2 mg in less than 40 minutes. The treatment time is therefore short enough to be feasibly administered the CO-hCFTR mRNA liposome composition of the invention in an outpatient setting or at home with a commercially available vibrating mesh nebulizer.

TABLE 8
Results Summary of Particle Size Characterization
with Simultaneous Emitted Dose
and Output Rate Determination (VMT nebulizer 1)

| | | Mean | SD | % RSD |
|---|---|---|---|---|
| VMD (μm) | | 4.35 | 0.46 | 10.49 |
| Fine Particle | <5.0 μm | 59.55% | 6.47% | 10.86 |
| Fraction | <3.3 μm | 32.56% | 6.76% | 20.78 |
| | <2.0 μm | 9.92% | 4.44% | 44.73% |

| | | | |
|---|---|---|---|
| Span | 1.51 | 0.11 | 7.07 |
| Delivery efficiency | 98.3% | 0.31% | 0.32 |
| Emitted mass over first | 218.5 | 88.65 | 40.58 |
| minute of nebulization | | | |
| (mg solution/min) | | | |
| Emitted mass rate, | 232.8 | 80.21 | 34.45 |
| entire treatment | | | |
| (mg solution/min) | | | |
| Total emitted mass | 8165.5 | 28.88 | 0.35 |
| (mg solution) | | | |
| Residual mass | 139.2 | 26.04 | 18.71 |
| (mg solution) | | | |
| Residual volume (mL) | 135.30 | 25.32 | 18.71 |
| Treatment time (s) | 2328.67 | 754.64 | 32.41 |

Abbreviations: mg = milligram(s); μm = micrometer(s); min = minute(s); mL = milliliter(s); s = second(s); RSD = relative standard deviation; SD = standard deviation; VMD = volume median diameter

### Example 13. Aerodynamic Particle Size Characterization Study by Next Generation Impactor (NGI) at 15 L/min (VMT Nebulizer 1)

Aerosol from each of three devices of VMT nebulizer 1 as described in Example 12 was examined in triplicate using a Next Generation Impactor (NGI) cooled to 5° C. with 15 L/min extraction to determine the following parameters:
Mass median aerodynamic diameter (MMAD)—the average aerosol particle size
Fine particle fraction (FPF); specifically, the percentage of the aerosol <5.0 μm
Delivered dose to the impactor (quantity of the composition collected in the impactor)
Geometric standard deviation (GSD)-a dimensionless number indicating variability in aerosol particle size
Mass balance

1 mL of the composition was nebulized for each analysis. A summary of the results is provided in Table 9.

The average particle size of the nebulized composition ranges from approximately 4.35 μm to approximately 5.58 μm when the VMD and MMAD are measured, respectively. These two methods of measuring the median particle size of the composition are therefore in broad agreement.

TABLE 9
MMAD, GSD, FPF, Mass Balance and
Delivered Dose Results (VMT nebulizer 1)

| | Mean | SD | % RSD |
|---|---|---|---|
| MMAD (μm) | 5.58 | 0.36 | 6.52 |
| GSD | 1.72 | 0.05 | 2.90 |
| FPF (% <5.0 μm) | 40.94 | 4.82 | 11.78 |
| Mass Balance (%) | 100.46 | 6.20 | 6.17 |
| Delivered Dose to | 542.72 | 38.38 | 7.07 |
| Impactor (μg)* | | | |

*Total amount of drug delivered to the NGI test apparatus (connector, throat, and NGI cups)
Abbreviations: FPF = fine particle fraction; GSD = geometric standard deviation; μg = microgram(s); μm = micrometer(s); MMAD = mass median aerodynamic diameter; RSD = relative standard deviation; SD = standard deviation

### Example 14. Study of Delivered Dose into Adult and Child Breathing Patterns (VMT Nebulizer 1)

To characterize the aerosol output for the CFTR mRNA/liposome composition described in Example 1 during breathing, three devices of VMT nebulizer 1 were each examined in triplicate using two breathing patterns. The breathing patterns utilized were:
Adult
   500 mL tidal volume
   inhalation/exhalation ratio (I/E)=1:1
   15 breaths per minute.
Child
   155 mL tidal volume
   inhalation/exhalation ratio (I/E)=1:2
   25 breaths per minute

1 mL of the composition was nebulized for each analysis. Using the fine particle fraction (FPF, %<5.0 μm) results determined via NGI testing, a number of parameters were calculated. Table 10 summarizes treatment time and nebulizer residual. Table 11 summarizes delivered dose, respirable delivered dose and rates. Table 12 summarizes emitted doses and rates. Table 13 summarizes respirable emitted doses and rates. Table 14 and Table 15 summarize efficiency as a percent of nominal dose.

These studies demonstrate that about 25%-40% of the total nebulized composition (39.6% and 26.3% for adult and child, respectively) will be delivered to the patient upon nebulization. This loss has to be taken into account when dosing patients with the CFTR mRNA/liposome composition.

TABLE 10
Treatment Time and Nebulizer Residual: Nebulization of 1 mL of
Composition into Two Breathing Patterns (VMT nebulizer 1)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Treatment Time (sec) | | 201.1 | 45.8 | 22.8 | 210.6 | 49.9 | 23.7 |
| Nebulizer | (μg CFTR | 124.6 | 23.8 | 19.1 | 187.0 | 44.0 | 23.5 |
| residual - | mRNA) | | | | | | |
| RiboGreen | (μL solution | 207.6 | 39.6 | 19.1 | 311.6 | 73.3 | 23.5 |
| assay | equivalent) | | | | | | |

| | | | | | | |
|---|---|---|---|---|---|---|
| Nebulizer residual- | 146.0 | 31.6 | 21.7 | 162.7 | 35.5 | 21.8 |
| gravimetric (mg solution) | | | | | | |

Abbreviations:
mg = milligram(s);
μg = microgram(s);
μL = microliter(s);
RSD = relative standard deviation;
SD = standard deviation

TABLE 11
Delivered Dose, Respirable Delivered Dose and Rates: Nebulization of
1 mL of Composition into Two Breathing Patterns (VMT nebulizer 1)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Delivered | (μg CFTR | 237.9 | 17.9 | 7.5 | 157.8 | 14.7 | 9.3 |
| Dose | mRNA) | | | | | | |
| | (μL solution | 396.6 | 29.8 | 7.5 | 262.9 | 24.5 | 9.3 |
| | equivalent) | | | | | | |
| Delivered | (μg CFTR | 74.2 | 16.9 | 22.7 | 46.4 | 7.7 | 16.5 |
| Dose Rate | mRNA/min) | | | | | | |
| | (μL solution | 123.7 | 28.1 | 22.7 | 77.4 | 12.8 | 16.5 |
| | equivalent/ | | | | | | |
| Respirable | (μg CFTR | 97.2 | 12.3 | 12.6 | 65.0 | 12.2 | 18.8 |
| Delivered | mRNA) | | | | | | |
| Dose (as per | (μL solution | 162.1 | 20.4 | 12.6 | 108.3 | 20.4 | 18.8 |
| FPF (% <5.0 | equivalent) | | | | | | |
| μm) by NGI | | | | | | | |
| at 15 L/min) | | | | | | | |
| Respirable | (μg CFTR | 29.8 | 4.8 | 16.0 | 18.8 | 2.5 | 13.2 |
| Delivered | mRNA/min) | | | | | | |
| Dose Rate (as | (μL solution | 49.7 | 7.9 | 16.0 | 31.3 | 4.1 | 13.2 |
| per FPF (% | equivalent/ | | | | | | |
| <5.0 μm) by | min) | | | | | | |
| NGI at 15 | | | | | | | |
| L/min) | | | | | | | |

Abbreviations:
FPF = fine particle fraction;
μg = microgram(s);
μL = microliter(s);
μm = micrometer(s);
L = liter(s);
min = minute(s);
NGI = Next Generation Impactor;
RSD = relative standard deviation;
sec = second(s);
SD = standard deviation

TABLE 12
Emitted Doses and Rates: Nebulization of 1 mL of the Composition
into Two Breathing Patterns (VMT nebulizer 1)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Emitted | (μg CFTR | 476.3 | 26.2 | 5.5 | 412.1 | 46.5 | 11.3 |
| Dose - | mRNA) | | | | | | |
| RiboGreen | (μL solution | 793.9 | 43.7 | 5.5 | 686.8 | 77.5 | 11.3 |
| Assay | equivalent) | | | | | | |
| Emitted | (mg | 855.4 | 36.8 | 4.3 | 835.7 | 39.2 | 4.7 |
| Dose - | solution) | | | | | | |
| gravimetric | | | | | | | |
| Emitted | (μg CFTR | 146.8 | 23.8 | 16.2 | 120.7 | 16.7 | 13.9 |
| Dose Rate - | mRNA/min) | | | | | | |
| RiboGreen | (μL | 244.7 | 39.7 | 16.2 | 201.1 | 27.9 | 13.9 |
| Assay | solution | | | | | | |
| | equivalent | | | | | | |
| Emitted | (mg | 264.2 | 44.8 | 17.0 | 248.4 | 51.6 | 20.8 |
| Dose Rate - | solution/min) | | | | | | |
| gravimetric | | | | | | | |

Abbreviations:
mg = milligram(s);
μg = microgram(s);
μL = microliter(s);
min = minute(s);
RSD = relative standard deviation;
SD = standarddeviation

TABLE 13
Respirable Emitted Doses and Rates: Nebulization of 1 mL of the
Composition into Two Breathing Patterns (VMT nebulizer 1)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Respirable | (μg CFTR | 196.0 | 33.8 | 17.2 | 170.5 | 38.6 | 22.6 |
| Emitted | mRNA) | | | | | | |
| Dose (as | (μL solution | 326.7 | 56.4 | 17.2 | 284.1 | 64.3 | 22.6 |
| per FPF | equivalent) | | | | | | |
| (% <5.0 μm) | | | | | | | |
| by NGI at | | | | | | | |
| 15 L/min) - | | | | | | | |
| RiboGreen | | | | | | | |
| Assay | | | | | | | |
| Respirable | (mg | 351.7 | 56.2 | 16.0 | 342.8 | 49.9 | 14.6 |
| Emitted | solution) | | | | | | |
| Dose - | | | | | | | |
| gravimetric | | | | | | | |
| Respirable | (μg CFTR | 59.3 | 6.4 | 10.7 | 49.1 | 6.4 | 13.1 |
| Emitted | mRNA/ | | | | | | |
| Dose Rate | min) | | | | | | |
| (as per FPF | (μL solution | 98.9 | 10.6 | 10.7 | 81.8 | 10.7 | 13.1 |
| by NGI at | equivalent/ | | | | | | |
| 15 L/min) - | min) | | | | | | |
| RiboGreen | | | | | | | |
| Assay | | | | | | | |
| Respirable | (mg | 106.6 | 11.7 | 11.0 | 100.2 | 16.1 | 16.1 |
| Emitted | solution/min) | | | | | | |
| Dose Rate - | | | | | | | |
| gravimetric | | | | | | | |

Abbreviations:
FPF = fine particle fraction;
μg = microgram(s);
μL = microliter(s);
μm = micrometer(s);
L = liter(s);
min = minute(s);
NGI = Next Generation Impactor;
RSD = relative standard deviation;
SD = standard deviation

TABLE 14
Efficiency as a Percentage of Nominal Dose: Nebulization of 1 mL of
the Composition into Two Breathing Patterns (VMT nebulizer 1)

| | Adult Breathing | | | Child Breathing | | |
|---|---|---|---|---|---|---|
| | Pattern | | | Pattern | | |
| | Mean | SD | % RSD | Mean | SD | % RSD |
| Emitted Dose (%) | 79.3 | 4.0 | 5.0 | 68.8 | 7.5 | 10.9 |
| Respirable Emitted Dose | 32.6 | 5.5 | 16.8 | 28.4 | 6.4 | 22.4 |
| (as per FPF (% <5.0 μm) | | | | | | |
| by NGI at 15 L/min) (%) | | | | | | |
| Delivered Dose (%) | 39.6 | 3.1 | 7.9 | 26.3 | 2.3 | 8.9 |
| Respirable Delivered Dose | 16.2 | 2.0 | 12.4 | 10.8 | 2.0 | 18.6 |
| (as per FPF (% <5.0 μm) | | | | | | |
| by NGI at 15 L/min) (%) | | | | | | |

Abbreviations:
FPF = fine particle fraction;
μm = micrometer(s);
L = liter(s);
min = minute(s);
NGI = Next Generation Impactor;
RSD = relative standard deviation;
SD = standard deviation

TABLE 15
Efficiency as a Percentage of Nominal Dose (Gravimetric): Nebulization of
1 mL of the Composition into Two Breathing Patterns (VMT nebulizer 1)

| | Adult Breathing | | | Child Breathing | | |
|---|---|---|---|---|---|---|
| | Pattern | | | Pattern | | |
| | Mean | SD | % RSD | Mean | SD | % RSD |
| Emitted Dose (%) | 85.4 | 3.2 | 3.7 | 83.7 | 3.6 | 4.3 |
| Respirable Emitted Dose | 35.1 | 5.4 | 15.5 | 34.3 | 4.9 | 14.4 |
| (as per FPF (% <5.0 μm) | | | | | | |
| by NGI at 15 L/min) (%) | | | | | | |

Abbreviations:
FPF = fine particle fraction;
μm = micrometer(s);
L = liter(s);
min = minute(s);
NGI = Next Generation Impactor;
RSD = relative standard deviation;
SD = standard deviation

### Example 15. Study of Post-Nebulization mRNA Integrity of the Composition (VMT Nebulizer 1)

To determine the mRNA integrity in the nebulized CFTR mRNA/liposome composition produced in during nebulization, three devices of VMT nebulizer 1 were used to each nebulize 8.0 mL of the composition. The composition was nebulized into a cold trap to produce three samples. Three control samples were also generated as follows:
Control 1: the composition as thawed.
Control 2: the composition held within the nebulizer drug chamber for the mean duration of time required for nebulization of 8 mL.
Control 3: the composition held within the cold trap glassware for the mean duration of time required for nebulization of 8 mL

8 mL of the composition was nebulized for each analysis. Samples were stored frozen at not more than −70° C. until mRNA integrity analysis was performed. The composition was deformulated and the resulting CFTR mRNA solutions were analyzed by capillary electrophoresis. The area percent falling within the range of 4,000 to 6,000 nt was quantified to provide an assessment of mRNA integrity. The following observations were made:
For all three aforementioned controls, the mRNA integrity ranged from 53.9% to 71.3%
For all nebulized the composition samples, the mRNA integrity ranged from 61.2% to 70.2%.

These data demonstrate that mRNA integrity of the CFTR mRNBA/liposome composition is not affected by nebulization, thereby supporting the use of a vibrating mesh nebulizer to administer the composition.

### Example 16. Particle Size Characterization with Simultaneous Emitted Dose and Output Rate Determination Study (VMT Nebulizer 2)

The studies described in Examples 16-19 evaluated nebulized particles of a CFTR mRNA/liposome composition as described in Example 1. The composition was nebulized using three devices of VMT nebulizer 2, a commercially available VMT nebulizer, and the resulting aerosol was examined in triplicate to determine the following parameters:
Volume median diameter (VMD)
Fine particle fraction (FPF); specifically, the percentage of the aerosol that has a particle size
   <5.0 μm
   <3.3 μm
   <2.0 μm
Span

Simultaneous gravimetric measurements (6 mL fill) were made using continuous 15

L/min extraction. Each nebulizer was run in triplicate to determine:
Delivery efficiency (emitted mass over whole treatment as a percentage of the fill mass [%])
Emitted mass over the first minute of nebulization (gravimetric weight loss [mg/min])
Emitted mass rate over the entire treatment (total output rate [mg dosing solution/min]).Total emitted mass (delivered dose over the entire treatment [mg])
Residual drug solution (mg and μL equivalent of dosing solution)
Treatment time (time to end of aerosol generation [s])

A data summary is provided in Table 16 below.

The results demonstrate that the composition can be delivered from VMT nebulizer 2 with a mean volume median diameter (VMD) of 4.55 μm (n=9, standard deviation 0.56 μm) and a treatment time of 22 minutes 4 seconds (standard deviation 2 minutes 48 seconds) for a 6-mL fill.

As the VMD of the generated particles is <5 μm, the majority of the composition is capable of penetrating into the lower airways of a patient and therefore reaching a large proportion of the relevant population of epithelial cells. At a CO-hCFTR mRNA concentration of 0.6 mg/mL, a single 6 mL fill can theoretically administer a total dose of 3.6 mg in less than 25 minutes. The treatment time is therefore short enough to be feasibly administered the CO-hCFTR mRNA liposome composition of the invention in an outpatient setting or at home with a commercially available vibrating mesh nebulizer.

TABLE 16
Results Summary of Particle Size Characterization
with Simultaneous Emitted Dose
and Output Rate Determination (VMT nebulizer 2)

| | | Mean | SD | % RSD |
|---|---|---|---|---|
| VMD (μm) | | 4.55 | 0.56 | 12.24 |
| Fine Particle | <5.0 μm | 57.48% | 9.68% | 16.84 |
| Fraction | <3.3 μm | 30.61% | 6.68% | 21.81 |
| | <2.0 μm | 8.62% | 1.87% | 21.73% |

| | | | |
|---|---|---|---|
| Span | 1.49 | 0.13 | 8.43 |
| Delivery efficiency | 96.48% | 0.61% | 0.63 |
| Emitted mass over first | 271.0 | 42.74 | 15.77 |
| minute of nebulization | | | |
| (mg solution/min) | | | |
| Emitted mass rate, | 276.6 | 35.21 | 12.73 |
| entire treatment | | | |
| (mg solution/min) | | | |
| Total emitted mass | 6016.0 | 49.88 | 0.83 |
| (mg solution) | | | |
| Residual mass | 219.2 | 37.72 | 17.21 |
| (mg solution) | | | |
| Residual volume (mL) | 213.12 | 36.67 | 17.21 |
| Treatment time (s) | 1324.11 | 168.18 | 12.70 |

Abbreviations: mg = milligram(s); μm = micrometer(s); min = minute(s); mL = milliliter(s); s = second(s); RSD = relative standard deviation; SD = standard deviation; VMD = volume median diameter

### Example 17. Aerodynamic Particle Size Characterization Study by Next Generation Impactor (NGI) at 15 L/min (VMT Nebulizer 2)

Aerosol from each of three devices of VMT nebulizer 2 as described in Example 16 was examined in triplicate using a Next Generation Impactor (NGI) cooled to 5° C. with 15 L/min extraction to determine the following parameters:
Mass median aerodynamic diameter (MMAD)—the average aerosol particle size
Fine particle fraction (FPF); specifically, the percentage of the aerosol <5.0 μm
Delivered dose to the impactor (quantity of the composition collected in the impactor)
Geometric standard deviation (GSD)-a dimensionless number indicating variability in aerosol particle size
Mass balance

1 mL of the composition was nebulized for each analysis. A summary of the results is provided in Table 17 below.

TABLE 17
MMAD, GSD, FPF, Mass Balance and
Delivered Dose Results (VMT nebulizer 2)

| | Mean | SD | % RSD |
|---|---|---|---|
| MMAD (μm) | 5.76 | 0.24 | 4.18 |
| GSD | 1.86 | 0.06 | 3.02 |
| FPF (% <5.0 μm) | 39.35 | 2.80 | 7.11 |
| Mass Balance (%) | 98.62 | 6.34 | 6.43 |
| Delivered Dose to | 466.86 | 53.50 | 11.46 |
| Impactor (μg)* | | | |

*Total amount of drug delivered to the NGI test apparatus (connector, throat, and NGI cups)
Abbreviations: FPF = fine particle fraction; GSD = geometric standard deviation; μg = microgram(s); μm = micrometer(s); MMAD = mass median aerodynamic diameter; RSD = relative standard deviation; SD = standard deviation

### Example 18. Study of Delivered Dose into Adult and Child Breathing Patterns (VMT Nebulizer 2)

To characterize the aerosol output for the CFTR mRNA/liposome composition described in Example 1 during breathing, three devices of VMT nebulizer 2 were each examined in triplicate using two breathing patterns to characterize aerosol output of the composition into the specified breathing patterns. The breathing patterns utilized were:
Adult
   500 mL tidal volume
   inhalation/exhalation ratio (I/E)=1:1
   15 breaths per minute
Child
   155 mL tidal volume
   inhalation/exhalation ratio (I/E)=1:2
   25 breaths per minute

1 mL of the composition was nebulized for each analysis. Using the fine particle fraction (FPF, %<5.0 μm) results determined via NGI testing, a number of parameters were calculated.

Table 18 summarizes treatment time and nebulizer residual.

Table 19 summarizes delivered dose, respirable delivered dose and rates. Table 20 summarizes emitted doses and rates.

Table 21 summarizes respirable emitted doses and rates.

Table 22 and Table 23 summarize efficiency as a percent of nominal dose.

These studies demonstrate that about 25%-42% of the total nebulized composition will be delivered to the patient upon nebulization (41.5% and 25.0% for adult and child, respectively). This loss has to be taken into account when dosing patients with the CFTR mRNA/liposome composition.

TABLE 18
Treatment Time and Nebulizer Residual: Nebulization of 1 mL of the
Composition into Two Breathing Patterns (VMT nebulizer 2)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Treatment Time (sec) | | 241.1 | 30.3 | 12.6 | 256.7 | 62.7 | 24.4 |
| Nebulizer | (μg CFTR | 91.7 | 45.4 | 49.5 | 94.0 | 36.6 | 38.9 |
| residual - | mRNA) | | | | | | |
| RiboGreen | (μL solution | 152.9 | 75.7 | 49.5 | 156.6 | 60.9 | 38.9 |
| assay | equivalent) | | | | | | |

| | | | | | | |
|---|---|---|---|---|---|---|
| Nebulizer residual - | 97.4 | 49.2 | 50.5 | 99.0 | 48.0 | 48.5 |
| gravimetric (mg solution) | | | | | | |

Abbreviations:
mg = milligram(s);
μg = microgram(s);
μL = microliter(s);
RSD = relative standard deviation;
SD = standard deviation

TABLE 19
Delivered Dose, Respirable Delivered Dose and Rates: Nebulization of 1
mL of the Composition into Two Breathing Patterns (VMT nebulizer 2)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Delivered | (μg CFTR | 249.9 | 48.7 | 19.5 | 148.5 | 31.5 | 21.2 |
| Dose | mRNA) | | | | | | |
| | (μL solution | 416.4 | 81.2 | 19.5 | 247.6 | 52.5 | 21.2 |
| | equivalent) | | | | | | |
| Delivered | (μg CFTR | 62.4 | 10.0 | 16.0 | 35.6 | 8.0 | 22.4 |
| Dose Rate | mRNA/min) | | | | | | |
| | (μL solution | 103.9 | 16.6 | 16.0 | 59.3 | 13.3 | 22.4 |
| | equivalent/ | | | | | | |
| | min) | | | | | | |
| Respirable | (μg CFTR | 98.9 | 24.5 | 24.7 | 58.8 | 15.3 | 26.0 |
| Delivered | mRNA) | | | | | | |
| Dose (as | (μL solution | 164.9 | 40.8 | 24.7 | 98.0 | 25.5 | 26.0 |
| per FPF (% | equivalent) | | | | | | |
| <5.0 μm) by | | | | | | | |
| NGI at 15 | | | | | | | |
| L/min) | | | | | | | |
| Respirable | (μg CFTR | 24.5 | 4.4 | 17.8 | 13.9 | 2.5 | 18.2 |
| Delivered | mRNA/min) | | | | | | |
| Dose Rate | (μL solution | 40.9 | 7.3 | 17.8 | 23.1 | 4.2 | 18.2 |
| (as per FPF | equivalent/ | | | | | | |
| μm) by | min) | | | | | | |
| NGI at 15 | | | | | | | |
| L/min) | | | | | | | |

Abbreviations:
FPF = fine particle fraction;
μg = microgram(s);
μL = microliter(s);
μm = micrometer(s);
L = liter(s);
min = minute(s);
NGI = Next Generation Impactor;
RSD = relative standard deviation;
sec = second(s);
SD = standard deviation

TABLE 20
Emitted Doses and Rates: Nebulization of 1 mL of the Composition
into Two Breathing Patterns (VMT nebulizer 2)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Emitted | (μg CFTR | 509.6 | 45.7 | 9.0 | 499.8 | 38.4 | 7.7 |
| Dose - | mRNA) | | | | | | |
| RiboGreen | (μL solution | 849.3 | 76.2 | 9.0 | 833.0 | 63.9 | 7.7 |
| Assay | equivalent) | | | | | | |
| Emitted | (mg | 904.7 | 51.5 | 5.7 | 890.6 | 52.2 | 5.9 |
| Dose - | solution) | | | | | | |
| gravimetric | | | | | | | |
| Emitted | (μg CFTR | 128.1 | 15.5 | 12.1 | 121.7 | 25.1 | 20.6 |
| Dose Rate - | mRNA/min) | | | | | | |
| RiboGreen | (μL | 213.5 | 25.8 | 12.1 | 202.8 | 41.9 | 20.6 |
| Assay | solution | | | | | | |
| | equivalent | | | | | | |
| Emitted | (mg | 227.5 | 23.7 | 10.4 | 217.2 | 44.6 | 20.6 |
| Dose Rate - | solution/min) | | | | | | |
| gravimetric | | | | | | | |

Abbreviations:
mg = milligram(s);
μg = microgram(s);
μL = microliter(s);
min = minute(s);
RSD = relativestandard deviation;
SD = standard deviation

TABLE 21
Respirable Emitted Doses and Rates: Nebulization of 1 mL of the
Composition into Two Breathing Patterns (VMT nebulizer 2)

| | | Adult Breathing | | | Child Breathing | | |
|---|---|---|---|---|---|---|---|
| | | Pattern | | | Pattern | | |
| | | Mean | SD | % RSD | Mean | SD | % RSD |
| Respirable | (μg CFTR | 201.1 | 29.0 | 14.4 | 197.3 | 26.6 | 13.5 |
| Emitted | mRNA) | | | | | | |
| Dose (as | (μL solution | 335.2 | 48.3 | 14.4 | 328.8 | 44.4 | 13.5 |
| per FPF (% | equivalent) | | | | | | |
| <5.0 μm) | | | | | | | |
| by NGI at | | | | | | | |
| 15 L/min) - | | | | | | | |
| RiboGreen | | | | | | | |
| Assay | | | | | | | |
| Respirable | (mg | 356.7 | 41.3 | 11.6 | 351.2 | 41.1 | 11.7 |
| Emitted | solution) | | | | | | |
| Dose - | | | | | | | |
| gravimetric | | | | | | | |
| Respirable | (μg CFTR | 50.3 | 6.0 | 11.9 | 47.4 | 7.7 | 16.2 |
| Emitted | mRNA/min) | | | | | | |
| Dose Rate | (μL solution | 83.8 | 10.0 | 11.9 | 79.0 | 12.8 | 16.2 |
| (as per FPF | equivalent/ | | | | | | |
| μm) by | min) | | | | | | |
| NGI at 15 | | | | | | | |
| L/min) - | | | | | | | |
| RiboGreen | | | | | | | |
| Assay | | | | | | | |
| Respirable | (mg | 89.2 | 8.2 | 9.2 | 84.5 | 13.3 | 15.7 |
| Emitted Dose | solution/min) | | | | | | |
| Rate - | | | | | | | |
| gravimetric | | | | | | | |

Abbreviations:
FPF = fine particle fraction;
mg = milligram(s);
μg = microgram(s);
μL = microliter(s);
μm = micrometer(s);
L = liter(s);
min = minute(s);
NGI = Next Generation Impactor;
RSD = relative standard deviation;
SD = standard deviation

TABLE 22
Efficiency as a Percentage of Nominal Dose: Nebulization of 1 mL
of the Composition into Two Breathing Patterns (VMT nebulizer 2)

| | Adult Breathing | | | Child Breathing | | |
|---|---|---|---|---|---|---|
| | Pattern | | | Pattern | | |
| | Mean | SD | % RSD | Mean | SD | % RSD |
| Emitted Dose (%) | 84.7 | 7.5 | 8.9 | 84.2 | 6.2 | 7.4 |
| Respirable Emitted Dose | 33.5 | 4.8 | 14.3 | 33.2 | 4.4 | 13.3 |
| (as per (% <5.0 μm) by | | | | | | |
| NGI at 15 L/min) (%) | | | | | | |
| Delivered Dose (%) | 41.5 | 7.9 | 19.1 | 25.0 | 5.2 | 20.7 |
| Respirable Delivered Dose | 16.4 | 4.0 | 24.4 | 9.9 | 2.5 | 25.6 |
| (as per FPF (% <5.0 μm) | | | | | | |
| by NGI at 15 L/min) (%) | | | | | | |

Abbreviations:
FPF = fine particle fraction;
μm = micrometer(s);
L = liter(s);
min = minute(s);
NGI = Next Generation Impactor;
RSD = relative standard deviation;
SD = standard deviation

TABLE 23
Efficiency as a Percentage of Nominal Dose (Gravimetric): Nebulization
of 1 mL of the Composition into Two Breathing Patterns (VMT nebulizer
2)

| | Adult Breathing | | | Child Breathing | | |
|---|---|---|---|---|---|---|
| | Pattern | | | Pattern | | |
| | Mean | SD | % RSD | Mean | SD | % RSD |
| Emitted Dose (%) | 90.3 | 4.9 | 5.5 | 90.0 | 4.9 | 5.4 |
| Respirable Emitted Dose | 35.6 | 4.1 | 11.5 | 35.5 | 4.1 | 11.5 |
| (as per FPF (% <5.0 μm) | | | | | | |
| by NGI at 15 L/min) (%) | | | | | | |

Abbreviations:
FPF = fine particle fraction;
μm = micrometer(s);
L = liter(s);
min = minute(s);
NGI = Next Generation Impactor;
RSD = relative standard deviation;
SD = standard deviation

### Example 19. Study of Post-Nebulization mRNA Integrity of the Composition (VMT Nebulizer 2)

To determine the mRNA integrity in the nebulized CFTR mRNA/liposome composition produced during nebulization, three devices of VMT nebulizer 2 were used to each nebulize 6.0 mL of the composition for post-nebulization mRNA integrity analysis. The composition was nebulized into a cold trap to produce three samples. Three control samples were also generated as follows:
Control 1: the composition as thawed.
Control 2: the composition held within the nebulizer drug chamber for the mean duration of time required for nebulization of 6 mL
Control 3: the composition held within the cold trap glassware for the mean duration of time required for nebulization of 6 mL

6 mL of the composition was nebulized for each analysis. Samples were stored frozen at NMT−70° C. until mRNA integrity analysis was performed. The composition was deformulated and the resulting CFTR mRNA solutions were analyzed by capillary electrophoresis. The area percent falling within the range of 4,000 to 6,000 nt was quantified to provide an assessment of mRNA integrity. The following observations were made:
For all three aforementioned controls, the mRNA integrity ranged from 49.7% to 72.7%.
For all nebulized samples of the composition, the mRNA integrity ranged from 60.8% to 67.3%.

These data demonstrate that mRNA integrity of the CFTR mRNA/liposome composition is not affected by nebulization, thereby supporting the use of a vibrating mesh nebulizer to administer the composition.

The data presented in Examples 16-19 is similar to that presented in Examples 12-15, demonstrating that similar results are achieved when the CFTR mRNA/liposome composition is nebulized using two different commercially available vibrating mesh nebulizers (VMT nebulizer 1 and VMT nebulizer 2).

### Example 20. Studies relating to diluents in formulations

In order to study the benefits of different diluents for the LNP formulations described herein, the LNP formulations of Table 24 were prepared.

TABLE 24
mRNA-containing LNPs

| | | Formulation factors | | | |
|---|---|---|---|---|---|
| | | | Lipid | | |
| | | | molar | | |
| Formulation | LNP | Component lipids | ratio | Cargo | Lyoprotectant |
| Ex. 8 | LNP1 | DLinKC2-DMA:DOPE: | 50:25:20:5 | FFL mRNA | 10% |
| | | CHOL:DMG-PEG2000 | | (mRNA1) | sucrose |
| Ex. 9 | LNP1 | DLinKC2-DMA:DOPE: | 50:25:20:5 | EPO mRNA | 10% |
| | | CHOL:DMG-PEG2000 | | (mRNA2) | sucrose |
| Ex. 10 | LNP2 | DLinKC2-DMA:DOPE: | 50:25:20:5 | EPO mRNA | 10% |
| | | CHOL:DMG-PEG2000 | | (mRNA2) | sucrose |
| A | LNP3 | ICE:DOPE:DMG- | 60:35:5 | CFTR mRNA | 10% |
| | | PEG2000 | | (mRNA3) | sucrose |
| B | LNP3 | ICE:DOPE:DMG- | 60:35:5 | CFTR mRNA | 10% |
| | | PEG2000 | | (mRNA3) | trehalose |
| C | LNP3 | ICE:DOPE:DMG- | 60:35:5 | CFTR mRNA | 10% |
| | | PEG2000 | | (mRNA3) + | trehalose |
| | | | | histidine | |
| D | LNP3 | ICE:DOPE:DMG- | 60:35:5 | CFTR mRNA | 10% |
| | | PEG2000 | | (mRNA3) | glucose |
| E | LNP4 | ML2:DOPE:CHOL: | 40:30:20:10 | AS SI mRNA | 10% |
| | | DMG-PEG2000 | | (mRNA4) | glucose |

Formulations Ex. 8, Ex. 9, and Ex. 10 respectively correspond to the formulations of Examples 8-10 in International Publication No. WO/2012/170889, which is hereby incorporated by reference. Formulations A-E were prepared according to the protocol of Table 25.

TABLE 25
Protocol for the preparation of Formulations A-E

| | | | Ramp | | | Total | |
|---|---|---|---|---|---|---|---|
| | | Setpoint | rate | Time | Time | time | Pressure |
| Phase | Type | (° C.) | (° C./min) | (min) | (h) | (h) | (mTorr) |
| Loading | | 5 | 0 | 0 | 0 | 0 | atm |
| Freeze | Hold | 5 | 0 | 60 | 1 | 1 | atm |
| Freeze | Rate | −5 | 0.3 | 33 | 0.6 | 1.6 | atm |
| Freeze | Hold | −5 | 0 | 30 | 0.5 | 2.1 | atm |
| Freeze | Rate | −25 | 0.3 | 67 | 1.1 | 3.2 | atm |
| Freeze | Hold | −25 | 0 | 30 | 0.5 | 3.7 | atm |
| Freeze | Rate | −50 | 0.3 | 83 | 1.4 | 5.1 | atm |
| Freeze | Hold | −50 | 0 | 180 | 3 | 8.1 | atm |
| Freeze | Rate | −32 | 0.3 | 60 | 1 | 9.1 | atm |
| Freeze | Hold | −32 | 0 | 60 | 1 | 10.1 | atm |
| Primary | Hold | −32 | 0 | 3600 | 60 | 70.1 | 50 |
| Drying | | | | | | | |
| Secondary | Rate | 4 | 0.05 | 720 | 12 | 82.1 | 50 |
| Drying | | | | | | | |
| Secondary | Hold | 4 | 0 | 360 | 6 | 88.1 | 50 |
| Drying | | | | | | | |
| Storage | Hold | 4 | 0 | NA | NA | NA | 1000 |

Physical Properties of the mRNA-LNPs

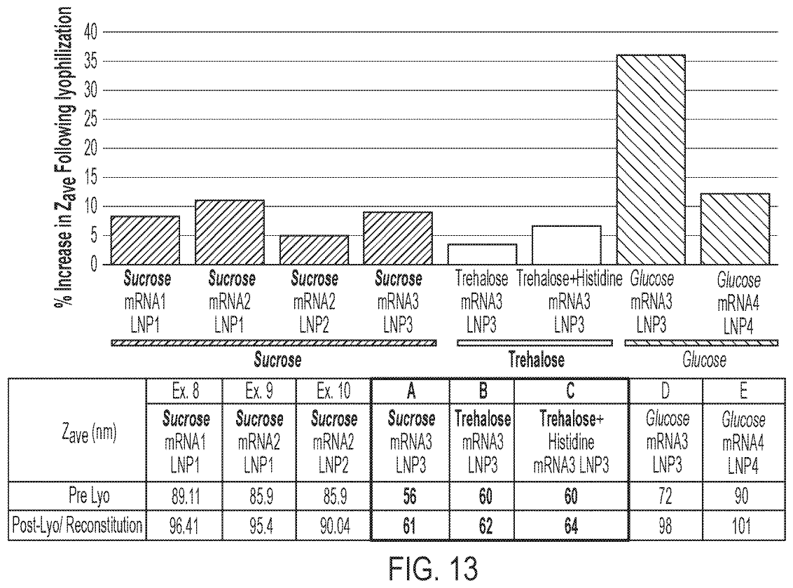

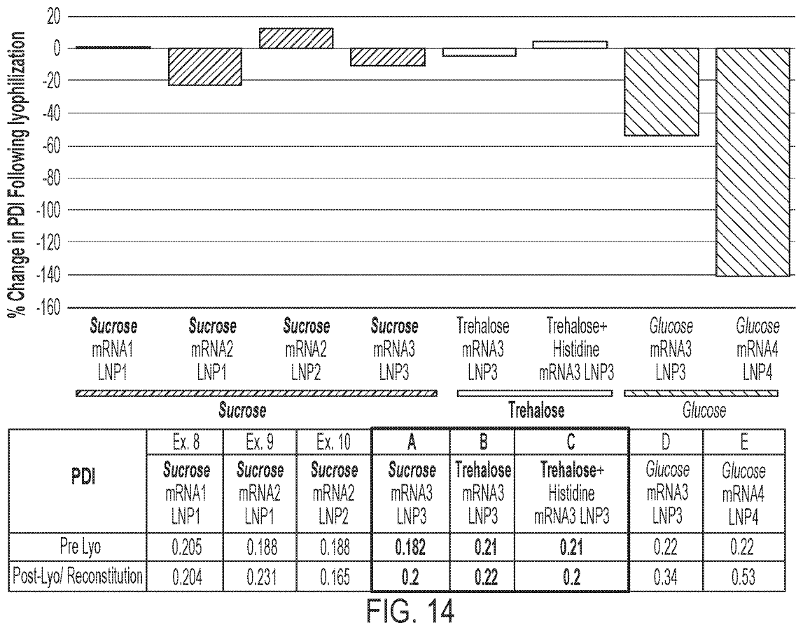

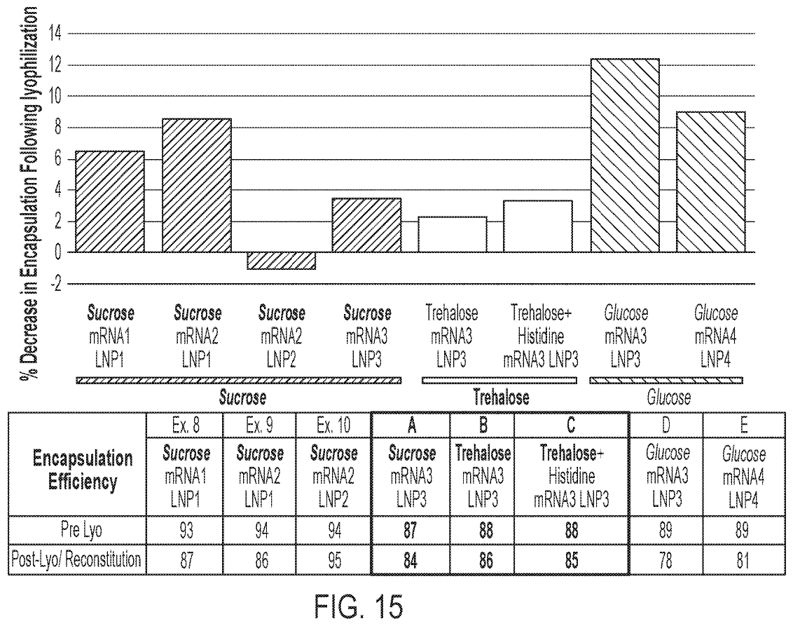

The physical properties of the fresh (unlyophilized) liposomes and the lyophilized mRNA-LNP formulations were compared in accordance with the methods provided in Example 6 of International Publication No. WO/2012/170889. Stability was assessed by comparing the average size of the particles (Zₐᵥₑ), their size homogeneity (indicated by the polydispersity index, PDI) and mRNA encapsulation levels before and after lyophilization. The data from these tests are provided in FIGS. 13-15.

Each of FIGS. 13-15 show that there were minimal changes in the physical properties of the mRNA-LNPs following their lyophilization and reconstitution when using sucrose or trehalose as a lyoprotectant. For example, the use of sucrose or trehalose as a lyoprotectant for formulations A and B (both of which comprise LNP3 and mRNA 3) resulted in an 8.9% and 3.3% increase in size respectively following lyophilization and reconstitution.

Comparing the data for formulation A (sucrose as a lyoprotectant, lyophilization protocol set out in Table 25 above) with that of Ex. 8, Ex. 9, and Ex. 10 (sucrose as a lyoprotectant, lyophilization protocol set out in Table 3 of International Publication No. WO/2012/170889) shows that similarly stable lyophilized mRNA-LNP compositions can be produced using both lyophilization protocols.

In Vivo and In Vitro Potency of the Lyophilized mRNA-LNPs

Wild type CD-1 mice were used to evaluate in vivo potency of an mRNA-LNP formulation comprising trehalose as a lyoprotectant, both before lyophilization and following lyophilization and reconstitution. The expression of the ASS1 protein from ASSI mRNA (mRNA 4) in an LNP comprising ML2: DOPE: CHOL: DMG-PEG2000 (LNP4) was measured following a single IV administration of the formulation.

In a second experiment, cells lacking normal CFTR expression in a Ussing chamber assay were used to evaluate the potency of a CFTR-mRNA (mRNA 3) encapsulated in an LNP comprising ICE: DOPE: DMG-PEG2000 (LNP 3) in a formulation comprising trehalose, both before lyophilization and following lyophilization and reconstitution.

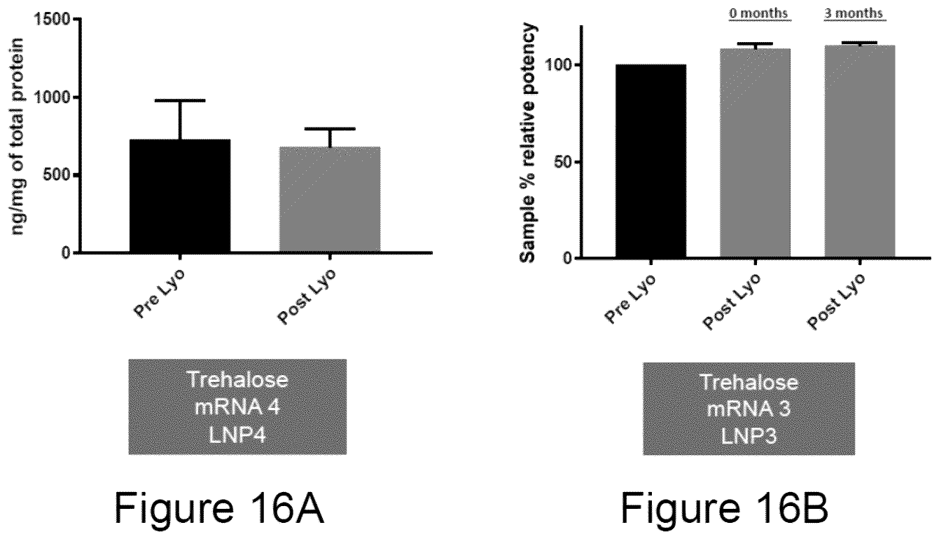

FIGS. 16A and 16B, which show the respective results of these two studies, indicate that such mRNA-LNP compositions can be successfully delivered to target cells and expressed following lyophilization and reconstitution. In particular, FIG. 16B shows that the activity of the CFTR mRNA-LNP formulation following lyophilization and reconstitution remained substantially unchanged as compared to pre-lyophilization, even after a storage period of three months at household freezer temperatures (−20° C.). The data therefore show that trehalose can be particularly suitable as a lyoprotectant for maintaining the in vivo potency of mRNA-LNP formulations during storage.

### Example 21: Safety and Tolerability of a Single Dose of Inhaled CFTR mRNA Therapeutic in Adult CF Patients

Not all people with CF respond to CFTR modulator therapy that is available or in development. A clinical study was conducted in which a biosynthetic mRNA coding for CFTR encapsulated in lipid nanoparticles was delivered by aerosol to adult CF patients. This form of treatment has the potential to be a mutation agnostic therapeutic regimen.

One objective of this study was to evaluate the safety and tolerability of a single dose of inhaled lipid-encapsulated CFTR mRNA or placebo in 12 adult CF patients followed for at least 1 month post-dose. To this end, adult CF patients with class I and/or II mutations and baseline FEV1 values between 50 and 90% predicted were randomized 3:1 to receive a single dose of 8, 16 or 24 mg lipid-encapsulated CFTR mRNA or placebo in a double-blinded study. All doses were administered via a hand-held nebulizer in a clinic setting and patients were followed for at least 1 month after the dose before unblinding and analysis. The concentration of the CO-hCFTR mRNA was 0.6 mg/ml. A nebulizer was used to administer the CO-hCFTR composition by nebulization at a nebulization rate of approximately 0.3 mL/minute.

Analysis of the patients showed that of the 12 enrolled patients, 8 were F508del homozygotes, 3 were F508del heterozygotes and I did not have an F508del mutation. Of the 8 F508del homozygotes, 7 were prescribed concomitant CFTR modulator therapy (5 ivacaftor/lumacaftor, 2 tezacaftor/lumacaftor). One subject had a genotype that is not eligible for any of the currently approved modulators or triple combination therapies in development. Mean (SD) baseline FEV1 was 66.3 (14.1) % predicted. Nebulization ranged from approximately 45 minutes to 2 hours and 25 minutes in duration, depending on dose, and was immediately well tolerated. The 8, 16 and 24 mg dose groups have completed dosing. All dose escalations were approved by the Safety Review Committee and implemented as planned. The study assessments included collection of adverse events (AEs), chest radiographs, spirometry and clinical laboratory findings, which will be reported after unblinding.

In summary, twelve subjects have received a single dose of lipid-encapsulated mRNA or placebo in the first-in-human study of nebulized mRNA therapeutic in CF. Detailed, unblinded safety results, as well as FEV1 data, is forthcoming. In parallel, dosing in the multiple dose arm of the study (5 weekly nebulizations) is ongoing.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method of treating cystic fibrosis (CF) in a human subject comprising administering a composition comprising an mRNA encoding a Cystic Fibrosis Transmembrane Conductance Regulator (CFTR) protein at a concentration of 0.5 mg/ml or greater to the human subject, wherein at least 80% of the mRNA encoding the CFTR protein is encapsulated within a nanoparticle, wherein the nanoparticle comprises one or more cationic lipids, one or more non-cationic lipids, and one or more PEG-modified lipids, wherein the step of administering comprises the following: (i) the composition is aerosolized using a nebulizer, and (ii) a nominal dose of the mRNA is administered to the human subject via the nebulizer over a period of time at least 30 minutes at a nebulization rate of at least 0.2 mL/minute.

2. The method of claim 1, wherein the concentration of the mRNA encoding the CFTR protein ranges from 0.5 mg/mL to 0.8 mg/mL, optionally wherein the concentration is 0.6 mg/mL.

3. The method of claim 1, wherein at least 25%, at least 35%, or at least 40% of the nominal dose is delivered to the lungs of the human subject.

4. The method of claim 1, wherein the period over which the aerosolized composition is administered exceeds 35 min, 45 min, 1 hour, 2 hours, or 3 hours.

5. The method of claim 4, wherein the period over which the aerosolized composition is administered is between 35 min to 4.5 hours, e.g., between 1.5 hours to 2 hours or between 2.5 hours to 4 hours.

6. The method of claim 1, wherein the nominal dose exceeds 5 mg.

7. The method of claim 6, wherein the nominal dose is between 4 mg and 40 mg, optionally wherein the nominal dose is between 6 mg and 30 mg, e.g., 8 mg, 16 mg or 24 mg.

8. The method of claim 1, wherein the nebulization rate ranges from 0.2 mL/minute to 0.5 mL/minute.

9. The method of claim 1, wherein the volume of the composition that is aerosolized in a single treatment session ranges from 13.0 mL to 42.0 mL.

10. The method of claim 9, wherein volume of the composition that is aerosolized does not exceed 20 mL in a single treatment.

11. The method of claim 1, wherein the median droplet size in the aerosolized composition is between 4 and 6 μm.

12-13. (canceled)

14. The method of claim 1, wherein the nanoparticle is a liposome and the liposome has a size of less than about 100 nm.

15-26. (canceled)

27. The method of claim 1, wherein the mRNA encoding the CFTR protein comprises a polynucleotide sequence at least 85%, at least 90%, at least 95%, or at least 99% identical to SEQ ID NO: 1.

28. The method of claim 27, wherein the mRNA encoding the CFTR protein comprises a polynucleotide sequence identical to SEQ ID NO: 1.

29-30. (canceled)

31. The method of claim 1, wherein the mRNA encoding the CFTR protein comprises a polynucleotide sequence at least 85%, at least 90% or at least 91% or at least 92% or at least 93% or at least 94% or at least 95% or at least 96% or at least 97% or at least 98% or at least 99% or 100% identical to any one of SEQ ID NO: 6 to SEQ ID NO: 27.

32. The method of claim 31, wherein the mRNA encoding the CFTR protein comprises a polynucleotide sequence identical to SEQ ID NO: 6.

33. (canceled)

34. The method of claim 1, wherein the composition comprises trehalose.

35. The method of claim 34, wherein the trehalose concentration is 10% (w/v).

36-43. (canceled)

44. The method of claim 1, wherein the human subject has a forced expiratory volume in one second (FEV1) of between about 50% and 90% of predicted normal.

45. The method of claim 44, wherein the human subject has an FEV1 of between about 60% and 70% predicted of predicted normal.
